(19) United States
(12) Patent Application Publication
(10) Pub. No.: US 2022/0177530 A1
(43) Pub. Date: Jun. 9, 2022

(54) RECOMBINANT SILK BASED PRODUCTS AND METHODS OF PREPARING THE SAME

(71) Applicant: EVOLVED BY NATURE, INC., Medford, MA (US)

(72) Inventor: Gregory H. Altman, Providence, RI (US)

(73) Assignee: EVOLVED BY NATURE, INC., Medford, MA (US)

(21) Appl. No.: 17/598,717

(22) PCT Filed: Mar. 27, 2020
(86) PCT No.: PCT/US20/25414

§ 371 Date: Sep. 27, 2021

Related U.S. Application Data
(60) Provisional application No. 62/824,601, filed on Mar. 27, 2019.

(51) Int. Cl. C07K 14/435; A61L 27/22; A61L 27/26; A61Q 19/00; A61K 8/64

(52) U.S. Cl. CPC C07K 14/43586; C07K 14/43518; A61K 8/64; A61L 27/26; A61Q 19/007; A61L 27/227

(57) ABSTRACT

Recombinant silk coated and/or infused materials and articles, and methods of preparing the same are disclosed herein. In some embodiments, articles include textiles, fabrics, consumer products, leather, and other materials that are coated with recombinant silk-based protein fragments, such as recombinant spider or silkworm silk-based protein fragments, having low, medium, and/or high molecular weight in various ratios. In some embodiments, articles and materials include dermal fillers and cosmetic materials such as moisturizers that include recombinant silk-based protein fragments, such as recombinant spider or silkworm silk-based protein fragments, having low, medium, and/or high molecular weight in various ratios.

### CROSS-REFERENCE TO RELATED APPLICATIONS

This application is an International Patent Application which claims the benefit of U.S. Provisional Patent Application No. 62/824,601, filed Mar. 27, 2019, which is hereby incorporated by reference in its entirety.

### FIELD OF THE INVENTION

The disclosure provides silk-coated and silk-embedding products for use in home and automotive applications, such as fabrics or leather coated with recombinant silk proteins or protein fragments thereof, personal care products and medical materials. In some embodiments, the disclosure provides cosmetic compositions comprising recombinant silk proteins or protein fragments thereof. In some embodiments, the disclosure provides medical materials, articles, and dermal fillers comprising recombinant silk proteins or protein fragments thereof.

### BACKGROUND OF THE INVENTION

Silk is a natural polymer produced by a variety of insects and spiders. Silk fibers are lightweight, breathable, and hypoallergenic. Silk is comfortable when worn next to the skin and insulates very well; keeping the wearer warm in cold temperatures and is cooler than many other fabrics in warm temperatures.

Spider's silk polypeptides are large (>150 kDa, >1000 amino acids) polypeptides that can be broken down into three domains: an N-terminal non-repetitive domain (NTD), the repeat domain (REP), and the C-terminal non-repetitive domain (CTD). The NTD and CTD are relatively small (˜150, ˜100 amino acids respectively), well studied, and are believed to confer to the polypeptide aqueous stability, pH sensitivity, and molecular alignment upon aggregation. NTD also has a strongly predicted secretion tag, which is often removed during heterologous expression. The repetitive region composes ˜90% of the natural polypeptide, and folds into the crystalline and amorphous regions that confer strength and flexibility to the silk fiber, respectively.

Some organisms make multiple silk fibers with unique sequences, structural elements, and mechanical properties. For example, orb-weaving spiders have six unique types of glands that produce different silk polypeptide sequences that are polymerized into fibers tailored to fit an environmental or lifecycle niche. The fibers are named for the gland they originate from and the polypeptides are labeled with the gland abbreviation (e.g. “Ma”) and “Sp” for spidroin (short for spider fibroin). In orb weavers, these types include Major Ampullate (MaSp, also called dragline), Minor Ampullate (MiSp), Flagelliform (Flag), Aciniform (AcSp), Tubuliform (TuSp), and Pyriform (PySp). This combination of polypeptide sequences across fiber types, domains, and variation amongst different genus and species of organisms leads to a vast array of potential properties that can be harnessed by commercial production of the recombinant fibers. To date, the vast majority of the work with recombinant silks has focused on the Major Ampullate Spidroins (MaSp).

There is a need in the field for products, such as threads, fibers, cloth, and other textiles, that may be coated or combined and/or embedded with recombinant silks.

### SUMMARY OF THE INVENTION

Recombinant silk performance materials and applications, such as apparel, and methods of preparing the same are disclosed herein. According to aspects illustrated herein, the present disclosure relates to a product, including, but not limited to, apparel, padding, shoes, gloves, luggage, furs, jewelry and bags, configured to be worn or carried on the body, that is at least partially surface treated with a solution of recombinant silk-based protein fragments of the present disclosure so as to result in a silk coating on the product. In some embodiments, the solutions of recombinant silk-based proteins or fragments thereof may be aqueous solutions, organic solutions, or emulsions. In an embodiment, the product is manufactured from a textile material. In an embodiment, the product is manufactured from a non-textile material. In an embodiment, desired additives can be added to an aqueous solution of recombinant silk-based protein fragments of the present disclosure so as to result in a silk coating having desired additives.

In some embodiments, the disclosure provides a method of making a recombinant silk coated material, comprising: preparing a recombinant silk solution comprising low molecular weight recombinant silk-based protein fragments; coating a surface of the material with the recombinant silk solution; and drying the surface of the material that has been coated with the recombinant silk solution to provide the recombinant silk coated material. In some embodiments, the recombinant silk solution comprises recombinant spider silk-based proteins or fragments thereof. In some embodiments, the low molecular weight recombinant silk-based protein fragments comprise recombinant spider silk-based proteins or fragments thereof. In some embodiments, the recombinant silk solution further comprises medium molecular weight recombinant silk-based protein fragments. In some embodiments, the medium molecular weight recombinant silk-based protein fragments comprise recombinant spider silk-based proteins or fragments thereof. In some embodiments, recombinant silk is as provided in any of the compositions #1001-2450, #3001-4450, and #5001-6595. In some embodiments, the w/w ratio between low molecular weight recombinant silk-based protein fragments and medium molecular weight recombinant silk-based protein fragments is about 90:10, about 80:20, about 75:25, about 70:30, about 66:34, about 60:40, about 50:50, about 40:60, about 34:66, about 30:70, about 25:75, about 20:80, or about 10:90. In some embodiments, the w/w ratio between low molecular weight recombinant silk-based protein fragments and medium molecular weight recombinant silk-based protein fragments is about 3:1. In some embodiments, the recombinant silk solution further comprises high molecular weight recombinant silk-based protein fragments. In some embodiments, the recombinant silk solution further comprises medium molecular weight recombinant silk-based protein fragments, and high molecular weight recombinant silk-based protein fragments. In some embodiments, drying the surface of the material comprises heating the surface of the material without substantially modifying recombinant silk coating performance. In some embodiments, the recombinant silk solution comprises recombinant silk-based protein fragments at less than about 0.001% by volume (v/v). In some embodiments, the recombinant silk solution comprises recombinant silk-based protein fragments at less than about 0.1% by volume (v/v). In some embodiments, the recombinant silk solution comprises recombinant silk-based protein fragments at less than about 1% by volume (v/v). In some embodiments, the recombinant silk solution comprises recombinant silk-based protein fragments at less than about 2.5% by volume (v/v). In some embodiments, the recombinant silk solution comprises recombinant silk-based protein fragments at less than about 5% by volume (v/v). In some embodiments, the step of preparing the recombinant silk solution comprises adding a chemical fabric softener to the solution. In some embodiments, the recombinant silk solution comprises a Brønsted acid. In some embodiments, the recombinant silk solution comprises one or more of citric acid and acetic acid. In some embodiments, the step of coating a surface of the material comprises one or more of a roller application process, a saturation and removal process, and a topical application process. In some embodiments, the step of coating a surface of the material comprises one or more of a bath coating process, a kiss rolling process, spray coating, and a two-sided rolling process. In some embodiments, the step of coating a surface of the material comprises coating one surface of the material. In some embodiments, the step of coating a surface of the material comprises coating two surfaces of the material. In some embodiments, the method further comprises the step of dyeing the surface of the material. In some embodiments, the step of dyeing the surface of the material occurs prior to coating a surface of the material with the recombinant silk solution. In some embodiments, the step of dyeing the surface of the material occurs after coating a surface of the material with the recombinant silk solution. In some embodiments, the material comprises one or more of a woven material, a non-woven material, a knit material, and a crochet material. In some embodiments, the material comprises fabric, thread, yarn, or a combination thereof. In some embodiments, the material comprises one or more of polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, and LYCRA. In some embodiments, the recombinant silk solution further comprises a chemical agent. In some embodiments, the chemical agent is selected from the group consisting of silicone, an antimicrobial agent, an antifungal agent, a softener, a water repellant agent, an oil repellant agent, a dye, a flame retardant, a fabric softener, a pH adjusting agent, an anticrocking agent, an antipilling agent, and an antifelting agent. In some embodiments, the chemical agent is selected to modify one or more of a first property and second property of the recombinant silk coated material. In some embodiments, the first property comprises one or more of an antimicrobial property, an antiodor property, a water repellant property, an oil repellant property, a flame retardant property, a coloring property, a fabric softening property, a stain repellant property, a pH adjusting property, an anticrocking property, an antipilling property, and an antifelting property. In some embodiments, the second property comprises one or more of wetting time, absorption rate, spreading speed, accumulative one-way transport, and overall moisture management capability.

In some embodiments, the disclosure provides an article comprising a fiber or yarn having a coating, wherein the coating comprises recombinant silk-based proteins or fragments thereof having a weight average molecular weight, or average weight average molecular weight range of about 5 kDa to about 144 kDa. In some embodiments, the article is a fabric. In some embodiments, the recombinant silk-based proteins or fragments thereof comprise recombinant spider silk-based proteins or fragments thereof. In some embodiments, the recombinant silk-based proteins or fragments thereof comprise a copolymer. In some embodiments, the recombinant silk-based proteins or protein fragments thereof have an average weight average molecular weight range selected from the group consisting of about 5 to about 10 kDa, about 6 kDa to about 16 kDa, about 17 kDa to about 38 kDa, about 39 kDa to about 80 kDa, about 60 to about 100 kDa, and about 80 kDa to about 144 kDa, wherein the silk based proteins or fragments thereof have a polydispersity of between about 1.0 and about 5.0. In some embodiments, recombinant silk is as provided in any of the compositions #1001-2450, #3001-4450, and #5001-6595. In some embodiments, the fiber or yarn is selected from the group consisting of natural fiber or yarn, synthetic fiber or yarn, or combinations thereof. In some embodiments, the fiber or yarn is natural fiber or yarn selected from the group consisting of cotton, alpaca fleece, alpaca wool, lama fleece, lama wool, cotton, cashmere, sheep fleece, sheep wool, and combinations thereof. In some embodiments, the fiber or yarn is synthetic fiber or yarn selected from the group consisting of polyester, nylon, polyester-polyurethane copolymer, and combinations thereof.

In an embodiment, a method is provided for coating a material with recombinant silk that may include silk-based proteins or fragments thereof (including any of the compositions #1001-2450, #3001-4450, and #5001-6595) to provide a recombinant silk coated material, wherein the recombinant silk coated upon the recombinant silk coated material may be heat resistant to a selected temperature. In some embodiments, the method may include preparing a recombinant silk solution (including, but not limited to, any of the compositions #1001-2450, #3001-4450, and #5001-6595) that may include a concentration of one or more of low molecular weight recombinant silk, medium molecular weight recombinant silk, and high molecular weight recombinant silk at less than about 1% by volume (v/v), or less than about 0.1% by volume (v/v), or less than about 0.01% by volume (v/v), or less than about 0.001% by volume (v/v). In some embodiments, the method may include, coating a surface of the material with the recombinant silk solution, including, but not limited to, any of the compositions #1001-2450, #3001-4450, and #5001-6595. In some embodiments, the method may include drying the surface of the material that has been coated with the recombinant silk solution to provide the recombinant silk coated material, wherein drying the surface of the material comprises heating the surface of the material without substantially decreasing recombinant silk coating performance.

In an embodiment, a method is provided for coating a textile with a recombinant silk solution that may include silk-based proteins or fragments thereof (including, but not limited to, any of the compositions #1001-2450, #3001-4450, and #5001-6595) to provide a recombinant silk coated article, wherein the recombinant silk coated upon the recombinant silk coated article may be heat resistant to a selected temperature. In some embodiments, the method may include preparing the recombinant silk solution with one or more of low molecular weight recombinant silk, medium molecular weight recombinant silk, and high molecular weight recombinant silk. In some embodiments, the method may include acidically adjusting the pH of the recombinant silk solution with an acidic agent. In some embodiments, the method may include coating a surface of the textile with the recombinant silk solution. In some embodiments, the method may include drying the surface of the textile that has been coated with the recombinant silk solution to provide the recombinant silk coated article, wherein drying the surface of the textile comprises heating the surface of the textile without substantially decreasing recombinant silk coating performance.

In some embodiments, a method is provided for manufacturing a recombinant silk coated textile that may include selected fabric properties. In some embodiments, the method may include admixing silk-based proteins or fragments thereof (including, but not limited to, any of the compositions #1001-2450, #3001-4450, and #5001-6595) with one or more chemical agents to provide a coating solution, wherein the one or more chemical agents may be selected to modify one or more of a first selected property and second selected property of the recombinant silk coated textile. In some embodiments, the method may include providing the coating solution to a textile to be coated with one or more of a bath coating process, a kiss rolling process, a spray process, and a two-sided rolling process. In some embodiments, the method may include removing excess coating solution from the recombinant silk coated textile. In some embodiments, the method may include heating the recombinant silk coated textile to modify a third selected property of the recombinant silk coated textile. In some embodiments, the first selected property may include one or more of an antimicrobial property, an antiodor property, a water repellant property, an oil repellant property, a flame retardant property, a coloring property, a fabric softening property, a stain repellant property, a pH adjusting property, an anticrocking property, an antipilling property, and an antifelting property. In some embodiments, the second selected property may include one or more of wetting time, absorption rate, spreading speed, accumulative one-way transport, and overall moisture management capability. In some embodiments, the third selected property may include one or more of fabric hand, fabric stretch, and drapability.

In an embodiment, the recombinant silk coated materials of the disclosure may be coated with one or more of low molecular weight recombinant silk, medium molecular weight recombinant silk, and high molecular weight recombinant silk to provide resulting coated materials having enhanced hydrophobic or hydrophilic properties.

In an embodiment, the recombinant silk coated materials of the disclosure, for example a fiber, a yarn, or a fabric, may be coated with compositions including one or more of low molecular weight silk, medium molecular weight silk, and high molecular weight silk (including, but not limited to, any of the compositions #1001-2450, #3001-4450, and #5001-6595), to provide resulting coated materials having enhanced hydrophobic or hydrophilic properties. In an embodiment, the recombinant silk coated materials of the disclosure, for example a fiber, a yarn, or a fabric, may be coated with compositions including low molecular weight silk and medium molecular weight silk. In an embodiment, the recombinant silk coated materials of the disclosure, for example a fiber, a yarn, or a fabric, may be coated with compositions including low molecular weight silk and high molecular weight silk. In an embodiment, the recombinant silk coated materials of the disclosure, for example a fiber, a yarn, or a fabric, may be coated with compositions including medium molecular weight silk and high molecular weight silk. In an embodiment, the recombinant silk coated materials of the disclosure, for example a fiber, a yarn, or a fabric, may be coated with compositions including low molecular weight silk, medium molecular weight silk, and high molecular weight silk.

In an embodiment, the recombinant silk coated materials of the disclosure, for example a fiber, a yarn, or a fabric, may be coated with compositions including low molecular weight silk and medium molecular weight silk (including, but not limited to, any of the compositions #1001-2450, #3001-4450, and #5001-6595). In some embodiments, the w/w ratio between low molecular weight silk and medium molecular weight silk is between about 99:1 to about 1:99, between about 95:5 to about 5:95, between about 90:10 to about 10:90, between about 75:25 to about 25:75, between about 65:35 to about 35:65, or between about 55:45 to about 45:55. In some embodiments, the w/w ratio between low molecular weight silk and medium molecular weight silk is between about 99:1 to about 55:45, between about 95:5 to about 45:55, between about 90:10 to about 35:65, between about 75:25 to about 15:85, between about 65:35 to about 10:90, or between about 55:45 to about 1:99. In an embodiment, the w/w ratio between low molecular weight silk and medium molecular weight silk is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99. In an embodiment, the w/w ratio between low molecular weight silk and medium molecular weight silk is about 3:1. In an embodiment, the w/w ratio between low molecular weight silk and medium molecular weight silk is about 1:3.

In an embodiment, the recombinant silk coated materials of the disclosure, for example a fiber, a yarn, or a fabric, may be coated with compositions including low molecular weight silk and high molecular weight silk (including, but not limited to, any of the compositions #1001-2450, #3001-4450, and #5001-6595). In some embodiments, the w/w ratio between low molecular weight silk and high molecular weight silk is between about 99:1 to about 1:99, between about 95:5 to about 5:95, between about 90:10 to about 10:90, between about 75:25 to about 25:75, between about 65:35 to about 35:65, or between about 55:45 to about 45:55. In some embodiments, the w/w ratio between low molecular weight silk and high molecular weight silk is between about 99:1 to about 55:45, between about 95:5 to about 45:55, between about 90:10 to about 35:65, between about 75:25 to about 15:85, between about 65:35 to about 10:90, or between about 55:45 to about 1:99. In an embodiment, the w/w ratio between low molecular weight silk and high molecular weight silk is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

In an embodiment, the recombinant silk coated materials of the disclosure, for example a fiber, a yarn, or a fabric, may be coated with compositions including medium molecular weight silk and high molecular weight silk (including, but not limited to, any of the compositions #1001-2450, #3001-4450, and #5001-6595). In some embodiments, the w/w ratio between medium molecular weight silk and high molecular weight silk is between about 99:1 to about 1:99, between about 95:5 to about 5:95, between about 90:10 to about 10:90, between about 75:25 to about 25:75, between about 65:35 to about 35:65, or between about 55:45 to about 45:55. In some embodiments, the w/w ratio between medium molecular weight silk and high molecular weight silk is between about 99:1 to about 55:45, between about 95:5 to about 45:55, between about 90:10 to about 35:65, between about 75:25 to about 15:85, between about 65:35 to about 10:90, or between about 55:45 to about 1:99. In an embodiment, the w/w ratio between medium molecular weight silk and high molecular weight silk is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

In an embodiment, the recombinant silk coated materials of the disclosure, for example a fiber, a yarn, or a fabric, may be coated with compositions including low molecular weight silk, medium molecular weight silk, and high molecular weight silk (including, but not limited to, any of the compositions #1001-2450, #3001-4450, and #5001-6595). In an embodiment, the w/w ratio between low molecular weight silk, medium molecular weight silk, and high molecular weight silk is about 1:1:8, 1:2:7, 1:3:6, 1:4:5, 1:5:4, 1:6:3, 1:7:2, 1:8:1, 2:1:7, 2:2:6, 2:3:5, 2:4:4, 2:5:3, 2:6:2, 2:7:1, 3:1:6, 3:2:5, 3:3:4, 3:4:3, 3:5:2, 3:6:1, 4:1:5, 4:2:4, 4:3:3, 4:4:2, 4:5:1, 5:1:4, 5:2:3, 5:3:2, 5:4:1, 6:1:3, 6:2:2, 6:3:1, 7:1:2, 7:2:1, or 8:1:1. In an embodiment, the w/w ratio between low molecular weight silk, medium molecular weight silk, and high molecular weight silk is about 3:0.1:0.9, 3:0.2:0.8, 3:0.3:0.7, 3:0.4:0.6, 3:0.5:0.5, 3:0.6:0.4, 3:0.7:0.3, 3:0.8:0.2, or 3:0.9:0.1.

In and embodiment, materials coated by recombinant silk coatings described herein may include one or more of textiles, woven materials, non-woven materials, knit materials, crochet materials, and leather materials.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa (including, but not limited to, any of the compositions #1001-2450, #3001-4450, and #5001-6595).

In an embodiment, the disclosure provides an article comprising a fiber or yarn having a coating, wherein the coating comprises recombinant silk based proteins or fragments thereof having a weight average molecular weight range of about 5 kDa to about 144 kDa (including, but not limited to, any of the compositions #1001-2450, #3001-4450, and #5001-6595).

In an embodiment, the disclosure provides an article comprising a fiber or yarn having a coating, wherein the coating comprises recombinant silk based proteins or fragments thereof having a number of amino acid residues of about 1 to 400 residues, or 1 to 300 residues, or 1 to 200 residues, or 1 to 100 residues, or 1 to 50 residues, or 5 to 25 residues, or 10 to 20 residues.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa (including, but not limited to, any of the compositions #1001-2450, #3001-4450, and #5001-6595), and wherein the article is a fabric.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the silk based proteins or fragments thereof comprise recombinant silk-based proteins or protein fragments having about 0.01% (w/w) to about 10% (w/w) added sericin.

In an embodiment, the disclosure provides an article comprising a fiber or yarn having a coating, wherein the coating comprises silk based proteins or fragments thereof having a weight average molecular weight, or average weight average molecular weight range of about 5 kDa to about 144 kDa, wherein the silk based proteins or fragments thereof are selected from the group consisting of natural silk based proteins or fragments thereof, recombinant silk based proteins or fragments thereof, and combinations thereof.

In an embodiment, the disclosure provides an article comprising a fiber or yarn having a coating, wherein the coating comprises silk based proteins or fragments thereof having a weight average molecular weight, or average weight average molecular weight range of about 5 kDa to about 144 kDa, wherein the silk based proteins or fragments thereof are selected from the group consisting of natural silk based proteins or fragments thereof, recombinant silk based proteins or fragments thereof, and combinations thereof, wherein the silk based proteins or fragments thereof are natural silk based proteins or fragments thereof that are selected from the group consisting of spider silk based proteins or fragments thereof, silkworm silk based proteins or fragments thereof, and combinations thereof (including, but not limited to, any of the compositions #1001-2450, #3001-4450, and #5001-6595). In an embodiment, the disclosure provides an article comprising a fiber or yarn having a coating, wherein the coating comprises silk based proteins or fragments thereof having a weight average molecular weight, or average weight average molecular weight range of about 5 kDa to about 144 kDa, wherein the silk based proteins or fragments thereof are selected from the group consisting of natural silk based proteins or fragments thereof, recombinant silk based proteins or fragments thereof, and combinations thereof, wherein the silk based proteins or fragments thereof are natural silk based proteins or fragments thereof that are selected from the group consisting of spider silk based proteins or fragments thereof, silkworm silk based proteins or fragments thereof, and combinations thereof, wherein the natural silk based proteins or fragments are silkworm silk based proteins or fragments thereof, and the silkworm silk based proteins or fragments thereof is Bombyx mori silk based proteins or fragments thereof.

In an embodiment, the disclosure provides an article comprising a fiber or yarn having a coating, wherein the coating comprises silk based proteins or fragments thereof having a weight average molecular weight, or average weight average molecular weight range of about 5 kDa to about 144 kDa, wherein the silk based proteins or fragments comprise silk and a copolymer.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or protein fragments thereof have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the silk based proteins or fragments thereof have a polydispersity of between 1 and about 5.0, or between about 1.5 and about 3.0, and wherein the proteins or protein fragments, prior to coating the fabric, do not spontaneously or gradually gelate and do not visibly change in color or turbidity when in a solution for at least 10 days.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa (including, but not limited to, as provided in any of the compositions #1001-2450, #3001-4450, and #5001-6595), wherein the article is a fabric, wherein the fabric exhibits an improved property, wherein the improved property is an accumulative one-way moisture transport index selected from the group consisting of greater than 40%, greater than 60%, greater than 80%, greater than 100%, greater than 120%, greater than 140%, greater than 160%, and greater than 180%. In an embodiment, the foregoing improved property is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa (including, but not limited to, as provided in any of the compositions #1001-2450, #3001-4450, and #5001-6595), wherein the article is a fabric, wherein the fabric exhibits an improved property, wherein the improved property is an accumulative one way transport capability increase relative to uncoated fabric selected from the group consisting of 1.2 fold, 1.5 fold, 2.0 fold, 3.0 fold, 4.0 fold, 5.0 fold, and 10 fold. In an embodiment, the foregoing improved property is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa (including, but not limited to, as provided in any of the compositions #1001-2450, #3001-4450, and #5001-6595), wherein the article is a fabric, wherein the fabric exhibits an improved property, wherein the improved property is an overall moisture management capability selected from the group consisting of greater than 0.05, greater than 0.10, greater than 0.15, greater than 0.20, greater than 0.25, greater than 0.30, greater than 0.35, greater than 0.40, greater than 0.50, greater than 0.60, greater than 0.70, and greater than 0.80. In an embodiment, the foregoing improved property is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa (including, but not limited to, as provided in any of the compositions #1001-2450, #3001-4450, and #5001-6595), wherein the article is a fabric, and wherein the fabric exhibits substantially no increase in microbial growth after a number of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa (including, but not limited to, as provided in any of the compositions #1001-2450, #3001-4450, and #5001-6595), wherein the article is a fabric, wherein the fabric exhibits substantially no increase in microbial growth after a number of machine washing cycles selected from the group consisting of 5 cycles, 10 cycles, 25 cycles, and 50 cycles, and wherein the microbial growth is microbial growth of a microbe selected from the group consisting of Staphylococcus aureus, Klebsiella pneumoniae, and combinations thereof.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa (including, but not limited to, as provided in any of the compositions #1001-2450, #3001-4450, and #5001-6595), wherein the article is a fabric, wherein the fabric exhibits substantially no increase in microbial growth after a number of machine washing cycles selected from the group consisting of 5 cycles, 10 cycles, 25 cycles, and 50 cycles, wherein the microbial growth is microbial growth of a microbe selected from the group consisting of Staphylococcus aureus, Klebsiella pneumoniae, and combinations thereof, wherein the microbial growth is reduced by a percentage selected from the group consisting of 50%, 100%, 500%, 1000%, 2000%, and 3000%, alternatively 1 log unit, 2 log unit, 3 log unit, 4 log unit or 5 log unit as compared to an uncoated fabric.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa (including, but not limited to, as provided in any of the compositions #1001-2450, #3001-4450, and #5001-6595), wherein the article is a fabric, and wherein the coating is applied to the fabric at the fiber level prior to forming the fabric.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa (including, but not limited to, as provided in any of the compositions #1001-2450, #3001-4450, and #5001-6595), wherein the article is a fabric, and wherein the coating is applied to the fabric at the fabric level.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level, and wherein the fabric is bath coated.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level, and wherein the fabric is spray coated.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level, and wherein the fabric is coated with a stencil.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level, wherein the coating is applied to at least one side of the fabric using a method selected from the group consisting of a bath coating process, a spray coating process, a stencil process, a silk-foam based process, and a roller-based process.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, and wherein the coating has a thickness of about one nanolayer.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, and wherein the coating has a thickness selected from the group consisting of about 5 nm, about 10 nm, about 15 nm, about 20 nm, about 25 nm, about 50 nm, about 100 nm, about 200 nm, about 500 nm, about 1 μm, about 5 μm, about 10 μm, and about 20 μm.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the coating is adsorbed on the fabric.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the coating is attached to the fabric through chemical, enzymatic, thermal, or irradiative cross-linking.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level, and wherein the hand of the coated fabric is improved relative to an uncoated fabric.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level, and wherein the hand of the coated fabric is improved relative to an uncoated fabric, wherein the hand of the coated fabric that is improved is selected from the group consisting of softness, crispness, dryness, silkiness, and combinations thereof.

According to aspects illustrated herein, an aqueous solution of recombinant silk-based protein fragments of the present disclosure is available for application to a product, including, but not limited to, apparel, padding, shoes, gloves, luggage, furs, jewelry and bags, or for directly spraying on the body of a consumer, to impart desired properties to the product. In an embodiment, the product is manufactured from a textile material. In an embodiment, the product is manufactured from a non-textile material. In an embodiment, desired additives can be added to an aqueous solution of recombinant silk-based protein fragments of the present disclosure so as to result in a silk coating having desired additives.

In an embodiment, a textile comprising a silk coating of the present disclosure is sold to a consumer. In an embodiment, a textile of the present disclosure is used in constructing action sportswear apparel. In an embodiment, a textile of the present disclosure is used in constructing fitness apparel. In an embodiment, a textile of the present disclosure is used in constructing performance apparel. In an embodiment, a textile of the present disclosure is used in constructing golf apparel. In an embodiment, a textile of the present disclosure is used in constructing lingerie. In an embodiment, a silk coating of the present disclosure is positioned on the underlining of action sportswear/apparel. In an embodiment, a silk coating of the present disclosure is positioned on the shell, the lining, or the interlining of action sportswear/apparel. In an embodiment, action sportswear/apparel is partially made from a silk coated textile of the present disclosure and partially made from an uncoated textile. In an embodiment, action sportswear/apparel partially made from a silk coated textile and partially made from an uncoated textile combines an uncoated inert synthetic material with a silk coated inert synthetic material. Examples of inert synthetic material include, but are not limited to, polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, LYCRA® (polyester-polyurethane copolymer, also known as SPANDEX® and elastomer), and mixtures thereof. In an embodiment, action sportswear/apparel partially made from a silk coated textile and partially made from an uncoated textile combines an elastomeric material at least partially covered with a silk coating of the present disclosure. In an embodiment, the percentage of silk to elastomeric material can be varied to achieve desired shrink or wrinkle resistant properties and desired moisture content against the skin surface. In an embodiment, a silk coating of the present disclosure is positioned on an internal layer of a shoe (textile or non-textile based). In an embodiment, a silk coating of the present disclosure positioned on an internal layer of a shoe helps maintain optimal feet microenvironment, such as temperature and humidity while reducing any excessive perspiration.

In an embodiment, a recombinant silk coating of the present disclosure is visible. In an embodiment, a silk coating of the present disclosure is transparent. In an embodiment, a recombinant silk coating of the present disclosure positioned on action sportswear/apparel helps control skin temperature of a person wearing the apparel. In an embodiment, a recombinant silk coating of the present disclosure positioned on action sportswear/apparel helps control fluid transfer away from the skin of a person wearing the apparel. In an embodiment, a recombinant silk coating of the present disclosure positioned on action sportswear/apparel has a soft feel against the skin decreasing abrasions from fabric on the skin. In an embodiment, a recombinant silk coating of the present disclosure positioned on a textile has properties that confer at least one of wrinkle resistance, shrinkage resistance, or machine washability to the textile. In an embodiment, a silk coated textile of the present disclosure is 100% machine washable and dry cleanable. In an embodiment, a recombinant silk coated textile of the present disclosure is 100% waterproof. In an embodiment, a recombinant silk coated textile of the present disclosure is wrinkle resistant. In an embodiment, a recombinant silk coated textile of the present disclosure is shrink resistant. In an embodiment, a recombinant silk coated fabric improves the health of the skin. In an embodiment, healthy skin can be determined by visibly seeing an even skin tone. In an embodiment, healthy skin can be determined by visibly seeing a smooth, glowing complexion. In an embodiment, a recombinant silk coated fabric decreases irritation of the skin. In an embodiment, a decrease in irritation of the skin can result in a decrease in skin bumps or sores. In an embodiment, a decrease in irritation of the skin can result in a decrease in scaly or red skin. In an embodiment, a decrease in irritation of the skin can result in a decrease in itchiness or burning. In an embodiment, a recombinant silk coated fabric decreases inflammation of the skin. In an embodiment, a recombinant silk coated textile of the present disclosure has the qualities of being waterproof, breathable, and elastic and possess a number of other qualities which are highly desirable in action sportswear. In an embodiment, a recombinant silk coated textile of the present disclosure manufactured from a recombinant silk fabric of the present disclosure further includes LYCRA® brand spandex fibers (polyester-polyurethane copolymer).

In an embodiment, a textile at least partially coated with an aqueous solution of recombinant silk-based protein fragments of the present disclosure is a breathable fabric. In an embodiment, a textile at least partially coated with an aqueous solution of recombinant silk-based protein fragments of the present disclosure is a water-resistant fabric. In an embodiment, a textile at least partially coated with an aqueous solution of recombinant silk-based protein fragments of the present disclosure is a shrink-resistant fabric. In an embodiment, a textile at least partially coated with an aqueous solution of recombinant silk-based protein fragments of the present disclosure is a machine-washable fabric. In an embodiment, a textile at least partially coated with an aqueous solution of recombinant silk-based protein fragments of the present disclosure is a wrinkle resistant fabric. In an embodiment, textile at least partially coated with an aqueous solution of recombinant silk-based protein fragments of the present disclosure provides moisture and vitamins to the skin.

In an embodiment, the textile at least partially coated with an aqueous solution of recombinant silk-based protein fragments of the present disclosure has an accumulative one-way transport index of greater than 140. In an embodiment, the textile at least partially coated with an aqueous solution of recombinant silk-based protein fragments of the present disclosure has an accumulative one-way transport index of greater than 120. In an embodiment, the textile at least partially coated with an aqueous solution of recombinant silk-based protein fragments of the present disclosure has an accumulative one-way transport index of greater than 100. In an embodiment, the textile at least partially coated with an aqueous solution of recombinant silk-based protein fragments of the present disclosure has an accumulative one-way transport index of greater than 80.

In an embodiment, the textile at least partially coated with an aqueous solution of recombinant silk-based protein fragments of the present disclosure has an overall moisture management capability of greater than 0.4. In an embodiment, the textile at least partially coated with an aqueous solution of recombinant silk-based protein fragments of the present disclosure has an overall moisture management capability of greater than 0.35. In an embodiment, the textile at least partially coated with an aqueous solution of recombinant silk-based protein fragments of the present disclosure has an overall moisture management capability of greater than 0.3. In an embodiment, the textile at least partially coated with an aqueous solution of recombinant silk-based protein fragments of the present disclosure has an overall moisture management capability of greater than 0.25.

In an embodiment, the textile at least partially coated with an aqueous solution of recombinant silk-based protein fragments of the present disclosure has a wetting time of at least 3 seconds. In an embodiment, the textile at least partially coated with an aqueous solution of recombinant silk-based protein fragments of the present disclosure has a wetting time of at least 2.5 seconds. In an embodiment, the textile at least partially coated with an aqueous solution of recombinant silk-based protein fragments of the present disclosure has a wetting time of at least 2 seconds. In an embodiment, the textile at least partially coated with an aqueous solution of recombinant silk-based protein fragments of the present disclosure has a wetting time of at least 1.5 seconds.

In an embodiment, the textile at least partially coated with an aqueous solution of recombinant silk-based protein fragments of the present disclosure has a top absorption time of at least 50 seconds. In an embodiment, the textile at least partially coated with an aqueous solution of recombinant silk-based protein fragments of the present disclosure has a top absorption time of at least 40 seconds. In an embodiment, the textile at least partially coated with an aqueous solution of recombinant silk-based protein fragments of the present disclosure has a top absorption time of at least 30 seconds.

In an embodiment, the textile at least partially coated with an aqueous solution of recombinant silk-based protein fragments of the present disclosure has a bottom absorption time of at least 80 seconds. In an embodiment, the textile at least partially coated with an aqueous solution of recombinant silk-based protein fragments of the present disclosure has a bottom absorption time of at least 70 seconds. In an embodiment, the textile at least partially coated with an aqueous solution of recombinant silk-based protein fragments of the present disclosure has a bottom absorption time of at least 60 seconds. In an embodiment, the textile at least partially coated with an aqueous solution of recombinant silk-based protein fragments of the present disclosure has a bottom absorption time of at least 50 seconds. In an embodiment, the textile at least partially coated with an aqueous solution of recombinant silk-based protein fragments of the present disclosure has a bottom absorption time of at least 40 seconds.

In an embodiment, the textile at least partially coated with an aqueous solution of recombinant silk-based protein fragments of the present disclosure has a spreading speed of at least 1.6 mm/second. In an embodiment, the textile at least partially coated with an aqueous solution of recombinant silk-based protein fragments of the present disclosure has a spreading speed of at least 1.4 mm/second. In an embodiment, the textile at least partially coated with an aqueous solution of recombinant silk-based protein fragments of the present disclosure has a spreading speed of at least 1.2 mm/second. In an embodiment, the textile at least partially coated with an aqueous solution of recombinant silk-based protein fragments of the present disclosure has a spreading speed of at least 1.0 mm/second. In an embodiment, the textile at least partially coated with an aqueous solution of recombinant silk-based protein fragments of the present disclosure has a spreading speed of at least 0.8 mm/second.

In an embodiment, the textile at least partially coated with an aqueous solution of recombinant silk-based protein fragments of the present disclosure shows less than 2000% microbial growth over 24 hours. In an embodiment, the textile at least partially coated with an aqueous solution of recombinant silk-based protein fragments of the present disclosure shows less than 1000% microbial growth over 24 hours. In an embodiment, the textile at least partially coated with an aqueous solution of recombinant silk-based protein fragments of the present disclosure shows less than 500% microbial growth over 24 hours. In an embodiment, the textile at least partially coated with an aqueous solution of recombinant silk-based protein fragments of the present disclosure shows less than 400% microbial growth over 24 hours. In an embodiment, the textile at least partially coated with an aqueous solution of recombinant silk-based protein fragments of the present disclosure shows less than 300% microbial growth over 24 hours. In an embodiment, the textile at least partially coated with an aqueous solution of recombinant silk-based protein fragments of the present disclosure shows less than 200% microbial growth over 24 hours. In some embodiments, as described herein, the reduction in microbial growth may be measured and provided after one or more wash cycles in non-chlorine bleach. In some embodiments, solutions that include recombinant silk-based protein fragments may include an additional chemical agent, as described herein, that may provide antimicrobial (e.g., antifungal and/or antibacterial) properties.

In an embodiment, the textile at least partially coated with an aqueous solution of recombinant silk-based protein fragments of the present disclosure shows less than 2000% bacterial growth over 24 hours. In an embodiment, the textile at least partially coated with an aqueous solution of recombinant silk-based protein fragments of the present disclosure shows less than 1000% bacterial growth over 24 hours. In an embodiment, the textile at least partially coated with an aqueous solution of recombinant silk-based protein fragments of the present disclosure shows less than 500% bacterial growth over 24 hours. In an embodiment, the textile at least partially coated with an aqueous solution of recombinant silk-based protein fragments of the present disclosure shows less than 400% bacterial growth over 24 hours. In an embodiment, the textile at least partially coated with an aqueous solution of recombinant silk-based protein fragments of the present disclosure shows less than 300% bacterial growth over 24 hours. In an embodiment, the textile at least partially coated with an aqueous solution of recombinant silk-based protein fragments of the present disclosure shows less than 200% bacterial growth over 24 hours.

In an embodiment, the textile at least partially coated with an aqueous solution of recombinant silk-based protein fragments of the present disclosure shows less than 2000% fungal growth over 24 hours. In an embodiment, the textile at least partially coated with an aqueous solution of recombinant silk-based protein fragments of the present disclosure shows less than 1000% fungal growth over 24 hours. In an embodiment, the textile at least partially coated with an aqueous solution of recombinant silk-based protein fragments of the present disclosure shows less than 500% fungal growth over 24 hours. In an embodiment, the textile at least partially coated with an aqueous solution of recombinant silk-based protein fragments of the present disclosure shows less than 400% fungal growth over 24 hours. In an embodiment, the textile at least partially coated with an aqueous solution of recombinant silk-based protein fragments of the present disclosure shows less than 300% fungal growth over 24 hours. In an embodiment, the textile at least partially coated with an aqueous solution of recombinant silk-based protein fragments of the present disclosure shows less than 200% fungal growth over 24 hours.

In an embodiment, the textile at least partially coated with an aqueous solution of recombinant silk-based protein fragments of the present disclosure shows less than 2000% growth of Staphylococcus aureus over 24 hours. In an embodiment, the textile at least partially coated with an aqueous solution of recombinant silk-based protein fragments of the present disclosure shows less than 1000% growth of Staphylococcus aureus over 24 hours. In an embodiment, the textile at least partially coated with an aqueous solution of recombinant silk-based protein fragments of the present disclosure shows less than 500% growth of Staphylococcus aureus over 24 hours. In an embodiment, the textile at least partially coated with an aqueous solution of recombinant silk-based protein fragments of the present disclosure shows less than 400% growth of Staphylococcus aureus over 24 hours. In an embodiment, the textile at least partially coated with an aqueous solution of recombinant silk-based protein fragments of the present disclosure shows less than 300% growth of Staphylococcus aureus over 24 hours. In an embodiment, the textile at least partially coated with an aqueous solution of recombinant silk-based protein fragments of the present disclosure shows less than 200% growth of Staphylococcus aureus over 24 hours.

In an embodiment, the textile at least partially coated with an aqueous solution of recombinant silk-based protein fragments of the present disclosure shows less than 2000% growth of Klebsiella pneumoniae over 24 hours. In an embodiment, the textile at least partially coated with an aqueous solution of recombinant silk-based protein fragments of the present disclosure shows less than 1000% growth of Klebsiella pneumoniae over 24 hours. In an embodiment, the textile at least partially coated with an aqueous solution of recombinant silk-based protein fragments of the present disclosure shows less than 500% growth of Klebsiella pneumoniae over 24 hours. In an embodiment, the textile at least partially coated with an aqueous solution of recombinant silk-based protein fragments of the present disclosure shows less than 400% growth of Klebsiella pneumoniae over 24 hours. In an embodiment, the textile at least partially coated with an aqueous solution of recombinant silk-based protein fragments of the present disclosure shows less than 300% growth of Klebsiella pneumoniae over 24 hours. In an embodiment, the textile at least partially coated with an aqueous solution of recombinant silk-based protein fragments of the present disclosure shows less than 200% growth of Klebsiella pneumoniae over 24 hours.

In an embodiment, an aqueous solution of recombinant silk-based protein fragments of the present disclosure is used to coat a textile. In an embodiment, the concentration of recombinant silk in the solution ranges from about 0.001 wt. % to about 20.0 wt. %. In an embodiment, the concentration of recombinant silk in the solution ranges from about 0.01 wt. % to about 15.0 wt. %. In an embodiment, the concentration of recombinant silk in the solution ranges from about 0.5 wt. % to about 10.0 wt. %. In an embodiment, the concentration of recombinant silk in the solution ranges from about 1.0 wt. % to about 5.0 wt. %. In an embodiment, an aqueous solution of recombinant silk-based protein fragments of the present disclosure is applied directly to a fabric. Alternatively, recombinant silk microsphere and any additives may be used for coating a fabric. In an embodiment, additives can be added to an aqueous solution of recombinant silk-based protein fragments of the present disclosure before coating (e.g., alcohols) to further enhance material properties. In an embodiment, a recombinant silk coating of the present disclosure can have a pattern to optimize properties of the recombinant silk on the fabric. In an embodiment, a coating is applied to a fabric under tension and/or lax to vary penetration in to the fabric. In an embodiment, a recombinant silk coating of the present disclosure can be applied at the yarn level, followed by creation of a fabric once the yarn is coated. In an embodiment, an aqueous solution of recombinant silk-based protein fragments of the present disclosure can be spun into fibers to make a recombinant silk fabric and/or recombinant silk fabric blend with other materials known in the apparel industry. In an embodiment, a method for recombinant silk coating a fabric includes immersion of the fabric in any of the aqueous solutions of recombinant silk-based protein fragments of the present disclosure. In an embodiment, a method for recombinant silk coating a fabric includes spraying. In an embodiment, a method for recombinant silk coating a fabric includes chemical vapor deposition. In an embodiment, a method for recombinant silk coating a fabric includes electrochemical coating. In an embodiment, a method for recombinant silk coating a fabric includes knife coating to spread any of the aqueous solutions of recombinant silk-based protein fragments of the present disclosure onto the fabric. The recombinant silk coated fabric may then be air dried, dried under heat/air flow, or cross-linked to the fabric surface. In an embodiment, a drying process includes curing with additives and/or ambient condition.

According to aspects illustrated herein, methods for preparing aqueous solutions of recombinant silk-based protein fragments are disclosed. In an embodiment, at least one recombinant silk-based protein fragment (RSPF) mixture solution having a specific average weight average molecular weight (MW) range and polydispersity is created (including, but not limited to, as provided in any of the compositions #1001-2450, #3001-4450, and #5001-6595). In an embodiment, at least a RSPF mixture composition (e.g., a solution) having a MW range between about 6 kDa and 17 kDa and a polydispersity range between 1 and about 5.0, or between about 1.5 and about 3.0 is created and used as described herein. In an embodiment, at least one RSPF mixture composition (e.g., a solution) having a MW between about 17 kDa and 39 kDa and a polydispersity range between 1 and about 5.0, or between about 1.5 and about 3.0 is created and used as described herein. In an embodiment, at least one RSPF mixture composition (e.g., a solution) having a MW range between about 39 kDa and 80 kDa and a polydispersity range between 1 and about 5.0, or between about 1.5 and about 3.0 is created and used as described herein.

In an embodiment, at least one RSPF mixture composition (e.g., a solution) having a MW range between higher than 0 kDa and about 5 kDa and a polydispersity range between 1 and about 5.0, or between about 1.5 and about 3.0 is created and used as described herein. In an embodiment, at least one RSPF mixture composition (e.g., a solution) having a MW range between about 5 kDa and about 10 kDa and a polydispersity range between 1 and about 5.0, or between about 1.5 and about 3.0 is created and used as described herein. In an embodiment, at least one RSPF mixture composition (e.g., a solution) having a MW range between about 10 kDa and about 15 kDa and a polydispersity range between 1 and about 5.0, or between about 1.5 and about 3.0 is created and used as described herein. In an embodiment, at least one RSPF mixture composition (e.g., a solution) having a MW range between about 15 kDa and about 20 kDa and a polydispersity range between 1 and about 5.0, or between about 1.5 and about 3.0 is created and used as described herein. In an embodiment, at least one RSPF mixture composition (e.g., a solution) having a MW range between about 20 kDa and about 25 kDa and a polydispersity range between 1 and about 5.0, or between about 1.5 and about 3.0 is created and used as described herein. In an embodiment, at least one RSPF mixture composition (e.g., a solution) having a MW range between about 25 kDa and about 30 kDa and a polydispersity range between 1 and about 5.0, or between about 1.5 and about 3.0 is created and used as described herein. In an embodiment, at least one RSPF mixture composition (e.g., a solution) having a MW range between about 30 kDa and about 35 kDa and a polydispersity range between 1 and about 5.0, or between about 1.5 and about 3.0 is created and used as described herein. In an embodiment, at least one RSPF mixture composition (e.g., a solution) having a MW range between about 35 kDa and about 40 kDa and a polydispersity range between 1 and about 5.0, or between about 1.5 and about 3.0 is created and used as described herein. In an embodiment, at least one RSPF mixture composition (e.g., a solution) having a MW range between about 40 kDa and about 45 kDa and a polydispersity range between 1 and about 5.0, or between about 1.5 and about 3.0 is created and used as described herein. In an embodiment, at least one RSPF mixture composition (e.g., a solution) having a MW range between about 45 kDa and about 50 kDa and a polydispersity range between 1 and about 5.0, or between about 1.5 and about 3.0 is created and used as described herein. In an embodiment, at least one RSPF mixture composition (e.g., a solution) having a MW range between about 50 kDa and about 55 kDa and a polydispersity range between 1 and about 5.0, or between about 1.5 and about 3.0 is created and used as described herein. In an embodiment, at least one RSPF mixture composition (e.g., a solution) having a MW range between about 55 kDa and about 60 kDa and a polydispersity range between 1 and about 5.0, or between about 1.5 and about 3.0 is created and used as described herein. In an embodiment, at least one RSPF mixture composition (e.g., a solution) having a MW range between about 60 kDa and about 65 kDa and a polydispersity range between 1 and about 5.0, or between about 1.5 and about 3.0 is created and used as described herein. In an embodiment, at least one RSPF mixture composition (e.g., a solution) having a MW range between about 65 kDa and about 70 kDa and a polydispersity range between 1 and about 5.0, or between about 1.5 and about 3.0 is created and used as described herein. In an embodiment, at least one RSPF mixture composition (e.g., a solution) having a MW range between about 70 kDa and about 75 kDa and a polydispersity range between 1 and about 5.0, or between about 1.5 and about 3.0 is created and used as described herein. In an embodiment, at least one RSPF mixture composition (e.g., a solution) having a MW range between about 75 kDa and about 80 kDa and a polydispersity range between 1 and about 5.0, or between about 1.5 and about 3.0 is created and used as described herein. In an embodiment, at least one RSPF mixture composition (e.g., a solution) having a MW range between about 80 kDa and about 85 kDa and a polydispersity range between 1 and about 5.0, or between about 1.5 and about 3.0 is created and used as described herein. In an embodiment, at least one RSPF mixture composition (e.g., a solution) having a MW range between about 85 kDa and about 90 kDa and a polydispersity range between 1 and about 5.0, or between about 1.5 and about 3.0 is created and used as described herein. In an embodiment, at least one RSPF mixture composition (e.g., a solution) having a MW range between about 90 kDa and about 95 kDa and a polydispersity range between 1 and about 5.0, or between about 1.5 and about 3.0 is created and used as described herein. In an embodiment, at least one RSPF mixture composition (e.g., a solution) having a MW range between about 95 kDa and about 100 kDa and a polydispersity range between 1 and about 5.0, or between about 1.5 and about 3.0 is created and used as described herein.

In an embodiment, at least one RSPF mixture composition (e.g., a solution) having a MW range between about 100 kDa and about 105 kDa and a polydispersity range between 1 and about 5.0, or between about 1.5 and about 3.0 is created and used as described herein. In an embodiment, at least one RSPF mixture composition (e.g., a solution) having a MW range between about 105 kDa and about 110 kDa and a polydispersity range between 1 and about 5.0, or between about 1.5 and about 3.0 is created and used as described herein. In an embodiment, at least one RSPF mixture composition (e.g., a solution) having a MW range between about 110 kDa and about 115 kDa and a polydispersity range between 1 and about 5.0, or between about 1.5 and about 3.0 is created and used as described herein. In an embodiment, at least one RSPF mixture composition (e.g., a solution) having a MW range between about 115 kDa and about 120 kDa and a polydispersity range between 1 and about 5.0, or between about 1.5 and about 3.0 is created and used as described herein. In an embodiment, at least one RSPF mixture composition (e.g., a solution) having a MW range between about 120 kDa and about 125 kDa and a polydispersity range between 1 and about 5.0, or between about 1.5 and about 3.0 is created and used as described herein. In an embodiment, at least one RSPF mixture composition (e.g., a solution) having a MW range between about 125 kDa and about 130 kDa and a polydispersity range between 1 and about 5.0, or between about 1.5 and about 3.0 is created and used as described herein. In an embodiment, at least one RSPF mixture composition (e.g., a solution) having a MW range between about 130 kDa and about 135 kDa and a polydispersity range between 1 and about 5.0, or between about 1.5 and about 3.0 is created and used as described herein. In an embodiment, at least one RSPF mixture composition (e.g., a solution) having a MW range between about 135 kDa and about 140 kDa and a polydispersity range between 1 and about 5.0, or between about 1.5 and about 3.0 is created and used as described herein. In an embodiment, at least one RSPF mixture composition (e.g., a solution) having a MW range between about 140 kDa and about 145 kDa and a polydispersity range between 1 and about 5.0, or between about 1.5 and about 3.0 is created and used as described herein. In an embodiment, at least one RSPF mixture composition (e.g., a solution) having a MW range between about 145 kDa and about 150 kDa and a polydispersity range between 1 and about 5.0, or between about 1.5 and about 3.0 is created and used as described herein. In an embodiment, at least one RSPF mixture composition (e.g., a solution) having a MW range between about 150 kDa and about 155 kDa and a polydispersity range between 1 and about 5.0, or between about 1.5 and about 3.0 is created and used as described herein. In an embodiment, at least one RSPF mixture composition (e.g., a solution) having a MW range between about 155 kDa and about 160 kDa and a polydispersity range between 1 and about 5.0, or between about 1.5 and about 3.0 is created and used as described herein. In an embodiment, at least one RSPF mixture composition (e.g., a solution) having a MW range between about 160 kDa and about 165 kDa and a polydispersity range between 1 and about 5.0, or between about 1.5 and about 3.0 is created and used as described herein. In an embodiment, at least one RSPF mixture composition (e.g., a solution) having a MW range between about 165 kDa and about 170 kDa and a polydispersity range between 1 and about 5.0, or between about 1.5 and about 3.0 is created and used as described herein. In an embodiment, at least one RSPF mixture composition (e.g., a solution) having a MW range between about 170 kDa and about 175 kDa and a polydispersity range between 1 and about 5.0, or between about 1.5 and about 3.0 is created and used as described herein. In an embodiment, at least one RSPF mixture composition (e.g., a solution) having a MW range between about 175 kDa and about 180 kDa and a polydispersity range between 1 and about 5.0, or between about 1.5 and about 3.0 is created and used as described herein. In an embodiment, at least one RSPF mixture composition (e.g., a solution) having a MW range between about 180 kDa and about 185 kDa and a polydispersity range between 1 and about 5.0, or between about 1.5 and about 3.0 is created and used as described herein. In an embodiment, at least one RSPF mixture composition (e.g., a solution) having a MW range between about 185 kDa and about 190 kDa and a polydispersity range between 1 and about 5.0, or between about 1.5 and about 3.0 is created and used as described herein. In an embodiment, at least one RSPF mixture composition (e.g., a solution) having a MW range between about 190 kDa and about 195 kDa and a polydispersity range between 1 and about 5.0, or between about 1.5 and about 3.0 is created and used as described herein. In an embodiment, at least one RSPF mixture composition (e.g., a solution) having a MW range between about 195 kDa and about 200 kDa and a polydispersity range between 1 and about 5.0, or between about 1.5 and about 3.0 is created and used as described herein.

In an embodiment, at least one RSPF mixture composition (e.g., a solution) having a MW range between about 200 kDa and about 205 kDa and a polydispersity range between 1 and about 5.0, or between about 1.5 and about 3.0 is created and used as described herein. In an embodiment, at least one RSPF mixture composition (e.g., a solution) having a MW range between about 205 kDa and about 210 kDa and a polydispersity range between 1 and about 5.0, or between about 1.5 and about 3.0 is created and used as described herein. In an embodiment, at least one RSPF mixture composition (e.g., a solution) having a MW range between about 210 kDa and about 215 kDa and a polydispersity range between 1 and about 5.0, or between about 1.5 and about 3.0 is created and used as described herein. In an embodiment, at least one RSPF mixture composition (e.g., a solution) having a MW range between about 215 kDa and about 220 kDa and a polydispersity range between 1 and about 5.0, or between about 1.5 and about 3.0 is created and used as described herein. In an embodiment, at least one RSPF mixture composition (e.g., a solution) having a MW range between about 220 kDa and about 225 kDa and a polydispersity range between 1 and about 5.0, or between about 1.5 and about 3.0 is created and used as described herein. In an embodiment, at least one RSPF mixture composition (e.g., a solution) having a MW range between about 225 kDa and about 230 kDa and a polydispersity range between 1 and about 5.0, or between about 1.5 and about 3.0 is created and used as described herein. In an embodiment, at least one RSPF mixture composition (e.g., a solution) having a MW range between about 230 kDa and about 235 kDa and a polydispersity range between 1 and about 5.0, or between about 1.5 and about 3.0 is created and used as described herein. In an embodiment, at least one RSPF mixture composition (e.g., a solution) having a MW range between about 235 kDa and about 240 kDa and a polydispersity range between 1 and about 5.0, or between about 1.5 and about 3.0 is created and used as described herein. In an embodiment, at least one RSPF mixture composition (e.g., a solution) having a MW range between about 240 kDa and about 245 kDa and a polydispersity range between 1 and about 5.0, or between about 1.5 and about 3.0 is created and used as described herein. In an embodiment, at least one RSPF mixture composition (e.g., a solution) having a MW range between about 245 kDa and about 250 kDa and a polydispersity range between 1 and about 5.0, or between about 1.5 and about 3.0 is created and used as described herein. In an embodiment, at least one RSPF mixture composition (e.g., a solution) having a MW range between about 250 kDa and about 255 kDa and a polydispersity range between 1 and about 5.0, or between about 1.5 and about 3.0 is created and used as described herein. In an embodiment, at least one RSPF mixture composition (e.g., a solution) having a MW range between about 255 kDa and about 260 kDa and a polydispersity range between 1 and about 5.0, or between about 1.5 and about 3.0 is created and used as described herein. In an embodiment, at least one RSPF mixture composition (e.g., a solution) having a MW range between about 260 kDa and about 265 kDa and a polydispersity range between 1 and about 5.0, or between about 1.5 and about 3.0 is created and used as described herein. In an embodiment, at least one RSPF mixture composition (e.g., a solution) having a MW range between about 265 kDa and about 270 kDa and a polydispersity range between 1 and about 5.0, or between about 1.5 and about 3.0 is created and used as described herein. In an embodiment, at least one RSPF mixture composition (e.g., a solution) having a MW range between about 270 kDa and about 275 kDa and a polydispersity range between 1 and about 5.0, or between about 1.5 and about 3.0 is created and used as described herein. In an embodiment, at least one RSPF mixture composition (e.g., a solution) having a MW range between about 275 kDa and about 280 kDa and a polydispersity range between 1 and about 5.0, or between about 1.5 and about 3.0 is created and used as described herein. In an embodiment, at least one RSPF mixture composition (e.g., a solution) having a MW range between about 280 kDa and about 285 kDa and a polydispersity range between 1 and about 5.0, or between about 1.5 and about 3.0 is created and used as described herein. In an embodiment, at least one RSPF mixture composition (e.g., a solution) having a MW range between about 285 kDa and about 290 kDa and a polydispersity range between 1 and about 5.0, or between about 1.5 and about 3.0 is created and used as described herein. In an embodiment, at least one RSPF mixture composition (e.g., a solution) having a MW range between about 290 kDa and about 295 kDa and a polydispersity range between 1 and about 5.0, or between about 1.5 and about 3.0 is created and used as described herein. In an embodiment, at least one RSPF mixture composition (e.g., a solution) having a MW range between about 295 kDa and about 300 kDa and a polydispersity range between 1 and about 5.0, or between about 1.5 and about 3.0 is created and used as described herein.

In an embodiment, at least one RSPF mixture composition (e.g., a solution) having a MW range between about 300 kDa and about 305 kDa and a polydispersity range between 1 and about 5.0, or between about 1.5 and about 3.0 is created and used as described herein. In an embodiment, at least one RSPF mixture composition (e.g., a solution) having a MW range between about 305 kDa and about 310 kDa and a polydispersity range between 1 and about 5.0, or between about 1.5 and about 3.0 is created and used as described herein. In an embodiment, at least one RSPF mixture composition (e.g., a solution) having a MW range between about 310 kDa and about 315 kDa and a polydispersity range between 1 and about 5.0, or between about 1.5 and about 3.0 is created and used as described herein. In an embodiment, at least one RSPF mixture composition (e.g., a solution) having a MW range between about 315 kDa and about 320 kDa and a polydispersity range between 1 and about 5.0, or between about 1.5 and about 3.0 is created and used as described herein. In an embodiment, at least one RSPF mixture composition (e.g., a solution) having a MW range between about 320 kDa and about 325 kDa and a polydispersity range between 1 and about 5.0, or between about 1.5 and about 3.0 is created and used as described herein. In an embodiment, at least one RSPF mixture composition (e.g., a solution) having a MW range between about 325 kDa and about 330 kDa and a polydispersity range between 1 and about 5.0, or between about 1.5 and about 3.0 is created and used as described herein. In an embodiment, at least one RSPF mixture composition (e.g., a solution) having a MW range between about 330 kDa and about 335 kDa and a polydispersity range between 1 and about 5.0, or between about 1.5 and about 3.0 is created and used as described herein. In an embodiment, at least one RSPF mixture composition (e.g., a solution) having a MW range between about 335 kDa and about 340 kDa and a polydispersity range between 1 and about 5.0, or between about 1.5 and about 3.0 is created and used as described herein. In an embodiment, at least one RSPF mixture composition (e.g., a solution) having a MW range between about 340 kDa and about 345 kDa and a polydispersity range between 1 and about 5.0, or between about 1.5 and about 3.0 is created and used as described herein. In an embodiment, at least one RSPF mixture composition (e.g., a solution) having a MW range between about 345 kDa and about 350 kDa and a polydispersity range between 1 and about 5.0, or between about 1.5 and about 3.0 is created and used as described herein. In an embodiment, at least one RSPF mixture composition (e.g., a solution) having a MW range between about 350 kDa and about 355 kDa and a polydispersity range between 1 and about 5.0, or between about 1.5 and about 3.0 is created and used as described herein. In an embodiment, at least one RSPF mixture composition (e.g., a solution) having a MW range between about 355 kDa and about 360 kDa and a polydispersity range between 1 and about 5.0, or between about 1.5 and about 3.0 is created and used as described herein. In an embodiment, at least one RSPF mixture composition (e.g., a solution) having a MW range between about 360 kDa and about 365 kDa and a polydispersity range between 1 and about 5.0, or between about 1.5 and about 3.0 is created and used as described herein. In an embodiment, at least one RSPF mixture composition (e.g., a solution) having a MW range between about 365 kDa and about 370 kDa and a polydispersity range between 1 and about 5.0, or between about 1.5 and about 3.0 is created and used as described herein. In an embodiment, at least one RSPF mixture composition (e.g., a solution) having a MW range between about 370 kDa and about 375 kDa and a polydispersity range between 1 and about 5.0, or between about 1.5 and about 3.0 is created and used as described herein. In an embodiment, at least one RSPF mixture composition (e.g., a solution) having a MW range between about 375 kDa and about 380 kDa and a polydispersity range between 1 and about 5.0, or between about 1.5 and about 3.0 is created and used as described herein. In an embodiment, at least one RSPF mixture composition (e.g., a solution) having a MW range between about 380 kDa and about 385 kDa and a polydispersity range between 1 and about 5.0, or between about 1.5 and about 3.0 is created and used as described herein. In an embodiment, at least one RSPF mixture composition (e.g., a solution) having a MW range between about 385 kDa and about 390 kDa and a polydispersity range between 1 and about 5.0, or between about 1.5 and about 3.0 is created and used as described herein. In an embodiment, at least one RSPF mixture composition (e.g., a solution) having a MW range between about 390 kDa and about 395 kDa and a polydispersity range between 1 and about 5.0, or between about 1.5 and about 3.0 is created and used as described herein. In an embodiment, at least one RSPF mixture composition (e.g., a solution) having a MW range between about 395 kDa and about 400 kDa and a polydispersity range between 1 and about 5.0, or between about 1.5 and about 3.0 is created and used as described herein.

In some embodiments, one or more RSPF mixture compositions (e.g., solutions) selected from compositions #1001 to 2450, having weight average molecular weights selected from about 1 kDa to about 145 kDa, and a polydispersity range selected from between 1 and about 5, between 1 and about 1.5, between about 1.5 and about 2, between about 1.5 and about 3, between about 2 and about 2.5, between about 2.5 and about 3, between about 3 and about 3.5, between about 3.5 and about 4, between about 4 and about 4.5, and between about 4.5 and about 5, are created and used as described herein:

| MW | PDI (about) | | | | | | | | | |
|---|---|---|---|---|---|---|---|---|---|---|
| (about) | 1-5 | 1-1.5 | 1.5-2 | 1.5-3 | 2-2.5 | 2.5-3 | 3-3.5 | 3.5-4 | 4-4.5 | 4.5-5 |
| 1 kDa | 1001 | 1002 | 1003 | 1004 | 1005 | 1006 | 1007 | 1008 | 1009 | 1010 |
| 2 kDa | 1011 | 1012 | 1013 | 1014 | 1015 | 1016 | 1017 | 1018 | 1019 | 1020 |
| 3 kDa | 1021 | 1022 | 1023 | 1024 | 1025 | 1026 | 1027 | 1028 | 1029 | 1030 |
| 4 kDa | 1031 | 1032 | 1033 | 1034 | 1035 | 1036 | 1037 | 1038 | 1039 | 1040 |
| 5 kDa | 1041 | 1042 | 1043 | 1044 | 1045 | 1046 | 1047 | 1048 | 1049 | 1050 |
| 6 kDa | 1051 | 1052 | 1053 | 1054 | 1055 | 1056 | 1057 | 1058 | 1059 | 1060 |
| 7 kDa | 1061 | 1062 | 1063 | 1064 | 1065 | 1066 | 1067 | 1068 | 1069 | 1070 |
| 8 kDa | 1071 | 1072 | 1073 | 1074 | 1075 | 1076 | 1077 | 1078 | 1079 | 1080 |
| 9 kDa | 1081 | 1082 | 1083 | 1084 | 1085 | 1086 | 1087 | 1088 | 1089 | 1090 |
| 10 kDa | 1091 | 1092 | 1093 | 1094 | 1095 | 1096 | 1097 | 1098 | 1099 | 1100 |
| 11 kDa | 1101 | 1102 | 1103 | 1104 | 1105 | 1106 | 1107 | 1108 | 1109 | 1110 |
| 12 kDa | 1111 | 1112 | 1113 | 1114 | 1115 | 1116 | 1117 | 1118 | 1119 | 1120 |
| 13 kDa | 1121 | 1122 | 1123 | 1124 | 1125 | 1126 | 1127 | 1128 | 1129 | 1130 |
| 14 kDa | 1131 | 1132 | 1133 | 1134 | 1135 | 1136 | 1137 | 1138 | 1139 | 1140 |
| 15 kDa | 1141 | 1142 | 1143 | 1144 | 1145 | 1146 | 1147 | 1148 | 1149 | 1150 |
| 16 kDa | 1151 | 1152 | 1153 | 1154 | 1155 | 1156 | 1157 | 1158 | 1159 | 1160 |
| 17 kDa | 1161 | 1162 | 1163 | 1164 | 1165 | 1166 | 1167 | 1168 | 1169 | 1170 |
| 18 kDa | 1171 | 1172 | 1173 | 1174 | 1175 | 1176 | 1177 | 1178 | 1179 | 1180 |
| 19 kDa | 1181 | 1182 | 1183 | 1184 | 1185 | 1186 | 1187 | 1188 | 1189 | 1190 |
| 20 kDa | 1191 | 1192 | 1193 | 1194 | 1195 | 1196 | 1197 | 1198 | 1199 | 1200 |
| 21 kDa | 1201 | 1202 | 1203 | 1204 | 1205 | 1206 | 1207 | 1208 | 1209 | 1210 |
| 22 kDa | 1211 | 1212 | 1213 | 1214 | 1215 | 1216 | 1217 | 1218 | 1219 | 1220 |
| 23 kDa | 1221 | 1222 | 1223 | 1224 | 1225 | 1226 | 1227 | 1228 | 1229 | 1230 |
| 24 kDa | 1231 | 1232 | 1233 | 1234 | 1235 | 1236 | 1237 | 1238 | 1239 | 1240 |
| 25 kDa | 1241 | 1242 | 1243 | 1244 | 1245 | 1246 | 1247 | 1248 | 1249 | 1250 |
| 26 kDa | 1251 | 1252 | 1253 | 1254 | 1255 | 1256 | 1257 | 1258 | 1259 | 1260 |
| 27 kDa | 1261 | 1262 | 1263 | 1264 | 1265 | 1266 | 1267 | 1268 | 1269 | 1270 |
| 28 kDa | 1271 | 1272 | 1273 | 1274 | 1275 | 1276 | 1277 | 1278 | 1279 | 1280 |
| 29 kDa | 1281 | 1282 | 1283 | 1284 | 1285 | 1286 | 1287 | 1288 | 1289 | 1290 |
| 30 kDa | 1291 | 1292 | 1293 | 1294 | 1295 | 1296 | 1297 | 1298 | 1299 | 1300 |
| 31 kDa | 1301 | 1302 | 1303 | 1304 | 1305 | 1306 | 1307 | 1308 | 1309 | 1310 |
| 32 kDa | 1311 | 1312 | 1313 | 1314 | 1315 | 1316 | 1317 | 1318 | 1319 | 1320 |
| 33 kDa | 1321 | 1322 | 1323 | 1324 | 1325 | 1326 | 1327 | 1328 | 1329 | 1330 |
| 34 kDa | 1331 | 1332 | 1333 | 1334 | 1335 | 1336 | 1337 | 1338 | 1339 | 1340 |
| 35 kDa | 1341 | 1342 | 1343 | 1344 | 1345 | 1346 | 1347 | 1348 | 1349 | 1350 |
| 36 kDa | 1351 | 1352 | 1353 | 1354 | 1355 | 1356 | 1357 | 1358 | 1359 | 1360 |
| 37 kDa | 1361 | 1362 | 1363 | 1364 | 1365 | 1366 | 1367 | 1368 | 1369 | 1370 |
| 38 kDa | 1371 | 1372 | 1373 | 1374 | 1375 | 1376 | 1377 | 1378 | 1379 | 1380 |
| 39 kDa | 1381 | 1382 | 1383 | 1384 | 1385 | 1386 | 1387 | 1388 | 1389 | 1390 |
| 40 kDa | 1391 | 1392 | 1393 | 1394 | 1395 | 1396 | 1397 | 1398 | 1399 | 1400 |
| 41 kDa | 1401 | 1402 | 1403 | 1404 | 1405 | 1406 | 1407 | 1408 | 1409 | 1410 |
| 42 kDa | 1411 | 1412 | 1413 | 1414 | 1415 | 1416 | 1417 | 1418 | 1419 | 1420 |
| 43 kDa | 1421 | 1422 | 1423 | 1424 | 1425 | 1426 | 1427 | 1428 | 1429 | 1430 |
| 44 kDa | 1431 | 1432 | 1433 | 1434 | 1435 | 1436 | 1437 | 1438 | 1439 | 1440 |
| 45 kDa | 1441 | 1442 | 1443 | 1444 | 1445 | 1446 | 1447 | 1448 | 1449 | 1450 |
| 46 kDa | 1451 | 1452 | 1453 | 1454 | 1455 | 1456 | 1457 | 1458 | 1459 | 1460 |
| 47 kDa | 1461 | 1462 | 1463 | 1464 | 1465 | 1466 | 1467 | 1468 | 1469 | 1470 |
| 48 kDa | 1471 | 1472 | 1473 | 1474 | 1475 | 1476 | 1477 | 1478 | 1479 | 1480 |
| 49 kDa | 1481 | 1482 | 1483 | 1484 | 1485 | 1486 | 1487 | 1488 | 1489 | 1490 |
| 50 kDa | 1491 | 1492 | 1493 | 1494 | 1495 | 1496 | 1497 | 1498 | 1499 | 1500 |
| 51 kDa | 1501 | 1502 | 1503 | 1504 | 1505 | 1506 | 1507 | 1508 | 1509 | 1510 |
| 52 kDa | 1511 | 1512 | 1513 | 1514 | 1515 | 1516 | 1517 | 1518 | 1519 | 1520 |
| 53 kDa | 1521 | 1522 | 1523 | 1524 | 1525 | 1526 | 1527 | 1528 | 1529 | 1530 |
| 54 kDa | 1531 | 1532 | 1533 | 1534 | 1535 | 1536 | 1537 | 1538 | 1539 | 1540 |
| 55 kDa | 1541 | 1542 | 1543 | 1544 | 1545 | 1546 | 1547 | 1548 | 1549 | 1550 |
| 56 kDa | 1551 | 1552 | 1553 | 1554 | 1555 | 1556 | 1557 | 1558 | 1559 | 1560 |
| 57 kDa | 1561 | 1562 | 1563 | 1564 | 1565 | 1566 | 1567 | 1568 | 1569 | 1570 |
| 58 kDa | 1571 | 1572 | 1573 | 1574 | 1575 | 1576 | 1577 | 1578 | 1579 | 1580 |
| 59 kDa | 1581 | 1582 | 1583 | 1584 | 1585 | 1586 | 1587 | 1588 | 1589 | 1590 |
| 60 kDa | 1591 | 1592 | 1593 | 1594 | 1595 | 1596 | 1597 | 1598 | 1599 | 1600 |
| 61 kDa | 1601 | 1602 | 1603 | 1604 | 1605 | 1606 | 1607 | 1608 | 1609 | 1610 |
| 62 kDa | 1611 | 1612 | 1613 | 1614 | 1615 | 1616 | 1617 | 1618 | 1619 | 1620 |
| 63 kDa | 1621 | 1622 | 1623 | 1624 | 1625 | 1626 | 1627 | 1628 | 1629 | 1630 |
| 64 kDa | 1631 | 1632 | 1633 | 1634 | 1635 | 1636 | 1637 | 1638 | 1639 | 1640 |
| 65 kDa | 1641 | 1642 | 1643 | 1644 | 1645 | 1646 | 1647 | 1648 | 1649 | 1650 |
| 66 kDa | 1651 | 1652 | 1653 | 1654 | 1655 | 1656 | 1657 | 1658 | 1659 | 1660 |
| 67 kDa | 1661 | 1662 | 1663 | 1664 | 1665 | 1666 | 1667 | 1668 | 1669 | 1670 |
| 68 kDa | 1671 | 1672 | 1673 | 1674 | 1675 | 1676 | 1677 | 1678 | 1679 | 1680 |
| 69 kDa | 1681 | 1682 | 1683 | 1684 | 1685 | 1686 | 1687 | 1688 | 1689 | 1690 |
| 70 kDa | 1691 | 1692 | 1693 | 1694 | 1695 | 1696 | 1697 | 1698 | 1699 | 1700 |
| 71 kDa | 1701 | 1702 | 1703 | 1704 | 1705 | 1706 | 1707 | 1708 | 1709 | 1710 |
| 72 kDa | 1711 | 1712 | 1713 | 1714 | 1715 | 1716 | 1717 | 1718 | 1719 | 1720 |
| 73 kDa | 1721 | 1722 | 1723 | 1724 | 1725 | 1726 | 1727 | 1728 | 1729 | 1730 |
| 74 kDa | 1731 | 1732 | 1733 | 1734 | 1735 | 1736 | 1737 | 1738 | 1739 | 1740 |
| 75 kDa | 1741 | 1742 | 1743 | 1744 | 1745 | 1746 | 1747 | 1748 | 1749 | 1750 |
| 76 kDa | 1751 | 1752 | 1753 | 1754 | 1755 | 1756 | 1757 | 1758 | 1759 | 1760 |
| 77 kDa | 1761 | 1762 | 1763 | 1764 | 1765 | 1766 | 1767 | 1768 | 1769 | 1770 |
| 78 kDa | 1771 | 1772 | 1773 | 1774 | 1775 | 1776 | 1777 | 1778 | 1779 | 1780 |
| 79 kDa | 1781 | 1782 | 1783 | 1784 | 1785 | 1786 | 1787 | 1788 | 1789 | 1790 |
| 80 kDa | 1791 | 1792 | 1793 | 1794 | 1795 | 1796 | 1797 | 1798 | 1799 | 1800 |
| 81 kDa | 1801 | 1802 | 1803 | 1804 | 1805 | 1806 | 1807 | 1808 | 1809 | 1810 |
| 82 kDa | 1811 | 1812 | 1813 | 1814 | 1815 | 1816 | 1817 | 1818 | 1819 | 1820 |
| 83 kDa | 1821 | 1822 | 1823 | 1824 | 1825 | 1826 | 1827 | 1828 | 1829 | 1830 |
| 84 kDa | 1831 | 1832 | 1833 | 1834 | 1835 | 1836 | 1837 | 1838 | 1839 | 1840 |
| 85 kDa | 1841 | 1842 | 1843 | 1844 | 1845 | 1846 | 1847 | 1848 | 1849 | 1850 |
| 86 kDa | 1851 | 1852 | 1853 | 1854 | 1855 | 1856 | 1857 | 1858 | 1859 | 1860 |
| 87 kDa | 1861 | 1862 | 1863 | 1864 | 1865 | 1866 | 1867 | 1868 | 1869 | 1870 |
| 88 kDa | 1871 | 1872 | 1873 | 1874 | 1875 | 1876 | 1877 | 1878 | 1879 | 1880 |
| 89 kDa | 1881 | 1882 | 1883 | 1884 | 1885 | 1886 | 1887 | 1888 | 1889 | 1890 |
| 90 kDa | 1891 | 1892 | 1893 | 1894 | 1895 | 1896 | 1897 | 1898 | 1899 | 1900 |
| 91 kDa | 1901 | 1902 | 1903 | 1904 | 1905 | 1906 | 1907 | 1908 | 1909 | 1910 |
| 92 kDa | 1911 | 1912 | 1913 | 1914 | 1915 | 1916 | 1917 | 1918 | 1919 | 1920 |
| 93 kDa | 1921 | 1922 | 1923 | 1924 | 1925 | 1926 | 1927 | 1928 | 1929 | 1930 |
| 94 kDa | 1931 | 1932 | 1933 | 1934 | 1935 | 1936 | 1937 | 1938 | 1939 | 1940 |
| 95 kDa | 1941 | 1942 | 1943 | 1944 | 1945 | 1946 | 1947 | 1948 | 1949 | 1950 |
| 96 kDa | 1951 | 1952 | 1953 | 1954 | 1955 | 1956 | 1957 | 1958 | 1959 | 1960 |
| 97 kDa | 1961 | 1962 | 1963 | 1964 | 1965 | 1966 | 1967 | 1968 | 1969 | 1970 |
| 98 kDa | 1971 | 1972 | 1973 | 1974 | 1975 | 1976 | 1977 | 1978 | 1979 | 1980 |
| 99 kDa | 1981 | 1982 | 1983 | 1984 | 1985 | 1986 | 1987 | 1988 | 1989 | 1990 |
| 100 kDa | 1991 | 1992 | 1993 | 1994 | 1995 | 1996 | 1997 | 1998 | 1999 | 2000 |
| 101 kDa | 2001 | 2002 | 2003 | 2004 | 2005 | 2006 | 2007 | 2008 | 2009 | 2010 |
| 102 kDa | 2011 | 2012 | 2013 | 2014 | 2015 | 2016 | 2017 | 2018 | 2019 | 2020 |
| 103 kDa | 2021 | 2022 | 2023 | 2024 | 2025 | 2026 | 2027 | 2028 | 2029 | 2030 |
| 104 kDa | 2031 | 2032 | 2033 | 2034 | 2035 | 2036 | 2037 | 2038 | 2039 | 2040 |
| 105 kDa | 2041 | 2042 | 2043 | 2044 | 2045 | 2046 | 2047 | 2048 | 2049 | 2050 |
| 106 kDa | 2051 | 2052 | 2053 | 2054 | 2055 | 2056 | 2057 | 2058 | 2059 | 2060 |
| 107 kDa | 2061 | 2062 | 2063 | 2064 | 2065 | 2066 | 2067 | 2068 | 2069 | 2070 |
| 108 kDa | 2071 | 2072 | 2073 | 2074 | 2075 | 2076 | 2077 | 2078 | 2079 | 2080 |
| 109 kDa | 2081 | 2082 | 2083 | 2084 | 2085 | 2086 | 2087 | 2088 | 2089 | 2090 |
| 110 kDa | 2091 | 2092 | 2093 | 2094 | 2095 | 2096 | 2097 | 2098 | 2099 | 2100 |
| 111 kDa | 2101 | 2102 | 2103 | 2104 | 2105 | 2106 | 2107 | 2108 | 2109 | 2110 |
| 112 kDa | 2111 | 2112 | 2113 | 2114 | 2115 | 2116 | 2117 | 2118 | 2119 | 2120 |
| 113 kDa | 2121 | 2122 | 2123 | 2124 | 2125 | 2126 | 2127 | 2128 | 2129 | 2130 |
| 114 kDa | 2131 | 2132 | 2133 | 2134 | 2135 | 2136 | 2137 | 2138 | 2139 | 2140 |
| 115 kDa | 2141 | 2142 | 2143 | 2144 | 2145 | 2146 | 2147 | 2148 | 2149 | 2150 |
| 116 kDa | 2151 | 2152 | 2153 | 2154 | 2155 | 2156 | 2157 | 2158 | 2159 | 2160 |
| 117 kDa | 2161 | 2162 | 2163 | 2164 | 2165 | 2166 | 2167 | 2168 | 2169 | 2170 |
| 118 kDa | 2171 | 2172 | 2173 | 2174 | 2175 | 2176 | 2177 | 2178 | 2179 | 2180 |
| 119 kDa | 2181 | 2182 | 2183 | 2184 | 2185 | 2186 | 2187 | 2188 | 2189 | 2190 |
| 120 kDa | 2191 | 2192 | 2193 | 2194 | 2195 | 2196 | 2197 | 2198 | 2199 | 2200 |
| 121 kDa | 2201 | 2202 | 2203 | 2204 | 2205 | 2206 | 2207 | 2208 | 2209 | 2210 |
| 122 kDa | 2211 | 2212 | 2213 | 2214 | 2215 | 2216 | 2217 | 2218 | 2219 | 2220 |
| 123 kDa | 2221 | 2222 | 2223 | 2224 | 2225 | 2226 | 2227 | 2228 | 2229 | 2230 |
| 124 kDa | 2231 | 2232 | 2233 | 2234 | 2235 | 2236 | 2237 | 2238 | 2239 | 2240 |
| 125 kDa | 2241 | 2242 | 2243 | 2244 | 2245 | 2246 | 2247 | 2248 | 2249 | 2250 |
| 126 kDa | 2251 | 2252 | 2253 | 2254 | 2255 | 2256 | 2257 | 2258 | 2259 | 2260 |
| 127 kDa | 2261 | 2262 | 2263 | 2264 | 2265 | 2266 | 2267 | 2268 | 2269 | 2270 |
| 128 kDa | 2271 | 2272 | 2273 | 2274 | 2275 | 2276 | 2277 | 2278 | 2279 | 2280 |
| 129 kDa | 2281 | 2282 | 2283 | 2284 | 2285 | 2286 | 2287 | 2288 | 2289 | 2290 |
| 130 kDa | 2291 | 2292 | 2293 | 2294 | 2295 | 2296 | 2297 | 2298 | 2299 | 2300 |
| 131 kDa | 2301 | 2302 | 2303 | 2304 | 2305 | 2306 | 2307 | 2308 | 2309 | 2310 |
| 132 kDa | 2311 | 2312 | 2313 | 2314 | 2315 | 2316 | 2317 | 2318 | 2319 | 2320 |
| 133 kDa | 2321 | 2322 | 2323 | 2324 | 2325 | 2326 | 2327 | 2328 | 2329 | 2330 |
| 134 kDa | 2331 | 2332 | 2333 | 2334 | 2335 | 2336 | 2337 | 2338 | 2339 | 2340 |
| 135 kDa | 2341 | 2342 | 2343 | 2344 | 2345 | 2346 | 2347 | 2348 | 2349 | 2350 |
| 136 kDa | 2351 | 2352 | 2353 | 2354 | 2355 | 2356 | 2357 | 2358 | 2359 | 2360 |
| 137 kDa | 2361 | 2362 | 2363 | 2364 | 2365 | 2366 | 2367 | 2368 | 2369 | 2370 |
| 138 kDa | 2371 | 2372 | 2373 | 2374 | 2375 | 2376 | 2377 | 2378 | 2379 | 2380 |
| 139 kDa | 2381 | 2382 | 2383 | 2384 | 2385 | 2386 | 2387 | 2388 | 2389 | 2390 |
| 140 kDa | 2391 | 2392 | 2393 | 2394 | 2395 | 2396 | 2397 | 2398 | 2399 | 2400 |
| 141 kDa | 2401 | 2402 | 2403 | 2404 | 2405 | 2406 | 2407 | 2408 | 2409 | 2410 |
| 142 kDa | 2411 | 2412 | 2413 | 2414 | 2415 | 2416 | 2417 | 2418 | 2419 | 2420 |
| 143 kDa | 2421 | 2422 | 2423 | 2424 | 2425 | 2426 | 2427 | 2428 | 2429 | 2430 |
| 144 kDa | 2431 | 2432 | 2433 | 2434 | 2435 | 2436 | 2437 | 2438 | 2439 | 2440 |
| 145 kDa | 2441 | 2442 | 2443 | 2444 | 2445 | 2446 | 2447 | 2448 | 2449 | 2450 |

In some embodiments, one or more RSPF mixture compositions (e.g., solutions) selected from compositions #3001 to #4450, having weight average molecular weights selected from about 1 kDa to about 145 kDa, and a polydispersity range selected from between 1 and about 1.1 (including e.g., a polydispersity of 1), between 1.1 and about 1.2, between about 1.2 and about 1.3, between about 1.3 and about 1.4, between about 1.4 and about 1.5, between about 1.5 and about 1.6, between about 1.6 and about 1.7, between about 1.7 and about 1.8, between about 1.8 and about 1.9, and between about 1.9 and about 2, are created and used as described herein:

| MW | PDI (about) | | | | | | | | | |
|---|---|---|---|---|---|---|---|---|---|---|
| (about) | 1-1.1 | 1.1-1.2 | 1.2-1.3 | 1.3-1.4 | 1.4-1.5 | 1.5-1.6 | 1.6-1.7 | 1.7-1.8 | 1.8-1.9 | 1.9-2 |
| 1 kDa | 3001 | 3002 | 3003 | 3004 | 3005 | 3006 | 3007 | 3008 | 3009 | 3010 |
| 2 kDa | 3011 | 3012 | 3013 | 3014 | 3015 | 3016 | 3017 | 3018 | 3019 | 3020 |
| 3 kDa | 3021 | 3022 | 3023 | 3024 | 3025 | 3026 | 3027 | 3028 | 3029 | 3030 |
| 4 kDa | 3031 | 3032 | 3033 | 3034 | 3035 | 3036 | 3037 | 3038 | 3039 | 3040 |
| 5 kDa | 3041 | 3042 | 3043 | 3044 | 3045 | 3046 | 3047 | 3048 | 3049 | 3050 |
| 6 kDa | 3051 | 3052 | 3053 | 3054 | 3055 | 3056 | 3057 | 3058 | 3059 | 3060 |
| 7 kDa | 3061 | 3062 | 3063 | 3064 | 3065 | 3066 | 3067 | 3068 | 3069 | 3070 |
| 8 kDa | 3071 | 3072 | 3073 | 3074 | 3075 | 3076 | 3077 | 3078 | 3079 | 3080 |
| 9 kDa | 3081 | 3082 | 3083 | 3084 | 3085 | 3086 | 3087 | 3088 | 3089 | 3090 |
| 10 kDa | 3091 | 3092 | 3093 | 3094 | 3095 | 3096 | 3097 | 3098 | 3099 | 3100 |
| 11 kDa | 3101 | 3102 | 3103 | 3104 | 3105 | 3106 | 3107 | 3108 | 3109 | 3110 |
| 12 kDa | 3111 | 3112 | 3113 | 3114 | 3115 | 3116 | 3117 | 3118 | 3119 | 3120 |
| 13 kDa | 3121 | 3122 | 3123 | 3124 | 3125 | 3126 | 3127 | 3128 | 3129 | 3130 |
| 14 kDa | 3131 | 3132 | 3133 | 3134 | 3135 | 3136 | 3137 | 3138 | 3139 | 3140 |
| 15 kDa | 3141 | 3142 | 3143 | 3144 | 3145 | 3146 | 3147 | 3148 | 3149 | 3150 |
| 16 kDa | 3151 | 3152 | 3153 | 3154 | 3155 | 3156 | 3157 | 3158 | 3159 | 3160 |
| 17 kDa | 3161 | 3162 | 3163 | 3164 | 3165 | 3166 | 3167 | 3168 | 3169 | 3170 |
| 18 kDa | 3171 | 3172 | 3173 | 3174 | 3175 | 3176 | 3177 | 3178 | 3179 | 3180 |
| 19 kDa | 3181 | 3182 | 3183 | 3184 | 3185 | 3186 | 3187 | 3188 | 3189 | 3190 |
| 20 kDa | 3191 | 3192 | 3193 | 3194 | 3195 | 3196 | 3197 | 3198 | 3199 | 3200 |
| 21 kDa | 3201 | 3202 | 3203 | 3204 | 3205 | 3206 | 3207 | 3208 | 3209 | 3210 |
| 22 kDa | 3211 | 3212 | 3213 | 3214 | 3215 | 3216 | 3217 | 3218 | 3219 | 3220 |
| 23 kDa | 3221 | 3222 | 3223 | 3224 | 3225 | 3226 | 3227 | 3228 | 3229 | 3230 |
| 24 kDa | 3231 | 3232 | 3233 | 3234 | 3235 | 3236 | 3237 | 3238 | 3239 | 3240 |
| 25 kDa | 3241 | 3242 | 3243 | 3244 | 3245 | 3246 | 3247 | 3248 | 3249 | 3250 |
| 26 kDa | 3251 | 3252 | 3253 | 3254 | 3255 | 3256 | 3257 | 3258 | 3259 | 3260 |
| 27 kDa | 3261 | 3262 | 3263 | 3264 | 3265 | 3266 | 3267 | 3268 | 3269 | 3270 |
| 28 kDa | 3271 | 3272 | 3273 | 3274 | 3275 | 3276 | 3277 | 3278 | 3279 | 3280 |
| 29 kDa | 3281 | 3282 | 3283 | 3284 | 3285 | 3286 | 3287 | 3288 | 3289 | 3290 |
| 30 kDa | 3291 | 3292 | 3293 | 3294 | 3295 | 3296 | 3297 | 3298 | 3299 | 3300 |
| 31 kDa | 3301 | 3302 | 3303 | 3304 | 3305 | 3306 | 3307 | 3308 | 3309 | 3310 |
| 32 kDa | 3311 | 3312 | 3313 | 3314 | 3315 | 3316 | 3317 | 3318 | 3319 | 3320 |
| 33 kDa | 3321 | 3322 | 3323 | 3324 | 3325 | 3326 | 3327 | 3328 | 3329 | 3330 |
| 34 kDa | 3331 | 3332 | 3333 | 3334 | 3335 | 3336 | 3337 | 3338 | 3339 | 3340 |
| 35 kDa | 3341 | 3342 | 3343 | 3344 | 3345 | 3346 | 3347 | 3348 | 3349 | 3350 |
| 36 kDa | 3351 | 3352 | 3353 | 3354 | 3355 | 3356 | 3357 | 3358 | 3359 | 3360 |
| 37 kDa | 3361 | 3362 | 3363 | 3364 | 3365 | 3366 | 3367 | 3368 | 3369 | 3370 |
| 38 kDa | 3371 | 3372 | 3373 | 3374 | 3375 | 3376 | 3377 | 3378 | 3379 | 3380 |
| 39 kDa | 3381 | 3382 | 3383 | 3384 | 3385 | 3386 | 3387 | 3388 | 3389 | 3390 |
| 40 kDa | 3391 | 3392 | 3393 | 3394 | 3395 | 3396 | 3397 | 3398 | 3399 | 3400 |
| 41 kDa | 3401 | 3402 | 3403 | 3404 | 3405 | 3406 | 3407 | 3408 | 3409 | 3410 |
| 42 kDa | 3411 | 3412 | 3413 | 3414 | 3415 | 3416 | 3417 | 3418 | 3419 | 3420 |
| 43 kDa | 3421 | 3422 | 3423 | 3424 | 3425 | 3426 | 3427 | 3428 | 3429 | 3430 |
| 44 kDa | 3431 | 3432 | 3433 | 3434 | 3435 | 3436 | 3437 | 3438 | 3439 | 3440 |
| 45 kDa | 3441 | 3442 | 3443 | 3444 | 3445 | 3446 | 3447 | 3448 | 3449 | 3450 |
| 46 kDa | 3451 | 3452 | 3453 | 3454 | 3455 | 3456 | 3457 | 3458 | 3459 | 3460 |
| 47 kDa | 3461 | 3462 | 3463 | 3464 | 3465 | 3466 | 3467 | 3468 | 3469 | 3470 |
| 48 kDa | 3471 | 3472 | 3473 | 3474 | 3475 | 3476 | 3477 | 3478 | 3479 | 3480 |
| 49 kDa | 3481 | 3482 | 3483 | 3484 | 3485 | 3486 | 3487 | 3488 | 3489 | 3490 |
| 50 kDa | 3491 | 3492 | 3493 | 3494 | 3495 | 3496 | 3497 | 3498 | 3499 | 3500 |
| 51 kDa | 3501 | 3502 | 3503 | 3504 | 3505 | 3506 | 3507 | 3508 | 3509 | 3510 |
| 52 kDa | 3511 | 3512 | 3513 | 3514 | 3515 | 3516 | 3517 | 3518 | 3519 | 3520 |
| 53 kDa | 3521 | 3522 | 3523 | 3524 | 3525 | 3526 | 3527 | 3528 | 3529 | 3530 |
| 54 kDa | 3531 | 3532 | 3533 | 3534 | 3535 | 3536 | 3537 | 3538 | 3539 | 3540 |
| 55 kDa | 3541 | 3542 | 3543 | 3544 | 3545 | 3546 | 3547 | 3548 | 3549 | 3550 |
| 56 kDa | 3551 | 3552 | 3553 | 3554 | 3555 | 3556 | 3557 | 3558 | 3559 | 3560 |
| 57 kDa | 3561 | 3562 | 3563 | 3564 | 3565 | 3566 | 3567 | 3568 | 3569 | 3570 |
| 58 kDa | 3571 | 3572 | 3573 | 3574 | 3575 | 3576 | 3577 | 3578 | 3579 | 3580 |
| 59 kDa | 3581 | 3582 | 3583 | 3584 | 3585 | 3586 | 3587 | 3588 | 3589 | 3590 |
| 60 kDa | 3591 | 3592 | 3593 | 3594 | 3595 | 3596 | 3597 | 3598 | 3599 | 3600 |
| 61 kDa | 3601 | 3602 | 3603 | 3604 | 3605 | 3606 | 3607 | 3608 | 3609 | 3610 |
| 62 kDa | 3611 | 3612 | 3613 | 3614 | 3615 | 3616 | 3617 | 3618 | 3619 | 3620 |
| 63 kDa | 3621 | 3622 | 3623 | 3624 | 3625 | 3626 | 3627 | 3628 | 3629 | 3630 |
| 64 kDa | 3631 | 3632 | 3633 | 3634 | 3635 | 3636 | 3637 | 3638 | 3639 | 3640 |
| 65 kDa | 3641 | 3642 | 3643 | 3644 | 3645 | 3646 | 3647 | 3648 | 3649 | 3650 |
| 66 kDa | 3651 | 3652 | 3653 | 3654 | 3655 | 3656 | 3657 | 3658 | 3659 | 3660 |
| 67 kDa | 3661 | 3662 | 3663 | 3664 | 3665 | 3666 | 3667 | 3668 | 3669 | 3670 |
| 68 kDa | 3671 | 3672 | 3673 | 3674 | 3675 | 3676 | 3677 | 3678 | 3679 | 3680 |
| 69 kDa | 3681 | 3682 | 3683 | 3684 | 3685 | 3686 | 3687 | 3688 | 3689 | 3690 |
| 70 kDa | 3691 | 3692 | 3693 | 3694 | 3695 | 3696 | 3697 | 3698 | 3699 | 3700 |
| 71 kDa | 3701 | 3702 | 3703 | 3704 | 3705 | 3706 | 3707 | 3708 | 3709 | 3710 |
| 72 kDa | 3711 | 3712 | 3713 | 3714 | 3715 | 3716 | 3717 | 3718 | 3719 | 3720 |
| 73 kDa | 3721 | 3722 | 3723 | 3724 | 3725 | 3726 | 3727 | 3728 | 3729 | 3730 |
| 74 kDa | 3731 | 3732 | 3733 | 3734 | 3735 | 3736 | 3737 | 3738 | 3739 | 3740 |
| 75 kDa | 3741 | 3742 | 3743 | 3744 | 3745 | 3746 | 3747 | 3748 | 3749 | 3750 |
| 76 kDa | 3751 | 3752 | 3753 | 3754 | 3755 | 3756 | 3757 | 3758 | 3759 | 3760 |
| 77 kDa | 3761 | 3762 | 3763 | 3764 | 3765 | 3766 | 3767 | 3768 | 3769 | 3770 |
| 78 kDa | 3771 | 3772 | 3773 | 3774 | 3775 | 3776 | 3777 | 3778 | 3779 | 3780 |
| 79 kDa | 3781 | 3782 | 3783 | 3784 | 3785 | 3786 | 3787 | 3788 | 3789 | 3790 |
| 80 kDa | 3791 | 3792 | 3793 | 3794 | 3795 | 3796 | 3797 | 3798 | 3799 | 3800 |
| 81 kDa | 3801 | 3802 | 3803 | 3804 | 3805 | 3806 | 3807 | 3808 | 3809 | 3810 |
| 82 kDa | 3811 | 3812 | 3813 | 3814 | 3815 | 3816 | 3817 | 3818 | 3819 | 3820 |
| 83 kDa | 3821 | 3822 | 3823 | 3824 | 3825 | 3826 | 3827 | 3828 | 3829 | 3830 |
| 84 kDa | 3831 | 3832 | 3833 | 3834 | 3835 | 3836 | 3837 | 3838 | 3839 | 3840 |
| 85 kDa | 3841 | 3842 | 3843 | 3844 | 3845 | 3846 | 3847 | 3848 | 3849 | 3850 |
| 86 kDa | 3851 | 3852 | 3853 | 3854 | 3855 | 3856 | 3857 | 3858 | 3859 | 3860 |
| 87 kDa | 3861 | 3862 | 3863 | 3864 | 3865 | 3866 | 3867 | 3868 | 3869 | 3870 |
| 88 kDa | 3871 | 3872 | 3873 | 3874 | 3875 | 3876 | 3877 | 3878 | 3879 | 3880 |
| 89 kDa | 3881 | 3882 | 3883 | 3884 | 3885 | 3886 | 3887 | 3888 | 3889 | 3890 |
| 90 kDa | 3891 | 3892 | 3893 | 3894 | 3895 | 3896 | 3897 | 3898 | 3899 | 3900 |
| 91 kDa | 3901 | 3902 | 3903 | 3904 | 3905 | 3906 | 3907 | 3908 | 3909 | 3910 |
| 92 kDa | 3911 | 3912 | 3913 | 3914 | 3915 | 3916 | 3917 | 3918 | 3919 | 3920 |
| 93 kDa | 3921 | 3922 | 3923 | 3924 | 3925 | 3926 | 3927 | 3928 | 3929 | 3930 |
| 94 kDa | 3931 | 3932 | 3933 | 3934 | 3935 | 3936 | 3937 | 3938 | 3939 | 3940 |
| 95 kDa | 3941 | 3942 | 3943 | 3944 | 3945 | 3946 | 3947 | 3948 | 3949 | 3950 |
| 96 kDa | 3951 | 3952 | 3953 | 3954 | 3955 | 3956 | 3957 | 3958 | 3959 | 3960 |
| 97 kDa | 3961 | 3962 | 3963 | 3964 | 3965 | 3966 | 3967 | 3968 | 3969 | 3970 |
| 98 kDa | 3971 | 3972 | 3973 | 3974 | 3975 | 3976 | 3977 | 3978 | 3979 | 3980 |
| 99 kDa | 3981 | 3982 | 3983 | 3984 | 3985 | 3986 | 3987 | 3988 | 3989 | 3990 |
| 100 kDa | 3991 | 3992 | 3993 | 3994 | 3995 | 3996 | 3997 | 3998 | 3999 | 4000 |
| 101 kDa | 4001 | 4002 | 4003 | 4004 | 4005 | 4006 | 4007 | 4008 | 4009 | 4010 |
| 102 kDa | 4011 | 4012 | 4013 | 4014 | 4015 | 4016 | 4017 | 4018 | 4019 | 4020 |
| 103 kDa | 4021 | 4022 | 4023 | 4024 | 4025 | 4026 | 4027 | 4028 | 4029 | 4030 |
| 104 kDa | 4031 | 4032 | 4033 | 4034 | 4035 | 4036 | 4037 | 4038 | 4039 | 4040 |
| 105 kDa | 4041 | 4042 | 4043 | 4044 | 4045 | 4046 | 4047 | 4048 | 4049 | 4050 |
| 106 kDa | 4051 | 4052 | 4053 | 4054 | 4055 | 4056 | 4057 | 4058 | 4059 | 4060 |
| 107 kDa | 4061 | 4062 | 4063 | 4064 | 4065 | 4066 | 4067 | 4068 | 4069 | 4070 |
| 108 kDa | 4071 | 4072 | 4073 | 4074 | 4075 | 4076 | 4077 | 4078 | 4079 | 4080 |
| 109 kDa | 4081 | 4082 | 4083 | 4084 | 4085 | 4086 | 4087 | 4088 | 4089 | 4090 |
| 110 kDa | 4091 | 4092 | 4093 | 4094 | 4095 | 4096 | 4097 | 4098 | 4099 | 4100 |
| 111 kDa | 4101 | 4102 | 4103 | 4104 | 4105 | 4106 | 4107 | 4108 | 4109 | 4110 |
| 112 kDa | 4111 | 4112 | 4113 | 4114 | 4115 | 4116 | 4117 | 4118 | 4119 | 4120 |
| 113 kDa | 4121 | 4122 | 4123 | 4124 | 4125 | 4126 | 4127 | 4128 | 4129 | 4130 |
| 114 kDa | 4131 | 4132 | 4133 | 4134 | 4135 | 4136 | 4137 | 4138 | 4139 | 4140 |
| 115 kDa | 4141 | 4142 | 4143 | 4144 | 4145 | 4146 | 4147 | 4148 | 4149 | 4150 |
| 116 kDa | 4151 | 4152 | 4153 | 4154 | 4155 | 4156 | 4157 | 4158 | 4159 | 4160 |
| 117 kDa | 4161 | 4162 | 4163 | 4164 | 4165 | 4166 | 4167 | 4168 | 4169 | 4170 |
| 118 kDa | 4171 | 4172 | 4173 | 4174 | 4175 | 4176 | 4177 | 4178 | 4179 | 4180 |
| 119 kDa | 4181 | 4182 | 4183 | 4184 | 4185 | 4186 | 4187 | 4188 | 4189 | 4190 |
| 120 kDa | 4191 | 4192 | 4193 | 4194 | 4195 | 4196 | 4197 | 4198 | 4199 | 4200 |
| 121 kDa | 4201 | 4202 | 4203 | 4204 | 4205 | 4206 | 4207 | 4208 | 4209 | 4210 |
| 122 kDa | 4211 | 4212 | 4213 | 4214 | 4215 | 4216 | 4217 | 4218 | 4219 | 4220 |
| 123 kDa | 4221 | 4222 | 4223 | 4224 | 4225 | 4226 | 4227 | 4228 | 4229 | 4230 |
| 124 kDa | 4231 | 4232 | 4233 | 4234 | 4235 | 4236 | 4237 | 4238 | 4239 | 4240 |
| 125 kDa | 4241 | 4242 | 4243 | 4244 | 4245 | 4246 | 4247 | 4248 | 4249 | 4250 |
| 126 kDa | 4251 | 4252 | 4253 | 4254 | 4255 | 4256 | 4257 | 4258 | 4259 | 4260 |
| 127 kDa | 4261 | 4262 | 4263 | 4264 | 4265 | 4266 | 4267 | 4268 | 4269 | 4270 |
| 128 kDa | 4271 | 4272 | 4273 | 4274 | 4275 | 4276 | 4277 | 4278 | 4279 | 4280 |
| 129 kDa | 4281 | 4282 | 4283 | 4284 | 4285 | 4286 | 4287 | 4288 | 4289 | 4290 |
| 130 kDa | 4291 | 4292 | 4293 | 4294 | 4295 | 4296 | 4297 | 4298 | 4299 | 4300 |
| 131 kDa | 4301 | 4302 | 4303 | 4304 | 4305 | 4306 | 4307 | 4308 | 4309 | 4310 |
| 132 kDa | 4311 | 4312 | 4313 | 4314 | 4315 | 4316 | 4317 | 4318 | 4319 | 4320 |
| 133 kDa | 4321 | 4322 | 4323 | 4324 | 4325 | 4326 | 4327 | 4328 | 4329 | 4330 |
| 134 kDa | 4331 | 4332 | 4333 | 4334 | 4335 | 4336 | 4337 | 4338 | 4339 | 4340 |
| 135 kDa | 4341 | 4342 | 4343 | 4344 | 4345 | 4346 | 4347 | 4348 | 4349 | 4350 |
| 136 kDa | 4351 | 4352 | 4353 | 4354 | 4355 | 4356 | 4357 | 4358 | 4359 | 4360 |
| 137 kDa | 4361 | 4362 | 4363 | 4364 | 4365 | 4366 | 4367 | 4368 | 4369 | 4370 |
| 138 kDa | 4371 | 4372 | 4373 | 4374 | 4375 | 4376 | 4377 | 4378 | 4379 | 4380 |
| 139 kDa | 4381 | 4382 | 4383 | 4384 | 4385 | 4386 | 4387 | 4388 | 4389 | 4390 |
| 140 kDa | 4391 | 4392 | 4393 | 4394 | 4395 | 4396 | 4397 | 4398 | 4399 | 4400 |
| 141 kDa | 4401 | 4402 | 4403 | 4404 | 4405 | 4406 | 4407 | 4408 | 4409 | 4410 |
| 142 kDa | 4411 | 4412 | 4413 | 4414 | 4415 | 4416 | 4417 | 4418 | 4419 | 4420 |
| 143 kDa | 4421 | 4422 | 4423 | 4424 | 4425 | 4426 | 4427 | 4428 | 4429 | 4430 |
| 144 kDa | 4431 | 4432 | 4433 | 4434 | 4435 | 4436 | 4437 | 4438 | 4439 | 4440 |
| 145 kDa | 4441 | 4442 | 4443 | 4444 | 4445 | 4446 | 4447 | 4448 | 4449 | 4450 |

In some embodiments, one or more RSPF mixture compositions (e.g., solutions) selected from compositions #5001 to #6595, having weight average molecular weights selected from about 1 kDa to about 145 kDa, and a polydispersity selected about 1, about 1.1, about 1.2, about 1.3, about 1.4, about 1.5, about 1.6, about 1.7, about 1.8, about 1.9, and about 2, are created and used as described herein:

| MW | PD1 (about) | | | | | | | | | | |
|---|---|---|---|---|---|---|---|---|---|---|---|
| (about) | 1.0 | 1.1 | 1.2 | 1.3 | 1.4 | 1.5 | 1.6 | 1.7 | 1.8 | 1.9 | 2.0 |
| 1 kDa | 5001 | 5002 | 5003 | 5004 | 5005 | 5006 | 5007 | 5008 | 5009 | 5010 | 5011 |
| 2 kDa | 5012 | 5013 | 5014 | 5015 | 5016 | 5017 | 5018 | 5019 | 5020 | 5021 | 5022 |
| 3 kDa | 5023 | 5024 | 5025 | 5026 | 5027 | 5028 | 5029 | 5030 | 5031 | 5032 | 5033 |
| 4 kDa | 5034 | 5035 | 5036 | 5037 | 5038 | 5039 | 5040 | 5041 | 5042 | 5043 | 5044 |
| 5 kDa | 5045 | 5046 | 5047 | 5048 | 5049 | 5050 | 5051 | 5052 | 5053 | 5054 | 5055 |
| 6 kDa | 5056 | 5057 | 5058 | 5059 | 5060 | 5061 | 5062 | 5063 | 5064 | 5065 | 5066 |
| 7 kDa | 5067 | 5068 | 5069 | 5070 | 5071 | 5072 | 5073 | 5074 | 5075 | 5076 | 5077 |
| 8 kDa | 5078 | 5079 | 5080 | 5081 | 5082 | 5083 | 5084 | 5085 | 5086 | 5087 | 5088 |
| 9 kDa | 5089 | 5090 | 5091 | 5092 | 5093 | 5094 | 5095 | 5096 | 5097 | 5098 | 5099 |
| 10 kDa | 5100 | 5101 | 5102 | 5103 | 5104 | 5105 | 5106 | 5107 | 5108 | 5109 | 5110 |
| 11 kDa | 5111 | 5112 | 5113 | 5114 | 5115 | 5116 | 5117 | 5118 | 5119 | 5120 | 5121 |
| 12 kDa | 5122 | 5123 | 5124 | 5125 | 5126 | 5127 | 5128 | 5129 | 5130 | 5131 | 5132 |
| 13 kDa | 5133 | 5134 | 5135 | 5136 | 5137 | 5138 | 5139 | 5140 | 5141 | 5142 | 5143 |
| 14 kDa | 5144 | 5145 | 5146 | 5147 | 5148 | 5149 | 5150 | 5151 | 5152 | 5153 | 5154 |
| 15 kDa | 5155 | 5156 | 5157 | 5158 | 5159 | 5160 | 5161 | 5162 | 5163 | 5164 | 5165 |
| 16 kDa | 5166 | 5167 | 5168 | 5169 | 5170 | 5171 | 5172 | 5173 | 5174 | 5175 | 5176 |
| 17 kDa | 5177 | 5178 | 5179 | 5180 | 5181 | 5182 | 5183 | 5184 | 5185 | 5186 | 5187 |
| 18 kDa | 5188 | 5189 | 5190 | 5191 | 5192 | 5193 | 5194 | 5195 | 5196 | 5197 | 5198 |
| 19 kDa | 5199 | 5200 | 5201 | 5202 | 5203 | 5204 | 5205 | 5206 | 5207 | 5208 | 5209 |
| 20 kDa | 5210 | 5211 | 5212 | 5213 | 5214 | 5215 | 5216 | 5217 | 5218 | 5219 | 5220 |
| 21 kDa | 5221 | 5222 | 5223 | 5224 | 5225 | 5226 | 5227 | 5228 | 5229 | 5230 | 5231 |
| 22 kDa | 5232 | 5233 | 5234 | 5235 | 5236 | 5237 | 5238 | 5239 | 5240 | 5241 | 5242 |
| 23 kDa | 5243 | 5244 | 5245 | 5246 | 5247 | 5248 | 5249 | 5250 | 5251 | 5252 | 5253 |
| 24 kDa | 5254 | 5255 | 5256 | 5257 | 5258 | 5259 | 5260 | 5261 | 5262 | 5263 | 5264 |
| 25 kDa | 5265 | 5266 | 5267 | 5268 | 5269 | 5270 | 5271 | 5272 | 5273 | 5274 | 5275 |
| 26 kDa | 5276 | 5277 | 5278 | 5279 | 5280 | 5281 | 5282 | 5283 | 5284 | 5285 | 5286 |
| 27 kDa | 5287 | 5288 | 5289 | 5290 | 5291 | 5292 | 5293 | 5294 | 5295 | 5296 | 5297 |
| 28 kDa | 5298 | 5299 | 5300 | 5301 | 5302 | 5303 | 5304 | 5305 | 5306 | 5307 | 5308 |
| 29 kDa | 5309 | 5310 | 5311 | 5312 | 5313 | 5314 | 5315 | 5316 | 5317 | 5318 | 5319 |
| 30 kDa | 5320 | 5321 | 5322 | 5323 | 5324 | 5325 | 5326 | 5327 | 5328 | 5329 | 5330 |
| 31 kDa | 5331 | 5332 | 5333 | 5334 | 5335 | 5336 | 5337 | 5338 | 5339 | 5340 | 5341 |
| 32 kDa | 5342 | 5343 | 5344 | 5345 | 5346 | 5347 | 5348 | 5349 | 5350 | 5351 | 5352 |
| 33 kDa | 5353 | 5354 | 5355 | 5356 | 5357 | 5358 | 5359 | 5360 | 5361 | 5362 | 5363 |
| 34 kDa | 5364 | 5365 | 5366 | 5367 | 5368 | 5369 | 5370 | 5371 | 5372 | 5373 | 5374 |
| 35 kDa | 5375 | 5376 | 5377 | 5378 | 5379 | 5380 | 5381 | 5382 | 5383 | 5384 | 5385 |
| 36 kDa | 5386 | 5387 | 5388 | 5389 | 5390 | 5391 | 5392 | 5393 | 5394 | 5395 | 5396 |
| 37 kDa | 5397 | 5398 | 5399 | 5400 | 5401 | 5402 | 5403 | 5404 | 5405 | 5406 | 5407 |
| 38 kDa | 5408 | 5409 | 5410 | 5411 | 5412 | 5413 | 5414 | 5415 | 5416 | 5417 | 5418 |
| 39 kDa | 5419 | 5420 | 5421 | 5422 | 5423 | 5424 | 5425 | 5426 | 5427 | 5428 | 5429 |
| 40 kDa | 5430 | 5431 | 5432 | 5433 | 5434 | 5435 | 5436 | 5437 | 5438 | 5439 | 5440 |
| 41 kDa | 5441 | 5442 | 5443 | 5444 | 5445 | 5446 | 5447 | 5448 | 5449 | 5450 | 5451 |
| 42 kDa | 5452 | 5453 | 5454 | 5455 | 5456 | 5457 | 5458 | 5459 | 5460 | 5461 | 5462 |
| 43 kDa | 5463 | 5464 | 5465 | 5466 | 5467 | 5468 | 5469 | 5470 | 5471 | 5472 | 5473 |
| 44 kDa | 5474 | 5475 | 5476 | 5477 | 5478 | 5479 | 5480 | 5481 | 5482 | 5483 | 5484 |
| 45 kDa | 5485 | 5486 | 5487 | 5488 | 5489 | 5490 | 5491 | 5492 | 5493 | 5494 | 5495 |
| 46 kDa | 5496 | 5497 | 5498 | 5499 | 5500 | 5501 | 5502 | 5503 | 5504 | 5505 | 5506 |
| 47 kDa | 5507 | 5508 | 5509 | 5510 | 5511 | 5512 | 5513 | 5514 | 5515 | 5516 | 5517 |
| 48 kDa | 5518 | 5519 | 5520 | 5521 | 5522 | 5523 | 5524 | 5525 | 5526 | 5527 | 5528 |
| 49 kDa | 5529 | 5530 | 5531 | 5532 | 5533 | 5534 | 5535 | 5536 | 5537 | 5538 | 5539 |
| 50 kDa | 5540 | 5541 | 5542 | 5543 | 5544 | 5545 | 5546 | 5547 | 5548 | 5549 | 5550 |
| 51 kDa | 5551 | 5552 | 5553 | 5554 | 5555 | 5556 | 5557 | 5558 | 5559 | 5560 | 5561 |
| 52 kDa | 5562 | 5563 | 5564 | 5565 | 5566 | 5567 | 5568 | 5569 | 5570 | 5571 | 5572 |
| 53 kDa | 5573 | 5574 | 5575 | 5576 | 5577 | 5578 | 5579 | 5580 | 5581 | 5582 | 5583 |
| 54 kDa | 5584 | 5585 | 5586 | 5587 | 5588 | 5589 | 5590 | 5591 | 5592 | 5593 | 5594 |
| 55 kDa | 5595 | 5596 | 5597 | 5598 | 5599 | 5600 | 5601 | 5602 | 5603 | 5604 | 5605 |
| 56 kDa | 5606 | 5607 | 5608 | 5609 | 5610 | 5611 | 5612 | 5613 | 5614 | 5615 | 5616 |
| 57 kDa | 5617 | 5618 | 5619 | 5620 | 5621 | 5622 | 5623 | 5624 | 5625 | 5626 | 5627 |
| 58 kDa | 5628 | 5629 | 5630 | 5631 | 5632 | 5633 | 5634 | 5635 | 5636 | 5637 | 5638 |
| 59 kDa | 5639 | 5640 | 5641 | 5642 | 5643 | 5644 | 5645 | 5646 | 5647 | 5648 | 5649 |
| 60 kDa | 5650 | 5651 | 5652 | 5653 | 5654 | 5655 | 5656 | 5657 | 5658 | 5659 | 5660 |
| 61 kDa | 5661 | 5662 | 5663 | 5664 | 5665 | 5666 | 5667 | 5668 | 5669 | 5670 | 5671 |
| 62 kDa | 5672 | 5673 | 5674 | 5675 | 5676 | 5677 | 5678 | 5679 | 5680 | 5681 | 5682 |
| 63 kDa | 5683 | 5684 | 5685 | 5686 | 5687 | 5688 | 5689 | 5690 | 5691 | 5692 | 5693 |
| 64 kDa | 5694 | 5695 | 5696 | 5697 | 5698 | 5699 | 5700 | 5701 | 5702 | 5703 | 5704 |
| 65 kDa | 5705 | 5706 | 5707 | 5708 | 5709 | 5710 | 5711 | 5712 | 5713 | 5714 | 5715 |
| 66 kDa | 5716 | 5717 | 5718 | 5719 | 5720 | 5721 | 5722 | 5723 | 5724 | 5725 | 5726 |
| 67 kDa | 5727 | 5728 | 5729 | 5730 | 5731 | 5732 | 5733 | 5734 | 5735 | 5736 | 5737 |
| 68 kDa | 5738 | 5739 | 5740 | 5741 | 5742 | 5743 | 5744 | 5745 | 5746 | 5747 | 5748 |
| 69 kDa | 5749 | 5750 | 5751 | 5752 | 5753 | 5754 | 5755 | 5756 | 5757 | 5758 | 5759 |
| 70 kDa | 5760 | 5761 | 5762 | 5763 | 5764 | 5765 | 5766 | 5767 | 5768 | 5769 | 5770 |
| 71 kDa | 5771 | 5772 | 5773 | 5774 | 5775 | 5776 | 5777 | 5778 | 5779 | 5780 | 5781 |
| 72 kDa | 5782 | 5783 | 5784 | 5785 | 5786 | 5787 | 5788 | 5789 | 5790 | 5791 | 5792 |
| 73 kDa | 5793 | 5794 | 5795 | 5796 | 5797 | 5798 | 5799 | 5800 | 5801 | 5802 | 5803 |
| 74 kDa | 5804 | 5805 | 5806 | 5807 | 5808 | 5809 | 5810 | 5811 | 5812 | 5813 | 5814 |
| 75 kDa | 5815 | 5816 | 5817 | 5818 | 5819 | 5820 | 5821 | 5822 | 5823 | 5824 | 5825 |
| 76 kDa | 5826 | 5827 | 5828 | 5829 | 5830 | 5831 | 5832 | 5833 | 5834 | 5835 | 5836 |
| 77 kDa | 5837 | 5838 | 5839 | 5840 | 5841 | 5842 | 5843 | 5844 | 5845 | 5846 | 5847 |
| 78 kDa | 5848 | 5849 | 5850 | 5851 | 5852 | 5853 | 5854 | 5855 | 5856 | 5857 | 5858 |
| 79 kDa | 5859 | 5860 | 5861 | 5862 | 5863 | 5864 | 5865 | 5866 | 5867 | 5868 | 5869 |
| 80 kDa | 5870 | 5871 | 5872 | 5873 | 5874 | 5875 | 5876 | 5877 | 5878 | 5879 | 5880 |
| 81 kDa | 5881 | 5882 | 5883 | 5884 | 5885 | 5886 | 5887 | 5888 | 5889 | 5890 | 5891 |
| 82 kDa | 5892 | 5893 | 5894 | 5895 | 5896 | 5897 | 5898 | 5899 | 5900 | 5901 | 5902 |
| 83 kDa | 5903 | 5904 | 5905 | 5906 | 5907 | 5908 | 5909 | 5910 | 5911 | 5912 | 5913 |
| 84 kDa | 5914 | 5915 | 5916 | 5917 | 5918 | 5919 | 5920 | 5921 | 5922 | 5923 | 5924 |
| 85 kDa | 5925 | 5926 | 5927 | 5928 | 5929 | 5930 | 5931 | 5932 | 5933 | 5934 | 5935 |
| 86 kDa | 5936 | 5937 | 5938 | 5939 | 5940 | 5941 | 5942 | 5943 | 5944 | 5945 | 5946 |
| 87 kDa | 5947 | 5948 | 5949 | 5950 | 5951 | 5952 | 5953 | 5954 | 5955 | 5956 | 5957 |
| 88 kDa | 5958 | 5959 | 5960 | 5961 | 5962 | 5963 | 5964 | 5965 | 5966 | 5967 | 5968 |
| 89 kDa | 5969 | 5970 | 5971 | 5972 | 5973 | 5974 | 5975 | 5976 | 5977 | 5978 | 5979 |
| 90 kDa | 5980 | 5981 | 5982 | 5983 | 5984 | 5985 | 5986 | 5987 | 5988 | 5989 | 5990 |
| 91 kDa | 5991 | 5992 | 5993 | 5994 | 5995 | 5996 | 5997 | 5998 | 5999 | 6000 | 6001 |
| 92 kDa | 6002 | 6003 | 6004 | 6005 | 6006 | 6007 | 6008 | 6009 | 6010 | 6011 | 6012 |
| 93 kDa | 6013 | 6014 | 6015 | 6016 | 6017 | 6018 | 6019 | 6020 | 6021 | 6022 | 6023 |
| 94 kDa | 6024 | 6025 | 6026 | 6027 | 6028 | 6029 | 6030 | 6031 | 6032 | 6033 | 6034 |
| 95 kDa | 6035 | 6036 | 6037 | 6038 | 6039 | 6040 | 6041 | 6042 | 6043 | 6044 | 6045 |
| 96 kDa | 6046 | 6047 | 6048 | 6049 | 6050 | 6051 | 6052 | 6053 | 6054 | 6055 | 6056 |
| 97 kDa | 6057 | 6058 | 6059 | 6060 | 6061 | 6062 | 6063 | 6064 | 6065 | 6066 | 6067 |
| 98 kDa | 6068 | 6069 | 6070 | 6071 | 6072 | 6073 | 6074 | 6075 | 6076 | 6077 | 6078 |
| 99 kDa | 6079 | 6080 | 6081 | 6082 | 6083 | 6084 | 6085 | 6086 | 6087 | 6088 | 6089 |
| 100 kDa | 6090 | 6091 | 6092 | 6093 | 6094 | 6095 | 6096 | 6097 | 6098 | 6099 | 6100 |
| 101 kDa | 6101 | 6102 | 6103 | 6104 | 6105 | 6106 | 6107 | 6108 | 6109 | 6110 | 6111 |
| 102 kDa | 6112 | 6113 | 6114 | 6115 | 6116 | 6117 | 6118 | 6119 | 6120 | 6121 | 6122 |
| 103 kDa | 6123 | 6124 | 6125 | 6126 | 6127 | 6128 | 6129 | 6130 | 6131 | 6132 | 6133 |
| 104 kDa | 6134 | 6135 | 6136 | 6137 | 6138 | 6139 | 6140 | 6141 | 6142 | 6143 | 6144 |
| 105 kDa | 6145 | 6146 | 6147 | 6148 | 6149 | 6150 | 6151 | 6152 | 6153 | 6154 | 6155 |
| 106 kDa | 6156 | 6157 | 6158 | 6159 | 6160 | 6161 | 6162 | 6163 | 6164 | 6165 | 6166 |
| 107 kDa | 6167 | 6168 | 6169 | 6170 | 6171 | 6172 | 6173 | 6174 | 6175 | 6176 | 6177 |
| 108 kDa | 6178 | 6179 | 6180 | 6181 | 6182 | 6183 | 6184 | 6185 | 6186 | 6187 | 6188 |
| 109 kDa | 6189 | 6190 | 6191 | 6192 | 6193 | 6194 | 6195 | 6196 | 6197 | 6198 | 6199 |
| 110 kDa | 6200 | 6201 | 6202 | 6203 | 6204 | 6205 | 6206 | 6207 | 6208 | 6209 | 6210 |
| 111 kDa | 6211 | 6212 | 6213 | 6214 | 6215 | 6216 | 6217 | 6218 | 6219 | 6220 | 6221 |
| 112 kDa | 6222 | 6223 | 6224 | 6225 | 6226 | 6227 | 6228 | 6229 | 6230 | 6231 | 6232 |
| 113 kDa | 6233 | 6234 | 6235 | 6236 | 6237 | 6238 | 6239 | 6240 | 6241 | 6242 | 6243 |
| 114 kDa | 6244 | 6245 | 6246 | 6247 | 6248 | 6249 | 6250 | 6251 | 6252 | 6253 | 6254 |
| 115 kDa | 6255 | 6256 | 6257 | 6258 | 6259 | 6260 | 6261 | 6262 | 6263 | 6264 | 6265 |
| 116 kDa | 6266 | 6267 | 6268 | 6269 | 6270 | 6271 | 6272 | 6273 | 6274 | 6275 | 6276 |
| 117 kDa | 6277 | 6278 | 6279 | 6280 | 6281 | 6282 | 6283 | 6284 | 6285 | 6286 | 6287 |
| 118 kDa | 6288 | 6289 | 6290 | 6291 | 6292 | 6293 | 6294 | 6295 | 6296 | 6297 | 6298 |
| 119 kDa | 6299 | 6300 | 6301 | 6302 | 6303 | 6304 | 6305 | 6306 | 6307 | 6308 | 6309 |
| 120 kDa | 6310 | 6311 | 6312 | 6313 | 6314 | 6315 | 6316 | 6317 | 6318 | 6319 | 6320 |
| 121 kDa | 6321 | 6322 | 6323 | 6324 | 6325 | 6326 | 6327 | 6328 | 6329 | 6330 | 6331 |
| 122 kDa | 6332 | 6333 | 6334 | 6335 | 6336 | 6337 | 6338 | 6339 | 6340 | 6341 | 6342 |
| 123 kDa | 6343 | 6344 | 6345 | 6346 | 6347 | 6348 | 6349 | 6350 | 6351 | 6352 | 6353 |
| 124 kDa | 6354 | 6355 | 6356 | 6357 | 6358 | 6359 | 6360 | 6361 | 6362 | 6363 | 6364 |
| 125 kDa | 6365 | 6366 | 6367 | 6368 | 6369 | 6370 | 6371 | 6372 | 6373 | 6374 | 6375 |
| 126 kDa | 6376 | 6377 | 6378 | 6379 | 6380 | 6381 | 6382 | 6383 | 6384 | 6385 | 6386 |
| 127 kDa | 6387 | 6388 | 6389 | 6390 | 6391 | 6392 | 6393 | 6394 | 6395 | 6396 | 6397 |
| 128 kDa | 6398 | 6399 | 6400 | 6401 | 6402 | 6403 | 6404 | 6405 | 6406 | 6407 | 6408 |
| 129 kDa | 6409 | 6410 | 6411 | 6412 | 6413 | 6414 | 6415 | 6416 | 6417 | 6418 | 6419 |
| 130 kDa | 6420 | 6421 | 6422 | 6423 | 6424 | 6425 | 6426 | 6427 | 6428 | 6429 | 6430 |
| 131 kDa | 6431 | 6432 | 6433 | 6434 | 6435 | 6436 | 6437 | 6438 | 6439 | 6440 | 6441 |
| 132 kDa | 6442 | 6443 | 6444 | 6445 | 6446 | 6447 | 6448 | 6449 | 6450 | 6451 | 6452 |
| 133 kDa | 6453 | 6454 | 6455 | 6456 | 6457 | 6458 | 6459 | 6460 | 6461 | 6462 | 6463 |
| 134 kDa | 6464 | 6465 | 6466 | 6467 | 6468 | 6469 | 6470 | 6471 | 6472 | 6473 | 6474 |
| 135 kDa | 6475 | 6476 | 6477 | 6478 | 6479 | 6480 | 6481 | 6482 | 6483 | 6484 | 6485 |
| 136 kDa | 6486 | 6487 | 6488 | 6489 | 6490 | 6491 | 6492 | 6493 | 6494 | 6495 | 6496 |
| 137 kDa | 6497 | 6498 | 6499 | 6500 | 6501 | 6502 | 6503 | 6504 | 6505 | 6506 | 6507 |
| 138 kDa | 6508 | 6509 | 6510 | 6511 | 6512 | 6513 | 6514 | 6515 | 6516 | 6517 | 6518 |
| 139 kDa | 6519 | 6520 | 6521 | 6522 | 6523 | 6524 | 6525 | 6526 | 6527 | 6528 | 6529 |
| 140 kDa | 6530 | 6531 | 6532 | 6533 | 6534 | 6535 | 6536 | 6537 | 6538 | 6539 | 6540 |
| 141 kDa | 6541 | 6542 | 6543 | 6544 | 6545 | 6546 | 6547 | 6548 | 6549 | 6550 | 6551 |
| 142 kDa | 6552 | 6553 | 6554 | 6555 | 6556 | 6557 | 6558 | 6559 | 6560 | 6561 | 6562 |
| 143 kDa | 6563 | 6564 | 6565 | 6566 | 6567 | 6568 | 6569 | 6570 | 6571 | 6572 | 6573 |
| 144 kDa | 6574 | 6575 | 6576 | 6577 | 6578 | 6579 | 6580 | 6581 | 6582 | 6583 | 6584 |
| 145 kDa | 6585 | 6586 | 6587 | 6588 | 6589 | 6590 | 6591 | 6592 | 6593 | 6594 | 6595 |

According to aspects illustrated herein, there is disclosed a composition that includes recombinant silk-based protein fragments, wherein the composition has an average weight average molecular weight ranging from about 6 kDa to about 17 kDa, wherein the composition has a polydispersity of between 1 and about 5.0, or between about 1.5 and about 3.0, wherein the composition is substantially homogenous, wherein the composition includes between 0 ppm and about 500 ppm of inorganic residuals, and wherein the composition includes between 0 ppm and about 500 ppm of organic residuals. In an embodiment, the recombinant silk-based protein fragments have between about 10 ppm and about 300 ppm of lithium bromide residuals and between about 10 ppm and about 100 ppm of sodium carbonate residuals. In an embodiment, the lithium bromide residuals are measurable using a high-performance liquid chromatography lithium bromide assay, and the sodium carbonate residuals are measurable using a high-performance liquid chromatography sodium carbonate assay. In an embodiment, the composition further includes less than 10 wt. % water. In an embodiment, the composition is in the form of a solution. In an embodiment, the composition includes from about 0.01 wt % to about 30.0 wt % recombinant silk-based protein fragments. The recombinant silk-based protein fragments are stable in the solution for at least 30 days. In an embodiment, the term “stable” refers to the absence of spontaneous or gradual gelation, with no visible change in the color or turbidity of the solution. In an embodiment, the term “stable” refers to no aggregation of fragments and therefore no increase in molecular weight over time. In an embodiment, the composition is in the form of an aqueous solution. In an embodiment, the composition is in the form of an organic solution. The composition may be provided in a sealed container. In some embodiments, the composition further includes one or more molecules selected from the group consisting of therapeutic agents, growth factors, antioxidants, proteins, vitamins, carbohydrates, polymers, nucleic acids, salts, acids, bases, biomolecules, glycosamino glycans, polysaccharides, extracellular matrix molecules, metals, metal ion, metal oxide, synthetic molecules, polyanhydrides, cells, fatty acids, fragrance, minerals, plants, plant extracts, preservatives and essential oils. In an embodiment, the added molecule or molecules are stable (i.e., retain activity over time) within the composition and can be released at a desired rate. In an embodiment, the one or more molecules is vitamin C or a derivative thereof. In an embodiment, the composition further includes an alpha hydroxy acid selected from the group consisting of glycolic acid, lactic acid, tartaric acid and citric acid. In an embodiment, the composition further includes hyaluronic acid or its salt form at a concentration of about 0.5% to about 10.0 wt. % to about 30.0 wt. % recombinant silk-based protein fragments.

According to aspects illustrated herein, there is disclosed a composition that includes recombinant silk-based protein fragments, wherein the composition has an average weight average molecular weight ranging from about 17 kDa to about 39 kDa, wherein the composition has a polydispersity of between 1 and about 5.0, or between about 1.5 and about 3.0, wherein the composition is substantially homogenous, wherein the composition includes between 0 ppm and about 500 ppm of inorganic residuals, and wherein the composition includes between 0 ppm and about 500 ppm of organic residuals. In an embodiment, the recombinant silk-based protein fragments have between about 10 ppm and about 300 ppm of lithium bromide residuals and between about 10 ppm and about 100 ppm of sodium carbonate residuals. In an embodiment, the lithium bromide residuals are measurable using a high-performance liquid chromatography lithium bromide assay, and the sodium carbonate residuals are measurable using a high-performance liquid chromatography sodium carbonate assay. In an embodiment, the composition further includes less than 10% water. In an embodiment, the composition is in the form of a solution. In an embodiment, the composition includes from about 0.01 wt % to about 30.0 wt % recombinant silk-based protein fragments. The recombinant silk-based protein fragments are stable in the solution for at least 30 days. In an embodiment, the term “stable” refers to the absence of spontaneous or gradual gelation, with no visible change in the color or turbidity of the solution. In an embodiment, the term “stable” refers to no aggregation of fragments and therefore no increase in molecular weight over time. In an embodiment, the composition is in the form of an aqueous solution. In an embodiment, the composition is in the form of an organic solution. The composition may be provided in a sealed container. In some embodiments, the composition further includes one or more molecules selected from the group consisting of therapeutic agents, growth factors, antioxidants, proteins, vitamins, carbohydrates, polymers, nucleic acids, salts, acids, bases, biomolecules, glycosamino glycans, polysaccharides, extracellular matrix molecules, metals, metal ion, metal oxide, synthetic molecules, polyanhydrides, cells, fatty acids, fragrance, minerals, plants, plant extracts, preservatives and essential oils. In an embodiment, the added molecule or molecules are stable (i.e., retain activity over time) within the composition and can be released at a desired rate. In an embodiment, the one or more molecules is vitamin C or a derivative thereof. In an embodiment, the composition further includes an alpha hydroxy acid selected from the group consisting of glycolic acid, lactic acid, tartaric acid and citric acid. In an embodiment, the composition further includes hyaluronic acid or its salt form at a concentration of about 0.5 wt. % to about 10.0 wt. %. In an embodiment, the composition further includes at least one of zinc oxide or titanium dioxide. In an embodiment, the recombinant silk-based protein fragments in the composition are hypoallergenic. In an embodiment, the recombinant silk-based protein fragments are biocompatible, non-sensitizing, and non-immunogenic.

According to aspects illustrated herein, there is disclosed a composition that includes recombinant silk-based protein fragments, wherein the composition has a weight average molecular weight ranging from about 39 kDa to about 80 kDa, wherein the composition has a polydispersity of between 1 and about 5.0, or between about 1.5 and about 3.0, wherein the composition is substantially homogenous, wherein the composition includes between 0 ppm and about 500 ppm of inorganic residuals, and wherein the composition includes between 0 ppm and about 500 ppm of organic residuals. In an embodiment, the recombinant silk-based protein fragments have between about 10 ppm and about 300 ppm of lithium bromide residuals and between about 10 ppm and about 100 ppm of sodium carbonate residuals. In an embodiment, the lithium bromide residuals are measurable using a high-performance liquid chromatography lithium bromide assay, and the sodium carbonate residuals are measurable using a high-performance liquid chromatography sodium carbonate assay. In an embodiment, the composition further includes less than 10% water. In an embodiment, the composition is in the form of a solution. In an embodiment, the composition includes from about 0.01 wt. % to about 30.0 wt. % recombinant silk-based protein fragments. The recombinant silk-based protein fragments are stable in the solution for at least 30 days. In an embodiment, the term “stable” refers to the absence of spontaneous or gradual gelation, with no visible change in the color or turbidity of the solution. In an embodiment, the term “stable” refers to no aggregation of fragments and therefore no increase in molecular weight over time. In an embodiment, the composition is in the form of an aqueous solution. In an embodiment, the composition is in the form of an organic solution. The composition may be provided in a sealed container. In some embodiments, the composition further includes one or more molecules selected from the group consisting of therapeutic agents, growth factors, antioxidants, proteins, vitamins, carbohydrates, polymers, nucleic acids, salts, acids, bases, biomolecules, glycosamino glycans, polysaccharides, extracellular matrix molecules, metals, metal ion, metal oxide, synthetic molecules, polyanhydrides, cells, fatty acids, fragrance, minerals, plants, plant extracts, preservatives and essential oils. In an embodiment, the added molecule or molecules are stable (i.e., retain activity over time) within the composition and can be released at a desired rate. In an embodiment, the one or more molecules is vitamin C or a derivative thereof. In an embodiment, the composition further includes an alpha hydroxy acid selected from the group consisting of glycolic acid, lactic acid, tartaric acid and citric acid. In an embodiment, the composition further includes hyaluronic acid or its salt form at a concentration of about 0.5 wt. % to about 10.0 wt. %. In an embodiment, the composition further includes at least one of zinc oxide or titanium dioxide. In an embodiment, the recombinant silk-based protein fragments in the composition are hypoallergenic. In an embodiment, the recombinant silk-based protein fragments are biocompatible, non-sensitizing, and non-immunogenic.

In an embodiment, the composition further includes at least one of zinc oxide or titanium dioxide. In an embodiment, the recombinant silk-based protein fragments in the composition are hypoallergenic. In an embodiment, the recombinant silk-based protein fragments are biocompatible, non-sensitizing, and non-immunogenic.

According to aspects illustrated herein, there is disclosed a gel that includes recombinant silk-based protein fragments comprising: an average weight average molecular weight ranging from about 6 kDa to about 17 kDa, or from about 17 kDa to about 39 kDa, or from about 39 kDa to about 80 kDa; and a polydispersity of between 1 and about 5.0, or between about 1.5 and about 3.0; and water from about 20 wt. % to about 99.9 wt. %, wherein the gel includes between 0 ppm and 500 ppm of inorganic residuals, and wherein the gel includes between 0 ppm and 500 ppm of organic residuals. In an embodiment, the gel includes between about 1.0% and about 50.0% crystalline protein domains. In an embodiment, the gel includes from about 0.1 wt. % to about 6.0 wt. % of recombinant silk-based protein fragments. In an embodiment, the gel has a pH from about 1.0 to about 7.0. In an embodiment, the gel further includes from about 0.5 wt. % to about 20.0 wt. % of vitamin C or a derivative thereof. In an embodiment, the vitamin C or a derivative thereof remains stable within the gel for a period of from about 5 days to about 5 years. In an embodiment, the vitamin C or a derivative thereof is stable within the gel so as to result in release of the vitamin C in a biologically active form. In an embodiment, the gel further includes an additive selected from the group consisting of vitamin E, rosemary oil, rose oil, lemon juice, lemon grass oil and caffeine. In an embodiment, the gel is packaged in an airtight container. In an embodiment, the recombinant silk-based protein fragments are hypoallergenic. In an embodiment, the gel has less than 10 colony forming units per milliliter.

According to aspects illustrated herein, a method is disclosed for producing recombinant silk gels having entrapped molecules or therapeutic agents such as those listed in the following paragraphs. In an embodiment, at least one molecule or therapeutic agent of interest is physically entrapped into a RSPF mixture solution of the present disclosure (including, but not limited to, as provided in any of the compositions #1001-2450, #3001-4450, and #5001-6595) during processing into aqueous gels. An aqueous recombinant silk gel of the present disclosure can be used to release at least one molecule or therapeutic agent of interest.

According to aspects illustrated herein, recombinant silk-based protein fragments from aqueous solutions of the present disclosure can be formed into yarns and fabrics including for example, woven or weaved fabrics, and these fabrics can be used in textiles, as described above.

According to aspects illustrated herein, recombinant silk fabric manufactured from RSPF mixture solutions of the present disclosure are disclosed. In an embodiment, at least one molecule or therapeutic agent of interest is physically entrapped into a RSPF mixture solution of the present disclosure. A recombinant silk film of the present disclosure can be used to release at least one molecule or therapeutic agent of interest.

In some embodiments, the disclosure may include an article having a fiber or yarn having a coating, wherein the coating may include recombinant silk based proteins or fragments thereof having a weight average molecular weight, or average weight average molecular weight range of about 5 kDa to about 144 kDa. In some embodiments, the article may be a fabric.

In some embodiments, the recombinant silk based proteins or fragments may include silk and a copolymer.

In some embodiments, the recombinant silk based proteins or protein fragments thereof may have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof may have a polydispersity of between 1.0 and about 5.0.

In some embodiments, the fiber or yarn may be selected from the group consisting of natural fiber or yarn, synthetic fiber or yarn, or combinations thereof.

In some embodiments, the fiber or yarn may be natural fiber or yarn selected from the group consisting of cotton, alpaca fleece, alpaca wool, lama fleece, lama wool, cotton, cashmere, sheep fleece, sheep wool, and combinations thereof.

In some embodiments, the fiber or yarn may be synthetic fiber or yarn selected from the group consisting of polyester, nylon, polyester-polyurethane copolymer, and combinations thereof.

In some embodiments, the fabric may exhibit an improved property, wherein the improved property may be an accumulative one-way moisture transport index selected from the group consisting of greater than 40%, greater than 60%, greater than 80%, greater than 100%, greater than 120%, greater than 140%, greater than 160%, and greater than 180%.

In some embodiments, the fabric may exhibit an improved property, wherein the improved property may be an accumulative one way transport capability increase relative to uncoated fabric selected from the group consisting of 1.2 fold, 1.5 fold, 2.0 fold, 3.0 fold, 4.0 fold, 5.0 fold, and 10 fold.

In some embodiments, the fabric may exhibit an improved property, wherein the improved property may be an overall moisture management capability selected from the group consisting of greater than 0.05, greater than 0.10, greater than 0.15, greater than 0.20, greater than 0.25, greater than 0.30, greater than 0.35, greater than 0.40, greater than 0.50, greater than 0.60, greater than 0.70, and greater than 0.80. In some embodiments, the improved property may be determined after a period of machine washing cycles selected from the group consisting of 5 cycles, 10 cycles, 25 cycles, and 50 cycles.

In some embodiments, the fabric may exhibit substantially no increase in microbial growth after a number of machine washing cycles selected from the group consisting of 5 cycles, 10 cycles, 25 cycles, and 50 cycles. In some embodiments, the microbial growth may be microbial growth of a microbe selected from the group consisting of Staphylococcus aureus, Klebsiella pneumoniae, and combinations thereof. In some embodiments, the microbial growth may be reduced by a percentage selected from the group consisting of 50%, 100%, 500%, 1000%, 2000%, and 3000% compared to an uncoated fabric.

In some embodiments, the coating may be applied to the fabric at the fiber level prior to forming the fabric.

In some embodiments, the coating may be applied to the fabric at the fabric level. In some embodiments, the fabric may be bath coated. In some embodiments, the fabric may be spray coated. In some embodiments, the fabric may be coated with a stencil. In some embodiments, the coating may be applied to at least one side of the fabric using a method selected from the group consisting of a bath coating process, a spray coating process, a stencil process, a silk-foam based process, and a roller-based process.

In some embodiments, the coating may have a thickness of about one nanolayer, two nanolayers, three nanolayers, four nanolayers, five nanolayers, six nanolayers, seven nanolayers, eight nanolayers, nine nanolayers, ten nanolayers, or the like.

In some embodiments, the coating may have a thickness selected from the group consisting of about 5 nm, about 10 nm, about 15 nm, about 20 nm, about 25 nm, about 50 nm, about 100 nm, about 200 nm, about 500 nm, about 1 μm, about 5 μm, about 10 μm, and about 20 μm.

In some embodiments, the coating may be adsorbed on the fabric.

In some embodiments, the coating may be attached to the fabric through chemical, enzymatic, thermal, or irradiative cross-linking.

In some embodiments, the hand of the coated fabric may be improved relative to an uncoated fabric.

In some embodiments, the hand of the coated fabric that may be improved may be selected from the group consisting of softness, crispness, dryness, silkiness, and combinations thereof.

In some embodiments, a flame retardation property of the coated fabric may be improved relative to an uncoated fabric.

In some embodiments, a flame retardation property of an uncoated fabric may not be adversely affected by the coating.

In some embodiments, the abrasion resistance may be improved relative to an uncoated fabric.

In an embodiment, the disclosure may include an article comprising a textile or leather having a coating, wherein the coating comprises recombinant silk based proteins or fragments thereof having a weight average molecular weight, or average weight average molecular weight range of about 5 kDa to about 144 kDa (including, but not limited to, as provided in any of the compositions #1001-2450, #3001-4450, and #5001-6595).

In some embodiments, the recombinant silk based proteins or protein fragments thereof have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof have a polydispersity of between about 1.0 and about 5.0.

In some embodiments, at least one property of the article may be improved, wherein the property that may be improved may be selected from the group consisting of color retention, resistance to microbial growth, resistance to bacterial growth, resistance to fungal growth, resistance to the buildup of static electrical charge, resistance to the growth of mildew, transparency of the coating, resistance to freeze-thaw cycle damage, resistance from abrasion, blocking of ultraviolet (UV) radiation, regulation of the body temperature of a wearer, resistance to tearing, elasticity of the article, rebound dampening, tendency to cause itching in the wearer, thermal insulation of the wearer, wrinkle resistance, stain resistance, stickiness to skin, and flame resistance.

In some embodiments, the article may be a textile used for apparel.

In some embodiments, the article may be fabricated as an item selected from the group consisting of an item of athletic apparel, an item of outdoor gear, a jacket, an overcoat, a shoe, a sneaker, a glove, an umbrella, a chair, a blanket, a towel, a surgical drape, a surgical gown, a laboratory coat, a wound dressing, a sterilization wrap, a surgical face mask, a surgical sleeve, a laboratory sleeve, a retention bandage, a support device, a compression bandage, a shoe cover, and a surgical blanket.

In some embodiments, the article may be a textile, leather, or foam used to fabricate an automotive product.

In some embodiments, the article may be fabricated as an item selected from the group consisting of an upholstery, a foam cushion, a fabric cushion, a floor mat, a vehicle carpet, an automotive trim, a children's car seat, a seat belt, a safety harness, a headrest, an armrest, a dashboard, a sunvisor, a seat, an interior panel, an airbag, an airbag cover, a wiring harness, or an insulation.

In an embodiment, the disclosure may include a method of coating a fabric that may include the step of optionally applying a pretreatment selected from the group consisting of a wetting agent, a detergent, a sequestering or dispersing agent, an enzyme, a bleaching agent, an antifoaming agent, an anti-creasing agent, a dye dispersing agent, a dye leveling agent, a dye fixing agent, a dye special resin agent, a dye anti-reducing agent, a pigment dye system anti-migrating agent, a pigment dye system binder, a delave agent, a wrinkle free treatment, a softener, a handle modifier, a waterborne polyurethane dispersion, a finishing resin, an oil or water repellant, a flame retardant, a crosslinker, a thickener for technical finishing, or any combination thereof. In an embodiment, the method may include the step of applying a coating that may include a solution of recombinant silk based proteins or fragments thereof that may have an average molecular weight range of about 5 kDa to about 144 kDa (including, but not limited to, as provided in any of the compositions #1001-2450, #3001-4450, and #5001-6595), using a process selected from the group consisting of a continuous spray process, a continuous screen or stencil process, a continuous bath process, a batch spray process, a batch screen or stencil process, and a batch bath process. In an embodiment, the method may include the step of drying and optionally curing the coating.

In an embodiment, the recombinant silk based proteins or protein fragments thereof may have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof may have a polydispersity of between about 1.0 and about 5.0.

### BRIEF DESCRIPTION OF THE DRAWINGS

The foregoing summary, as well as the following detailed description of embodiments of the invention, will be better understood when read in conjunction with the appended drawings and figures.

FIG. 1 is a flow chart showing various embodiments for producing silk fibroin protein fragments of the present disclosure.

FIG. 2 is a flow chart showing various parameters that can be modified during the process of producing a silk protein fragment solutions of the present disclosure during the extraction and the dissolution steps.

### DETAILED DESCRIPTION OF THE DISCLOSURE

Raw silk from silkworm Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. The crude silkworm fiber consists of a double thread of fibroin. The adhesive substance holding these double fibers together is sericin. The silk fibroin is composed of a heavy chain having a weight average molecular weight of about 350,000 Da (H chain), and a light chain having a weight average molecular weight about 25,000 Da (L chain). Silk fibroin is an amphiphilic polymer with large hydrophobic domains occupying the major component of the polymer, which has a high molecular weight. The hydrophobic regions are interrupted by small hydrophilic spacers, and the N- and C-termini of the chains are also highly hydrophilic. The hydrophobic domains of the H-chain contain a repetitive hexapeptide sequence of Gly-Ala-Gly-Ala-Gly-Ser and repeats of Gly-Ala/Ser/Tyr dipeptides, which can form stable anti-parallel-sheet crystallites. The amino acid sequence of the L-chain is non-repetitive, so the L-chain is more hydrophilic and relatively elastic. The hydrophilic (Tyr, Ser) and hydrophobic (Gly, Ala) chain segments in silk fibroin molecules are arranged alternatively such that allows self-assembling of silk fibroin molecules.

Spider silks are natural polymers that consist of three domains: a repetitive middle core domain that dominates the protein chain, and non-repetitive N-terminal and C-terminal domains. The large core domain is organized in a block copolymer-like arrangement, in which two basic sequences, crystalline [poly(A) or poly(GA)] and less crystalline (GGX or GPGXX) polypeptides alternate. Dragline silk is the protein complex composed of major ampullate dragline silk protein 1 (MaSp1) and major ampullate dragline silk protein 2 (MaSp2). Both silks are approximately 3500 amino acid long. MaSp1 can be found in the fibre core and the periphery, whereas MaSp2 forms clusters in certain core areas. The large central domains of MaSp1 and MaSp2 are organized in block copolymer-like arrangements, in which two basic sequences, crystalline [poly(A) or poly(GA)] and less crystalline (GGX or GPGXX) polypeptides alternate in core domain. Specific secondary structures have been assigned to poly(A)/(GA), GGX and GPGXX motifs including β-sheet, α-helix and β-spiral respectively. The primary sequence, composition and secondary structural elements of the repetitive core domain are responsible for mechanical properties of spider silks; whereas, non-repetitive N- and C-terminal domains are essential for the storage of liquid silk dope in a lumen and fibre formation in a spinning duct.

The main difference between MaSp1 and MaSp2 is the presence of proline (P) residues accounting for 15% of the total amino acid content in MaSp2, whereas MaSp1 is proline-free. By calculating the number of proline residues in N. clavipes dragline silk, it is possible to estimate the presence of the two proteins in fibres; 81% MaSp1 and 19% MaSp2. Different spiders have different ratios of MaSp1 and MaSp2. For example, a dragline silk fibre from the orb weaver Argiope aurantia contains 41% MaSp1 and 59% MaSp2. Such changes in the ratios of major ampullate silks can dictate the performance of the silk fibre.

At least seven different types of silk proteins are known for one orb-weaver species of spider. Silks differ in primary sequence, physical properties and functions. For example, dragline silks used to build frames, radii and lifelines are known for outstanding mechanical properties including strength, toughness and elasticity. On an equal weight basis, spider silk has a higher toughness than steel and Kevlar.

Flageliform silk found in capture spirals has extensibility of up to 500%. Minor ampullate silk, which is found in auxiliary spirals of the orb-web and in prey wrapping, possesses high toughness and strength almost similar to major ampullate silks, but does not supercontract in water.

Spider silks are known for their high tensile strength and toughness. The recombinant silk proteins also confer advantageous properties to cosmetic or dermatological compositions, in particular to be able to improve the hydrating or softening action, good film forming property and low surface density. Diverse and unique biomechanical properties together with biocompatibility and a slow rate of degradation make spider silks excellent candidates as biomaterials for tissue engineering, guided tissue repair and drug delivery, for cosmetic products (e.g. nail and hair strengthener, skin care products), and industrial materials (e.g. nanowires, nanofibers, surface coatings).

### Definition

As used in the preceding sections and throughout the rest of this specification, unless defined otherwise, all technical and scientific terms used herein have the same meaning as is commonly understood by one skilled in the art to which this disclosure belongs. All patents and publications referred to herein are incorporated by reference in their entireties.

All percentages, parts and ratios are based upon the total weight of the hair care compositions of the present disclosure, unless otherwise specified. All such weights as they pertain to listed ingredients are based on the active level and, therefore, do not include solvents or by-products that may be included in commercially available materials, unless otherwise specified. The term “weight percent” may be denoted as “wt. %” or % w/w herein.

As used herein, the term “a”, “an”, or “the” generally is construed to cover both the singular and the plural forms.

As used herein, the term “about” generally refers to a particular numeric value that is within an acceptable error range as determined by one of ordinary skill in the art, which will depend in part on how the numeric value is measured or determined, i.e., the limitations of the measurement system. For example, “about” can mean a range of ±20%, ±10%, or ±5% of a given numeric value.

As used herein, the terms “polydispersity,” “PD,” or “PDI” are generally used as a measure of the broadness of a molecular weight distribution of a polymer, and is defined by the formula polydispersity (PD or PDI)=Mw/Mn.

As used herein, “recombinant protein” refers to a type of modified protein whose code is encoded by a recombinant DNA, that has been cloned in a system that supports expression of the gene and translation of messenger RNA (see expression system). In some embodiments, a recombinant DNA is composed of two segments of DNA joined together in a plasmid. The formation of recombinant protein is carried out in specialized vehicles known as vectors. When the recombinant DNA is inserted into the vectors (e.g., bacteria plasmid), they will translate these DNA into proteins which bear the new sets of characteristics.

The following abbreviations are used to identify amino acids:

| Amino Acid | Three Letter Code | One Letter Code |
|---|---|---|
| Alanine | Ala | A |
| Arginine | Arg | R |
| Asparagine | Asn | N |
| Aspartic Acid | Asp | D |
| Asparagine Or | Asx | B |
| Aspartic Acid | | |
| Cysteine | Cys | C |
| Glutamic Acid | Glu | E |
| Glutamine | Gln | Q |
| Glutamine Or | Glx | Z |
| Glutamic Acid | | |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Recombinant Silk-Based Protein Fragments and Solutions Thereof

Provided herein are methods for producing pure and highly scalable recombinant silk protein fragment mixture solutions (including, but not limited to, as provided in any of the compositions #1001-2450, #3001-4450, and #5001-6595) that may be used to coat at least a portion of textiles or may be formed into usable fibers for weaving into yarn. In some embodiments, RSPF mixture solutions may also refer to recombinant silk solutions (RSS), and vice versa.

In some embodiments, the recombinant silk protein refers to recombinant spider silk polypeptides, recombinant insect silk polypeptides, or recombinant mussel silk polypeptides. In some embodiments, the recombinant silk protein fragment disclosed herein include recombinant spider silk polypeptides of Araneidae or Araneoids, or recombinant insect silk polypeptides of Bombyx mori. In some embodiments, the recombinant silk protein fragment disclosed herein include recombinant spider silk polypeptides of Araneidae or Araneoids. In some embodiments, the recombinant silk protein fragment disclosed herein include block copolymer having repetitive units derived from natural spider silk polypeptides of Araneidae or Araneoids. In some embodiments, the recombinant silk protein fragment disclosed herein include block copolymer having synthetic repetitive units derived from spider silk polypeptides of Araneidae or Araneoids and non-repetitive units derived from natural repetitive units of spider silk polypeptides of Araneidae or Araneoids. In some embodiments, the recombinant silk protein fragment disclosed herein has a weight average molecular weight ranging from about 6 kDa to about 145 kDa, or about 6 kDa to about 17 kDa, or about 17 kDa to about 39 kDa, or about 39 kDa to about 80 kDa.

As used herein, in some embodiments the term “silk protein fragment” also refers to a silk protein that comprises or consists of at least two identical repetitive units which each independently selected from naturally-occurring silk polypeptides or of variations thereof, amino acid sequences of naturally-occurring silk polypeptides, or of combinations of both.

Recent advances in genetic engineering have provided a route to produce various types of recombinant silk proteins. Recombinant DNA technology has been used to provide a more practical source of silk proteins. As used herein “recombinant silk protein” refers to synthetic proteins produced heterologously in prokaryotic or eukaryotic expression systems using genetic engineering methods.

Various methods for synthesizing recombinant silk peptides are known and have been described by Ausubel et al., Current Protocols in Molecular Biology § 8 (John Wiley & Sons 1987, (1990)), incorporated herein by reference. A gram-negative, rod-shaped bacterium E. coli is a well-established host for industrial scale production of proteins. Therefore, the majority of recombinant silks have been produced in E. coli. E. coli which is easy to manipulate, has a short generation time, is relatively low cost and can be scaled up for larger amounts protein production.

The recombinant silk proteins can be produced by transformed prokaryotic or eukaryotic systems containing the cDNA coding for a silk protein, for a fragment of this protein or for an analog of such a protein. The recombinant DNA approach enables the production of recombinant silks with programmed sequences, secondary structures, architectures and precise molecular weight. There are four main steps in the process: (i) design and assembly of synthetic silk-like genes into genetic ‘cassettes’, (ii) insertion of this segment into a DNA recombinant vector, (iii) transformation of this recombinant DNA molecule into a host cell and (iv) expression and purification of the selected clones.

The term “recombinant vectors”, as used herein, includes any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, or plant) or in in vitro expression systems. Cloning vectors are generally used to engineer and amplify a certain desired DNA fragment and may lack functional sequences needed for expression of the desired DNA fragments.

The prokaryotic systems include Gram-negative bacteria or Gram-positive bacteria. The prokaryotic expression vectors can include an origin of replication which can be recognized by the host organism, a homologous or heterologous promoter which is functional in the said host, the DNA sequence coding for the spider silk protein, for a fragment of this protein or for an analogous protein. Nonlimiting examples of prokaryotic expression organisms are Escherichia coli, Bacillus subtilis, Bacillus megaterium, Corynebacterium glutamicum, Anabaena, Caulobacter, Gluconobacter, Rhodobacter, Pseudomonas, Para coccus, Bacillus (e.g. Bacillus subtilis) Brevibacterium, Corynebacterium, Rhizobium (Sinorhizobium), Flavobacterium, Klebsiella, Enterobacter, Lactobacillus, Lactococcus, Methylobacterium, Propionibacterium, Staphylococcus or Streptomyces cells.

The eukaryotic systems include yeasts and insect, mammalian or plant cells. In this case, the expression vectors can include a yeast plasmid origin of replication or an autonomous replication sequence, a promoter, a DNA sequence coding for a spider silk protein, for a fragment or for an analogous protein, a polyadenylation sequence, a transcription termination site and, lastly, a selection gene. Nonlimiting examples of eukaryotic expression organisms include yeasts, such as Saccharomyces cerevisiae, Pichia pastoris, basidiosporogenous, ascosporogenous, filamentous fungi, such as Aspergillus niger, Aspergillus oryzae, Aspergillus nidulans, Trichoderma reesei, Acremonium chrysogenum, Candida, Hansenula, Kluyveromyces, Saccharomyces (e.g. Saccharomyces cerevisiae), Schizosaccharomyces, Pichia (e.g. Pichia pastoris) or Yarrowia cells etc., mammalian cells, such as HeLa cells, COS cells, CHO cells etc., insect cells, such as Sf9 cells, MEL cells, etc., “insect host cells” such as Spodoptera frugiperda or Trichoplusia ni cells. SF9 cells, SF-21 cells or High-Five cells, wherein SF-9 and SF-21 are ovarian cells from Spodoptera frugiperda, and High-Five cells are egg cells from Trichoplusia ni., “plant host cells”, such as tobacco, potato or pea cells.

A variety of heterologous host systems have been explored to produce different types of recombinant silks. Recombinant partial spidroins as well as engineered silks have been cloned and expressed in bacteria (Escherichia coli), yeast (Pichia pastoris), insects (silkworm larvae), plants (tobacco, soybean, potato, Arabidopsis), mammalian cell lines (BHT/hamster) and transgenic animals (mice, goats). Most of the silk proteins are produced with an N- or C-terminal His-tags to make purification simple and produce enough amounts of the protein.

In some embodiments, the host suitable for expressing the recombinant spider silk protein using heterogeneous system may include transgenic animals and plants. In some embodiments, the host suitable for expressing the recombinant spider silk protein using heterogeneous system comprises bacteria, yeasts, mammalian cell lines. In some embodiments, the host suitable for expressing the recombinant spider silk protein using heterogeneous system comprises E. coli. In some embodiments, the host suitable for expressing the recombinant spider silk protein using heterogeneous system comprises transgenic B. mori silkworm generated using genome editing technologies (e.g. CRISPR).

The recombinant silk protein in this disclosure comprises synthetic proteins which are based on repeat units of natural silk proteins. Besides the synthetic repetitive silk protein sequences, these can additionally comprise one or more natural nonrepetitive silk protein sequences.

In some embodiments, “recombinant silk protein” refers to recombinant silkworm silk protein or fragments thereof. The recombinant production of silk fibroin and silk sericin has been reported. A variety of hosts are used for the production including E. coli, Saccharomyces cerevisiae, Pseudomonas sp., Rhodopseudomonas sp., Bacillus sp., and Strepomyces. See EP 0230702, which is incorporate by reference herein by its entirety.

Provided herein also include design and biological-synthesis of silk fibroin protein-like multiblock polymer comprising GAGAGX hexapeptide (X is A, Y, V or S) derived from the repetitive domain of B. mori silk heavy chain (H chain) In some embodiments, this disclosure provides silk protein-like multiblock polymers derived from the repetitive domain of B. mori silk heavy chain (H chain) comprising the GAGAGS hexapeptide repeating units. The GAGAGS hexapeptide is the core unit of H-chain and plays an important role in the formation of crystalline domains. The silk protein-like multiblock polymers containing the GAGAGS hexapeptide repeating units spontaneously aggregate into β-sheet structures, similar to natural silk fibroin protein, where in the silk protein-like multiblock polymers having a weight average molecular weight ranging from about 6 kDa to about 145 kDa, or about 6 kDa to about 17 kDa, or about 17 kDa to about 39 kDa, or about 39 kDa to about 80 kDa.

In some embodiments, this disclosure provides silk-peptide like multiblock copolymers composed of the GAGAGS hexapeptide repetitive fragment derived from H chain of B. mori silk heavy chain and mammalian elastin VPGVG motif produced by E. coli. In some embodiments, this disclosure provides fusion silk fibroin proteins composed of the GAGAGS hexapeptide repetitive fragment derived from H chain of B. mori silk heavy chain and GVGVP produced by E. coli, where in the silk protein-like multiblock polymers having a weight average molecular weight ranging from about 6 kDa to about 145 kDa, or about 6 kDa to about 17 kDa, or about 17 kDa to about 39 kDa, or about 39 kDa to about 80 kDa.

In some embodiments, this disclosure provides B. mori silkworm recombinant proteins composed of the (GAGAGS)₁₆ repetitive fragment. In some embodiments, this disclosure provides recombinant proteins composed of the (GAGAGS)₁₆ repetitive fragment and the non-repetitive (GAGAGS)₁₆-F—COOH, (GAGAGS)₁₆-F—F—COOH, (GAGAGS)₁₆-F—F—F—COOH, (GAGAGS)₁₆-F—F—F—F—COOH, (GAGAGS)₁₆-F—F—F—F—F—F—F—F—COOH, (GAGAGS)₁₆-F—F—F—F—F—F—F—F—F—F—F—F—COOH produced by E. coli, where F has the following amino acid sequence SGFGPVANGGSGEASSESDFGSSGFGPVANASSGEASSESDFAG, and where in the silk protein-like multiblock polymers having a weight average molecular weight ranging from about 6 kDa to about 145 kDa, or about 6 kDa to about 17 kDa, or about 17 kDa to about 39 kDa, or about 39 kDa to about 80 kDa (including, but not limited to, as provided in any of the compositions #1001-2450, #3001-4450, and #5001-6595).

In some embodiments, “recombinant silk protein” refers to recombinant spider silk protein or fragments thereof.

The productions of recombinant spider silk proteins based on a partial cDNA clone have been reported. The recombinant spider silk proteins produced as such comprise a portion of the repetitive sequence derived from a dragline spider silk protein, Spidroin 1, from the spider Nephila clavipes. see Xu et al. (Proc. Natl. Acad. Sci. U.S.A., 87:7120-7124 (1990). cDNA clone encoding a portion of the repeating sequence of a second fibroin protein, Spidroin 2, from dragline silk of Nephila clavipes and the recombinant synthesis thereof is described in J. Biol. Chem., 1992, volume 267, pp. 19320-19324. The recombinant synthesis of spider silk proteins including protein fragments and variants of Nephila clavipes from transformed E. coli is described in U.S. Pat. Nos. 5,728,810 and 5,989,894. cDNA clones encoding minor ampullate spider silk proteins and the expression thereof is described in U.S. Pat. Nos. 5,733,771 and 5,756,677. cDNA clone encoding the flagelliform silk protein from an orb-web spinning spider is described in U.S. Pat. No. 5,994,099. U.S. Pat. No. 6,268,169 describes the recombinant synthesis of spider silk like proteins derived from the repeating peptide sequence found in the natural spider dragline of Nephila clavipes by E. coli, Bacillus subtilis, and Pichia pastoris recombinant expression systems. WO 03/020916 describes the cDNA clone encoding and recombinant production of spider spider silk proteins having repeative sequences derived from the major ampullate glands of Nephila madagascariensis, Nephila senegalensis, Tetragnatha kauaiensis, Tetragnatha versicolor, Argiope aurantia, Argiope trifasciata, Gasteracantha mammosa, and Latrodectus geometricus, the flagelliform glands of Argiope trifasciata, the ampullate glands of Dolomedes tenebrosus, two sets of silk glands from Plectreurys tristis, and the silk glands of the mygalomorph Euagrus chisoseus. Each of the above reference is incorporated herein by reference in its entirety.

In some embodiments, the recombinant spider silk protein is a hybrid protein of a spider silk protein and an insect silk protein, a spider silk protein and collagen, a spider silk protein and resilin, or a spider silk protein and keratin. The spider silk repetitive unit comprises or consists of an amino acid sequence of a region that comprises or consists of at least one peptide motif that repetitively occurs within a naturally occurring major ampullate gland polypeptide, such as a dragline spider silk polypeptide, a minor ampullate gland polypeptide, a flagelliform polypeptide, an aggregate spider silk polypeptide, an aciniform spider silk polypeptide or a pyriform spider silk polypeptide.

In some embodiments, the recombinant spider silk protein in this disclosure comprises synthetic spider silk proteins derived from repetitive units of natural spider silk proteins, consensus sequence, and optionally one or more natural non-repetitive spider silk protein sequences. The repeated units of natural spider silk polypeptide may include dragline spider silk polypeptides or flagelliform spider silk polypeptides of Araneidae or Araneoids.

As used herein, the spider silk “repetitive unit” comprises or consists of at least one peptide motif that repetitively occurs within a naturally occurring major ampullate gland polypeptide, such as a dragline spider silk polypeptide, a minor ampullate gland polypeptide, a flagelliform polypeptide, an aggregate spider silk polypeptide, an aciniform spider silk polypeptide or a pyriform spider silk polypeptide. A “repetitive unit” refers to a region which corresponds in amino acid sequence to a region that comprises or consists of at least one peptide motif (e.g. AAAAAA) or GPGQQ) that repetitively occurs within a naturally occurring silk polypeptide (e.g. MaSpI, ADF-3, ADF-4, or Flag) (i.e. identical amino acid sequence) or to an amino acid sequence substantially similar thereto (i.e. variational amino acid sequence). A “repetitive unit” having an amino acid sequence which is “substantially similar” to a corresponding amino acid sequence within a naturally occurring silk polypeptide (i.e. wild-type repetitive unit) is also similar with respect to its properties, e.g. a silk protein comprising the “substantially similar repetitive unit” is still insoluble and retains its insolubility. A “repetitive unit” having an amino acid sequence which is “identical” to the amino acid sequence of a naturally occurring silk polypeptide, for example, can be a portion of a silk polypeptide corresponding to one or more peptide motifs of MaSpI, MaSpII, ADF-3 and/or ADF-4. A “repetitive unit” having an amino acid sequence which is “substantially similar” to the amino acid sequence of a naturally occurring silk polypeptide, for example, can be a portion of a silk polypeptide corresponding to one or more peptide motifs of MaSpI, MaSpII, ADF-3 and/or ADF-4, but having one or more amino acid substitution at specific amino acid positions.

As used herein, the term “consensus peptide sequence” refers to an amino acid sequence which contains amino acids which frequently occur in a certain position (e.g. “G”) and wherein, other amino acids which are not further determined are replaced by the place holder “X”. In some embodiments, the consensus sequence is at least one of (i) GPGXX, wherein X is an amino acid selected from A, S, G, Y, P and Q; (ii) GGX, wherein X is an amino acid selected from Y, P, R, S, A, T, N and Q, preferably Y, P and Q; (iii) Ax, wherein X is an integer from 5 to 10.

The consensus peptide sequences GPGXX and GGX, i.e. glycine rich motifs, provide flexibility to the silk polypeptide and thus, to the thread formed from the silk protein containing said motifs. In detail, the iterated GPGXX motif forms turn spiral structures, which imparts elasticity to the silk polypeptide. Major ampullate and flagelliform silks both have a GPGXX motif. The iterated GGX motif is associated with a helical structure having three amino acids per turn and is found in most spider silks. The GGX motif may provide additional elastic properties to the silk. The iterated polyalanine Ax (peptide) motif forms a crystalline β-sheet structure that provides strength to the silk polypeptide, as described for example in WO 03/057727.

In some embodiments, the recombinant spider silk protein in this disclosure comprises two identical repetitive units each comprising at least one, preferably one, amino acid sequence selected from the group consisting of: GGRPSDTYG and GGRPSSSYG derived from Resilin. Resilin is an elastomeric protein found in most arthropods that provides low stiffness and high strength.

As used herein, “non-repetitive units” refers to an amino acid sequence which is “substantially similar” to a corresponding non-repetitive (carboxy terminal) amino acid sequence within a naturally occurring dragline polypeptide (i.e. wild-type non-repetitive (carboxy terminal) unit), preferably within ADF-3 (SEQ ID NO:1), ADF-4 (SEQ ID NO:2), NR3 (SEQ ID NO:41), NR4 (SEQ ID NO:42), ADF-4 of the spider Araneus diadematus as described in U.S. Pat. No. 8,367,803, C16 peptide (spider silk protein eADF4, molecular weight of 47.7 kDa, AMSilk) comprising the 16 repeats of the sequence GSSAAAAAAAASGPGGYGPENQGPSGPGGYGPGGP, an amino acid sequence adapted from the natural sequence of ADF4 from A. diadematus. Non-repetitive ADF-4 and variants thereof display efficient assembly behavior.

Among the synthetic spider silk proteins, the recombinant silk protein in this disclosure comprises in some embodiments the C16-protein having the polypeptide sequence SEQ ID NO: 1 as described in U.S. Pat. No. 8,288,512. Besides the polypeptide sequence shown in SEQ ID NO:1, particularly functional equivalents, functional derivatives and salts of this sequence are also included.

As used herein, “functional equivalents” refers to mutant which, in at least one sequence position of the abovementioned amino acid sequences, have an amino acid other than that specifically mentioned.

In some embodiments, the recombinant spider silk protein in this disclosure comprises, in an effective amount, at least one natural or recombinant silk protein including spider silk protein, corresponding to Spidroin major 1 described by Xu et al., PNAS, USA, 87, 7120, (1990), Spidroin major 2 described by Hinman and Lewis, J. Biol. Chem., 267, 19320, (1922), recombinant spider silk protein as described in U.S. Patent Application No. 2016/0222174 and U.S. Pat. Nos. 9,051,453, 9,617,315, 9,689,089, 8,173,772, 8,642,734, 8,367,803 8,097,583, 8,030,024, 7,754,851, 7,148,039, 7,060,260, or alternatively the minor Spidroins described in patent application WO 95/25165. Each of the above-cited references is incorporated herein by reference in its entirety. Additional recombinant spider silk proteins suitable for the recombinant RSPF of this disclosure include ADF3 and ADF4 from the “Major Ampullate” gland of Araneus diadematus.

Recombinant silk is also described in other patents and patent applications, incorporated by reference herein: US 2004590196, U.S. Pat. No. 7,754,851, US 2007654470, U.S. Pat. No. 7,951,908, US 2010785960, U.S. Pat. No. 8,034,897, US 20090263430, US 2008226854, US 20090123967, US 2005712095, US 2007991037, US 20090162896, US 200885266, U.S. Pat. No. 8,372,436, US 2007989907, US 2009267596, US 2010319542, US 2009265344, US 2012684607, US 2004583227, U.S. Pat. No. 8,030,024, US 2006643569, U.S. Pat. No. 7,868,146, US 2007991916, U.S. Pat. No. 8,097,583, US 2006643200, U.S. Pat. Nos. 8,729,238, 8,877,903, US 20190062557, US 20160280960, US 20110201783, US 2008991916, US 2011986662, US 2012697729, US 20150328363, U.S. Pat. No. 9,034,816, US 20130172478, U.S. Pat. No. 9,217,017, US 20170202995, U.S. Pat. No. 8,721,991, US 2008227498, U.S. Pat. Nos. 9,233,067, 8,288,512, US 2008161364, U.S. Pat. No. 7,148,039, US 1999247806, US 2001861597, US 2004887100, U.S. Pat. Nos. 9,481,719, 8,765,688, US 200880705, US 2010809102, U.S. Pat. No. 8,367,803, US 2010664902, U.S. Pat. No. 7,569,660, US 1999138833, US 2000591632, US 20120065126, US 20100278882, US 2008161352, US 20100015070, US 2009513709, US 20090194317, US 2004559286, US 200589551, US 2008187824, US 20050266242, US 20050227322, and US 20044418.

Recombinant silk is also described in other patents and patent applications, incorporated by reference herein: US 20190062557, US 20150284565, US 20130225476, US 20130172478, US 20130136779, US 20130109762, US 20120252294, US 20110230911, US 20110201783, US 20100298877, U.S. Pat. Nos. 10,478,520, 10,253,213, 10,072,152, 9,233,067, 9,217,017, 9,034,816, 8,877,903, 8,729,238, 8,721,991, 8,097,583, 8,034,897, U.S. Pat. Nos. 8,030,024, 7,951,908, 7,868,146, and 7,754,851.

In some embodiments, the recombinant spider silk protein in this disclosure comprises or consists of 2 to 80 repetitive units, each independently selected from GPGXX, GGX and Aₓ as defined above, wherein the recombinant spider silk protein having a weight average molecular weight ranging from about 6 kDa to about 145 kDa, or about 6 kDa to about 17 kDa, or about 17 kDa to about 39 kDa, or about 39 kDa to about 80 kDa, about 6 kDa to about 145 kDa, or about 6 kDa to about 17 kDa, or about 17 kDa to about 39 kDa, or about 39 kDa to about 80 kDa, or about 6 kDa to about 17 kDa, or about 17 kDa to about 39 kDa, or about 39 kDa to about 80 kDa (including, but not limited to, as provided in any of the compositions #1001-2450, #3001-4450, and #5001-6595).

In some embodiments, the recombinant spider silk protein in this disclosure comprises or consists of repetitive units each independently selected from selected from the group consisting of GPGAS, GPGSG, GPGGY, GPGGP, GPGGA, GPGQQ, GPGGG, GPGQG, GPGGS, GGY, GGP, GGA, GGR, GGS, GGT, GGN, GGQ, AAAAA, AAAAAA, AAAAAAA, AAAAAAAA, AAAAAAAAA, AAAAAAAAAA, GGRPSDTYG and GGRPSSSYG, (i) GPYGPGASAAAAAAGGYGPGSGQQ, (ii) GSSAAAAAAAASGPGGYGPENQGPSGPGGYGPGGP, (iii) GPGQQGPGQQGPGQQGPGQQ: (iv) GPGGAGGPYGPGGAGGPYGPGGAGGPY, (v) GGTTIIEDLDITIDGADGPITISEELTI, (vi) PGSSAAAAAAAASGPGQGQGQGQGQGGRPSDTYG, (vii) SAAAAAAAAGPGGGNGGRPSDTYGAPGGGNGGRPSSSYG, (viii) GGAGGAGGAGGSGGAGGS (SEQ ID NO: 27), (ix) GPGGAGPGGYGPGGSGPGGYGPGGSGPGGY, (x) GPYGPGASAAAAAAGGYGPGCGQQ, (xi) GPYGPGASAAAAAAGGYGPGKGQQ, (xii) GSSAAAAAAAASGPGGYGPENQGPCGPGGYGPGGP, (xiii) GSSAAAAAAAASGPGGYGPKNQGPSGPGGYGPGGP, (xiv) GSSAAAAAAAASGPGGYGPKNQGPSGPGGYGPGGP, or variants thereof as described in U.S. Pat. No. 8,877,903, for example, a synthetic spider peptide having sequential order of GPGAS, GGY, GPGSG in the peptide chain, or sequential order of AAAAAAAA, GPGGY, GPGGP in the peptide chain, sequential order of AAAAAAAA, GPGQG, GGR in the peptide chain; wherein the recombinant spider silk protein having a weight average molecular weight ranging from about 6 kDa to about 145 kDa, or about 6 kDa to about 17 kDa, or about 17 kDa to about 39 kDa, or about 39 kDa to about 80 kDa (including, but not limited to, as provided in any of the compositions #1001-2450, #3001-4450, and #5001-6595).

In some embodiments, this disclosure provides silk protein-like multiblock peptides that imitate the repeating units of amino acids derived from natural spider silk proteins such as Spidroin major 1 domain, Spidroin major 2 domain or Spidroin minor 1 domain and the profile of variation between the repeating units without modifying their three-dimensional conformation, wherein these silk protein-like multiblock peptides comprise a repeating unit of amino acids corresponding to one of the sequences (I), (II), (III) and/or (IV) below.

[(XGG)_{w}(XGA)(GXG)ₓ(AGA)_{y}(G)_{z}AG]ₚ Formula (I) in which: X corresponds to tyrosine or to glutamine, w is an integer equal to 2 or 3, x is an integer from 1 to 3, y is an integer from 5 to 7, z is an integer equal to 1 or 2, and p is an integer and having a weight average molecular weight ranging from about 6 kDa to about 145 kDa, or about 6 kDa to about 17 kDa, or about 17 kDa to about 39 kDa, or about 39 kDa to about 80 kDa (and including, but not limited to, as provided in any of the compositions #1001-2450, #3001-4450, and #5001-6595), and/or [(GPG₂YGPGQ₂)ₐ(X′)₂S(A)_{b}]ₚ Formula (II) in which: X′ corresponds to the amino acid sequence GPS or GPG, a is equal to 2 or 3, b is an integer from 7 to 10, and p is an integer and having a weight average molecular weight ranging from about 6 kDa to about 145 kDa, or about 6 kDa to about 17 kDa, or about 17 kDa to about 39 kDa, or about 39 kDa to about 80 kDa (and including, but not limited to, as provided in any of the compositions #1001-2450, #3001-4450, and #5001-6595), and/or

[(GR)(GA)₁(A)ₘ(GGX)ₙ(GA)₁(A)ₘ]ₚ Formula (III) and/or [(GGX)ₙ(GA)ₘ(A)₁]ₚ Formula (IV) in which: X″ corresponds to tyrosine, glutamine or alanine, 1 is an integer from 1 to 6, m is an integer from 0 to 4, n is an integer from 1 to 4, and p is an integer and having a weight average molecular weight ranging from about 6 kDa to about 145 kDa, or about 6 kDa to about 17 kDa, or about 17 kDa to about 39 kDa, or about 39 kDa to about 80 kDa (and including, but not limited to, as provided in any of the compositions #1001-2450, #3001-4450, and #5001-6595).

In some embodiments, the recombinant spider silk protein or an analog of a spider silk protein comprising an amino acid repeating unit of sequence (IV):

[(Xaa Gly Gly)_{w}(Xaa Gly Ala)(Gly Xaa Gly)ₓ(Ala Gly Ala)_{y}(Gly)_{z}Ala Gly]ₚ Formula (IV), wherein Xaa is tyrosine or glutamine, w is an integer equal to 2 or 3, x is an integer from 1 to 3, y is an integer from 5 to 7, z is an integer equal to 1 or 2, and p is an integer and having a weight average molecular weight ranging from about 6 kDa to about 145 kDa, or about 6 kDa to about 17 kDa, or about 17 kDa to about 39 kDa, or about 39 kDa to about 80 kDa.

In some embodiments, the recombinant spider silk protein in this disclosure is selected from the group consisting of ADF-3 or variants thereof, ADF-4 or variants thereof, MaSpI (SEQ ID NO: 43) or variants thereof, MaSpII (SEQ ID NO: 44) or variants thereof as described in U.S. Pat. No. 8,367,803.

In some embodiments, this disclosure provides water soluble recombinant spider silk proteins produced in mammalian cells. The solubility of the spider silk proteins produced in mammalian cells was attributed to the presence of the COOH-terminus in these proteins, which makes them more hydrophilic. These COOH-terminal amino acids are absent in spider silk proteins expressed in microbial hosts.

In some embodiments, the recombinant spider silk protein in this disclosure comprises water soluble recombinant spider silk protein C16 modified with an amino or caboxy terminal selected from the amino acid sequences consisting of: GCGGGGGG, GKGGGGGG, GCGGSGGGGSGGGG, GKGGGGGGSGGGG, and GCGGGGGGSGGGG. In some embodiments, the recombinant spider silk protein in this disclosure comprises C₁₆NR4, C₃₂NR4, C16, C32, NR4C₁₆NR4, NR4C₃₂NR4, NR3C₁₆NR3, or NR3C₃₂NR3 such that the molecular weight of the protein ranges from about 6 kDa to about 145 kDa, or about 6 kDa to about 17 kDa, or about 17 kDa to about 39 kDa, or about 39 kDa to about 80 kDa (and including, but not limited to, as provided in any of the compositions #1001-2450, #3001-4450, and #5001-6595).

In some embodiments, the recombinant spider silk protein in this disclosure comprises recombinant spider silk protein having a synthetic repeative peptide segments and an amino acid sequence adapted from the natural sequence of ADF4 from A. diadematus as described in U.S. Pat. No. 8,877,903. In some embodiments, the RSPF in this disclosure comprises the recombinant spider silk proteins having repeating peptide units derived from natural spider silk proteins such as Spidroin major 1 domain, Spidroin major 2 domain or Spidroin minor 1 domain, wherein the repeating peptide sequence is GSSAAAAAAAASGPGQGQGQGQGQGGRPSDTYG or SAAAAAAAAGPGGGNGGRPSDTYGAPGGGNGGRPSSSYG, as described in U.S. Pat. No. 8,367,803.

In some embodiments, this disclosure provides recombinant spider proteins composed of the GPGGAGPGGYGPGGSGPGGYGPGGSGPGGY repetitive fragment and having a weight average molecular weight ranging from about 6 kDa to about 145 kDa, or about 6 kDa to about 17 kDa, or about 17 kDa to about 39 kDa, or about 39 kDa to about 80 kDa (and including, but not limited to, as provided in any of the compositions #1001-2450, #3001-4450, and #5001-6595). The solutions are prepared from recombinant silk protein material and prepared to provide weight average molecular weight (MW) and polydispersity characteristics. Select preparation parameters may be altered to achieve distinct final recombinant silk protein fragment characteristics depending upon the intended use. The resulting final fragment solution is recombinant silk protein fragments and water with PPM to non-detectable levels of process contaminants. The concentration, size and polydispersity of recombinant silk protein fragments in the solution may further be altered depending upon the desired use and performance requirements. In an embodiment, the recombinant silk-based protein fragments in the solution have an average weight average molecular weight ranging from about 6 kDa to about 17 kDa, and have a polydispersity ranging from 1.0 and about 5.0. In an embodiment, the recombinant silk-based protein fragments in the solution have an average weight average molecular weight ranging from about 17 kDa to about 39 kDa, and have a polydispersity ranging from 1.0 and about 5.0. In an embodiment, the recombinant silk-based protein fragments in the solution have an average weight average molecular weight ranging from about 39 kDa to about 80 kDa, and have a polydispersity ranging from 1.0 and about 5.0. In an embodiment, the solutions may be used to generate articles, such as recombinant silk gels of varying gel and liquid consistencies by varying water content/concentration, or sold as a raw ingredient into the consumer market. As used herein, the term “silk solution” may refer to solutions of silk proteins, including solutions of recombinant spider silk-based protein fragments.

As used herein, “low molecular weight” recombinant silk solutions may include those SFS solutions that include recombinant silk-based protein fragments having a weight average molecular weight, or average weight average molecular weight in a range of about 5 kDa to 20 kDa. In some embodiments, a target low molecular weight for certain recombinant silk-based protein fragments may be weight average molecular weight of about 11 kDa.

As used herein, “medium molecular weight” recombinant silk solutions may include those SFS solutions that include recombinant silk based protein fragments having a weight average molecular weight, or average weight average molecular weight in a range of about 20 kDa to about 55 kDa. In some embodiments, a target medium molecular weight for certain recombinant silk-based protein fragments may be weight average molecular weight of about 40 kDa.

As used herein, “high molecular weight” recombinant silk solutions may include those SFS solutions that include recombinant silk based protein fragments having a weight average molecular weight, or average weight average molecular weight that is in a range of about 55 kDa to about 150 kDa. In some embodiments, a target high molecular weight for certain recombinant silk-based protein fragments may be about 100 kDa to about 145 kDa.

As used herein, symbol for percentage “%” for composition ingredients is wt. % by total weight of the composition except for otherwise specifically defined as % w/v, or % v/v.

In some embodiments, the molecular weights described herein (e.g., low molecular weight silk, medium molecular weight silk, high molecular weight silk) may be converted to the approximate number of amino acids contained within the respective natural or recombinant proteins, such as natural or recombinant silk proteins, as would be understood by a person having ordinary skill in the art. For example, the average weight of an amino acid may be about 110 daltons (i.e., 110 g/mol). Therefore, in some embodiments, dividing the molecular weight of a linear protein by 110 daltons may be used to approximate the number of amino acid residues contained therein.

As used herein, the term “substantially homogeneous” may refer to recombinant silk-based protein fragments that are distributed in a normal distribution about an identified molecular weight. As used herein, the term “substantially homogeneous” may refer to an even distribution of additive, for example vitamin C, throughout a composition of the present disclosure.

As used herein, the term “substantially free of inorganic residuals” means that the composition exhibits residuals of 0.1% (w/w) or less. In an embodiment, substantially free of inorganic residuals refers to a composition that exhibits residuals of 0.05% (w/w) or less. In an embodiment, substantially free of inorganic residuals refers to a composition that exhibits residuals of 0.01% (w/w) or less. In an embodiment, the amount of inorganic residuals is between 0 ppm (“non-detectable” or “ND”) and 1000 ppm. In an embodiment, the amount of inorganic residuals is ND to about 500 ppm. In an embodiment, the amount of inorganic residuals is ND to about 400 ppm. In an embodiment, the amount of inorganic residuals is ND to about 300 ppm. In an embodiment, the amount of inorganic residuals is ND to about 200 ppm. In an embodiment, the amount of inorganic residuals is ND to about 100 ppm. In an embodiment, the amount of inorganic residuals is between 10 ppm and 1000 ppm.

As used herein, the term “substantially free of organic residuals” means that the composition exhibits residuals of 0.1% (w/w) or less. In an embodiment, substantially free of organic residuals refers to a composition that exhibits residuals of 0.05% (w/w) or less. In an embodiment, substantially free of organic residuals refers to a composition that exhibits residuals of 0.01% (w/w) or less. In an embodiment, the amount of organic residuals is between 0 ppm (“non-detectable” or “ND”) and 1000 ppm. In an embodiment, the amount of organic residuals is ND to about 500 ppm. In an embodiment, the amount of organic residuals is ND to about 400 ppm. In an embodiment, the amount of organic residuals is ND to about 300 ppm. In an embodiment, the amount of organic residuals is ND to about 200 ppm. In an embodiment, the amount of organic residuals is ND to about 100 ppm. In an embodiment, the amount of organic residuals is between 10 ppm and 1000 ppm. Compositions of the present disclosure are “biocompatible” or otherwise exhibit “biocompatibility” meaning that the compositions are compatible with living tissue or a living system by not being toxic, injurious, or physiologically reactive and not causing immunological rejection or an inflammatory response. Such biocompatibility can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely. For example, in some embodiments, the coatings described herein are biocompatible coatings.

In some embodiments, compositions described herein, which may be biocompatible compositions (e.g., biocompatible coatings that include silk), may be evaluated and comply with International Standard ISO 10993-1, titled the “Biological evaluation of medical devices—Part 1: Evaluation and testing within a risk management process.” In some embodiments, compositions described herein, which may be biocompatible compositions, may be evaluated under ISO 106993-1 for one or more of cytotoxicity, sensitization, hemocompatibility, pyrogenicity, implantation, genotoxicity, carcinogenicity, reproductive and developmental toxicity, and degradation.

In some embodiments, compositions and articles described herein, and methods of preparing the same, include silk coated fabrics and textiles wherein the silk coating is partially dissolved in the fabric or textile. The fabric or textile may be a polymeric material such as those described elsewhere herein. The term “partially dissolved” includes mixing to form a dispersion of, e.g., a portion of a polymeric fabric or textile with a portion of the silk based coating. In some embodiments, the dispersion may be a solid suspension (i.e., a dispersion comprising domains on the order of 10 nm) or a solid solution (i.e., a molecular dispersion) of silk in the polymeric fabric or textile. In some embodiments, the dispersion may be localized at the surface interface between the silk coating and the polymeric fabric or textile, and may have a depth of 1 nm, 2 nm, 5 nm, 10 nm, 25 nm, 50 nm, 75 nm, 100 nm, or greater than 100 nm, depending on the method of preparation. In some embodiments, the dispersion may be a layer sandwiched between the polymeric fabric or textile and the silk coating. In some embodiments, the dispersion may be prepared by coating silk, including recombinant silk with the characteristics described herein, onto the polymeric fabric or textile, and then performing an additional process to form the dispersion, including heating at a temperature of 100° C., 125° C., 150° C., 175° C., 200° C., 225° C., or 250° C. for a time period selected from the group consisting of 1 minute, 2 minutes, 5 minutes, 10 minutes, 15 minutes, 20 minutes, 30 minutes, 1 hour, 2 hours, 4 hours, 8 hours, 16 hours, or 24 hours. In some embodiments, heating may be performed at or above the glass transition temperature (T_{g}) of silk and/or the polymeric fabric or textile, which may be assessed by methods known in the art. In some embodiments, the dispersion may be formed by coating silk, including recombinant silk with the characteristics described herein, onto the polymeric fabric or textile, and then performing an additional process to impregnate the silk coating into the polymeric fabric or textile, including treatment with an organic solvent. Methods for characterizing the properties of polymers dissolved in one another are well known in the art and include differential scanning calorimetry and surface analysis methods capable of depth profiling, including spectroscopic methods.

Compositions of the present disclosure are “hypoallergenic” meaning that they are relatively unlikely to cause an allergic reaction. Such hypoallergenicity can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely.

In some embodiments, where aqueous solutions are used to prepare RSPF compositions or RSPF containing coatings, the aqueous solutions may be prepared with DI water or tap water. As used herein, “tap water” refers to potable water provided by public utilities and water of comparable quality, regardless of the source, without further refinement such as by reverse osmosis, distillation, and/or deionization. Therefore, the use of “DI water,” “RODI water,” or “water,” as set forth herein, may be understood to be interchangeable with “tap water” according to the processes described herein without deleterious effects to such processes.

Cosmetic Compositions Containing Recombinant Silk-Based Protein Fragments

In some embodiments, the disclosure provides a recombinant silk personal care composition comprising recombinant silk fibroin fragments having an average weight average molecular weight selected from the group consisting of from about 1 kDa to about 5 kDa, from about 5 kDa to about 10 kDa, from about 6 kDa to about 17 kDa, from about 10 kDa to about 15 kDa, from about 15 kDa to about 20 kDa, from about 17 kDa to about 39 kDa, from about 20 kDa to about 25 kDa, from about 25 kDa to about 30 kDa, from about 30 kDa to about 35 kDa, from about 35 kDa to about 40 kDa, from about 39 kDa to about 80 kDa, from about 40 kDa to about 45 kDa, from about 45 kDa to about 50 kDa, from about 60 kDa to about 100 kDa, and from about 80 kDa to about 144 kDa, a polydispersity ranging from 1 to about 5. In some embodiments, recombinant silk is as provided in any of the compositions #1001-2450, #3001-4450, and #5001-6595. In some embodiments, the recombinant silk fibroin fragments are included in an aqueous solution. In some embodiments, the silk personal care composition further comprises silk amino acids resulted from the hydrolysis of silk of Bombyx mori and silk powders resulted from drying of the silk solution. In some embodiments of the recombinant silk personal care composition, the recombinant silk fibroin fragments are present at an amount ranging from about 0.01 wt. % to about 10.0 wt. % by the total weight of the recombinant silk personal care composition. In some embodiments of the recombinant silk personal care composition, the recombinant silk fibroin fragments are present at an amount ranging from about 0.01 wt. % to about 1.0 wt. % by the total weight of the recombinant silk personal care composition. In some embodiments of the recombinant silk personal care composition, the recombinant silk fibroin fragments are present at an amount ranging from about 1.0 wt. % to about 2.0 wt. % by the total weight of the recombinant silk personal care composition. In some embodiments of the recombinant silk personal care composition, the recombinant silk fibroin fragments are present at an amount ranging from about 2.0 wt. % to about 3.0 wt. % by the total weight of the recombinant silk personal care composition. In some embodiments of the recombinant silk personal care composition, the recombinant silk fibroin fragments are present at an amount ranging from about 3.0 wt. % to about 4.0 wt. % by the total weight of the recombinant silk personal care composition. In some embodiments of the recombinant silk personal care composition, the recombinant silk fibroin fragments are present at an amount ranging from about 4.0 wt. % to about 5.0 wt. % by the total weight of the recombinant silk personal care composition. In some embodiments of the recombinant silk personal care composition, the recombinant silk fibroin fragments are present at an amount ranging from about 5.0 wt. % to about 10.0 wt. % by the total weight of the recombinant silk personal care composition. In some embodiments of the recombinant silk personal care composition, the recombinant silk fibroin fragments in the recombinant silk personal care composition do not spontaneously or gradually gelate and do not visibly change in color or turbidity when in an aqueous solution for at least 10 days prior to be formulated into the recombinant silk personal care composition. In some embodiments of the recombinant silk personal care composition, there is included a carrier comprising an oil phase. In some embodiments of the recombinant silk personal care composition, there is included a carrier comprising an aqueous phase. In some embodiments of the recombinant silk personal care composition, the recombinant silk personal care composition further comprises an emulsifier. In some embodiments of the recombinant silk personal care composition, the recombinant silk personal care composition comprises an “oil-in-water” type emulsion or a “water-in-oil” type emulsion. In some embodiments of the recombinant silk personal care composition, the recombinant silk personal care composition is oral care composition.

In some embodiments of the recombinant silk personal care composition, the recombinant silk personal care composition is an oral care composition further comprising an additive selected from the group consisting of a filler, a diluent, a remineralizing agent, an anti-calculus agent, an anti-plaque agent, a buffer, an abrasive, an alkali metal bicarbonate salt, a binder, a thickening agent, a humectant, a whitening agent, a bleaching agent, a stain removing agent, a surfactant, titanium dioxide, a flavoring agent, xylitol, a coloring agent, a foaming agent, a sweetener, an antibacterial agent, a preservative, a vitamin, a pH-adjusting agent, an anti-caries agent, a desensitizing agent, a coolant, a salivating agent, a warming agent, a numbing agent, a chelating agent, and combinations thereof. In some embodiments, the recombinant silk oral care composition is formulated as a product selected from the group consisting of a toothpaste, a dentifrice, a tooth powder, an oral gel, an aqueous gel, a non-aqueous gel, a mouth rinse, a mouth spray, a plaque removing liquid, a denture product, a dental solution, a lozenge, an oral tablet, a chewing gum, a candy, a fast-dissolving film, a strip, a dental floss, a tooth glossing product, a finishing product, and an impregnated dental implement. In some embodiments, the recombinant silk oral care composition is formulated as a toothpaste comprising a tooth care active agent selected from the group consisting of an abrasive, an anti-calculus agent, an anti-plaque agent, a humectant, a whitening agent, an anti-caries agent, a desensitizing agent, a coolant, a salivating agent, a warming agent, a numbing agent, and combinations thereof. In some embodiments, the recombinant silk oral care composition is formulated as a tooth remineralization product comprising a therapeutically effective amount of a remineralizing agent. In some embodiments, the remineralizing agent is selected from the group consisting of fluoride, calcium source compound, phosphate source compound, calcium carbonate, sodium hydrogen phosphate, sodium dihydrogen phosphate, potassium hydrogen phosphate, potassium dihydrogen phosphate, amorphous calcium phosphate (ACP), tricalcium phosphate, casein phosphoprotein-ACP, bioactive glass, calcium sodium phosphosilicate, arginine bicarbonate-calcium carbonate complex, and combinations thereof. In some embodiments, the recombinant silk tooth remineralization composition is formulated as a product selected from the group consisting of remineralizing gel, a remineralizing mouthwash, a remineralizing tooth powder, a remineralizing chewing gums, a remineralizing lozenge, and a remineralizing toothpaste. In some embodiments of the recombinant silk personal care composition, the recombinant silk personal care composition is formulated as a skin cleansing composition. In some embodiments, the recombinant silk skin cleansing composition further comprises an additive selected from the group consisting of a cleansing surfactant, a soap base, a detergent, a lathering surfactant, a skin conditioning agent, an oil control agent, an anti-acne agent, an astringent, an exfoliating particle or agent, a skin calming agent, a plant extract, an essential oil, a coolant, a humectant, a moisturizer, a structurant, a gelling agent, an antioxidant, an anti-aging compound, a skin lightening agent, a preservative, a filler, a fragrance, a thickener, a coloring agent, an antimicrobial agent, and combinations thereof. In some embodiments, the recombinant silk skin cleansing composition is formulated as a product selected from the group consisting of a cleansing lotion, a cleansing milk, a cleansing gel, a cleansing soap bar, an exfoliating product, a bath and shower soap in bar, a body wash, a hand wash, a cleansing wipe, a cleansing pad, and a bath product. In some embodiments of the recombinant silk personal care composition, the recombinant silk personal care composition is formulated as a makeup composition. In some embodiments, the recombinant silk makeup composition further comprises a cosmetic ingredient selected from the group consisting of an oil control agent, a plant extract, an essential oil, a humectant, a moisturizer, a structurant, a gelling agent, an antioxidant, an anti-aging compound, a sunscreen, a skin lightening agent, a sequestering agent, a preservative, a filler, a fragrance, a thickener, a wetting agent, a coloring agent, a cosmetic powder, and a combination thereof. In some embodiments, the recombinant silk makeup composition is formulated as a product selected from the group consisting of a color cosmetic, a mascara, a lipstick, a lip liner, an eye shadow, an eye-liner, a rouge, a face powder, a foundation, and a blush. In some embodiments of the recombinant silk personal care composition, the recombinant silk personal care composition is formulated as a cosmetic composition and the carrier is a cosmetically acceptable medium. In some embodiments, the recombinant silk cosmetic composition further comprises a cosmetic ingredient selected from the group consisting of a surfactant, a skin conditioning agent, an oil control agent, an anti-acne agent, an astringent, an exfoliating particle or agent, a skin calming agent, a plant extract, an essential oil, a coolant, a humectant, a moisturizer, a structurant, a gelling agent, an antioxidant, an anti-aging compound, a sunscreen, a skin lightening agent, a sequestering agent, a preservative, a filler, a fragrance, a thickener, a wetting agent, a coloring agent, a glitter, and combinations thereof. In some embodiments, the recombinant silk cosmetic composition is formulated as a product selected from the group consisting of a cream, an emulsion, a foam, an ointment, a lotion, a liquid, a hydrogel, a shaving or after-shave cream, a conditioner, a cologne, a shaving or after-shave lotion, a perfume, a cosmetic oil, a facial mask, a moisturizer, an anti-wrinkle treatment cream, an eye treatment lotion, a tanning cream, a tanning lotion, a tanning emulsion, a sunscreen cream, a sunscreen lotion, a sunscreen emulsion, a tanning oil, a sunscreen oil, a hand lotion, a tonic, and a body lotion. In some embodiments of the recombinant silk personal care composition, the recombinant silk personal care composition is formulated as a deodorant or antiperspirant composition and the carrier is a dermatologically acceptable medium. In some embodiments, the recombinant silk deodorant or antiperspirant composition further comprises an additive selected from the group consisting of a deodorant active, an antiperspirant active, an emollient, a humectant, a moisturizer, an astringent, an antiseptic agent, a gellant, a surfactant, a thickening agent, a cosmetic powder, a fragrance, an antimicrobial agent, a preservative, a coloring agent, a filler, a co-emulsifier, a hardener, a strengthener, a chelating agent, a thixotropic agent, an oil absorbing agent, an antioxidant, and combinations thereof. In some embodiments, the recombinant silk deodorant or antiperspirant composition is formulated as a product selected from the group consisting of a stick, a roll-on, a powder, a gel, an aerosol, a paste, and a cream. In some embodiments, the recombinant silk deodorant or antiperspirant composition is clear, transparent, or translucent. In some embodiments of the recombinant silk personal care composition, the recombinant silk personal care composition is formulated as a nail care composition and the carrier is a dermatologically acceptable medium. In some embodiments, the recombinant silk nail care composition further comprises an additive selected from the group consisting of a film-forming agent, a suspending agent, a thixotropic agent, a coloring agent, a pigment, a glitter, a plasticizer, a thickening agent, a nail hydrating agent, a nail hardening agent, boric acid, a vitamin, a plant extract, an essential oil, a preservative, a mineral salt, a fruit extract, an algae extract, a fungus extract, a caviar extract, a vegetable oil, an amino acid, a peptide, a protein, a ceramide, allantoin or an allantoin derivative, an organosilicon derivative, an antioxidant, a UV light absorber, a moisturizer, a stabilizer, a fragrance, a micronutrient, a solvent, and combinations thereof. In some embodiments, the recombinant silk nail care composition is formulated as a product selected from the group consisting of a nail polish, and a nail polish remover.

In some embodiments, the disclosure provides a recombinant silk fibroin fragment composition comprising recombinant silk fibroin fragments having an average weight average molecular weight selected from the group consisting of from about 1 kDa to about 5 kDa, from about 5 kDa to about 10 kDa, from about 6 kDa to about 17 kDa, from about 10 kDa to about 15 kDa, from about 15 kDa to about 20 kDa, from about 17 kDa to about 39 kDa, from about 20 kDa to about 25 kDa, from about 25 kDa to about 30 kDa, from about 30 kDa to about 35 kDa, from about 35 kDa to about 40 kDa, from about 39 kDa to about 80 kDa, from about 40 kDa to about 45 kDa, from about 45 kDa to about 50 kDa, from about 60 kDa to about 100 kDa, and from about 80 kDa to about 144 kDa, and a polydispersity ranging from about 1 to about 5, and at least one emulsifiable component. In some embodiments, recombinant silk is as provided in any of the compositions #1001-2450, #3001-4450, and #5001-6595. In some embodiments of the recombinant silk fibroin fragment composition, the recombinant silk fibroin fragments are present at an amount ranging from about 0.01 wt. % to about 10.0 wt. % by the total weight of the recombinant silk fibroin fragment composition. In some embodiments of the recombinant silk fibroin fragment composition, the recombinant silk fibroin fragments are present at an amount ranging from about 0.01 wt. % to about 1.0 wt. % by the total weight of the recombinant silk fibroin fragment composition. In some embodiments of the recombinant silk fibroin fragment composition, the recombinant silk fibroin fragments are present at an amount ranging from about 1.0 wt. % to about 2.0 wt. % by the total weight of the recombinant silk fibroin fragment composition. In some embodiments of the recombinant silk fibroin fragment composition, the recombinant silk fibroin fragments are present at an amount ranging from about 2.0 wt. % to about 3.0 wt. % by the total weight of the recombinant silk fibroin fragment composition. In some embodiments of the recombinant silk fibroin fragment composition, the recombinant silk fibroin fragments are present at an amount ranging from about 3.0 wt. % to about 4.0 wt. % by the total weight of the recombinant silk fibroin fragment composition. In some embodiments of the recombinant silk fibroin fragment composition, the recombinant silk fibroin fragments are present at an amount ranging from about 4.0 wt. % to about 5.0 wt. % by the total weight of the recombinant silk fibroin fragment composition. In some embodiments of the recombinant silk fibroin fragment composition, the recombinant silk fibroin fragments are present at an amount ranging from about 5.0 wt. % to about 10.0 wt. % by the total weight of the recombinant silk fibroin fragment composition. In some embodiments of the recombinant silk fibroin fragment composition, the recombinant silk fibroin fragments do not spontaneously or gradually gelate and do not visibly change in color or turbidity when in an aqueous solution for at least 10 days prior to be formulated into the recombinant silk fibroin fragment composition. In some embodiments of the recombinant silk fibroin fragment composition, the recombinant silk fibroin fragments composition further comprises an additive selected from the group consisting of butanediol, propanediol, ethanediol, glycerol, butantetraol, xylitol, D-sorbitol, inositol, polyethylene glycol, hydroxyethyl cellulose, hydroxypropyl methylcellulose, dextran, gelatin, carboxymethyl cellulose, propylene glycol, polysorbate 80, polyvinyl alcohol, povidone, saponin, sucrose, fructose, maltose, carrageenan, chitosan, alginate, hyaluronic acid, and combinations thereof. In some embodiments of the recombinant silk fibroin fragment composition, the recombinant silk fibroin fragments composition comprises one or more solvents selected from the group consisting of methanol, ethanol, propanol, isopropanol, acetonitrile, and combinations thereof. In some embodiments of the recombinant silk fibroin fragment composition, the emulsifiable component comprises a hydrophobic emulsifiable component, a hydrophilic emulsifiable component, an amphiphilic emulsifiable component, or a combination thereof. In some embodiments of the recombinant silk fibroin fragment composition, the emulsifiable component comprises a hydrophobic emulsifiable component. In some embodiments of the recombinant silk fibroin fragment composition, the emulsifiable component comprises one or more of an oil, a fat, a wax, a lipid, and combinations thereof. In some embodiments of the recombinant silk fibroin fragment composition, the oil is selected from the group consisting of hydrocarbon oil, mineral oil, silicon oil, fatty acid having 8 to 32 carbon atoms, fatty alcohol having 8 to 32 carbon atoms, synthetic ester oil derived from the esterification product of fatty acid having 8 to 32 carbon atoms and an alcohol, fatty acid glyceride, glyceryl trioctanoate, glyceryl triisopalmitate, cholesteryl isostearate, isopropyl palmitate, isopropyl myristate, neopentyl glycol dicaprate, isopropyl isostearate, octadecyl myristate, cetyl 2-ethylhexanoate, cetearyl isononanoate, cetearyl octanoate, isononyl isononanoate, isotridecyl isononanoate, glyceryl tri-2-ethylhexanoate, glyceryl tri(caprylatelcaprate), diethylene glycol monoethyl ether oleate, dicaprylyl ether, caprylic acid/capric acid propylene glycol diester, isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexylate, dipentaerythritol fatty acid ester, N-alkyl glycol monoisostearate, neopentyl glycol dicaprate, diisostearyl malate, glyceryl di-2-heptylundecanoate, trimethylolpropane tri-2-ethylhexylate, trimethylolpropane triisostearate, pentaneerythritol tetra-2-ethylhexylate, glyceryl tri-2-ethylhexylate, trimethylolpropane triisostearate, cetyl 2-ethylhexanoate, 2-ethylhexyl palmitate, glyceryl trimyristate, tri-2-heptylundecanoic glyceride, oleyl oleate, cetostearyl alcohol, 2-heptylundecyl palmitate, diisopropyl adipate, N-lauroyl-L-glutamic acid-2-octyldodecyl ester, di-2-heptylundecyl adipate, ethyl laurate, di-2-ethylhexyl cebatate. 2-hexyldecyl myristate, 2-hexyldecyl palmitate, 2-hexyldecyl adipate, diisopropyl cebatate, 2-ethylhexyl succinate, ethyl acetate, butyl acetate, amyl acetate and triethyl (citrate. In some embodiments of the recombinant silk fibroin fragment composition, the fat is selected from the group consisting of liquid fat, solid fat, avocado oil, tsubaki oil, turtle oil, macadamia nut oil, corn oil, mink oil, olive oil, rape seed oil, egg yolk oil, sesame seed oil, persic oil, wheat germ oil, sasanqua oil, castor oil, linseed oil, safflower oil, cotton seed oil, perilla oil, soybean oil, peanut oil, tea seed oil, kaya oil, rice bran oil, Chinese wood oil, Japanese wood oil, jojoba oil, germ oil, sweet almond oil, rosehip seed oil, calendula oil, grape seed oil, apricot kernel oil, flaxseed oil, hazelnut oil, walnut oil, pecan nut oil, sesame oil, emu oil, coconut oil, sunflower oil, canola oil, algae oil, cacao butter, horse tallow, hardened coconut oil, palm oil, beef tallow, sheep tallow, pork tallow, hardened beef tallow, palm kernel oil, Japanese core wax, hydrogenated castor oil, and combinations thereof. In some embodiments of the recombinant silk fibroin fragment composition, the wax is selected from the group consisting of butter, petrolatum, polyethylene wax, polypropylene wax, Japanese wax, beeswax, candelilla wax, paraffin wax, ozokerite, microcrystalline wax, carnauba wax, cotton wax, esparto wax, bayberry wax, tree wax, whale wax, montan wax, bran wax, lanolin wax, kapok wax, lanolin acetate, sugar cane wax, lanolin fatty acid isopropyl ester, hexyl laurate, reduced lanolin, jojoba wax, hard lanolin, shellac wax, POE lanolin alcohol ether, lanolin alcohols with 40 moles ethylene oxide, lanolin alcohols with 65-70 moles ethylene oxide, POE lanolin alcohol acetate, POE cholesterol ether, lanolin fatty acid, POE hydrogenated lanolin alcohol ether, and combinations thereof. In some embodiments of the recombinant silk fibroin fragment composition, the lipid is selected from the group consisting of phospholipid, polymer-lipid conjugate, carbohydrate-lipid conjugate, dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-sn-glycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), cholesterol, 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phosphol-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof. In some embodiments of the recombinant silk fibroin fragment composition, the lipid is a phospholipid selected from soy lecithin and egg lecithin. In some embodiments of the recombinant silk fibroin fragment composition, the recombinant silk fibroin fragments composition further comprises a thickening agent or gelling agent selected from the group consisting of hydroxyethyl cellulose, hydroxypropyl methylcellulose, dextran, gelatin, carboxymethyl cellulose, propylene glycol, polysorbate 80, polyvinyl alcohol, povidone, sucrose, fructose, maltose, carrageenan, chitosan, alginate, hyaluronic acid, gum arabic, galactomannans, xanthan gum, pectin, and combinations thereof. In some embodiments of the recombinant silk fibroin fragment composition, the recombinant silk fibroin fragments composition further comprises a density matching agent or a weighting agent selected from the group consisting of ester gum (EG), damar gum (DG), sucrose acetate isobutyrate (SAIB), brominated vegetable oil (BVO), and combinations thereof. In some embodiments of the recombinant silk fibroin fragment composition, the weighting agent concentrations required to match the oil and aqueous phase densities is of about 10.0 wt. % to about 25.0 wt. % for BVO, about 35.0 wt. % to about 55.0 wt. % for EG, about 35.0 wt. % to about 55.0 wt. % for DG, and about 25.0 wt. % to about 45.0 wt. % for SAIB. In some embodiments of the recombinant silk fibroin fragment composition, a portion of the recombinant silk fibroin fragments has a hydrophilic-lipophilic balance (HLB) value selected from the group consisting of from 0 to about 3, from about 3 to about 6, from about 6 to about 9, from about 9 to about 12, from about 12 to about 15, from about 15 to about 18, and greater than 18. In some embodiments of the recombinant silk fibroin fragment composition, a portion of the recombinant silk fibroin fragments has a HLB value selected from the group consisting of 0, about 1, about 2, about 3, about 4, about 5, about 6, about 7, about 8, about 9, about 10, about 11, about 12, about 13, about 14, about 15, about 16, about 17, about 18, about 19, and about 20. In some embodiments of the recombinant silk fibroin fragment composition, a portion of the recombinant silk fibroin fragments has a HLB value ranging from about 8 to about 18. In some embodiments of the recombinant silk fibroin fragment composition, a portion of the recombinant silk fibroin fragments has a HLB value ranging from 0 to about 8. In some embodiments of the recombinant silk fibroin fragment composition, the recombinant silk fibroin fragments composition further comprises about 0.1 wt. % to about 5.0 wt. % of a natural surfactant having a HLB value of about 0 to about 6 that is capable of forming a gel network in a continuous aqueous phase and, wherein the silk fibroin fragment composition forms oil-in-water emulsion containing about 0.8 wt. % to about 10.0 wt. % silk fibroin fragments, wherein the wt. % is relative to the total weight of the silk fibroin fragment composition. In some embodiments, the natural surfactant is selected from the group consisting of C16-C24 fatty alcohol, soy lecithin, egg lecithin, sucrose ester, cetearyl glucoside, caprylyl/capryl glucoside, sucrose laurate, sucrose palmitate, sucrose stearate, sucrose cocoate, sorbitan monostearate, and combinations thereof. In some embodiments of the recombinant silk fibroin fragment composition, a portion of the recombinant silk fibroin fragments composition is an “oil-in-water” type emulsion. In some embodiments of the recombinant silk fibroin fragment composition, a portion of the recombinant silk fibroin fragments composition is a “water-in-oil” type emulsion. In some embodiments of the recombinant silk fibroin fragment composition, the recombinant silk fibroin fragments composition comprises one or more personal care active ingredients, wherein the recombinant silk personal care composition is formulated as an oral care composition, a skin care composition, a hair care composition, a cosmetic composition, a makeup composition, a sun care composition, a deodorant, an antiperspirant composition, a nail cosmetic composition, a skin cleansing composition, an aromatic cosmetic, or a bath cosmetic composition. In some embodiments, the recombinant silk personal care product is selected from the group consisting of a beauty soap, a soap bar, a facial wash, a hand wash, a body wash, a cleansing wipe, a feminine hygiene product, a cleansing pad, a cleansing foam, a rinse, a cleansing lotion, a cleansing milk, a cleansing gel, a cleansing soap bar, an exfoliating product, a bath and shower soap in bar, a cream, an emulsion, a shaving or after-shave cream, a foam, a conditioner, a cologne, a shaving or after-shave lotion, a perfume, a cosmetic oil, a facial mask, a moisturizer, an anti-wrinkle, an eye treatment, a tanning cream, a tanning lotion, a tanning emulsion, a sunscreen cream, a sunscreen lotion, a sunscreen emulsion, a tanning oil, a sunscreen oil, a hand lotion, a body lotion, a color cosmetic, a mascara, a lipstick, a lip liner, an eye shadow, an eye-liner, a rouge, a face powder, a foundation, a blush, perfume, bath soap in bar, bath product, a toothpaste, a dentifrice, a tooth powder, an oral gel, an aqueous gel, a non-aqueous gel, a mouth rinse, a mouth spray, a plaque removing liquid, a denture product, a dental solution, a lozenge, oral tablet, a chewing gum, a candy, a fast-dissolving film, a strip, a dental floss, a tooth glossing product, a finishing product, an impregnated dental implement, a remineralizing gel, a remineralizing mouthwash, a remineralizing tooth powder, a remineralizing chewing gum, a remineralizing lozenge, a remineralizing toothpaste, a antiperspirant stick, a roll-on deodorant, a powder deodorant, a gel deodorant, an aerosol deodorant, a paste deodorant, and a cream nail polish, and a nail polish remover. In some embodiments of the recombinant silk fibroin fragment composition, the recombinant silk fibroin fragments composition comprises one or more personal care active ingredients and at least one rheology modifier, wherein the recombinant silk personal care product contains at most 13 different ingredients in total. In some embodiments of the recombinant silk fibroin fragment composition, the recombinant silk fibroin fragments composition contains less than twelve different ingredients in total.

In some embodiments, the disclosure provides a recombinant silk oral care composition comprising recombinant silk fibroin fragments having an average weight average molecular weight selected from the group consisting of from about 1 kDa to about 5 kDa, from about 5 kDa to about 10 kDa, from about 6 kDa to about 17 kDa, from about 10 kDa to about 15 kDa, from about 15 kDa to about 20 kDa, from about 17 kDa to about 39 kDa, from about 20 kDa to about 25 kDa, from about 25 kDa to about 30 kDa, from about 30 kDa to about 35 kDa, from about 35 kDa to about 40 kDa, from about 39 kDa to about 80 kDa, from about 40 kDa to about 45 kDa, from about 45 kDa to about 50 kDa, from about 60 kDa to about 100 kDa, and from about 80 kDa to about 144 kDa, a polydispersity ranging from 1 to about 5; a dental care active agent; and one or more dentally acceptable excipients. In some embodiments, recombinant silk is as provided in any of the compositions #1001-2450, #3001-4450, and #5001-6595. In some embodiments of the recombinant silk oral care composition, the recombinant silk fibroin fragments are present at an amount ranging from about 0.01 wt. % to about 10.0 wt. % by the total weight of the recombinant silk oral care composition. In some embodiments of the recombinant silk oral care composition, the recombinant silk fibroin fragments are present at an amount ranging from about 0.01 wt. % to about 1.0 wt. % by the total weight of the recombinant silk oral care composition. In some embodiments of the recombinant silk oral care composition, the recombinant silk fibroin fragments are present at an amount ranging from about 1.0 wt. % to about 2.0 wt. % by the total weight of the recombinant silk oral care composition. In some embodiments of the recombinant silk oral care composition, the recombinant silk fibroin fragments are present at an amount ranging from about 2.0 wt. % to about 3.0 wt. % by the total weight of the recombinant silk oral care composition. In some embodiments of the recombinant silk oral care composition, the recombinant silk fibroin fragments are present at an amount ranging from about 3.0 wt. % to about 4.0 wt. % by the total weight of the recombinant silk oral care composition. In some embodiments of the recombinant silk oral care composition, the recombinant silk fibroin fragments are present at an amount ranging from about 4.0 wt. % to about 5.0 wt. % by the total weight of the recombinant silk oral care composition. In some embodiments of the recombinant silk oral care composition, the recombinant silk fibroin fragments are present at an amount ranging from about 5.0 wt. % to about 10.0 wt. % by the total weight of the recombinant silk oral care composition. In some embodiments of the recombinant silk oral care composition, the recombinant silk fibroin do not spontaneously or gradually gelate and do not visibly change in color or turbidity when in an aqueous solution for at least 10 days prior to be formulated into the recombinant silk oral care composition. In some embodiments of the recombinant silk oral care composition, the recombinant silk oral care composition is formulated as a solution. In some embodiments of the recombinant silk oral care composition, the recombinant silk oral care composition is formulated as an emulsion. In some embodiments of the recombinant silk oral care composition, the recombinant silk oral care composition is formulated as a powder. In some embodiments of the recombinant silk oral care composition, the recombinant silk oral care composition is formulated as a plurality of granules. In some embodiments of the recombinant silk oral care composition, the recombinant silk oral care composition is formulated as a gel. In some embodiments of the recombinant silk oral care composition, the recombinant silk oral care composition is formulated as a film. In some embodiments of the recombinant silk oral care composition, the recombinant silk oral care composition is formulated as a suspension. In some embodiments of the recombinant silk oral care composition, the recombinant silk oral care composition comprises an active agent selected from the group consisting of therapeutic agent, plaque removal agent, germicidal agent, anticalculus agents, abrasive polishing agent, whitening/bleaching/stain removing agent, anti-plaque agent, anti-tartar agents, anti-caries agents, remineralizing agent, humectant, and combinations thereof. In some embodiments, the remineralizing agent is selected from the group consisting of fluoride, calcium source compound, phosphate source compound, calcium carbonate, sodium hydrogen phosphate, sodium dihydrogen phosphate, potassium hydrogen phosphate, potassium dihydrogen phosphate, amorphous calcium phosphate (ACP), tricalcium phosphate, casein phosphoprotein-ACP, bioactive glass, calcium sodium phosphosilicate, arginine bicarbonate-calcium carbonate complex, and combinations thereof. In some embodiments, the therapeutic agent is selected from the group consisting of fluoride salt (sodium fluoride, stannous fluoride, sodium monofluorophosphate, ammonium fluoride), strontium salt, potassium salt, stannous fluoride, phosphate fluoride, hydrogen peroxide, potassium chlorate, potassium permanganates, clove oil, wintergreen, pontacaine, hemostatic agent, zinc salt, antioxidant, antibiotic, antimicrobials, antiseptic agent, antifungal agent, anesthetic agent, antiviral agent, anti-ulcer active agent, anti-allergic agent, anti-analgesic agent, analgesic, hemostatic agent, anti-inflammatory agent (flubiprofen, naproxen, ketoprofen, aspirin), growth factor, anti-tumor agent, desensitizing agent, hormones, Vitamin, amino acid, vaccine, caffeine, monoclonal antibody, enzyme, and combinations thereof. In some embodiments, the zinc salt is selected from the group consisting of Zinc chloride, Zinc acetate, Zinc phenol, Sulfonate, Zinc borate, Zinc bromide, Zinc nitrate, Zinc glycerophosphate, Zinc benzoate, Zinc carbonate, Zinc citrate, Zinc hexafluorosilicate, Zinc diacetate trihydrate, Zinc oxide, Zinc peroxide, Zinc Salicylate, Zinc silicate, Zinc Stannate, Zinc tannate, Zinc titanate, Zinc tetrafluoroborate, Zinc gluconate, and Zinc glycinate. In some embodiments, the a desensitizing agent is one or more strontium salts selected from the group consisting of strontium chloride, strontium bromide, strontium iodide, strontium acetate, strontium edetate, strontium nitrate, strontium salicylate, strontium lactate, and combinations thereof. In some embodiments, the antioxidant is selected from the group consisting of vitamin A, vitamin E, pyruvate B-carotene, selenium, N-acetylcysteine, vitamin C, superoxide dismutase (SOD), catalase, glutathione peroxidase, glutathione reductase, and combinations thereof. In some embodiments of the recombinant silk oral care composition, the recombinant silk oral care composition is combined with a support. In some embodiments, the support comprises a pellet, wood stick, metal stick, paper, a yarn, a thread, a fiber, a fabric layer, a film, and a hydrogel. In some embodiments, the fabric layer comprises one or more of a natural fiber or yarn comprising one or more of cotton and wool, or a synthetic fiber or yarn comprising one or more of polyester, nylon, polyester-polyurethane copolymer, polyamide, polyaramid, polytetrafluoroethylene, polyethylene, polypropylene, polyurethane, silicone, polyurethane, polyethyleneglycol, polypropylene (PP), thermoplastic polyurethane (TPU), polyethylene (PE), Nylon and combinations thereof. In some embodiments, the fabric layer comprises a nonwoven portion. In some embodiments, the nonwoven portion comprises one or more of cellulose, cotton, rayon, regenerated cellulose, chitosan, silk, polypropylene (PP), thermoplastic polyurethane (TPU), polyethylene (PE), Nylon and combinations thereof. In some embodiments of the recombinant silk oral care composition, the recombinant silk oral care composition is formulated into a product selected from the group consisting of a dental sheath, a dental patch, a floss, a tooth powder, a tooth tablet, capsule, lozenge, pastille, a toothpick, a whitening strip, a confectionary, a chewing gum, a tooth brushing sheet, toothpaste bite, an impregnated implement, a mouth piece, and an oral care strip. In some embodiments, the article is selected from a dental sheath, a dental patch, a floss, a tooth powder, a tooth tablet, capsule, lozenge, pastille, a toothpick, a whitening strip, a confectionary, a chewing gum, a tooth brushing sheet, toothpaste bite, an impregnated implement, a mouth piece, and an oral care strip.

According to aspects illustrated herein, there is disclosed a film that includes recombinant silk-based proteins or fragments thereof comprising: the recombinant silk protein or fragments thereof having an average weight average molecular weight ranging from about 17 kDa to about 38 kDa; and a polydispersity of between 1 and about 5.0, or between about 1.5 and about 3.0, wherein the film has a water content ranging from about 2.0 wt. % to about 20.0 wt. %, wherein the film includes between 0 ppm and 500 ppm of inorganic residuals, wherein the film includes between 0 ppm and 500 ppm of organic residuals, and wherein the film is sufficiently flexible to conform to anatomical topographies. In some embodiments, a film can include recombinant silk as provided in any of the compositions #1001-2450, #3001-4450, and #5001-6595. In an embodiment, the film includes between about 1.0% and about 50.0% crystalline protein domains and being soluble when submersed in water at room temperature. In an embodiment, the film includes from about 30.0 wt. % to about 99.5 wt. % of recombinant silk protein fragments. In an embodiment, the silk film has a pH from about 1.0 to about 7.0. In an embodiment, the silk film further includes from about 0.5 wt. % to about 2.5 wt. % of caffeine. In an embodiment, the silk film further includes from about 1.0 wt. % to about 50.0 wt. % of vitamin C or a derivative thereof. In an embodiment, the vitamin C or a derivative thereof remains stable within the film for a period of from about 5 days to about 5 years. In an embodiment, the vitamin C or a derivative thereof is stable within the silk film so as to result in release of the vitamin C in a biologically active form. In an embodiment, the silk film further includes one or more molecules selected from the group consisting of therapeutic agents, growth factors, antioxidants, proteins, carbohydrates, polymers, nucleic acids, salts, acids, bases, biomolecules, glycosamino glycans, polysaccharides, extracellular matrix molecules, metals, metal ion, metal oxide, synthetic molecules, polyanhydrides, cells, fatty acids, fragrance, minerals, plants, plant extracts, preservatives and essential oils. In an embodiment, the silk film further includes an alpha hydroxy acid selected from the group consisting of glycolic acid, lactic acid, tartaric acid and citric acid. In an embodiment, the silk film further includes hyaluronic acid or its salt form at a concentration ranging from about 0.5 wt. to about 10.0 wt. %. In an embodiment, the silk film further includes at least one of zinc oxide or titanium dioxide. In an embodiment, the silk film is packaged in a foil based package that is air tight and light proof. In an embodiment, the silk film is sufficiently designed for topical application. In an embodiment, the topical application is for cosmetic use. In an embodiment, the topical application is for wound dressing. In an embodiment, the silk film is sufficiently designed for administration within a body. In an embodiment, the recombinant silk protein fragments are hypoallergenic. In an embodiment, a method of reducing fine lines and wrinkles includes applying a silk film of the present disclosure daily to human skin for a period of at least one week and observing a reduction in fine lines and wrinkles on the human skin.

In an embodiment, the percent water content in the silk films of the present disclosure is 20 wt. %. In an embodiment, the percent water content in the silk films of the present disclosure is less than 20 wt. %. In an embodiment, the percent water content in the silk films of the present disclosure is less than 18 wt. %. In an embodiment, the percent water content in the silk films of the present disclosure is less than 16 wt. %. In an embodiment, the percent water content in the silk films of the present disclosure is less than 14 wt. %. In an embodiment, the percent water content in the silk films of the present disclosure is less than 12 wt. %. In an embodiment, the percent water content in the silk films of the present disclosure is less than 10 wt. %. In an embodiment, the percent water content in the silk films of the present disclosure is between about 2 wt. % and about 20 wt. % by the total weight of the silk film.

According to aspects illustrated herein, there is disclosed a dark spot silk film that includes recombinant silk-based proteins or fragments thereof includes about 1 wt. % to about 50 wt. % 1-ascorbic acid, a recombinant silk of 3 mg/cm² to 10 mg/cm², optionally the dark spot silk film can be clear/transparent. In an embodiment, a dark spot silk film of the present disclosure includes water, recombinant silk protein or fragments thereof, and vitamin C (L-ascorbic acid). In an embodiment, a dark spot silk film of the present disclosure includes 40 wt. % vitamin C. In an embodiment, a dark spot silk film of the present disclosure reduces skin pigmentation and evens skin tone in a targeted area with daily use. Vitamin C can inhibit pigment transfer from pigment producing cells, called melanocytes, to skin surface cells with continual application. In an embodiment, a dark spot silk film of the present disclosure can be applied to clean, dampened skin for 20 minutes. In an embodiment, additional water can be applied to an adhered film. The recombinant silk protein stabilization matrix in a dark spot silk film of the present disclosure protects the active ingredients from the air, to deliver their full benefits without the use of harsh chemicals or preservatives, such as paraben and phthalate. Thus, a dark spot film of the present disclosure is paraben and phthalate-free.

According to aspects illustrated herein, there is disclosed a fine line lifting film that includes recombinant silk-based proteins or fragments thereof comprising the silk quantities selected from the group consisting of 0.01 mg/cm² to 100 mg/cm², 0.1 mg/cm² to 50 mg/cm², 0.5 mg/cm² to 30 mg/cm², 1 mg/cm² to 20 mg/cm², and 3 mg/cm² to 10 mg/cm², about 1 wt. % to about 50 wt. % L-ascorbic acid. In an embodiment, a fine line lifting film of the present disclosure includes a plurality of perforations (e.g., 2, 3, 4, 5, 10, 15, 20, 25, etc.). In an embodiment, a fine line lifting film is perforated and/or shaped to conform to a portion of the human anatomy, wherein the portion of the human anatomy is selected from the group consisting of a neck, an elbow, a shoulder, a hip, a knee, an ankle, and a foot. In the foregoing embodiments, the fine line lifting film may be shaped without perforations, or may be perforated and not shaped, or may be both perforated and shaped. In an embodiment, a perforated and/or shaped fine line lifting film may contain a therapeutic agent. In an embodiment, a perforated and/or shaped fine line lifting film may contain a therapeutic agent, wherein the therapeutic agent is topically or transdermally delivered. In an embodiment, a perforated and/or shaped fine line lifting film may contain a therapeutic agent, wherein the therapeutic agent is selected from the group consisting of a chemotherapeutic agent, a pigment, an antibacterial agent, an antifungal agent, an antibiotic, an antimicrobial, an antimycotic, an antihistamine, an antiarrhythmic agent, an antihypertensive agent, a corticosteroid, an anti-viral agent, an antidepressant, an analgesic agent, an anesthetic agent, an anti-inflammatory agent, an attention-deficit hyperactivity disorder agent, an agent for the treatment of Parkinson's disease, an agent for the treatment of dementia, a smoking cessation agent, a pain relieving agent, a hormone therapy, an agent for the treatment of migraine disorders, an agent for the treatment of menopausal symptoms, an agents for contraception, an agent for chronic pain relief, an agent for angina prophylaxis, an agent for the treatment of osteoarthritis, an agent for postherpetic neuralgia, an agent for the treatment of a skin disorder, an agent for the treatment of acne, and an agent for the treatment of psoriasis.

In an embodiment, a perforated and/or shaped film may be used to topically or transdermally deliver any of the foregoing therapeutic agents. In an embodiment, the disclosure provides a method of treating a disease comprising the steps of (a) providing a perforated and/or shaped silk film, (b) optionally detaching and/or folding the perforated and/or shaped film, (c) applying the perforated and/or shaped silk film to a portion of the human anatomy, and (d) affixing the perforated and/or shaped silk film such that one or more therapeutic agents is delivered to the skin.

According to aspects illustrated herein, there is disclosed a silk gel that includes recombinant silk-based protein or fragments thereof comprising: a weight average molecular weight, or average weight average molecular weight ranging from about 17 kDa to about 39 kDa; and a polydispersity of between 1 and about 5.0, or between about 1.5 and about 3.0; and water from about 20 wt. % to about 99.9 wt. %, wherein the gel includes between 0 ppm and 500 ppm of inorganic residuals, and wherein the gel includes between 0 ppm and 500 ppm of organic residuals. In some embodiments, a gel described herein can include recombinant silk as provided in any of the compositions #1001-2450, #3001-4450, and #5001-6595. In an embodiment, the gel includes between about 1.0 wt. % and about 50.0 wt. % crystalline protein domains. In an embodiment, the gel includes from about 0.5 wt. % to about 8.0 wt. % of recombinant silk-based protein fragments. In an embodiment, the gel includes from about 0.1 wt. % to about 6.0 wt. % of recombinant silk-based protein fragments. In an embodiment, the gel has a pH from about 1.0 to about 7.0. In an embodiment, the gel further includes from about 0.5 wt. % to about 20.0 wt. %, preferably 0.67% w/v to 15% w/v of vitamin C or a derivative thereof. In an embodiment, the vitamin C or a derivative thereof remains stable within the gel for a period of from about 5 days to about 5 years. In an embodiment, the vitamin C or a derivative thereof is stable within the gel so as to result in release of the vitamin C in a biologically active form. In an embodiment, the gel further includes an additive selected from the group consisting of vitamin E, rosemary oil, rose oil, lemon juice, lemon grass oil and caffeine. In an embodiment, the gel is packaged in an airtight container. In an embodiment, the recombinant silk-based protein fragments are hypoallergenic. In an embodiment, the gel has less than 10 colony forming units per milliliter. In an embodiment, the ratio of silk to vitamin C did affect the ability of a solution to gel as ratios above 1:2 did not gel and a 1:2 ratio took twice as long as other lower ratios (5:1, 2.5:1, 1:1).

Gels of the present disclosure be clear/white in color. The silk gels of the present disclosure can have a consistency that is easily spread and absorbed by the skin. The silk gels of the present disclosure can produce no visual residue or oily feel after application. The silk gels of the present disclosure do not brown over time.

According to aspects illustrated herein, there is disclosed a silk gels that includes recombinant silk-based proteins or fragments thereof comprising: the recombinant silk protein or fragments thereof an average weight average molecular weight ranging from about 17 kDa to about 38 kDa; and a polydispersity of between 1 and about 5.0, or between about 1.5 and about 3.0; and water from about 20 wt. % to about 99.9 wt. %, wherein the gel includes between 0 ppm and 500 ppm of inorganic residuals, and wherein the gel includes between 0 ppm and 500 ppm of organic residuals. In some embodiments, a gel described herein includes recombinant silk as provided in any of the compositions #1001-2450, #3001-4450, and #5001-6595. In an embodiment, the gel includes between about 1.0 wt. % and about 50.0 wt. % crystalline protein domains. In an embodiment, the gel includes from about 0.1 wt. % to about 6.0 wt. % of recombinant silk fibroin protein fragments. In an embodiment, the gel has a pH from about 1.0 to about 7.0. In an embodiment, the gel further includes from about 0.5 wt. % to about 20.0 wt. % of vitamin C or a derivative thereof. In an embodiment, the vitamin C or a derivative thereof remains stable within the gel for a period of from about 5 days to about 5 years. In an embodiment, the vitamin C or a derivative thereof is stable within the gel so as to result in release of the vitamin C in a biologically active form. In an embodiment, the gel further includes an additive selected from the group consisting of vitamin E, rosemary oil, rose oil, lemon juice, lemon grass oil and caffeine. In an embodiment, the silk gel is packaged in an airtight container. In an embodiment, the recombinant silk protein fragments are hypoallergenic. In an embodiment, the silk gel has less than 10 colony forming units per milliliter. In an embodiment, a method of smoothing and rejuvenating human skin includes applying a silk gel of the present disclosure daily to human skin for a period of at least one week and observing an improvement in skin texture.

In an embodiment, the percent water content in gels of the present disclosure is 20 wt. % to 99.9 wt. %. In an embodiment, the percent water content in gels of the present disclosure is 20 wt. % to 25 wt. %. In an embodiment, the percent water content in gels of the present disclosure is 25 wt. % to 30 wt. %. In an embodiment, the percent water content in gels of the present disclosure is 30 wt. % to 35 wt. %. In an embodiment, the percent water content in gels of the present disclosure is 35 wt. % to 40 wt. %. In an embodiment, the percent water content in gels of the present disclosure is 40 wt. % to 45 wt. %. In an embodiment, the percent water content in gels of the present disclosure is 45 wt. % to 50 wt. %. In an embodiment, the percent water content in gels of the present disclosure is 50 wt. % to 55 wt. %. In an embodiment, the percent water content in gels of the present disclosure is 55 wt. % to 60 wt. %. In an embodiment, the percent water content in gels of the present disclosure is 60 wt. % to 65 wt. %. In an embodiment, the percent water in gel cosmetic gels of the present disclosure s is 65 wt. % to 70 wt. %. In an embodiment, the percent water content in gels of the present disclosure is 70 wt. % to 75 wt. %. In an embodiment, the percent water content in gels of the present disclosure is 75 wt. % to 80 wt. %. In an embodiment, the percent water content in gels of the present disclosure is 80 wt. % to 85 wt. %. In an embodiment, the percent water content in gels of the present disclosure is 85 wt. % to 90 wt. %. In an embodiment, the percent water content in gels of the present disclosure is 90 wt. % to 95 wt. %. In an embodiment, the percent water content in gels of the present disclosure is 95 wt. % to 99 wt. %.

Gels of the present disclosure can be made with about 0.5 wt. % to about 8 wt % recombinant silk solutions (including, but not limited to, as provided in any of the compositions #1001-2450, #3001-4450, and #5001-6595). Gels of the present disclosure can be made with ascorbyl glucoside at concentrations of about 0.67% v/v to about 15% w/v. Gels of the present disclosure be clear/white in color. Gels of the present disclosure can have a consistency that is easily spread and absorbed by the skin. Gels of the present disclosure can produce no visual residue or oily feel after application. Gels of the present disclosure do not brown over time. According to aspects illustrated herein, a method is disclosed for producing recombinant silk gels having entrapped molecules or therapeutic agents such as those listed in the following paragraphs. In an embodiment, at least one molecule or therapeutic agent of interest is physically entrapped into a RSPF mixture solution of the present disclosure during processing into aqueous gels. An aqueous recombinant silk gel of the present disclosure can be used to release at least one molecule or therapeutic agent of interest.

According to aspects illustrated herein, there is disclosed a high concentration vitamin C silk gel that includes recombinant silk-based proteins or fragments thereof comprises 5.0 wt. %, 10.0 wt. %, or 15.0 wt. % vitamin C and 2.0 wt. %, 3.0 wt. %, or 3.8 wt. % of recombinant silk protein or fragments thereof respectively.

According to aspects illustrated herein, there is disclosed a caffeine gel with vitamin C that includes recombinant silk-based proteins or fragments thereof comprising: 2 wt. % recombinant silk protein and fragment thereof and 100 mg L-ascorbic acid/15 mL solution. In an embodiment, a caffeine gel of the present disclosure is used for reducing puffy eyes. A range of essential oils can be used including, but not limited to, lemongrass, vanilla, geranium, and green tea.

According to aspects illustrated herein, there is disclosed a green tea gel with vitamin C that includes recombinant silk-based proteins or fragments thereof comprising: green tea prep (1 tea bag/250 mL water), 2 wt. % recombinant silk and 100 mg L-ascorbic acid/15 mL solution and 50 mg caffeine/15 mL solution. In an embodiment, the vitamin C gel include preservative and chelating agent. The preservative added was Verstatil SL by Kinetic (Water, Sodium Levulinate, Potassium Sorbate) at 1.5 wt. % and the chelating agent was Dermofeel-PA3 by Kinetic (Sodium Phytate) at 0.1 wt. %. The addition of preservatives extended gelation time to 7 days. Green tea gel is being observed for discoloration and integrity with L-ascorbic acid and ascorbic acid-2-glucoside gel comparisons.

According to aspects illustrated herein, there is disclosed a serum that includes recombinant silk-based proteins or fragments thereof and comprising: the recombinant silk protein or fragments thereof having a weight average molecular weight, or average weight average molecular weight ranging from about 17 kDa to about 38 kDa; and a polydispersity of between 1 and about 5.0, or between about 1.5 and about 3.0; and hyaluronic acid or its salt form from about 0.5 wt. % to about 10.0 wt. %, wherein the serum includes between 0 ppm and 500 ppm of inorganic residuals, and wherein the serum includes between 0 ppm and 500 ppm of organic residuals. In some embodiments, a serum described herein includes recombinant silk as provided in any of the compositions #1001-2450, #3001-4450, and #5001-6595. In an embodiment, the serum includes between about 1.0 wt. % and about 50.0 wt. % crystalline protein domains. In an embodiment, the serum includes from about 0.1 wt. % to about 6.0 wt. % of recombinant silk protein fragments. In an embodiment, the serum has a pH from about 1.0 to about 7.0. In an embodiment, the serum further includes an additive selected from the group consisting of vitamin E, rosemary oil, rose oil, lemon juice, lemon grass oil, vanilla, geranium, and green tea. In an embodiment, the serum further includes from about 0.5 wt. % to about 30.0 wt. % of vitamin C or a derivative thereof. In an embodiment, the vitamin C or a derivative thereof remains stable within the serum for a period of from about 5 days to about 5 years. In an embodiment, the vitamin C or a derivative thereof is stable within the serum so as to result in release of the vitamin C in a biologically active form. In an embodiment, the serum is packaged in an airtight container. In an embodiment, the recombinant silk fibroin protein fragments are hypoallergenic. In an embodiment, a method of moisturizing human skin includes applying daily a serum of the present disclosure to human skin for a period of at least one week and observing an improvement in skin hydration.

According to aspects illustrated herein, there is disclosed a silk hydrating serum that includes recombinant silk-based proteins or fragments thereof and comprising: 0.1% w/v recombinant silk, 0.25 wt. % sodium hyaluronate, 0.25 wt. % Aspen bark, and 0.01 wt. % lemongrass essential oil; 0.2% w/v recombinant silk, 0.25 wt. % sodium hyaluronate, 0.25 wt. % Aspen bark, and 0.01 wt. % lemongrass essential oil; 0.2% w/v recombinant silk, 0.25 wt. % sodium hyaluronate, 0.25 wt. % Aspen bark, and 0.02 wt. % lemongrass essential oil; 0.2% w/v recombinant silk, 0.25 wt. % sodium hyaluronate, 0.25 wt. % Aspen bark, and 0.05 wt. % lemongrass essential oil; 0.2% w/v recombinant silk, 0.25 wt. % sodium hyaluronate, 0.25 wt. % Aspen bark, and 0.10 wt. % lemongrass essential oil; 0.3% w/v recombinant silk, 0.25 wt. % sodium hyaluronate, 0.25 wt. % Aspen bark, and 0.10 wt. % lemongrass essential oil; 0.3% w/v recombinant silk, 0.25 wt. % sodium hyaluronate, 0.25 wt. % Aspen bark, and 0.10 wt. % lemongrass essential oil; 0.5% w/v recombinant silk, 0.25 wt. % sodium hyaluronate, 0.25 wt. % Aspen bark, and 0.10 wt. % lemongrass essential oil; 0.8% w/v recombinant silk, 0.50 wt. % sodium hyaluronate, 0.25 wt. % Aspen bark, and 0.10 wt. % lemongrass essential oil; 0.8% w/v recombinant silk, 0.50 wt. % sodium hyaluronate, 0.33 wt. % Aspen bark, and 0.13 wt. % lemongrass essential oil; 1.0% w/v recombinant silk, 0.75 wt. % sodium hyaluronate, 0.50 wt. % Aspen bark, and 0.13 wt. % lemongrass essential oil; 1.0% w/v recombinant silk, 1.0 wt. % sodium hyaluronate, 0.50 wt. % Aspen bark, and 0.13 wt. % lemongrass essential oil; 2.0% w/v recombinant silk, 2.0 wt. % sodium hyaluronate, 0.50 wt. % Aspen bark, and 0.13 wt. % lemongrass essential oil; 2.0% w/v recombinant silk, 2.0 wt. % sodium hyaluronate, 1.0 wt. % Aspen bark, and 0.13 wt. % lemongrass essential oil; 3.0% w/v recombinant silk, 2.0 wt. % sodium hyaluronate, 1.0 wt. % Aspen bark, and 0.13 wt. % lemongrass essential oil; 3.0% w/v recombinant silk, 3.0 wt. % sodium hyaluronate, 1.5 wt. % Aspen bark, and 0.13 wt. % lemongrass essential oil; 3.0% w/v recombinant silk, 4.0 wt. % sodium hyaluronate, 2.0 wt. % Aspen bark, and 0.2 wt. % lemongrass essential oil; 3.0% w/v recombinant silk, 5.0 wt. % sodium hyaluronate, 2.0 wt. % Aspen bark, and 0.2 wt. % lemongrass essential oil; and 3.0% w/v recombinant silk, 5.0 wt. % sodium hyaluronate, 3.0 wt. % Aspen bark, and 0.2 wt. % lemongrass essential oil.

According to aspects illustrated herein, there is disclosed an ultrasensitive silk hydrating serum that includes recombinant silk-based proteins or fragments thereof and comprising: 0.1% w/v silk, 0.25 wt. % sodium hyaluronate, 0.25 wt. % Aspen bark, 0.01 wt. % rosehip oil, and 0.05% w/v sodium anisate; 0.2% w/v recombinant silk, 0.25 wt. % sodium hyaluronate, 0.25 wt. % Aspen bark, 0.01 wt. % rosehip oil, and 0.05% w/v sodium anisate; 0.3% w/v recombinant silk, 0.25 wt. % sodium hyaluronate, 0.25 wt. % Aspen bark, 0.01 wt. % rosehip oil, and 0.05% w/v sodium anisate; 0.5% w/v recombinant silk, 0.25 wt. % sodium hyaluronate, 0.25 wt. % Aspen bark, 0.01 wt. % rosehip oil, and 0.05% w/v sodium anisate; 0.5% w/v recombinant silk, 0.35 wt. % sodium hyaluronate, 0.25 wt. % Aspen bark, 0.01 wt. % rosehip oil, and 0.05% w/v sodium anisate; 0.5% w/v silk, 0.35 wt. % sodium hyaluronate, 0.25 wt. % Aspen bark, 0.01 wt. % rosehip oil, and 0.1% w/v sodium anisate; 0.8% w/v recombinant silk, 0.35 wt. % sodium hyaluronate, 0.25 wt. % Aspen bark, 0.01 wt. % rosehip oil, and 0.1% w/v sodium anisate; 1% w/v recombinant silk, 0.35 wt. % sodium hyaluronate, 0.25 wt. % Aspen bark, 0.01 wt. % rosehip oil, and 0.1% w/v sodium anisate; 1% w/v recombinant silk, 0.50 wt. % sodium hyaluronate, 0.25 wt. % Aspen bark, 0.03 wt. % rosehip oil, and 0.1% w/v sodium anisate; 1% w/v recombinant silk, 0.50 wt. % sodium hyaluronate, 0.50 wt. % Aspen bark, 0.05 wt. % rosehip oil, and 0.1% w/v sodium anisate; 1.0% w/v recombinant silk, 0.75 wt. % sodium hyaluronate, 0.50 wt. % Aspen bark, 0.07 wt. % rosehip oil, and 0.1% w/v sodium anisate; 2.0% w/v recombinant silk, 0.75 wt. % sodium hyaluronate, 0.50 wt. % Aspen bark, 0.07 wt. % rosehip oil, and 0.1% w/v sodium anisate; 3.0% w/v recombinant silk, 2.0% sodium hyaluronate, 0.50 wt. % Aspen bark, 0.07 wt. % rosehip oil, and 1% w/v sodium anisate; and 3.0% w/v recombinant silk, 0.75 wt. % sodium hyaluronate, 0.75 wt. % Aspen bark, 0.07 wt. % rosehip oil, and 3% w/v sodium anisate.

According to aspects illustrated herein, there is disclosed a UV hydrating serum suitable for protection against ultraviolet radiation (UV) that includes recombinant silk-based proteins or fragments thereof at about 0.5% w/v to about 10% w/v, preferably 1.0% w/v of aqueous solution of recombinant silk protein or fragment thereof, 0.25% w/v to about 10% w/v, preferably 0.75% w/v of hyaluronic acid, 20 μL/15 mL silk solution of lemongrass oil, 6 g of sodium ascorbyl phosphate, zinc oxide at a concentration varied from 2.5 wt. %, 3.75 wt. %, wt. 5 wt. %, 5.625 wt. %, 10 wt. %, 12 wt. % and 15 wt. %, titanium dioxide at a concentrations varied from 1.25 wt. %, 1.875 wt. %, 3 wt. %, 5 wt. % and 10 wt. %. In an embodiment, the UV hydrating serum of the present disclosure can have a lubricious texture that is rubbed in easily without residue.

Increasing the concentration of UV filter additives resulted in minor increases of white residue and how well dispersed the additives were, however if mixed well enough the effects were negligible. Zinc oxide and titanium dioxide were mixed together into serums in order to achieve broad spectrum protection. Zinc oxide is a broad spectrum UV filter additive capable of protecting against long and short UVA and UVB rays. However, titanium dioxide is better at UVB protection and often added with zinc oxides for best broad spectrum protection. Combinations included 3.75 wt. %/1.25 wt. % ZnO/TiO₂, 5.625 wt. %/1.875 wt. % ZnO/TiO₂, 12 wt. %/3 wt. % ZnO/TiO₂, 15 wt. %/5 wt. % ZnO/TiO₂. The 3.75 wt. %/1.25 wt. % ZnO/TiO₂ resulted in RSPF 5 and the 5.625 wt. %/1.875 wt. % ZnO/TiO₂ produced RSPF 8.

A UV hydrating serum of the present disclosure can include one, or a combination of two or more, of these active organic chemical UV filter ingredients: oxybenzone, avobenzone, octisalate, octocrylene, homosalate and octinoxate. A UV hydrating serum of the present disclosure can also include a combination of zinc oxide with organic chemical UV filters.

In an embodiment, a hydrating UV serum of the present disclosure delivers moisture for immediate and long-term hydration throughout the day with concentrated hyaluronic acid. A range of essential oils can be used in a hydrating serum of the present disclosure including, but not limited to, lemongrass, vanilla, geranium, and green tea. In an embodiment, one or two drops of a hydrating UV serum of the present disclosure can be smoothed over the face and neck. In an embodiment, a hydrating UV serum of the present disclosure includes water, aqueous recombinant silk fibroin fragment solution, hyaluronic acid, and lemongrass oil. In an embodiment, the recombinant silk-based proteins or fragments thereof in a hydrating UV serum of the present disclosure has the ability to stabilize and protect skin while sealing in moisture, all without the use of harsh chemical preservatives or synthetic additives. In an embodiment, the hyaluronic acid in a hydrating UV serum of the present disclosure nourishes skin and delivers moisture for lasting hydration. In an embodiment, the lemongrass essential oil in a hydrating UV serum of the present disclosure yields antioxidant and anti-inflammatory properties that support skin rejuvenation. In an embodiment, a hydrating UV serum of the present disclosure has a pH of about 6.0.

In an embodiment, a hydrating UV serum of the present disclosure protects the skin and seals in moisture with the power of recombinant silk-based proteins or fragments thereof. In an embodiment, a hydrating UV serum of the present disclosure is designed to protect, hydrate, and diminish fine lines while shielding skin from harsh UVA and UVB rays. In an embodiment, the recombinant silk protein in a hydrating UV serum of the present disclosure stabilizes and protects skin while sealing in moisture, without the use of harsh chemical preservatives or synthetic additives. In an embodiment, the vitamin C derivative in a hydrating UV serum of the present disclosure acts as a powerful antioxidant that supports skin rejuvenation. In an embodiment, the sodium hyaluronate in a hydrating UV serum of the present disclosure nourishes the skin and delivers moisture for long-lasting hydration.

In an embodiment, the zinc oxide and titanium dioxide in a hydrating UV serum of the present disclosure shields skin from harmful UVA and UVB rays. The silk protein stabilization matrix in a hydrating UV serum of the present disclosure protects the active ingredients from the air, to deliver their full benefits without the use of harsh chemicals or preservatives. The recombinant silk matrix also traps moisture within the skin furthering the hydrating effect of the sodium hyaluronate.

According to aspects illustrated herein, there is disclosed a skin peel composition that includes recombinant silk-based proteins or fragments thereof, the fragments having an average weight average molecular weight ranging from about 17 kDa to about 38 kDa and a polydispersity of between 1 and about 5.0, or between about 1.5 and about 3.0 in combination with at least one skin exfoliating agent. In some embodiments, a skin peel described herein includes recombinant silk as provided in any of the compositions #1001-2450, #3001-4450, and #5001-6595. In an embodiment, the skin peel composition includes at least one skin exfoliating agent selected from the group consisting of glycolic acid and lactic acid. In an embodiment, the skin peel composition includes between about 1.0% and about 50.0% crystalline protein domains. In an embodiment, the skin peel composition has a pH from about 1.0 to about 6.0. In an embodiment, the recombinant silk protein or fragments thereof are hypoallergenic.

According to aspects illustrated herein, there is disclosed a skin barrier composition that includes recombinant silk-based proteins or fragments thereof, the fragments having an average weight average molecular weight ranging from about 6 kDa to about 17 kDa, or from about 17 kDa to about 39 kDa, or from about 39 kDa to about 80 kDa, and a polydispersity of between 1 and about 5.0, or between about 1.5 and about 3.0. In some embodiments, a skin barrier described herein includes recombinant silk as provided in any of the compositions #1001-2450, #3001-4450, and #5001-6595. In some embodiments, a skin barrier comprises one or more of phosphatidylcholine (a phospholipid, for example from lecithin), one or more ceramides, one or more triglycerides (e.g., from coconut oil), squalane (e.g., from olives), one or more fatty acids, and/or one or more phytosterols (e.g., from evening primrose, borage, and/or shea). In an embodiment, the skin barrier composition includes between about 1.0% and about 50.0% crystalline protein domains. In an embodiment, the skin barrier composition has a pH from about 1.0 to about 6.0. In an embodiment, the recombinant silk protein or fragments thereof are hypoallergenic.

According to aspects illustrated herein, there is disclosed a skin penetrant composition that includes recombinant silk-based proteins or fragments thereof, the fragments having an average weight average molecular weight ranging from about 6 kDa to about 17 kDa, or from about 17 kDa to about 39 kDa, or from about 39 kDa to about 80 kDa, and a polydispersity of between 1 and about 5.0, or between about 1.5 and about 3.0. In some embodiments, a skin penetrant described herein includes recombinant silk as provided in any of the compositions #1001-2450, #3001-4450, and #5001-6595. In some embodiments, a skin penetrant comprises one or more of isopropyl myristate, decyl oleate, oleyl alcohol, octyldodecanol, propylene glycol, triacetin, cocoyl caprylocaprate, one or more sulphoxides and similar chemicals (e.g., DMSO), azone, one or more pyrrolidones, one or more fatty acids, one or more essential oils, terpenes and terpenoids, one or more oxazolidinones, and/or urea. In an embodiment, the skin penetrant composition includes between about 1.0% and about 50.0% crystalline protein domains. In an embodiment, the skin penetrant composition has a pH from about 1.0 to about 6.0. In an embodiment, the recombinant silk protein or fragments thereof are hypoallergenic.

A skin peel of the present disclosure can have the concentration of the recombinant silk protein or fragments thereof ranging from about 0.5 wt. % to about 8 wt. %. The pH of a skin peel of the present disclosure can be adjusted with varying quantities of lactic and glycolic acid. The skin peels can also be made with lactic acid only or glycolic acid only. A skin peel of the present disclosure can be clear or white in color. A skin peel of the present disclosure can have a gel consistency that is easily spread and absorbed by the skin. A skin peel of the present disclosure does not brown or change colors.

In an embodiment, a chemical peel of the present disclosure can be applied weekly to reveal healthy, vibrant skin. In an embodiment, a chemical peel of the present disclosure can be applied weekly to diminish fine lines. In an embodiment, a chemical peel of the present disclosure can be applied weekly to firm the skin.

Chemical peels are intended to burn the top layers of the skin in a controlled manner, to remove superficial dermal layers and dead skin in order to improve appearance. Alpha-hydroxyl acids (AHAs) are common in chemical peels due to low risk of adverse reactions and high control of strength (control pH and time applied). Glycolic acid is most commonly used and has a very small molecular size, enabling deep penetration into the epidermis. Lactic acid is another commonly used AHA and offers a more gentle peel with higher control due to its larger molecular size. Any number of chemicals known in the art that lower pH and are physical exfoliates can be used in place of AHAs.

According to aspects illustrated herein, there is disclosed a skin reviewing peel composition that includes recombinant silk-based proteins or fragments thereof comprising: 0.2% w/v recombinant silk, 0.10% w/v glycolic acid, 0.10% v/v lactic acid (88% solution), and 0.01 w/v % lemongrass essential oil; 0.2% w/v recombinant silk, 0.20% w/v glycolic acid, 0.20% v/v lactic acid (88% solution), and 0.02% w/v lemongrass essential oil; 0.4% w/v recombinant silk, 0.20% w/v glycolic acid, 0.30% v/v lactic acid (88% solution), and 0.03% w/v lemongrass essential oil; 0.8% w/v recombinant silk, 0.20% w/v glycolic acid, 0.30% v/v lactic acid (88% solution), and 0.04% lemongrass essential oil; 1.0% w/v recombinant silk, 0.40% w/v glycolic acid, 0.30% v/v lactic acid (88% solution), and 0.04% lemongrass essential oil; 1.5% w/v recombinant silk, 0.60% w/v glycolic acid, 0.40% v/v lactic acid (88% solution), and 0.08% lemongrass essential oil; 2.0% w/v recombinant silk, 0.80% w/v glycolic acid, 0.60% v/v lactic acid (88% solution), and 0.13% lemongrass essential oil; 2.0% w/v recombinant silk, 0.80% w/v glycolic acid, 0.60% v/v lactic acid (88% solution), and 0.18% lemongrass essential oil; 2.5% w/v recombinant silk, 1.00% w/v glycolic acid, 0.80% v/v lactic acid (88% solution), and 0.25% lemongrass essential oil; 3.0% w/v recombinant silk, 1.00% w/v glycolic acid, 0.80% v/v lactic acid (88% solution), and 0.25% lemongrass essential oil; and 6.0% w/v recombinant silk, 1.00% w/v glycolic acid, 0.80% v/v lactic acid (88% solution), and 0.33% lemongrass essential oil.

According to aspects illustrated herein, there is disclosed a UV foam composition that includes recombinant silk protein fragments comprising 1%, 3%, and 5% of recombinant silk by weight having weight average molecular weight, or average weight average molecular weight at about 25 KDa, or 60 KDa, 2.5 wt. %-3.5 wt. % of hyaluronate, 3.0 wt. % ZnO and 0.825 wt. %-1.65 wt. % TiO₂.

According to aspects illustrated herein, there is disclosed a silk intensive C composition that includes recombinant silk-based proteins or fragments thereof and comprising: 0.1% w/v recombinant silk, 1.0% w/v sodium hyaluronate, 5.0% ascorbyl glucoside w/v, 0.25% w/v Aspen bark, 3.0% v/v NaOH, and 0.1% lemongrass essential oil; 0.1% w/v recombinant silk, 1.0% w/v sodium hyaluronate, 11.13% ascorbyl glucoside w/v, 0.50% w/v Aspen bark, 6.25% v/v NaOH, and 0.13% lemongrass essential oil; 0.3% w/v recombinant silk, 1.2% w/v sodium hyaluronate, 5.15% ascorbyl glucoside w/v, 0.50% w/v Aspen bark, 4.0% v/v NaOH, and 0.13% lemongrass essential oil; 0.5% w/v recombinant silk, 1.45% w/v sodium hyaluronate, 8.82% ascorbyl glucoside w/v, 0.50% w/v Aspen bark, 5.0% v/v NaOH, and 0.21% lemongrass essential oil; 1.0% w/v recombinant silk, 3.03% w/v sodium hyaluronate, 9.0% ascorbyl glucoside w/v, 0.75% w/v Aspen bark, 5.5% v/v NaOH, and 0.23% lemongrass essential oil; and 2.5% w/v recombinant silk, 4.5% w/v sodium hyaluronate, 11.13% ascorbyl glucoside w/v, 0.75% w/v Aspen bark, 5.5% v/v NaOH, and 0.23% lemongrass essential oil.

According to aspects illustrated herein, there is disclosed an ultrasensitive silk intensive C composition that includes recombinant silk-based proteins or fragments thereof and comprising: 0.10% w/v recombinant silk, 1.00% w/v sodium hyaluronate, 3.33% w/v ascorbyl glucoside, 6.05% v/v NaOH, 0.50% w/v Aspen bark, 0.10% w/v sodium anisate, and 0.07% rosehip oil; 0.10% w/v recombinant silk, 1.00% w/v sodium hyaluronate, 7.50% w/v ascorbyl glucoside, 6.05% v/v NaOH, 0.50% w/v Aspen bark, 0.10% w/v sodium anisate, and 0.07% rosehip oil; 0.10% w/v recombinant silk, 1.00% w/v sodium hyaluronate, 11.13% w/v ascorbyl glucoside, 6.05% v/v NaOH, 0.50% w/v Aspen bark, 0.10% w/v sodium anisate, and 0.07% rosehip oil; 0.30% w/v recombinant silk, 1.00% w/v sodium hyaluronate, 11.13% w/v ascorbyl glucoside, 6.05% v/v NaOH, 0.50% w/v Aspen bark, 0.10% w/v sodium anisate, and 0.07% rosehip oil; 0.30% w/v recombinant silk, 1.00% w/v sodium hyaluronate, 13.40% w/v ascorbyl glucoside, 5.67% v/v NaOH, 0.75% w/v Aspen bark, 0.10% w/v sodium anisate, and 0.15% rosehip oil; and 1.00% w/v recombinant silk, 2.50% w/v sodium hyaluronate, 8.33% w/v ascorbyl glucoside, 4.00% v/v NaOH, 2.00% w/v Aspen bark, 0.50% w/v sodium anisate, and 0.35% rosehip oil. According to aspects illustrated herein, there is disclosed a silk eye revive composition that includes recombinant silk protein fragments comprising 0.1% w/v recombinant silk, 0.1% ascorbyl glucoside, 0.1% sodium anisate, 0.1% caffeine powder, and 0.01% lemongrass essential oil; 0.3% w/v recombinant silk, 0.3% ascorbyl glucoside, 0.2% sodium anisate, 0.1% caffeine powder, and 0.02% lemongrass essential oil; 0.5% w/v recombinant silk, 0.5% ascorbyl glucoside, 0.2% sodium anisate, 0.3% caffeine powder, and 0.03% lemongrass essential oil; 0.8% w/v recombinant silk, 0.7% ascorbyl glucoside, 0.2% sodium anisate, 0.3% caffeine powder, and 0.06% lemongrass essential oil; 1.0% w/v recombinant silk, 0.8% ascorbyl glucoside, 0.3% sodium anisate, 0.5% caffeine powder, and 0.06% lemongrass essential oil; 1.5% w/v recombinant silk, 1.0% ascorbyl glucoside, 0.5% sodium anisate, 0.5% caffeine powder, and 0.13% lemongrass essential oil; 2.0% w/v recombinant silk, 0.7% ascorbyl glucoside, 0.5% sodium anisate, 0.5% caffeine powder, and 0.13% lemongrass essential oil; 2.5% w/v recombinant silk, 0.7% ascorbyl glucoside, 0.5% sodium anisate, 0.5% caffeine powder, and 0.13% lemongrass essential oil; 2.5% w/v recombinant silk, 0.9% ascorbyl glucoside, 0.5% sodium anisate, and 0.13% lemongrass essential oil; 3.0% w/v recombinant silk, 0.9% ascorbyl glucoside, 0.5% sodium anisate, 0.5% caffeine powder, and 0.13% lemongrass essential oil; 3.0% w/v recombinant silk, 0.9% ascorbyl glucoside, 1.0% sodium anisate, 0.5% caffeine powder, and 0.2% lemongrass essential oil; 4.0% w/v recombinant silk, 1.5% ascorbyl glucoside, 1.0% sodium anisate, 1.0% caffeine powder, and 0.2% lemongrass essential oil; 4.0% w/v recombinant silk, 2.0% ascorbyl glucoside, 2.0% sodium anisate, 1.0% caffeine powder, and 0.3% lemongrass essential oil; 5.0% w/v recombinant silk, 3.0% ascorbyl glucoside, 3.0% sodium anisate, 1.0% caffeine powder, and 0.3% lemongrass essential oil; 6.0% w/v recombinant silk, 4.0% ascorbyl glucoside, 4.0% sodium anisate, 1.0% caffeine powder, and 0.3% lemongrass essential oil; 6.0% w/v recombinant silk, 6.0% ascorbyl glucoside, 5.0% sodium anisate, 2.0% caffeine powder, and 0.2% lemongrass essential oil; 5.0% w/v recombinant silk, 7.0% ascorbyl glucoside, 4.0% sodium anisate, 1.0% caffeine powder, and 0.2% lemongrass essential oil; 4.0% w/v recombinant silk, 8.0% ascorbyl glucoside, 4.0% sodium anisate, and 0.2% lemongrass essential oil; 3.0% w/v recombinant silk, 9.0% ascorbyl glucoside, 3.0% sodium anisate, 1.0% caffeine powder, and 0.1% lemongrass essential oil; and 3.0% w/v recombinant silk, 10.0% ascorbyl glucoside, 3.0% sodium anisate, 0.8% caffeine powder, and 0.1% lemongrass essential oil.

According to aspects illustrated herein, there is disclosed an ultrasensitive silk eye revive composition that includes recombinant silk protein fragments comprising 0.1% w/v recombinant silk, 0.1% ascorbyl glucoside, 0.1% sodium anisate, 0.1% caffeine powder, and 0.01% rosehip oil; 0.3% w/v recombinant silk, 0.3% ascorbyl glucoside, 0.2% sodium anisate, 0.1% caffeine powder, and 0.02% rosehip oil; 0.5% w/v recombinant silk, 0.5% ascorbyl glucoside, 0.2% sodium anisate, 0.3% caffeine powder, and 0.03% rosehip oil; 0.8% w/v recombinant silk, 0.7% ascorbyl glucoside, 0.2% sodium anisate, 0.3% caffeine powder, and 0.06% rosehip oil; 1.0% w/v recombinant silk, 0.8% ascorbyl glucoside, 0.3% sodium anisate, 0.5% caffeine powder, and 0.06% rosehip oil; 1.5% w/v recombinant silk, 1.0% ascorbyl glucoside, 0.5% sodium anisate, 0.5% caffeine powder, and 0.13% rosehip oil; 2.0% w/v recombinant silk, 0.7% ascorbyl glucoside, 0.5% sodium anisate, 0.5% caffeine powder, and 0.13% rosehip oil; 2.5% w/v recombinant silk, 0.7% ascorbyl glucoside, 0.5% sodium anisate, 0.5% caffeine powder, and 0.13% rosehip oil; 2.5% w/v recombinant silk, 0.9% ascorbyl glucoside, 0.5% sodium anisate, and 0.13% rosehip oil; 3.0% w/v recombinant silk, 0.9% ascorbyl glucoside, 0.5% sodium anisate, 0.5% caffeine powder, and 0.13% rosehip oil; 3.0% w/v recombinant silk, 0.9% ascorbyl glucoside, 1.0% sodium anisate, 0.5% caffeine powder, and 0.2% rosehip oil; 4.0% w/v recombinant silk, 1.5% ascorbyl glucoside, 1.0% sodium anisate, 1.0% caffeine powder, and 0.2% rosehip oil; 4.0% w/v recombinant silk, 2.0% ascorbyl glucoside, 2.0% sodium anisate, 1.0% caffeine powder, and 0.3% rosehip oil; 5.0% w/v recombinant silk, 3.0% ascorbyl glucoside, 3.0% sodium anisate, 1.0% caffeine powder, and 0.3% rosehip oil; 6.0% w/v recombinant silk, 4.0% ascorbyl glucoside, 4.0% sodium anisate, 1.0% caffeine powder, and 0.3% rosehip oil; 6.0% w/v recombinant silk, 6.0% ascorbyl glucoside, 5.0% sodium anisate, 2.0% caffeine powder, and 0.2% rosehip oil; 5.0% w/v recombinant silk, 7.0% ascorbyl glucoside, 4.0% sodium anisate, 1.0% caffeine powder, and 0.2% rosehip oil; 4.0% w/v recombinant silk, 8.0% ascorbyl glucoside, 4.0% sodium anisate, and 0.2% rosehip oil; 3.0% w/v recombinant silk, 9.0% ascorbyl glucoside, 3.0% sodium anisate, 1.0% caffeine powder, and 0.1% rosehip oil; and 3.0% w/v recombinant silk, 10.0% ascorbyl glucoside, 3.0% sodium anisate, 0.8% caffeine powder, and 0.1% rosehip oil. According to aspects illustrated herein, there is disclosed a silk moisturizer comprising recombinant silk protein or fragments thereof. Silk fragments moisturizers have been described for example in WO 2016/176633, US 2018/0280274, and 2018/0008522, which are incorporated by reference herein in their entirety. In some embodiments, recombinant silk moisturizers can be made by any methods described in WO 2016/176633 or US 2018/0280274, and include recombinant silk as provided in any of the compositions #1001-2450, #3001-4450, and #5001-6595. In an embodiment, a silk moisturizer of the present disclosure can be used to address fine lines and wrinkles of the skin, for example fine lines and wrinkles around the mouth and nose. In an embodiment, a silk moisturizer of the present disclosure can be used to address dark spots on the skin. In an embodiment, a silk moisturizer of the present disclosure is used for reducing puffy eyes. In an embodiment, a silk moisturizer of the present disclosure is used for reducing dark circles around the eyes. In an embodiment, a silk gel of the present disclosure can be used as a firming eye moisturizer. In an embodiment, a silk moisturizer of the present disclosure can replenish moisture and increase cell renewal while restoring radiance. In an embodiment, a silk moisturizer of the present disclosure can be used as a hydrating moisturizer to restore hydration to the skin. In an embodiment, a silk moisturizer of the present disclosure can be used to treat redness, acne and hyperpigmentation of the skin. In an embodiment, an article of the present disclosure is a silk sunscreen moisturizer.

According to aspects illustrated herein, there is disclosed a moisturizing composition including a recombinant silk protein solution, hyaluronic acid, an oil or butter, and a pH adjusting agent. In some embodiments the recombinant silk protein solution may include about 1% to about 10% (w/v) of recombinant silk fibroin protein or fragments thereof. In some embodiments the recombinant silk fibroin protein or fragments thereof have a weight average molecular weight, or average weight average molecular weight ranging from about 6 kDa to about 16 kDa, from about 17 kDa to about 38 kDa, or from about 39 kDa to about 80 kDa. In some embodiments the recombinant silk fibroin protein fragments have a polydispersity of between 1 and about 5.0, or between about 1.5 and about 3.0. In some embodiments, recombinant silk is as provided in any of the compositions #1001-2450, #3001-4450, and #5001-6595. In some embodiments the oil or butter is jojoba oil, rosehip oil, glycerin, coconut oil, lemongrass oil, or shea butter. In some embodiments a moisturizing compositions may further include a second oil or butter. In some embodiments the second oil or butter is jojoba oil, rosehip oil, glycerin, coconut oil, lemongrass oil, or shea butter. In some embodiments the first oil or butter is present in an amount of about 0.1% to about 25% (v/v) of the moisturizing composition. In some embodiments the second oil or butter is present in an amount of about 0.1% to about 25% (v/v) of the moisturizing composition. In some embodiments the pH adjusting agent is NaOH. In other embodiments the pH adjusting agent is HCl. In still other embodiments the pH adjusting agent includes a second pH adjusting agent. In some embodiments one of the first pH adjusting agent and the second pH adjusting agent is NaOH and the other of the first pH adjusting agent and the second pH adjusting agent is HCl. In some embodiments a moisturizing composition further includes an additive. Example additives include vitamin E, aspen bark, sodium anisate, oat flour, titanium dioxide, and combinations thereof. In some embodiments the additive is a combination of vitamin E, aspen bark, and sodium anisate. In some embodiments a moisturizing composition further comprises water.

According to aspects illustrated herein, there is disclosed a method for preparing a moisturizer composition of recombinant silk protein fragments including: introducing water into a vessel; adding hyaluronic acid powder is added to the water;

mixing the hyaluronic acid and water to form a solution; adding a solution of recombinant silk fibroin based protein fragments to the hyaluronic acid solution;

mixing the hyaluronic acid and recombinant silk fibroin based protein fragments introducing one or more oils and/or butters and a pH adjusting agent to the hyaluronic acid/pure recombinant silk fibroin protein solution; mixing until a white, lotion-like homogeneous mixture is formed. In some embodiments a method further includes adding an additive to the hyaluronic acid/pure silk fibroin protein solution, and/or adding an additive to the white, white, lotion-like homogenous mixture and mixing. In some embodiments the oil and/or butter is jojoba oil, rosehip oil, glycerin, coconut oil, lemongrass oil, shea butter, or a combination thereof. In some embodiments the recombinant silk fibroin protein and fragments thereof have a weight molecular weight ranging from about 6 kDa to about 17 kDa, from about 17 kDa to about 39 kDa, or from about 39 kDa to about 80 kDa. In some embodiments the recombinant silk protein and fragments thereof have a polydispersity of between 1 and about 5.0, or between about 1.5 and about 3.0. In some embodiments, recombinant silk is as provided in any of the compositions #1001-2450, #3001-4450, and #5001-6595.

In an embodiment, the recombinant silk fibroin protein or fragments thereof in the moisturizer composition are in the form of a solution. In an embodiment, the recombinant silk solution composition includes from about 0.1 wt. % to about 30 wt. % recombinant silk fibroin protein or fragments thereof. In other embodiments, the silk solution composition includes from about 0.1 wt. % to about 20 wt. %, 1 wt. % to about 15 wt. %, about 2 wt. % to about 10 wt. %, about 5 wt. %, about 6 wt. %, or about 7 wt. % recombinant silk protein or fragments thereof. The recombinant silk fibroin protein or fragments thereof may be stable in the solution for at least 30 days. In an embodiment, the term “stable” refers to the absence of spontaneous or gradual gelation, with no visible change in the color or turbidity of the solution. In an embodiment, the term “stable” refers to no aggregation of fragments and therefore no increase in molecular weight over time. In an embodiment, the recombinant silk solution composition is in the form of an aqueous solution. In an embodiment, the silk solution composition is in the form of an organic solution. The recombinant silk solution composition may be provided in a sealed container. In some embodiments, the composition further includes one or more molecules selected from the group consisting of therapeutic agents, growth factors, antioxidants, proteins, vitamins, carbohydrates, polymers, nucleic acids, salts, acids, bases, biomolecules, glycosamino glycans, polysaccharides, extracellular matrix molecules, metals, metal ion, metal oxide, synthetic molecules, polyanhydrides, ceils, fatty acids, fatty alcohols, emollients, humectants, acid salts, emulsifiers, chelating agents fragrance, minerals, plants, plant extracts, preservatives, proteoglycans, essential oils, peptides, alcohols, tinting agents, titanium dioxide, zinc oxide, oat flour, and chemical UV filters. In an embodiment, the added molecule or molecules are stable (i.e., retain activity over time) within the composition and can be released at a desired rate. In an embodiment, the one or more molecules is vitamin C, Vitamin B, Vitamin A, or a derivative thereof. In an embodiment, the composition further includes an alpha hydroxy acid selected from the group consisting of glycolic acid, lactic acid, tartaric acid and citric acid. In an embodiment, the composition further includes hyaluronic acid or its salt form at a concentration of about 0.5 wt. % to about 10.0 wt. %. In an embodiment, the composition further includes at least one of zinc oxide or titanium dioxide. In an embodiment, the recombinant silk protein or fragments thereof in the composition are hypoallergenic. In an embodiment, the recombinant silk protein or fragments thereof are biocompatible, non-sensitizing, and non-immunogenic. In an embodiment, the recombinant silk protein or fragments thereof are bioresorbable or biodegradable following implantation or application. In an embodiment, the recombinant silk protein or fragments thereof are hypoallergenic.

According to aspects illustrated herein, there is disclosed a silk solution composition that includes recombinant silk protein or fragments thereof, wherein the silk solution composition has a weight average molecular weight, or average weight average molecular weight ranging from about 39 kDa to about 80 kDa, wherein the silk solution composition has a polydispersity of between 1 and about 5.0, or between about 1.5 and about 3.0, wherein the silk solution composition is substantially homogenous, wherein the silk solution composition includes between 0 ppm and about 500 ppm of inorganic residuals, and wherein the silk solution composition includes between 0 ppm and about 500 ppm of organic residuals, in an embodiment, the recombinant silk protein or fragments thereof have between about 10 ppm and about 300 ppm of lithium bromide residuals and between about 10 ppm and about 100 ppm of sodium carbonate residuals. In some embodiments, recombinant silk is as provided in any of the compositions #1001-2450, #3001-4450, and #5001-6595. In an embodiment, the silk solution composition lithium bromide residuals are measurable using a high-performance liquid chromatography lithium bromide assay, and the sodium carbonate residuals are measurable using a high-performance liquid chromatography sodium carbonate assay. In an embodiment, the silk solution composition is in the form of a solution. In an embodiment, the silk solution composition includes from about 0.1 wt. % to about 30.0 wt. % recombinant silk protein or fragments thereof. In other embodiments, the silk solution composition includes from about 0.1 wt. % to about 20 wt. %, 1 wt. % to about 15 wt. %, about 2 wt. % to about 10 wt. %, about 5 wt. %, about 6 wt. %, or about 7 wt. % recombinant silk protein fragments. The recombinant silk protein fragments are stable in the solution for at least 30 days. In an embodiment, the term “stable” refers to the absence of spontaneous or gradual gelation, with no visible change in the color or turbidity of the solution. In an embodiment, the term “stable” refers to no aggregation of fragments and therefore no increase in molecular weight over time. In an embodiment, the composition is in the form of an aqueous solution. In an embodiment, the composition is in the form of an organic solution. The composition may be provided in a sealed container. In some embodiments, the composition further includes one or more molecules selected from the group consisting of therapeutic agents, growth factors, antioxidants, proteins, vitamins, carbohydrates, polymers, nucleic acids, salts, acids, bases, biomolecules, glycosamino glycans, polysaccharides, extracellular matrix molecules, metals, metal ion, metal oxide, synthetic molecules, polyanhydrides, cells, fatty acids, fragrance, minerals, plants, plant extracts, preservatives and essential oils. In an embodiment, the added molecule or molecules are stable (i.e., retain activity over time) within the composition and can be released at a desired rate. In an embodiment, the one or more molecules is vitamin C, Vitamin B, Vitamin A, or a derivative thereof. In an embodiment, the composition further includes an alpha hydroxy acid selected from the group consisting of glycolic acid, lactic acid, tartaric acid and citric acid. In an embodiment, the composition further includes hyaluronic acid or its salt form at a concentration of about 0.5 wt. % to about 10.0 wt. %). In an embodiment, the composition further includes at least one of zinc oxide or titanium dioxide. In an embodiment, the recombinant silk protein fragments in the composition are hypoallergenic. In an embodiment, the recombinant silk protein fragments are biocompatible, non-sensitizing, and non-immunogenic. In an embodiment, the silk fibroin-based protein fragments are bioresorbable or biodegradable following implantation or application.

According to aspects illustrated herein, there is disclosed a moisturizing composition that includes recombinant silk protein or fragments thereof comprising: the recombinant protein or fragments thereof having weight average molecular weight, or average weight average molecular weight ranging from about 17 kDa to about 38 kDa; and a polydispersity of between 1 and about 5.0, or between about 1.5 and about 3.0, wherein the moisturizing composition has a water content ranging from about 2.0 wt. % to about 20.0 wt. %, wherein the moisturizing composition includes between about 0 ppm and about 500 ppm of inorganic residuals, wherein the moisturizing composition includes between about 0 ppm and about 500 ppm of organic residuals. In some embodiments, recombinant silk is as provided in any of the compositions #1001-2450, #3001-4450, and #5001-6595. In an embodiment, the moisturizing composition includes between about 1.0 wt. % and about 50.0 wt. % crystalline protein domains and being soluble when submersed in water at room temperature. In an embodiment, the moisturizing composition includes from about 1 wt. % to about 30 wt. % of recombinant silk protein or fragments thereof. In other embodiments, the silk solution composition includes from about 0.1 wt. % to about 20 wt. %, 1 wt. % to about 15 wt. %, about 2 wt. % to about 10 wt. %, about 5 wt. %, about 6 wt. %, or about 7 wt. % recombinant silk fibroin protein or fragments thereof.

In an embodiment, the moisturizing composition has a pH from about 1.0 to about 8.0. In an embodiment, the moisturizing composition further includes one or more molecules selected from the group consisting of therapeutic agents, growth factors, antioxidants, proteins, carbohydrates, polymers, nucleic acids, salts, acids, bases, biomolecules, glycosamino glycans, polysaccharides, extracellular matrix molecules, metals, metal ion, metal oxide, synthetic molecules, polyanhydrides, cells, fatty acids, fragrance, minerals, plants, plant extracts, preservatives and essential oils. In an embodiment, the moisturizing composition further includes an alpha hydroxy acid selected from the group consisting of glycolic acid, lactic acid, tartaric acid and citric acid. In an embodiment, the moisturizing composition further includes hyaluronic acid or its salt form at a concentration ranging from about 0.5 wt. % to about 10.0 wt. %. In an embodiment, the moisturizing composition further includes at least one of zinc oxide or titanium dioxide. In an embodiment, the moisturizing composition further includes an additive selected from vitamin E, aspen bark, sodium anisate, oat flour, titanium dioxide, honeysuckle blend, or combinations thereof. In an embodiment, the moisturizing composition is packaged in an airtight container. In an embodiment, the moisturizing composition is sufficiently designed for topical application. In an embodiment, the topical application is for cosmetic use. In an embodiment, the topical application is for wound dressing.

In an embodiment, at least one preservation mechanisms/preservatives is used in a cosmetic product of the present disclosure. In some embodiments, including a preservative can reduce growth of bacteria and/or fungus in a cosmetic composition of the present disclosure (i.e., anti-bacterial and/or anti-fungal). In some embodiments, products can include, but are not limited to, chemical peels, silk serums, silk gels, or any combination thereof. In an embodiment, a chemical peel of the present disclosure includes at least one preservative selected from the group consisting of pH and Lemongrass essential oil. In an embodiment, a silk serum of the present disclosure includes at least one preservative selected from the group consisting of Aspen Bark Extract, Lemongrass essential oil, Dermosoft® anisate, sodium benzoate, potassium sorbate, polylysine, or any combination thereof. In an embodiment, a silk gel of the present disclosure includes at least one preservative such as Lemongrass essential oil, and a particular manufacturing method results in the generation of a homogeneous distribution of the Lemongrass essential oil throughout the silk gel, where the silk gel does not include an emollient.

In an embodiment, a cosmetic composition of the present disclosure can include Aspen Bark (i.e., Populus Tremuloides (Aspen) Bark Extract) at a use level of between 0.2-3.0 wt. % of a cosmetic composition. In an embodiment, Aspen Bark includes salicylate content of about 54.0-60.0 wt. %. In an embodiment, the use level of Aspen Bark is between 0.2-2.5 wt. %. In an embodiment, the use level of Aspen Bark is between 0.2-2.0 wt. %. In an embodiment, the use level of Aspen Bark is between 1.0-3.0 wt. %. In an embodiment, the use level of Aspen Bark is between 1.5-3.0 wt. %. In an embodiment, the use level of Aspen Bark is between 2.0-3.0 wt. %. In an embodiment, the use level of Aspen Bark is between 2.5-3.0 wt. %. In an embodiment, the use level of Aspen Bark is between 1.0-2.0 wt. %.

In an embodiment, a cosmetic composition of the present disclosure can include Dermosoft® anisate at a concentration of between about 0.05 wt. % to about 0.3 wt. %. In an embodiment, Dermosoft® anisate is included in the product at a concentration of between about 0.05 wt. % to about 0.25 wt. %. In an embodiment, Dermosoft® anisate is included in the product at a concentration of between about 0.1 wt. % to about 0.20 wt. %. In an embodiment, Dermosoft® anisate is included in the product at a concentration of between about 0.15 wt. % to about 0.25 wt. %.

In an embodiment, a cosmetic composition of the present disclosure can include sodium benzoate at a concentration between about 0.0001 wt. % to about 1.0 wt. %. In an embodiment, a cosmetic composition of the present disclosure can include sodium benzoate between about 0.001 wt. % to about 1.0 wt. %. In an embodiment, a cosmetic composition of the present disclosure can include sodium benzoate between about 0.01 wt. % to about 1.0 wt. %. In an embodiment, a cosmetic composition of the present disclosure can include sodium benzoate between about 0.1 wt. % to about 1.0 wt. %. In an embodiment, a cosmetic composition of the present disclosure can include sodium benzoate between about 0.01 wt. % to about 0.1 wt. %. In an embodiment, a cosmetic composition of the present disclosure can include potassium sorbate at a concentration between about 0.1 wt. % to about 0.5 wt. %. In an embodiment, a cosmetic composition of the present disclosure can include polylysine at a concentration between about 0.0001% to about 1%. In an embodiment, a cosmetic composition of the present disclosure can include polylysine at a concentration between about 0.0001 wt. % to about 1 wt. %. In an embodiment, a cosmetic composition of the present disclosure can include polylysine at a concentration between about 0.001 wt. % to about 1 wt. %. In an embodiment, a cosmetic composition of the present disclosure can include polylysine at a concentration between about 0.01 wt. % to about 1 wt. %. In an embodiment, a cosmetic composition of the present disclosure can include polylysine at a concentration between about 0.1 wt. % to about 1 wt. %. In an embodiment, a cosmetic composition of the present disclosure can include polylysine at a concentration between about 0.0001 wt. % to about 0.1 wt. %. In an embodiment, a cosmetic composition of the present disclosure can include Lemongrass essential oil at a concentration between about 0.1 wt. % to about 0.5 wt. %. In an embodiment, a cosmetic composition of the present disclosure can be substantially scentless (i.e., “unscented product for use on sensitive skin”). In an embodiment, the use of an unscented product can result in substantially less irritation as compared with a cosmetic composition including scent.

In an embodiment, the use of an unscented product can result in substantially less inflammation as compared with a cosmetic composition including scent. In an embodiment, the use of an unscented product can result in a substantially less affliction triggered by an immunological response as compared with a cosmetic composition including scent. In an embodiment, a scentless product of the present disclosure is a hydrating serum, a vitamin C serum, and a silk smoothing gel. In an embodiment, a scentless product of the present disclosure includes rosehips essential oil, and does not include Lemongrass essential oil.

In an embodiment, the percent recombinant silk protein or fragments thereof in the solution is less than 30 wt. %. In an embodiment, the percent recombinant silk protein or fragments thereof in the solution is less than 25 wt. %. In an embodiment, the percent recombinant silk protein or fragments thereof in the solution is less than 20 wt. %. In an embodiment, the percent recombinant silk protein or fragments thereof in the solution is between 0.1 wt. % and 30 wt. %. In an embodiment, the percent recombinant silk protein or fragments thereof in the solution is between 0.1 wt. % and 25 wt. %. In an embodiment, the percent recombinant silk protein or fragments thereof in the solution is between 0.1 wt. % and 20 wt. %. In an embodiment, the percent recombinant silk in the solution is between 0.1 wt. % and 15 wt. %. In an embodiment, the percent recombinant silk in the solution is between 0.1 wt. % and 10 wt. %. In an embodiment, the percent recombinant silk in the solution is between 0.1 wt. % and 9 wt. %. In an embodiment, the percent recombinant silk in the solution is between 0.1 wt. % and 8 wt. %. In an embodiment, the percent recombinant silk in the solution is between 0.1 wt. % and 7 wt. %. In an embodiment, the percent recombinant silk in the solution is between 0.1 wt. % and 6.5 wt. %. In an embodiment, the percent recombinant silk in the solution is between 0.1 wt. % and 6 wt. %. In an embodiment, the percent recombinant silk in the solution is between 0.1 wt. % and 5.5 wt. %. In an embodiment, the percent recombinant silk in the solution is between 0.1 wt. % and 5 wt. %. In an embodiment, the percent recombinant silk in the solution is between 20 wt. % and 30 wt. %. In an embodiment, the percent recombinant silk in the solution is between 0.1 wt. % and 6 wt. %. In an embodiment, the percent recombinant silk in the solution is between 6 wt. % and 10 wt. %. In an embodiment, the percent recombinant silk in the solution is between 6 wt. % and 8 wt. %. In an embodiment, the percent recombinant silk in the solution is between 6 wt. % and 9 wt. %. In an embodiment, the percent recombinant silk in the solution is between 10 wt. % and 20 wt. %. In an embodiment, the percent recombinant silk in the solution is between 11 wt. % and 19 wt. %. In an embodiment, the percent recombinant silk in the solution is between 12 wt. % and 18 wt. %. In an embodiment, the percent recombinant silk in the solution is between 13 wt. % and 17 wt. %. In an embodiment, the percent recombinant silk in the solution is between 14 wt. % and 16 wt. %. In an embodiment, the percent recombinant silk in the solution is 2.4 wt. %. In an embodiment, the percent recombinant silk in the solution is 2.0 wt. %.

In an embodiment, the solubility of recombinant silk protein or fragments thereof of the present disclosure in organic solutions is 50 to 100%. In an embodiment, the solubility of recombinant silk protein or fragments thereof of the present disclosure in organic solutions is 60 to 100%. In an embodiment, the solubility of recombinant silk protein or fragments thereof of the present disclosure in organic solutions is 70 to 100%. In an embodiment, the solubility of recombinant silk protein or fragments thereof of the present disclosure in organic solutions is 80 to 100%. In an embodiment, the solubility of recombinant silk protein or fragments thereof of the present disclosure in organic solutions is 90 to 100%. In an embodiment, the recombinant silk protein or fragments thereof of the present disclosure are non-soluble in organic solutions.

In an embodiment, a preserved recombinant silk protein or fragments thereof solution or gel exhibits a log 10 reduction in bacterial content selected from the group consisting of 0, 1, 2, 3, 4, 5, 6, 7, 8, and 9 log 10 reductions. In an embodiment, a solution or gel of preserved recombinant silk protein or fragments thereof exhibits no bacterial growth. In an embodiment, a preserved recombinant silk protein or fragments thereof solution or gel exhibits a log 10 reduction in fungal content selected from the group consisting of 0, 1, 2, 3, 4, 5, 6, 7, 8, and 9 log 10 reductions. In an embodiment, a preserved recombinant silk protein or fragments thereof solution or gel exhibits no fungal growth. In an embodiment, the log 10 reduction is observed with respect to a starting amount of bacterial and/or fungal content provided by standard, such as the standard used in ISO 11930.

### Salt Leached 3D Scaffolds

In an embodiment, the disclosure provides a salt leached scaffolds were made in accordance with the published methods of Rockwood. Salt with particle sizes of interest was prepared by stacking the sieves with the largest mesh on top and the smallest mesh on the bottom. Salt was added and sieves shaken vigorously collecting the salt. With a 5-ml syringe, 6% (w/v) fibroin solution was aliquoted into plastic containers, 2 ml per mold and 5-600 microns salt particles were slowly added on top of the fibroin solution in the mold while rotating the container so that the salt was uniform. The ratio of salt to silk in solution was maintained at 25:1.

### Medical Material/Dermal Filler

In one embodiment, the disclosure relates to a method of treating a condition in a subject in need thereof, and/or a method of cosmetic treatment in a subject in need thereof, the method comprising administering to the subject a therapeutically effective amount of a biocompatible tissue filler comprising: a glycosaminoglycan selected from the group consisting of hyaluronic acid (HA), carboxymethyl cellulose (CMC), starch, alginate, chondroitin-4-sulfate, chondroitin-δ-sulfate, xanthan gum, chitosan, pectin, agar, carrageenan, and guar gum; and an anesthetic agent; wherein a portion of the glycosaminoglycan is cross-linked by cross-linking moieties comprising one or more of an alkane or alkyl chain, an ether group, and a secondary alcohol; and wherein cross-linking is obtained using a cross-linking agent, a cross-linking precursor, or an activating agent; the tissue filler optionally comprising recombinant silk protein or silk protein fragments (RSPF), wherein a portion of the RSPF are cross-linked. In some embodiments, recombinant silk is as provided in any of the compositions #1001-2450, #3001-4450, and #5001-6595. Dermal fillers including silk fragments are described for example in WO 2019/005848, which is incorporated herein by reference in its entirety. In some embodiments, the condition is a skin condition. In some embodiments, the skin condition is selected from the group consisting of skin dehydration, lack of skin elasticity, skin roughness, lack of skin tautness, a skin stretch line, a skin stretch mark, skin paleness, a dermal divot, a sunken cheek, a thin lip, a retro-orbital defect, a facial fold, and a wrinkle. In some embodiments the tissue filler is administered into a dermal region of the subject. In some embodiments, the method is an augmentation, a reconstruction, treating a disease, treating a disorder, correcting a defect or imperfection of a body part, region or area. In some embodiments, the method is a facial augmentation, a facial reconstruction, treating a facial disease, treating a facial disorder, treating a facial defect, or treating a facial imperfection. In some embodiments, the tissue filler resists biodegradation, bioerosion, bioabsorption, and/or bioresorption, for at least about 3 days, about 7 days, about 14 days, about 21 days, about 28 days, about 1 month, about 2 months, about 3 months, about 4 months, about 5 months, or about 6 months. In some embodiments, administration of the tissue filler to the subject results in a reduced inflammatory response compared to the inflammatory response induced by a control tissue filler comprising a polysaccharide and lidocaine, wherein the control tissue filler does not include recombinant silk protein fragments (RSPF). In some embodiments, administration of the tissue filler to the subject results in increased collagen production compared to the collagen production induced by a control tissue filler comprising a polysaccharide and lidocaine, wherein the control tissue filler does not include recombinant silk protein fragments (RSPF).

In one embodiment, the disclosure relates to a biocompatible tissue filler including recombinant silk protein fragments (RSPF) having a polydispersity of between 1 and about 5.0, or between about 1.5 and about 3.0, and a polysaccharide. In some embodiments, recombinant silk is as provided in any of the compositions #1001-2450, #3001-4450, and #5001-6595. In some embodiments, the polysaccharide is hyaluronic acid (HA). In an embodiment, the disclosure includes tissue fillers that may be prepared from silk and hyaluronic acid. In one embodiment, the disclosure relates to a biocompatible tissue filler comprising: a glycosaminoglycan selected from the group consisting of hyaluronic acid (HA), carboxymethyl cellulose (CMC), starch, alginate, chondroitin-4-sulfate, chondroitin-δ-sulfate, xanthan gum, chitosan, pectin, agar, carrageenan, and guar gum; and an anesthetic agent; wherein a portion of the glycosaminoglycan is cross-linked by cross-linking moieties comprising one or more of an alkane or alkyl chain, an ether group, and a secondary alcohol; and wherein cross-linking is obtained using a cross-linking agent, a cross-linking precursor, or an activating agent; the tissue filler comprising recombinant silk protein or fragments (RSPF) thereof

In some embodiments, the silk protein is a recombinant silk fibroin. In some embodiments, the recombinant silk fibroin fragments have a weight average molecular weight, or average weight average molecular weight ranging from about 1 kDa to about 250 kDa. In some embodiments, the recombinant silk fibroin fragments have a weight average molecular weight, or average weight average molecular weight ranging from about 5 kDa to about 150 kDa. In some embodiments, the recombinant silk fibroin fragments have a weight average molecular weight ranging from about 6 kDa to about 17 kDa. In some embodiments, the recombinant silk fibroin fragments have a weight average molecular weight, or average weight average molecular weight ranging from about 17 kDa to about 39 kDa. In some embodiments, the recombinant silk fibroin fragments have a weight average molecular weight, or average weight average molecular weight ranging from about 39 kDa to about 80 kDa. In some embodiments, the recombinant silk fibroin fragments have a molecular weight ranging from about 80 kDa to about 150 kDa. In some embodiments, the recombinant silk fibroin fragments have low molecular weight. In some embodiments, the recombinant silk fibroin fragments have medium molecular weight. In some embodiments, the recombinant silk fibroin fragments have high molecular weight. In some embodiments, the recombinant silk fibroin fragments have a polydispersity of between 1 and about 5.0, or between about 1.5 and about 3.0. In some embodiments, recombinant silk is as provided in any of the compositions #1001-2450, #3001-4450, and #5001-6595. In some embodiments, the recombinant silk fibroin fragments have a degree of crystallinity of up to 60%. In some embodiments, a portion of the recombinant silk fibroin fragments are cross-linked. In some embodiments, the degree of cross-linking of the cross-linked recombinant silk fibroin fragments is between about 1% and about 100%. In some embodiments, the degree of cross-linking of the cross-linked recombinant silk fibroin fragments is between about 1% and about 15%. In some embodiments, the degree of cross-linking of the cross-linked recombinant silk fibroin fragments is one or more of about 1%, about 2%, about 3%, about 4%, about 5%, about 6%, about 7%, about 8%, about 9%, about 10%, about 11%, about 12%, about 13%, about 14%, and about 15%. In some embodiments, the recombinant silk fibroin fragments were cross-linked to recombinant silk fibroin fragments using cross-linking agents such as BDDE, or one of the other cross-linking agents described herein. In some embodiments, the degree of cross-linking is up to about 100%.

In one embodiment, the disclosure relates to a biocompatible tissue filler including recombinant silk fibroin fragments having a polydispersity of between 1 and about 5.0, or between about 1.5 and about 3.0, and hyaluronic acid (HA), wherein up to about 0% to 100% of the RSPF, preferably at least 0.1% of recombinant silk fibroin fragments are cross-linked to HA and the recombinant silk fibroin fragments were cross-linked to recombinant silk fibroin fragments using a cross-linking agent such as BDDE, or one of the other cross-linking agents described herein, and the recombinant silk fibroin fragments degree of cross-linking is up to about 100%. In some embodiments, recombinant silk is as provided in any of the compositions #1001-2450, #3001-4450, and #5001-6595. In some embodiments, at least 0.1% of HA is non-cross-linked. In some embodiments, all of the HA is non-cross-linked. In some embodiments, all of the HA is non-cross-linked.

In one embodiment, the disclosure relates to a biocompatible hydrogel tissue filler including recombinant silk fibroin fragments having a polydispersity of between 1 and about 5.0, or between about 1.5 and about 3.0, and a polysaccharide, optionally water, an active agent selected from an enzyme inhibitor, an anesthetic agent (e.g. lidocaine), a medicinal neurotoxin, an antioxidant, an anti-infective agents, vasodilators, a reflective agent, an anti-inflammatory agent, an ultraviolet (UV) light blocking agent, a dye, a hormone, an immunosuppressant, or an anti-inflammatory agent, wherein recombinant silk fibroin fragments have a degree of crystallinity of about 0% to about 60%. In some embodiments, ‘G’ is measured by means of an oscillatory stress of about 0.1 to about 10 Hz. In one embodiment, G′ is measured by means of an oscillatory stress of about 1 Hz.

In one embodiment, the disclosure relates to a biocompatible tissue filler comprising: a glycosaminoglycan selected from the group consisting of hyaluronic acid (HA), carboxymethyl cellulose (CMC), starch, alginate, chondroitin-4-sulfate, chondroitin-δ-sulfate, xanthan gum, chitosan, pectin, agar, carrageenan, and guar gum; and an anesthetic agent; wherein a portion of the glycosaminoglycan is cross-linked by cross-linking moieties comprising one or more of an alkane or alkyl chain, an ether group, and a secondary alcohol; and wherein cross-linking is obtained using a cross-linking agent, a cross-linking precursor, or an activating agent; the tissue filler comprising recombinant silk protein or recombinant silk protein fragments (RSPF), wherein a portion of the RSPF are cross-linked. In some embodiments, recombinant silk is as provided in any of the compositions #1001-2450, #3001-4450, and #5001-6595.

In some embodiments, the cross-linked recombinant silk fibroin fragments comprises a cross-linking moiety comprising an alkane or alkyl chain, and/or an ether group. In some embodiments, the cross-linked recombinant silk fibroin fragments comprises a cross-linking moiety comprising a polyethylene glycol (PEG) chain. In some embodiments, the cross-linked recombinant silk fibroin fragments comprises a cross-linking moiety comprising a secondary alcohol. In some embodiments, cross-linking is obtained using a cross-linking agent, a cross-linking precursor, or an activating agent. In some embodiments, the cross-linking agent and/or the cross-linking precursor comprises an epoxy group. In some embodiments, cross-linking is obtained using a cross-linking agent, a cross-linking precursor, or an activating agent selected from the group consisting of a polyepoxy linker, a diepoxy linker, a polyepoxy-PEG, a diepoxy-PEG, a polyglycidyl-PEG, a diglycidyl-PEG, a poly acrylate PEG, a diacrylate PEG, 1,4-bis(2,3-epoxypropoxy)butane, 1,4-bisglycidyloxybutane, divinyl sulfone (DVS), 1,4-butanediol diglycidyl ether (BDDE), UV light, glutaraldehyde, 1,2-bis(2,3-epoxypropoxy)ethylene (EGDGE), 1,2,7,8-diepoxyoctane (DEO), biscarbodiimide (BCDI), pentaerythritol tetraglycidyl ether (PETGE), adipic dihydrazide (ADH), bis(sulfosuccinimidyl)suberate (BS), hexamethylenediamine (HMDA), 1-(2,3-epoxypropyl)-2,3-epoxycyclohexane, a carbodiimide, and any combinations thereof. In some embodiments, recombinant silk is as provided in any of the compositions #1001-2450, #3001-4450, and #5001-6595.

In some embodiments, cross-linking is obtained using a polyfunctional epoxy compound selected from the group consisting of 1,4-butanediol diglycidyl ether (BDDE), ethylene glycol diglycidyl ether (EGDGE), 1,6-hexanediol diglycidyl ether, polyethylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, tri-methylolpropane polyglycidyl ether, pentaerythritol polyglycidyl ether, and sorbitol polyglycidyl ether. In some embodiments, cross-linking is obtained using a cross-linking agent and/or a cross-linking precursor selected from the group consisting of polyethylene glycol diglycidyl ether, diepoxy PEG, PEG diglycidyl ether, polyoxyethylene bis-glycidyl ether, PEGDE, and PEGDGE. In some embodiments, cross-linking is obtained using polyethylene glycol diglycidyl ether having a number average molecular weight (Mn) of about 500, about 1000, about 2000, or about 6000. In some embodiments, cross-linking is obtained using polyethylene glycol diglycidyl ether having from 2 to 25 ethylene glycol groups. In some embodiments, cross-linking is obtained using a cross-linking agent and/or a cross-linking precursor selected from the group consisting of a polyepoxy recombinant silk fibroin linker, a diepoxy recombinant silk fibroin linker, a polyepoxy recombinant silk fibroin fragment linker, a diepoxy recombinant silk fibroin fragment linker, a polyglycidyl s recombinant ilk fibroin linker, a diglycidyl recombinant silk fibroin linker, a polyglycidyl recombinant silk fibroin fragment linker, and a diglycidyl recombinant silk fibroin fragment linker. In some embodiments, a portion of recombinant silk fibroin fragments is cross linked to HA. In some embodiments, recombinant silk is as provided in any of the compositions #1001-2450, #3001-4450, and #5001-6595. In some embodiments, a portion of the recombinant silk fibroin fragments are cross-linked to recombinant silk fibroin fragments. In some embodiments, the tissue filler is a gel. In some embodiments, the tissue filler is a hydrogel. In some embodiments, the tissue filler further comprises water. In some embodiments, the total concentration of recombinant silk fibroin fragments in the tissue filler is from about 0.1 mg/mL to about 15 mg/mL.

In one embodiment, the disclosure relates to a biocompatible tissue filler comprising: a glycosaminoglycan selected from the group consisting of hyaluronic acid (HA), carboxymethyl cellulose (CMC), starch, alginate, chondroitin-4-sulfate, chondroitin-δ-sulfate, xanthan gum, chitosan, pectin, agar, carrageenan, and guar gum; and an anesthetic agent; wherein a portion of the glycosaminoglycan is cross-linked by cross-linking moieties comprising one or more of an alkane or alkyl chain, an ether group, and a secondary alcohol; and wherein cross-linking is obtained using a cross-linking agent, a cross-linking precursor, or an activating agent; the tissue filler optionally comprising recombinant silk protein or recombinant silk protein fragments, wherein a portion of the fragments are cross-linked. In some embodiments, the tissue filler is a dermal filler. In some embodiments, the tissue filler is biodegradable. In some embodiments, the tissue filler is injectable. In some embodiments, the tissue filler has a storage modulus (G′) of from about 25 Pa to about 1500 Pa.

In some embodiments, G′ is measured by means of an oscillatory stress of about 0.1 to about 10 Hz. In some embodiments, ‘G’ is measured by means of an oscillatory stress of about 1 Hz. In some embodiments, ‘G’ is measured by means of an oscillatory stress of about 5 Hz. In some embodiments, G′ is measured by means of an oscillatory stress of about 10 Hz. In some embodiments, the tissue filler has a complex viscosity from about 1 Pa·s to about 10 Pa·s. In some embodiments, the tissue filler has a complex viscosity of about 1 Pa·s, about 1.5 Pa·s, about 2 Pa·s, about 2.5 Pa·s, about 3 Pa·s, about 3.5 Pa·s, about 4 Pa·s, about 4.5 Pa·s, about 5 Pa·s, about 5.5 Pa·s, about 6 Pa·s, about 6.5 Pa·s, about 7 Pa·s, about 7.5 Pa·s, about 8 Pa·s, about 8.5 Pa·s, about 9 Pa·s, about 9.5 Pa·s, or about 10 Pa·s. In some embodiments, the complex viscosity is measured by means of an oscillatory stress of about 0.1 to about 10 Hz. In some embodiments, the complex viscosity is measured by means of an oscillatory stress of about 1 Hz. In some embodiments, the complex viscosity is measured by means of an oscillatory stress of about 5 Hz.

In one embodiment, the disclosure relates to a biocompatible tissue filler, e.g., a dermal filler, including recombinant silk fibroin fragments having a polydispersity of between 1 and about 5.0, or between about 1.5 and about 3.0, and a polysaccharide, the recombinant silk fibroin fragments having a weight average molecular weight, or average weight average molecular weight ranging from about 1 kDa to about 250 kDa, about 5 kDa to about 150 kDa, from about 6 kDa to about 17 kDa, from about 17 kDa to about 39 kDa, or from about 39 kDa to about 80 kDa. In some embodiments, recombinant silk is as provided in any of the compositions #1001-2450, #3001-4450, and #5001-6595. In some embodiments, the tissue filler is biodegradable. In some embodiments, a portion of recombinant silk fibroin fragments are cross-linked. In some embodiments, a portion of the recombinant silk fibroin fragments are cross-linked to polysaccharide. In some embodiments, a portion of the recombinant silk fibroin fragments are cross-linked to recombinant silk fibroin fragments. In some embodiments, a portion of the polysaccharide is cross-linked to polysaccharide. In some embodiments, cross-linking includes chemical bond cross-linking. In some embodiments, a portion of cross-linking is zero-length cross-linking. In some embodiments, a portion of cross-linking is auto-cross-linking. In some embodiments, the portion of cross-linked recombinant silk fibroin fragments is up to about 100%. In some embodiments, the portion of cross-linked polysaccharide is up to about 100%. In some embodiments, the polysaccharide is hyaluronic acid (HA). In some embodiments, cross-linking is obtained using a cross-linking agent, a cross-linking precursor, or an activating agent. In some embodiments, the cross-linking agent and/or the cross-linking precursor comprise an epoxy group.

In one embodiment, the disclosure relates to a biocompatible tissue filler, e.g., a dermal filler, including recombinant silk fibroin fragments having a polydispersity of between 1 and about 5.0, or between about 1.5 and about 3.0, and a polysaccharide, the recombinant silk fibroin fragments having low molecular weight, medium molecular weight, and/or high molecular weight. In some embodiments, recombinant silk is as provided in any of the compositions #1001-2450, #3001-4450, and #5001-6595. In some embodiments, the tissue filler is biodegradable. In some embodiments, a portion of recombinant silk fibroin fragments are cross-linked. In some embodiments, a portion of the recombinant silk fibroin fragments are cross-linked to polysaccharide. In some embodiments, a portion of the recombinant silk fibroin fragments are cross-linked to recombinant silk fibroin fragments. In some embodiments, a portion of the polysaccharide is cross-linked to polysaccharide. In some embodiments, cross-linking includes chemical bond cross-linking. In some embodiments, a portion of cross-linking is zero-length cross-linking. In some embodiments, a portion of cross-linking is auto-cross-linking. In some embodiments, the portion of cross-linked recombinant silk fibroin fragments is up to about 100%. In some embodiments, the portion of cross-linked polysaccharide is up to about 100%. In some embodiments, the polysaccharide is hyaluronic acid (HA). In some embodiments, cross-linking is obtained using a cross-linking agent, a cross-linking precursor, or an activating agent. In some embodiments, the cross-linking agent and/or the cross-linking precursor comprise an epoxy group.

In one embodiment, the disclosure relates to a biocompatible tissue filler, e.g., a dermal filler, including recombinant silk fibroin fragments having a polydispersity of between 1 and about 5.0, or between about 1.5 and about 3.0, and a polysaccharide, the RSPF having a weight average molecular weight, or average weight average molecular weight ranging from about 1 kDa to about 250 kDa, about 5 kDa to about 150 kDa, from about 6 kDa to about 17 kDa, from about 17 kDa to about 39 kDa, or from about 39 kDa to about 80 kDa. In some embodiments, recombinant silk is as provided in any of the compositions #1001-2450, #3001-4450, and #5001-6595. In some embodiments, the tissue filler is biodegradable. In some embodiments, a portion of recombinant silk fibroin fragments are cross-linked. In some embodiments, a portion of the recombinant silk fibroin fragments are cross-linked to polysaccharide. In some embodiments, a portion of the RSPF are cross-linked to RSPF. In some embodiments, a portion of the polysaccharide is cross-linked to polysaccharide. In some embodiments, cross-linking includes chemical bond cross-linking. In some embodiments, a portion of cross-linking is zero-length cross-linking. In some embodiments, a portion of cross-linking is auto-cross-linking. In some embodiments, the portion of cross-linked recombinant silk fibroin fragments is up to about 100%. In some embodiments, the portion of cross-linked polysaccharide is up to about 100%. In some embodiments, the polysaccharide is hyaluronic acid (HA). In some embodiments, cross-linking is obtained using a cross-linking agent, a cross-linking precursor, or an activating agent selected from the group consisting of 1,4-bis(2,3-epoxypropoxy)butane, 1,4-bisglycidyloxybutane, divinyl sulfone (DVS), 1,4-butanediol diglycidyl ether (BDDE), UV light, glutaraldehyde, 1,2-bis(2,3-epoxypropoxy)ethylene (EGDGE), 1,2,7,8-diepoxyoctane (DEO), biscarbodiimide (BCDI), pentaerythritol tetraglycidyl ether (PETGE), adipic dihydrazide (ADH), bis(sulfosuccinimidyl)suberate (BS), hexamethylenediamine (HMDA), epoxypropyl)-2,3-epoxycyclohexane, a carbodiimide, and any combinations thereof.

In one embodiment, the disclosure relates to a biocompatible tissue filler, e.g., a dermal filler, including recombinant silk fibroin fragments having a polydispersity of between 1 and about 5.0, or between about 1.5 and about 3.0, and a polysaccharide, the recombinant silk fibroin fragments having low molecular weight, medium molecular weight, and/or high molecular weight. In some embodiments, recombinant silk is as provided in any of the compositions #1001-2450, #3001-4450, and #5001-6595. In some embodiments, the tissue filler is biodegradable. In some embodiments, a portion of recombinant silk fibroin fragments are cross-linked. In some embodiments, a portion of the recombinant silk fibroin fragments are cross-linked to polysaccharide. In some embodiments, a portion of the recombinant silk fibroin fragments are cross-linked to recombinant silk fibroin fragments. In some embodiments, a portion of the polysaccharide is cross-linked to polysaccharide. In some embodiments, cross-linking includes chemical bond cross-linking. In some embodiments, a portion of cross-linking is zero-length cross-linking. In some embodiments, a portion of cross-linking is auto-cross-linking. In some embodiments, the portion of cross-linked RSPF is up to about 100%. In some embodiments, the portion of cross-linked polysaccharide is up to about 100%. In some embodiments, the polysaccharide is hyaluronic acid (HA). In some embodiments, cross-linking is obtained using a cross-linking agent, a cross-linking precursor, or an activating agent selected from the group consisting of 1,4-bis(2,3-epoxypropoxy)butane, 1,4-bisglycidyloxybutane, divinyl sulfone (DVS), 1,4-butanediol diglycidyl ether (BDDE), UV light, glutaraldehyde, 1,2-bis(2,3-epoxypropoxy)ethylene (EGDGE), 1,2,7,8-diepoxyoctane (DEO), biscarbodiimide (BCDI), pentaerythritol tetraglycidyl ether (PETGE), adipic dihydrazide (ADH), bis(sulfosuccinimidyl)suberate (BS), hexamethylenediamine (HMDA), 1-(2,3-epoxypropyl)-2,3-epoxycyclohexane, a carbodiimide, and any combinations thereof.

In one embodiment, the disclosure relates to a biocompatible tissue filler gel, e.g., a dermal filler gel, including recombinant silk fibroin fragments having a polydispersity of between 1 and about 5.0, or between about 1.5 and about 3.0, and a polysaccharide, the recombinant silk fibroin fragments having a weight average molecular weight, or average weight average molecular weight ranging from about 1 kDa to about 250 kDa, about 5 kDa to about 150 kDa, from about 6 kDa to about 17 kDa, from about 17 kDa to about 39 kDa, or from about 39 kDa to about 80 kDa. In some embodiments, recombinant silk is as provided in any of the compositions #1001-2450, #3001-4450, and #5001-6595. In some embodiments, the tissue filler is biodegradable. In some embodiments, a portion of recombinant silk fibroin fragments are cross-linked. In some embodiments, a portion of the recombinant silk fibroin fragments are cross-linked to polysaccharide. In some embodiments, a portion of the recombinant silk fibroin fragments are cross-linked to recombinant silk fibroin fragments. In some embodiments, a portion of the polysaccharide is cross-linked to polysaccharide. In some embodiments, cross-linking includes chemical bond cross-linking. In some embodiments, a portion of cross-linking is zero-length cross-linking. In some embodiments, a portion of cross-linking is auto-cross-linking. In some embodiments, the portion of cross-linked RSPF is up to about 100%. In some embodiments, the portion of cross-linked polysaccharide is up to about 100%. In some embodiments, the polysaccharide is hyaluronic acid (HA). In some embodiments, the gel further comprises water.

In one embodiment, the disclosure relates to a biocompatible tissue filler gel, e.g., a dermal filler gel, including recombinant silk fibroin fragments having a polydispersity of between 1 and about 5.0, or between about 1.5 and about 3.0, and a polysaccharide, the recombinant silk fibroin fragments having low molecular weight, medium molecular weight, and/or high molecular weight. In some embodiments, recombinant silk is as provided in any of the compositions #1001-2450, #3001-4450, and #5001-6595. In some embodiments, the tissue filler is biodegradable. In some embodiments, a portion of recombinant silk fibroin fragments are cross-linked. In some embodiments, a portion of the recombinant silk fibroin fragments are cross-linked to polysaccharide. In some embodiments, a portion of the recombinant silk fibroin fragments are cross-linked to recombinant silk fibroin fragments. In some embodiments, a portion of the polysaccharide is cross-linked to polysaccharide. In some embodiments, cross-linking includes chemical bond cross-linking. In some embodiments, a portion of cross-linking is zero-length cross-linking. In some embodiments, a portion of cross-linking is auto-cross-linking. In some embodiments, the portion of cross-linked recombinant silk fibroin fragments is up to about 100%. In some embodiments, the portion of cross-linked polysaccharide is up to about 100%.

In some embodiments, the polysaccharide is hyaluronic acid (HA). In some embodiments, the gel further comprises water.

In one embodiment, the disclosure relates to a biocompatible tissue filler hydrogel, e.g., a dermal filler hydrogel, including recombinant silk fibroin fragments having a polydispersity of between 1 and about 5.0, or between about 1.5 and about 3.0, and a polysaccharide, the recombinant silk fibroin fragments having a weight average molecular weight, or average weight average molecular weight ranging from about 1 kDa to about 250 kDa, about 5 kDa to about 150 kDa, from about 6 kDa to about 17 kDa, from about 17 kDa to about 39 kDa, or from about 39 kDa to about 80 kDa. In some embodiments, recombinant silk is as provided in any of the compositions #1001-2450, #3001-4450, and #5001-6595. In some embodiments, the tissue filler is biodegradable. In some embodiments, a portion of recombinant silk fibroin fragments are cross-linked. In some embodiments, a portion of the recombinant silk fibroin fragments are cross-linked to polysaccharide. In some embodiments, a portion of the recombinant silk fibroin fragments are cross-linked to recombinant silk fibroin fragments. In some embodiments, a portion of the polysaccharide is cross-linked to polysaccharide. In some embodiments, cross-linking includes chemical bond cross-linking. In some embodiments, a portion of cross-linking is zero-length cross-linking. In some embodiments, a portion of cross-linking is auto-cross-linking. In some embodiments, the portion of cross-linked recombinant silk fibroin fragments is up to about 100%. In some embodiments, the portion of cross-linked polysaccharide is up to about 100%. In some embodiments, the polysaccharide is hyaluronic acid (HA). In some embodiments, the hydrogel further comprises water.

In one embodiment, the disclosure relates to a biocompatible tissue filler hydrogel, e.g., a dermal filler hydrogel, including recombinant silk fibroin fragments having a polydispersity of between 1 and about 5.0, or between about 1.5 and about 3.0, and a polysaccharide, the recombinant silk fibroin fragments having low molecular weight, medium molecular weight, and/or high molecular weight. In some embodiments, recombinant silk is as provided in any of the compositions #1001-2450, #3001-4450, and #5001-6595. In some embodiments, the tissue filler is biodegradable. In some embodiments, a portion of recombinant silk fibroin fragments are cross-linked. In some embodiments, a portion of the recombinant silk fibroin fragments are cross-linked to polysaccharide. In some embodiments, a portion of the recombinant silk fibroin fragments are cross-linked to recombinant silk fibroin fragments. In some embodiments, a portion of the polysaccharide is cross-linked to polysaccharide. In some embodiments, cross-linking includes chemical bond cross-linking. In some embodiments, a portion of cross-linking is zero-length cross-linking.

In some embodiments, a portion of cross-linking is auto-cross-linking. In some embodiments, the portion of cross-linked recombinant silk fibroin fragments is up to about 100%. In some embodiments, the portion of cross-linked polysaccharide is up to about 100%. In some embodiments, the polysaccharide is hyaluronic acid (HA). In some embodiments, the hydrogel further comprises water.

In one embodiment, the disclosure relates to a biocompatible tissue filler, e.g., dermal filler, including recombinant silk fibroin fragments having a polydispersity of between 1 and about 5.0, or between about 1.5 and about 3.0, and a polysaccharide, the RSPF having a weight average molecular weight, or average weight average molecular weight ranging from about 1 kDa to about 250 kDa, about 5 kDa to about 150 kDa, from about 6 kDa to about 17 kDa, from about 17 kDa to about 39 kDa, or from about 39 kDa to about 80 kDa. In some embodiments, recombinant silk is as provided in any of the compositions #1001-2450, #3001-4450, and #5001-6595. In some embodiments, the tissue filler is biodegradable. In some embodiments, a portion of recombinant silk fibroin fragments are cross-linked. In some embodiments, a portion of the recombinant silk fibroin fragments are cross-linked to polysaccharide. In some embodiments, a portion of the recombinant silk fibroin fragments are cross-linked to recombinant silk fibroin fragments. In some embodiments, a portion of the polysaccharide is cross-linked to polysaccharide. In some embodiments, cross-linking includes chemical bond cross-linking. In some embodiments, a portion of cross-linking is zero-length cross-linking. In some embodiments, a portion of cross-linking is auto-cross-linking. In some embodiments, the portion of cross-linked recombinant silk fibroin fragments is up to about 100%. In some embodiments, the portion of cross-linked polysaccharide is up to about 100%. In some embodiments, the polysaccharide is hyaluronic acid (HA).

In some embodiments, the disclosure relates to a method of making a biocompatible tissue filler, e.g., a dermal filler, including recombinant silk fibroin fragments having a polydispersity of between 1 and about 5.0, or between about 1.5 and about 3.0, and a polysaccharide, the method including providing a composition comprising recombinant silk fibroin fragments and a polysaccharide, and adding to the solution a cross-linking agent, a cross-linking precursor, an activating agent, or a gelation enhancer, the RSPF having an average weight average molecular weight ranging from about 1 kDa to about 250 kDa, about 5 kDa to about 150 kDa, from about 6 kDa to about 17 kDa, from about 17 kDa to about 39 kDa, or from about 39 kDa to about 80 kDa. In some embodiments, recombinant silk is as provided in any of the compositions #1001-2450, #3001-4450, and #5001-6595. In some embodiments, the tissue filler is biodegradable. In some embodiments, a portion of recombinant silk fibroin fragments are cross-linked. In some embodiments, a portion of the recombinant silk fibroin fragments are cross-linked to polysaccharide. In some embodiments, a portion of the recombinant silk fibroin fragments are cross-linked to recombinant silk fibroin fragments. In some embodiments, a portion of the polysaccharide is cross-linked to polysaccharide. In some embodiments, the tissue filler further includes cross-linking moieties, e.g., epoxy derived cross-linking moieties. In some embodiments, a portion of cross-linking is auto-cross-linking. In some embodiments, the portion of cross-linked recombinant silk fibroin fragments is up to about 100%. In some embodiments, the portion of cross-linked polysaccharide is up to about 100%. In some embodiments, the polysaccharide is hyaluronic acid (HA). In some embodiments, the tissue filler further comprises water.

Textiles and Leathers Coated with Recombinant Silk-Based Protein Fragments

As used herein, the term “washable” and “exhibiting washability” means that a silk coated fabric of the present disclosure is capable of being washed without shrinking, fading, or the like.

As used herein, the term “textile” refers to a flexible woven or non-woven material consisting of a network of natural or artificial fibers often referred to as fabric, thread, or yarn. In an embodiment, textiles can be used to fabricate clothing, shoes and bags. In an embodiment, textiles can be used to fabricate carpeting, upholstered furnishings, window shades, towels, and coverings for tables, beds, and other flat surfaces. In an embodiment, textiles can be used to fabricate flags, backpacks, tents, nets, handkerchiefs, balloons, kites, sails, and parachutes.

As used herein, the term “leather” refers to natural leather and synthetic leather. Natural leather includes chrome-tanned leather (e.g., tanned using chromium sulfate and other chromium salts), vegetable-tanned leather (e.g., tanned using tannins), aldehyde-tanned leather (also known as wet-white leather, e.g., tanned using glutaraldehyde or oxazolidine compounds), brain-tanned leather, formaldehyde-tanned leather, Chamois leather (e.g., tanned using cod oils), rose-tanned leather (e.g., tanned using rose otto oils), synthetic-tanned leather (e.g., tanned using aromatic polymers), alum-tanned leather, patent leather, Vachetta leather, nubuck leather, and rawhide leather. Natural leather also includes split leather, full-grain leather, top-grain leather, and corrected-grain leather, the properties and preparation of which are known to those of skill in the art. Synthetic leather includes poromeric imitation leathers (e.g., polyurethane on polyester), vinyl and polyamide felt fibers, polyurethane, polyvinyl chloride, polyethylene (PE), polypropylene (PP), vinyl acetate copolymer (EVA), polyamide, polyester, textile-polymer composite microfibers, corfan, koskin, leatherette, BIOTHANE®, BIRKIBUC®, BIRKO-FLOR®, CLARINO®, ECOLORICA®, KYDEX®, LORICA®, NAUGAHYDE®, REXINE®, VEGETAN®, FABRIKOID®, or combinations thereof.

As used herein, the term “hand” refers to the feel of a fabric, which may be further described as the feeling of softness, crispness, dryness, silkiness, and combinations thereof. Fabric hand is also referred to as “drape.” A fabric with a hard hand is coarse, rough, and generally less comfortable for the wearer. A fabric with a soft hand is fluid and smooth, such as fine silk or wool, and generally more comfortable for the wearer. Fabric hand can be determined by comparison to collections of fabric samples, or by use of methods such as the Kawabata Evaluation System (KES) or the Fabric Assurance by Simple Testing (FAST) methods. Behera and Hari, Ind. J. Fibre & Textile Res., 1994, 19, 168-71.

As used herein, the term “yarn” refers to a single or multi-fiber construct. As used herein, a “coating” refers to a material, or combination of materials, that form a substantially continuous layer or film on an exterior surface of a substrate, such as a textile. In some embodiments, a portion of the coating may penetrate at least partially into the substrate. In some embodiments, the coating may penetrate at least partially into the interstices of a substrate. In some embodiments, the coating may be infused into a surface of the substrate such that the application of the coating, or coating process, may include infusing (at the melting temperature of the substrate) at least one coating component at least partially into a surface of the substrate. A coating may be applied to a substrate by one or more of the processes described herein.

In embodiments described where the coating may be infused into a surface of the substrate, the coating may be codissolved in a surface of the substrate such that a component of the coating may be intermixed in the surface of the substrate to a depth of at least about 1 nm, or at least about 2 nm, or at least about 3 nm, or at least about 4 nm, or at least about 5 nm, or at least about 6 nm, or at least about 7 nm, or at least about 8 nm, or at least about 9 nm, or at least about 10 nm, or at least about 20 nm, or at least about 30 nm, or at least about 40 nm, or at least about 50 nm, or at least about 60 nm, or at least about 70 nm, or at least about 80 nm, or at least about 90 nm, or at least about 100 nm. In some embodiments, the coating may be infused into a surface of the substrate where the substrate includes one or more polymers including, but not limited to, polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, and LYCRA.

As used herein, the term “bath coating” encompasses coating a fabric in a batch, immersing a fabric in a bath, and submerging a fabric in a bath. Concepts of bath coating are set forth in U.S. Pat. No. 4,521,458, the entirety of which is incorporated by reference.

As used herein, and unless more specifically described, the term “drying” may refer to drying a coated material as described herein at a temperature greater than room temperature (i.e., 20° C.).

In an embodiment, the disclosure provides a textile or leather product coated with recombinant silk-based proteins or fragments thereof. In some embodiments, recombinant silk is as provided in any of the compositions #1001-2450, #3001-4450, and #5001-6595. Leather coated with silk fragments has been described for example in WO 2020/018821, which is incorporated herein by reference in its entirety. In an embodiment, the disclosure provides a textile or leather product coated with recombinant silk-based proteins or fragments thereof, wherein the textile is a textile used for human apparel, including performance and/or athletic apparel. In an embodiment, the disclosure provides a textile or leather product coated with recombinant silk-based proteins or fragments thereof, and wherein the textile or leather product exhibits improved moisture management properties and/or resistance to microbial growth. In an embodiment, the disclosure provides a textile or leather product coated with recombinant silk-based proteins or fragments thereof, wherein the textile is a textile or leather product used for home upholstery. In an embodiment, the disclosure provides a textile or leather product coated with recombinant silk-based proteins or fragments thereof, wherein the textile or leather product is used for automobile upholstery. In an embodiment, the disclosure provides a textile or leather product coated with recombinant silk-based proteins or fragments thereof, wherein the textile or leather product is used for aircraft upholstery. In an embodiment, the disclosure provides a textile or leather product coated with recombinant silk-based proteins or fragments thereof, wherein the textile or leather product is used for upholstery in transportation vehicles for public, commercial, military, or other use, including buses and trains. In an embodiment, the disclosure provides a textile or leather product coated with recombinant silk-based proteins or fragments thereof, wherein the textile or leather product is used for upholstery of a product that requires a high degree of resistance to wear as compared to normal upholstery.

In an embodiment, the disclosure provides a textile or leather product coated with recombinant silk-based proteins or fragments thereof, wherein the textile is a textile or leather product fabricated as trim on automobile upholstery. In some embodiments, recombinant silk is as provided in any of the compositions #1001-2450, #3001-4450, and #5001-6595. In an embodiment, the disclosure provides a textile or leather product coated with recombinant silk-based proteins or fragments thereof, wherein the textile is a textile or leather product fabricated as a steering wheel. In an embodiment, the disclosure provides a textile or leather product coated with recombinant silk-based proteins or fragments thereof, wherein the textile is a textile or leather product fabricated as a headrest. In an embodiment, the disclosure provides a textile or leather product coated with recombinant silk-based proteins or fragments thereof, wherein the textile is a textile or leather product fabricated as an armrest. In an embodiment, the disclosure provides a textile or leather product coated with recombinant silk-based proteins or fragments thereof, wherein the textile is a textile or leather product fabricated as an automobile floor mat. In an embodiment, the disclosure provides a textile or leather product coated with recombinant silk-based proteins or fragments thereof, wherein the textile is a textile or leather product fabricated as automobile or vehicle carpet. In an embodiment, the disclosure provides a textile or leather product coated with recombinant silk-based proteins or fragments thereof, wherein the textile is a textile or leather product fabricated as automotive trim. In an embodiment, the disclosure provides a textile or leather product coated with recombinant silk-based proteins or fragments thereof, wherein the textile is a textile or leather product fabricated as a children's car seat. In an embodiment, the disclosure provides a textile or leather product coated with recombinant silk-based proteins or fragments thereof, wherein the textile is a textile or leather product fabricated as a seat belt or safety harness. In an embodiment, the disclosure provides a textile or leather product coated with recombinant silk-based proteins or fragments thereof, wherein the textile is a textile or leather product fabricated as a dashboard. In an embodiment, the disclosure provides a textile or leather product coated with recombinant silk-based proteins or fragments thereof, wherein the textile is a textile or leather product fabricated as a seat. In an embodiment, the disclosure provides a textile or leather product coated with recombinant silk-based proteins or fragments thereof, wherein the textile is a textile or leather product fabricated as a seat panel. In an embodiment, the disclosure provides a textile or leather product coated with recombinant silk-based proteins or fragments thereof, wherein the textile is a textile or leather product fabricated as an interior panel. In an embodiment, the disclosure provides a textile or leather product coated with recombinant silk-based proteins or fragments thereof, wherein the textile is a textile or leather product fabricated as an airbag cover. In an embodiment, the disclosure provides a textile or leather product coated with recombinant silk-based proteins or fragments thereof, wherein the textile is a textile or leather product fabricated as an airbag. In an embodiment, the disclosure provides a textile or leather product coated with recombinant silk-based proteins or fragments thereof, wherein the textile is a textile or leather product fabricated as a sunvisor. In an embodiment, the disclosure provides a textile or leather product coated with recombinant silk-based proteins or fragments thereof, wherein the textile is a textile or leather product fabricated as a wiring harness. In an embodiment, the disclosure provides a product coated with recombinant silk-based proteins or fragments thereof, wherein the product is a cushion. In an embodiment, the disclosure provides a product coated with recombinant silk-based proteins or fragments thereof, wherein the product is automotive, aircraft, or other vehicular insulation. The coating comprises recombinant silk based proteins or fragments thereof having a weight average molecular weight, or average weight average molecular weight range of about 5 kDa to about 144 kDa, wherein the recombinant silk based proteins or protein fragments thereof have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the silk based proteins or fragments thereof have a polydispersity of between 1 and about 5.0, or between about 1.5 and about 3.0, and optionally wherein the recombinant proteins or protein fragments, prior to coating the fabric, do not spontaneously or gradually gelate and do not visibly change in color or turbidity when in a solution for at least 10 days. In some embodiments, recombinant silk is as provided in any of the compositions #1001-2450, #3001-4450, and #5001-6595.

In an embodiment, the disclosure provides an article comprising a textile or leather coated with recombinant silk-based proteins or fragments thereof. In an embodiment, the textile or leather is a textile or leather used in the manufacture of tents, sleeping bags, ponchos, and soft-walled coolers. In an embodiment, the textile or leather is a textile or leather used in the manufacture of athletic equipment. In an embodiment, the textile or leather is a textile or leather used in the manufacture of outdoor gear. In an embodiment, the textile or leather is a textile or leather used in the manufacture of hiking gear, such as harnesses and backpacks. In an embodiment, the textile or leather is a textile or leather used in the manufacture of climbing gear. In an embodiment, the textile or leather is canvass. In an embodiment, the textile or leather is a textile or leather used in the manufacture of a hat. In an embodiment, the textile or leather is a textile or leather used in the manufacture of an umbrella. In an embodiment, the textile or leather is a textile or leather used in the manufacture of a tent. In an embodiment, the textile or leather is a textile or leather used in the manufacture of a baby sleeper, a baby blanket, or a baby pajama. In an embodiment, the textile or leather is a textile or leather used in the manufacture of a glove, such as a driving glove or an athletic glove. In an embodiment, the textile or leather is a textile or leather used in the manufacture of athletic pants, such as sweat pants, jogging pants, yoga pants, or pants for use in competitive sports. In an embodiment, the textile or leather is a textile or leather used in the manufacture of athletic shirts, such as sweat shirts, jogging shirts, yoga shirts, or shirts for use in competitive sports. In an embodiment, the textile or leather is a textile or leather used in the manufacture of beach equipment, such as beach umbrellas, beach chairs, beach blankets, and beach towels. In an embodiment, the textile or leather is a textile or leather used in the manufacture of jackets or overcoats. In an embodiment, the textile or leather is a textile or leather used in the manufacture of medical garments, such as surgical drapes, surgical gowns, surgical sleeves, laboratory sleeves, laboratory coats, wound dressings, sterilization wraps, surgical face masks, retention bandages, support devices, compression bandages, shoe covers, surgical blankets, and the like. The coating comprises recombinant silk based proteins or fragments thereof having a weight average molecular weight, or average weight average molecular weight range of about 5 kDa to about 144 kDa, wherein the recombinant silk based proteins or protein fragments thereof have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof have a polydispersity of between 1 and about 5.0, or between about 1.5 and about 3.0, and optionally wherein the recombinant proteins or protein fragments, prior to coating the fabric, do not spontaneously or gradually gelate and do not visibly change in color or turbidity when in a solution for at least 10 days. In some embodiments, recombinant silk is as provided in any of the compositions #1001-2450, #3001-4450, and #5001-6595.

In an embodiment, the disclosure provides a shoe coated with recombinant silk-based proteins or fragments thereof. In an embodiment, the disclosure provides a shoe coated with recombinant silk-based proteins or fragments thereof, wherein the shoe exhibits an improved property relative to an uncoated shoe. In an embodiment, the disclosure provides a shoe coated with recombinant silk-based proteins or fragments thereof, wherein the shoe exhibits an improved property relative to an uncoated shoe, and wherein the improved property is stain resistance. In an embodiment, the disclosure provides a shoe coated with recombinant silk-based proteins or fragments thereof, wherein the shoe exhibits an improved property relative to an uncoated shoe, and wherein the shoe is made of natural leather or synthetic leather. The coating comprises recombinant silk based proteins or fragments thereof having a weight average molecular weight, or average weight average molecular weight range of about 5 kDa to about 144 kDa, wherein the recombinant silk based proteins or protein fragments thereof have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof have a polydispersity of between 1 and about 5.0, or between about 1.5 and about 3.0, and optionally wherein the proteins or protein fragments, prior to coating the fabric, do not spontaneously or gradually gelate and do not visibly change in color or turbidity when in a solution for at least 10 days. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, and wherein the article is a textile or leather.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa.

In an embodiment, the disclosure provides an article comprising a fiber or yarn having a coating, wherein the coating comprises recombinant silk based proteins or fragments thereof having a weight average molecular weight range of about 5 kDa to about 144 kDa, wherein the recombinant silk based proteins or fragments comprise recombinant silk and a copolymer.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or protein fragments thereof have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof have a polydispersity of between about 1.0 and about 5.0, and wherein the recombinant proteins or protein fragments, prior to coating the fabric, do not spontaneously or gradually gelate and do not visibly change in color or turbidity when in a solution for at least 10 days. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the fabric exhibits an improved property, wherein the improved property is an accumulative one-way moisture transport index selected from the group consisting of greater than 40%, greater than 60%, greater than 80%, greater than 100%, greater than 120%, greater than 140%, greater than 160%, and greater than 180%. In an embodiment, the foregoing improved property is determined after a period of machine washing cycles selected from the group consisting of 5 cycles, 10 cycles, 25 cycles, and 50 cycles. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the fabric exhibits an improved property, wherein the improved property is an accumulative one way transport capability increase relative to uncoated fabric selected from the group consisting of 1.2 fold, 1.5 fold, 2.0 fold, 3.0 fold, 4.0 fold, 5.0 fold, and 10 fold. In an embodiment, the foregoing improved property is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the fabric exhibits an improved property, wherein the improved property is an overall moisture management capability selected from the group consisting of greater than 0.05, greater than 0.10, greater than 0.15, greater than 0.20, greater than 0.25, greater than 0.30, greater than 0.35, greater than 0.40, greater than 0.50, greater than 0.60, greater than 0.70, and greater than 0.80. In an embodiment, the foregoing improved property is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric exhibits substantially no increase in microbial growth after a number of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the fabric exhibits substantially no increase in microbial growth after a number of machine washing cycles selected from the group consisting of 5 cycles, 10 cycles, 25 cycles, and 50 cycles, and wherein the microbial growth is microbial growth of a microbe selected from the group consisting of Staphylococcus aureus, Klebsiella pneumoniae, and combinations thereof.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the fabric exhibits substantially no increase in microbial growth after a number of machine washing cycles selected from the group consisting of 5 cycles, 10 cycles, 25 cycles, and 50 cycles, wherein the microbial growth is microbial growth of a microbe selected from the group consisting of Staphylococcus aureus, Klebsiella pneumoniae, and combinations thereof, wherein the microbial growth is reduced by a percentage selected from the group consisting of 50%, 100%, 500%, 1000%, 2000%, and 3000% compared to an uncoated fabric.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the coating is applied to the fabric at the fiber level prior to forming the fabric.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the coating is applied to the fabric at the fabric level or garment level (e.g., after manufacture of a garment from fabrics, leathers, and/or other materials).

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level or garment level, and wherein the fabric is bath coated.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level or garment level, and wherein the fabric is spray coated.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level or garment level, and wherein the fabric is coated with a stencil.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level or garment level, and wherein the coating is applied to at least one side of the fabric using a method selected from the group consisting of a bath coating process, a spray coating process, a stencil (i.e., screen) process, a silk-foam based process, a roller-based process, a magnetic roller process, a knife process, a transfer process, a foam process, a lacquering process, and a printing process. In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level, and wherein the coating is applied to both sides of the fabric using a method selected from the group consisting of a bath coating process, a spray coating process, a stencil (i.e., screen) process, a silk-foam based process, a roller-based process, a magnetic roller process, a knife process, a transfer process, a foam process, a lacquering process, and a printing process. In some embodiments, recombinant silk is as provided in any of the compositions #1001-2450, #3001-4450, and #5001-6595.

In any of the foregoing embodiments, the coating may be applied at the fabric garment level by any of the methods disclosed herein to recondition fabrics or garments. For example, such reconditioning using a coating comprising recombinant silk based proteins or fragments thereof may be performed as part of washing or cleaning a fabric or garment.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, and wherein the coating has a thickness of about one nanolayer. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, and wherein the coating has a thickness selected from the group consisting of about 5 nm, about 10 nm, about 15 nm, about 20 nm, about 25 nm, about 50 nm, about 100 nm, about 200 nm, about 500 nm, about 1 μm, about 5 μm, about 10 μm, and about 20 μm.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the coating is adsorbed on the fabric. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the coating is attached to the fabric through chemical, enzymatic, thermal, or irradiative cross-linking. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level, and wherein the hand of the coated fabric is improved relative to an uncoated fabric. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level, and wherein the hand of the coated fabric is improved relative to an uncoated fabric, wherein the hand of the coated fabric that is improved is selected from the group consisting of softness, crispness, dryness, silkiness, and combinations thereof. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is applied to the fabric at the fabric level, and wherein the pilling of the fabric is improved relative to an uncoated fabric. In some embodiments, recombinant silk is as provided in any of the compositions #1001-2450, #3001-4450, and #5001-6595.

In an embodiment, the recombinant silk coating is applied using a bath process, a screen (or stencil) process, a spray process, a recombinant silk-foam based process, and a roller based process.

In an embodiment, a fiber or a yarn comprises a synthetic fiber or yarn, including polyester, Mylar, cotton, nylon, polyester-polyurethane copolymer, rayon, acetate, aramid (aromatic polyamide), acrylic, ingeo (polylactide), lurex (polyamide-polyester), olefin (polyethylene-polypropylene), and combinations thereof.

In an embodiment, a fiber or a yarn comprises a natural fiber or yarn (e.g., from animal or plant sources), including alpaca fiber, alpaca fleece, alpaca wool, lama fiber, lama fleece, lama wool, cotton, cashmere and sheep fiber, sheep fleece, sheep wool, byssus, chiengora, qiviut, yak, rabbit, lambswool, mohair wool, camel hair, angora wool, silkworm silk, abaca fiber, coir fiber, flax fiber, jute fiber, kapok fiber, kenaf fiber, raffia fiber, bamboo fiber, hemp, modal fiber, pina, ramie, sisal, and soy protein fiber.

In an embodiment, a fiber or a yarn comprises a mineral fiber, also known as mineral wool, mineral cotton, or man-made mineral fiber, including fiberglass, glass, glasswool, stone wool, rock wool, slagwool, glass filaments, asbestos fibers, and ceramic fibers.

In an embodiment, a water-soluble recombinant silk coating may be used as an adhesive or binder for binding particles to fabrics or for binding fabrics. In an embodiment, an article comprises a fabric bound to another fabric using a recombinant silk coating. In an embodiment, an article comprises a fabric with particles bound to the fabric using a silk adhesive.

In an embodiment, the coating is applied to an article including a fabric at the yarn level. In an embodiment, the coating is applied at the fabric level. In an embodiment, the coating has a thickness selected from the group consisting of about 5 nm, about 10 nm, about 15 nm, about 20 nm, about 25 nm, about 50 nm, about 100 nm, about 200 nm, about 500 nm, about 1 μm, about 5 μm, about 10 μm, and about 20 μm. In an embodiment, the coating has a thickness range selected from the group consisting of about 5 nm to about 100 nm, about 100 nm to about 200 nm, about 200 nm to about 500 nm, about 1 μm to about 2 μm, about 2 μm to about 5 μm, about 5 μm to about 10 μm, and about 10 μm to about 20 μm.

In an embodiment, a fiber or a yarn is treated with a polymer, such as polyglycolide (PGA), polyethylene glycols, copolymers of glycolide, glycolide/L-lactide copolymers (PGA/PLLA), glycolide/trimethylene carbonate copolymers (PGA/TMC), polylactides (PLA), stereocopolymers of PLA, poly-L-lactide (PLLA), poly-DL-lactide (PDLLA), L-lactide/DL-lactide copolymers, co-polymers of PLA, lactide/tetramethylglycolide copolymers, lactide/trimethylene carbonate copolymers, lactide/δ-valerolactone copolymers, lactide/E-caprolactone copolymers, polydepsipeptides, PLA/polyethylene oxide copolymers, unsymmetrically 3,6-substituted poly-1,4-dioxane-2,5-diones, poly-β-hydroxybutyrate (PHBA), PHBA/β-hydroxyvalerate copolymers (PHBA/HVA), poly-β-hydroxypropionate (PHPA), poly-p-dioxanone (PDS), poly-δ-valerolactone, poly-ε-caprolactone, methylmethacrylate-N-vinyl pyrrolidine copolymers, polyesteramides, polyesters of oxalic acid, polydihydropyrans, polyalkyl-2-cyanoacrylates, polyurethanes (PU), polyvinylalcohols (PVA), polypeptides, poly-β-malic acid (PMLA), poly-β-alkanoic acids, polyvinylalcohol (PVA), polyethyleneoxide (PEO), chitine polymers, polyethylene, polypropylene, polyasetal, polyamides, polyesters, polysulphone, polyether ether ketone, polyethylene terephthalate, polycarbonate, polyaryl ether ketone, and polyether ketone ketone.

In an embodiment, the silk coating surface can be modified recombinant silk crystals that range in size from nm to μm.

The criterion for “visibility” is satisfied by any one of the following: a change in the surface character of the textile; the silk coating fills the interstices where the yarns intersect; or the silk coating blurs or obscures the weave.

In an embodiment, a recombinant silk based protein or fragment solution may be utilized to coat at least a portion of a fabric which can be used to create a textile. In an embodiment, a recombinant silk based protein or fragment solution may be weaved into yarn that can be used as a fabric in a textile. In an embodiment, a recombinant silk based protein or fragment solution may be used to coat a fiber. In an embodiment, the disclosure provides an article comprising a recombinant silk based protein or fragment solution coating at least a portion of a fabric or a textile. In an embodiment, the disclosure provides an article comprising a recombinant silk based protein or fragment solution coating a yarn. In an embodiment, the disclosure provides an article comprising a recombinant silk based protein or fragment solution coating a fiber.

There is disclosed a textile that is at least partially surface treated with an aqueous solution of recombinant silk-based protein fragments of the present disclosure so as to result in a recombinant silk coating on the textile. In an embodiment, the recombinant silk coating of the present disclosure is available in a spray can and can be sprayed on any textile by a consumer. In an embodiment, a textile comprising a recombinant silk coating of the present disclosure is sold to a consumer. In an embodiment, a textile of the present disclosure is used in constructing action sportswear/apparel. In an embodiment, a recombinant silk coating of the present disclosure is positioned on the underlining of apparel. In an embodiment, a recombinant silk coating of the present disclosure is positioned on the shell, the lining, or the interlining of apparel. In an embodiment, apparel is partially made from a recombinant silk coated textile of the present disclosure and partially made from an uncoated textile. In an embodiment, apparel partially made from a recombinant silk coated textile and partially made from an uncoated textile combines an uncoated inert synthetic material with a recombinant silk coated inert synthetic material. Examples of inert synthetic material include, but are not limited to, polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, and mixtures thereof. In an embodiment, apparel partially made from a recombinant silk coated textile and partially made from an uncoated textile combines an elastomeric material at least partially covered with a recombinant silk coating of the present disclosure. In an embodiment, the percentage of recombinant silk to elastomeric material can be varied to achieve desired shrink or wrinkle resistant properties.

In an embodiment, a recombinant silk coating of the present disclosure is visible. In an embodiment, a recombinant silk coating of the present disclosure positioned on apparel helps control skin temperature. In an embodiment, a recombinant silk coating of the present disclosure positioned on apparel helps control fluid transfer away from the skin. In an embodiment, a recombinant silk coating of the present disclosure positioned on apparel has a soft feel against the skin decreasing abrasions from fabric on skin. In an embodiment, a recombinant silk coating of the present disclosure positioned on a textile has properties that confer at least one of wrinkle resistance, shrinkage resistance, or machine washability to the textile. In an embodiment, a recombinant silk coated textile of the present disclosure is 100% machine washable and dry cleanable. In an embodiment, a recombinant silk coated textile of the present disclosure is 100% waterproof. In an embodiment, a recombinant silk coated textile of the present disclosure is wrinkle resistant. In an embodiment, a recombinant silk coated textile of the present disclosure is shrink resistant. In an embodiment, a recombinant silk coated textile of the present disclosure has the qualities of being waterproof, breathable, and elastic and possess a number of other qualities which are highly desirable in action sportswear. In an embodiment, a recombinant silk coated textile of the present disclosure manufactured from a recombinant silk fabric of the present disclosure further includes LYCRA® brand spandex fibers.

In an embodiment, a textile at least partially coated with an aqueous solution of recombinant silk-based protein fragments of the present disclosure is a breathable fabric. In an embodiment, a textile at least partially coated with an aqueous solution of recombinant silk-based protein fragments of the present disclosure is a water-resistant fabric. In an embodiment, a textile at least partially coated with an aqueous solution of recombinant silk-based protein fragments of the present disclosure is a shrink-resistant fabric. In an embodiment, a textile at least partially coated with an aqueous solution of recombinant silk-based protein fragments of the present disclosure is a machine-washable fabric. In an embodiment, a textile at least partially coated with an aqueous solution of recombinant silk-based protein fragments of the present disclosure is a wrinkle resistant fabric. In an embodiment, textile at least partially coated with an aqueous solution of recombinant silk-based protein fragments of the present disclosure provides moisture and vitamins to the skin.

In an embodiment, an aqueous solution of recombinant silk-based protein fragments of the present disclosure is used to coat a textile or leather. In an embodiment, the concentration of recombinant silk in the solution ranges from about 0.1% to about 20.0%. In an embodiment, the concentration of recombinant silk in the solution ranges from about 0.1% to about 15.0%. In an embodiment, the concentration of recombinant silk in the solution ranges from about 0.5% to about 10.0%. In an embodiment, the concentration of recombinant silk in the solution ranges from about 1.0% to about 5.0%. In an embodiment, an aqueous solution of recombinant silk-based protein fragments of the present disclosure is applied directly to a fabric.

Alternatively, recombinant silk microsphere and any additives may be used for coating a fabric. In an embodiment, additives can be added to an aqueous solution of recombinant silk-based protein fragments of the present disclosure before coating (e.g., alcohols) to further enhance material properties. In an embodiment, a recombinant silk coating of the present disclosure can have a pattern to optimize properties of the recombinant silk on the fabric. In an embodiment, a coating is applied to a fabric under tension and/or lax to vary penetration in to the fabric.

In an embodiment, a recombinant silk coating of the present disclosure can be applied at the yarn level, followed by creation of a fabric once the yarn is coated. In an embodiment, an aqueous solution of recombinant silk-based protein fragments of the present disclosure can be spun into fibers to make a recombinant silk fabric and/or recombinant silk fabric blend with other materials known in the apparel industry.

Uses of Textiles and Leathers Coated with Recombinant Silk-Based Protein Fragments in Apparel and Garment Applications

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article exhibits an improved color retention property. In some embodiments, recombinant silk is as provided in any of the compositions #1001-2450, #3001-4450, and #5001-6595. Without being bound by any specific theory, it is postulated that the coating prevents the article from color degradation by separating the fiber or yarn from air or from detergents during washing.

Methods of testing the color retention property of an article are well within the knowledge of one skilled in the art. A specific method of testing of the color retention property of a fabric is described in U.S. Pat. No. 5,142,292, which is incorporated herein by reference in its entirety.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article exhibits an improved color retention property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments thereof comprise recombinant silk-based proteins or protein fragments, wherein the article exhibits an improved color retention property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the article exhibits an improved color retention property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the article exhibits an improved color retention property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article exhibits an improved color retention property. In some embodiments, recombinant silk is as provided in any of the compositions #1001-2450, #3001-4450, and #5001-6595. In an embodiment, the foregoing color retention property of the fabric is determined after a period of machine washing cycles selected from the group consisting of 5 cycles, 10 cycles, 25 cycles, and 50 cycles.

In an embodiment, a textile or leather of the present disclosure exhibits an improved color retention property. In an embodiment, the foregoing improved color retention property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is resistant to microbial (including bacterial and fungal) growth. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is resistant to microbial (including bacterial and fungal) growth. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, wherein the article is resistant to microbial (including bacterial and fungal) growth. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the article is resistant to microbial (including bacterial and fungal) growth. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the article is resistant to microbial (including bacterial and fungal) growth. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is resistant to microbial (including bacterial and fungal) growth. In some embodiments, recombinant silk is as provided in any of the compositions #1001-2450, #3001-4450, and #5001-6595. In an embodiment, the foregoing resistant to microbial (including bacterial and fungal) growth property of the fabric is determined after a period of machine washing cycles selected from the group consisting of 5 cycles, 10 cycles, 25 cycles, and 50 cycles.

In an embodiment, a textile or leather of the present disclosure exhibits resistant to microbial (including bacterial and fungal) growth property. In an embodiment, the foregoing resistant to microbial (including bacterial and fungal) growth property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is resistant to the buildup of static electrical charge. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is resistant to the buildup of static electrical charge. In some embodiments, recombinant silk is as provided in any of the compositions #1001-2450, #3001-4450, and #5001-6595.

In an embodiment, a textile or leather of the present disclosure exhibits resistant to the buildup of static electrical charge property. In an embodiment, the foregoing resistant to the buildup of static electrical charge property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is mildew resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is mildew resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight range of about 5 kDa to about 144 kDa, wherein the recombinant silk based proteins or fragments comprise recombinant silk and a copolymer, wherein the article is mildew resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the article is mildew resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the article is mildew resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is mildew resistant. In some embodiments, recombinant silk is as provided in any of the compositions #1001-2450, #3001-4450, and #5001-6595. In an embodiment, the foregoing mildew resistant property of the fabric is determined after a period of machine washing cycles selected from the group consisting of 5 cycles, 10 cycles, 25 cycles, and 50 cycles.

In an embodiment, a textile or leather of the present disclosure exhibits mildew resistant property. In an embodiment, the foregoing mildew resistant property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the coating is transparent. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the coating is transparent. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, wherein the coating is transparent. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the coating is transparent. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the coating is transparent. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating is transparent. In some embodiments, recombinant silk is as provided in any of the compositions #1001-2450, #3001-4450, and #5001-6595. In an embodiment, the foregoing transparent property of the coating is determined after a period of machine washing cycles selected from the group consisting of 5 cycles, 10 cycles, 25 cycles, and 50 cycles.

In an embodiment, a textile or leather comprises a recombinant silk coating of the present disclosure, wherein the recombinant silk coating is transparent. In an embodiment, the foregoing transparent property of the coating is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is resistant to freeze-thaw cycle damage. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is resistant to freeze-thaw cycle damage. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, wherein the article is resistant to freeze-thaw cycle damage. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the article is resistant to freeze-thaw cycle damage. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the article is resistant to freeze-thaw cycle damage. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is resistant to freeze-thaw cycle damage. In some embodiments, recombinant silk is as provided in any of the compositions #1001-2450, #3001-4450, and #5001-6595. In an embodiment, the foregoing resistant to freeze-thaw cycle damage property of the fabric is determined after a period of machine washing cycles selected from the group consisting of 5 cycles, 10 cycles, 25 cycles, and 50 cycles.

In an embodiment, a textile or leather of the present disclosure exhibits resistant to freeze-thaw cycle damage. In an embodiment, the foregoing resistant to freeze-thaw cycle damage property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the coating provides protection from abrasion. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating provides protection from abrasion. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, wherein the coating provides protection from abrasion. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the coating provides protection from abrasion. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the coating provides protection from abrasion. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the coating provides protection from abrasion. In some embodiments, recombinant silk is as provided in any of the compositions #1001-2450, #3001-4450, and #5001-6595. In an embodiment, the foregoing abrasion resistant property of the fabric is determined after a period of machine washing cycles selected from the group consisting of 5 cycles, 10 cycles, 25 cycles, and 50 cycles.

In an embodiment, a textile or leather of the present disclosure exhibits abrasion resistant. In an embodiment, the foregoing abrasion resistant property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article exhibits the property of blocking ultraviolet (UV) radiation. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article exhibits the property of blocking ultraviolet (UV) radiation. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, wherein the article exhibits the property of blocking ultraviolet (UV) radiation. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the article exhibits the property of blocking ultraviolet (UV) radiation. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the article exhibits the property of blocking ultraviolet (UV) radiation. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article exhibits the property of blocking ultraviolet (UV) radiation. In some embodiments, recombinant silk is as provided in any of the compositions #1001-2450, #3001-4450, and #5001-6595. In an embodiment, the foregoing UV blocking property of the fabric is determined after a period of machine washing cycles selected from the group consisting of 5 cycles, 10 cycles, 25 cycles, and 50 cycles.

In an embodiment, a textile or leather of the present disclosure exhibits UV blocking property. In an embodiment, the foregoing UV blocking property of the textile is determined after a period of machine washing cycles selected from the group consisting of 5 cycles, 10 cycles, 25 cycles, and 50 cycles.

In an embodiment, the disclosure provides a garment comprising a fiber or yarn having a coating, wherein the coating comprises recombinant silk based proteins or fragments thereof having a weight average molecular weight, or average weight average molecular weight range of about 5 kDa to about 144 kDa, wherein the garment regulates the body temperature of a wearer. In some embodiments, recombinant silk is as provided in any of the compositions #1001-2450, #3001-4450, and #5001-6595.

In an embodiment, the disclosure provides a garment comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the garment regulates the body temperature of a wearer. In some embodiments, recombinant silk is as provided in any of the compositions #1001-2450, #3001-4450, and #5001-6595.

In an embodiment, the disclosure provides a garment comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, wherein the garment regulates the body temperature of a wearer. In some embodiments, recombinant silk is as provided in any of the compositions #1001-2450, #3001-4450, and #5001-6595.

In an embodiment, the disclosure provides a garment comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the garment regulates the body temperature of a wearer. In some embodiments, recombinant silk is as provided in any of the compositions #1001-2450, #3001-4450, and #5001-6595.

In an embodiment, the disclosure provides a garment comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the garment regulates the body temperature of a wearer. In some embodiments, recombinant silk is as provided in any of the compositions #1001-2450, #3001-4450, and #5001-6595.

In an embodiment, the disclosure provides a garment comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the garment regulates the body temperature of a wearer. In some embodiments, recombinant silk is as provided in any of the compositions #1001-2450, #3001-4450, and #5001-6595. In an embodiment, the foregoing temperature regulation property of the fabric is determined after a period of machine washing cycles selected from the group consisting of 5 cycles, 10 cycles, 25 cycles, and 50 cycles.

In an embodiment, a textile or leather of the present disclosure exhibits a temperature regulation property. In an embodiment, the foregoing temperature regulation property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, and wherein the article is tear resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the article is tear resistant. In some embodiments, recombinant silk is as provided in any of the compositions #1001-2450, #3001-4450, and #5001-6595. In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, and wherein the article is tear resistant.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, and wherein the article is tear resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, and wherein the article is tear resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight range of about 5 kDa to about 144 kDa, wherein the article is a fabric, and wherein the article is tear resistant. In some embodiments, recombinant silk is as provided in any of the compositions #1001-2450, #3001-4450, and #5001-6595. In an embodiment, the foregoing tear resistant property of the fabric is determined after a period of machine washing cycles selected from the group consisting of 5 cycles, 10 cycles, 25 cycles, and 50 cycles.

In an embodiment, a textile or leather of the present disclosure exhibits a tear resistant property. In an embodiment, the foregoing tear resistant property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the elasticity of the article is improved. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the elasticity of the article is reduced. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article exhibits a rebound dampening property. In some embodiments, recombinant silk is as provided in any of the compositions #1001-2450, #3001-4450, and #5001-6595. Without being bound by any specific theory, it is postulated that the coating prevents the article from returning to the original shape or orientation, and results in the rebound dampening property.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article exhibits a rebound dampening property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, wherein the article exhibits a rebound dampening property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the article exhibits a rebound dampening property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the article exhibits a rebound dampening property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article exhibits a rebound dampening property. In some embodiments, recombinant silk is as provided in any of the compositions #1001-2450, #3001-4450, and #5001-6595. In an embodiment, the foregoing rebound dampening property of the fabric is determined after a period of machine washing cycles selected from the group consisting of 5 cycles, 10 cycles, 25 cycles, and 50 cycles.

In an embodiment, a textile or leather of the present disclosure exhibits a rebound dampening property. In an embodiment, the foregoing rebound dampening property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article exhibits an anti-itch property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article exhibits an anti-itch property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, wherein the article exhibits an anti-itch property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the article exhibits an anti-itch property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the article exhibits an anti-itch property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article exhibits an anti-itch property. In some embodiments, recombinant silk is as provided in any of the compositions #1001-2450, #3001-4450, and #5001-6595. In an embodiment, the foregoing anti-itch property of the fabric is determined after a period of machine washing cycles selected from the group consisting of 5 cycles, 10 cycles, 25 cycles, and 50 cycles.

In an embodiment, a textile or leather of the present disclosure exhibits an anti-itch property. In an embodiment, the foregoing anti-itch property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article exhibits an improved insulation/warmth property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article exhibits an improved insulation/warmth property. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article exhibits an improved insulation/warmth property. In some embodiments, recombinant silk is as provided in any of the compositions #1001-2450, #3001-4450, and #5001-6595. In an embodiment, the foregoing improved insulation/warmth property of the fabric is determined after a period of machine washing cycles selected from the group consisting of 5 cycles, 10 cycles, 25 cycles, and 50 cycles.

In an embodiment, a textile or leather of the present disclosure exhibits improved an insulation/warmth property. In an embodiment, the foregoing improved insulation/warmth property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is wrinkle resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is wrinkle resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, wherein the article is wrinkle resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the article is wrinkle resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the article is wrinkle resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is wrinkle resistant. In an embodiment, the foregoing wrinkle resistant property of the fabric is determined after a period of machine washing cycles selected from the group consisting of 5 cycles, 10 cycles, 25 cycles, and 50 cycles. In some embodiments, recombinant silk is as provided in any of the compositions #1001-2450, #3001-4450, and #5001-6595.

In an embodiment, a textile or leather of the present disclosure exhibits wrinkle resistant property. In an embodiment, the foregoing wrinkle resistant property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is stain resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is stain resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, wherein the article is stain resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the article is stain resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the article is stain resistant.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is stain resistant. In an embodiment, the foregoing stain resistant property of the fabric is determined after a period of machine washing cycles selected from the group consisting of 5 cycles, 10 cycles, 25 cycles, and 50 cycles.

In an embodiment, a textile or leather of the present disclosure exhibits stain resistant property. In an embodiment, the foregoing stain resistant property of the textile is determined after a period of machine washing cycles selected from the group consisting of 5 cycles, 10 cycles, 25 cycles, and 50 cycles.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is sticky. In some embodiments, recombinant silk is as provided in any of the compositions #1001-2450, #3001-4450, and #5001-6595. Without being bound to any specific theory, it is postulated that the coating provides stickiness and maintains stickiness.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is sticky. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is sticky. In some embodiments, recombinant silk is as provided in any of the compositions #1001-2450, #3001-4450, and #5001-6595. In an embodiment, the foregoing sticky property of the fabric is determined after a period of machine washing cycles selected from the group consisting of 5 cycles, 10 cycles, 25 cycles, and 50 cycles.

In an embodiment, a textile or leather of the present disclosure exhibits sticky property. In an embodiment, the foregoing sticky property of the textile is determined after a period of machine washing cycles selected from the group consisting of 5 cycles, 10 cycles, 25 cycles, and 50 cycles.

In an embodiment, the disclosure provides an article comprising a textile or leather coated with recombinant silk-based proteins or fragments thereof, wherein the article exhibits improved flame resistance relative to an uncoated textile. In some embodiments, recombinant silk is as provided in any of the compositions #1001-2450, #3001-4450, and #5001-6595. In an embodiment, the disclosure provides an article comprising a textile or leather coated with recombinant silk-based proteins or fragments thereof, wherein the article exhibits equal flame resistance relative to an uncoated textile or leather. In an embodiment, the disclosure provides an article comprising a textile or leather coated with recombinant silk-based proteins or fragments thereof, wherein the article exhibits equal flame resistance relative to an uncoated textile or leather, wherein an alternative textile or leather coating exhibits reduced flame resistance. In an embodiment, the disclosure provides an article comprising a textile or leather coated with recombinant silk-based proteins or fragments thereof, wherein the article exhibits improved resistance to fire relative to an uncoated textile or leather, wherein the improved resistance to fire is determined by a flammability test. In an embodiment, the flammability test measures afterflame time, afterglow time, char length, and the observation of fabric melting or dripping.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is flame resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the article is flame resistant. In some embodiments, recombinant silk is as provided in any of the compositions #1001-2450, #3001-4450, and #5001-6595.

In an embodiment, the disclosure provides an article comprising a polyester having a coating, wherein the coating comprises recombinant silk based proteins or fragments thereof having a weight average molecular weight, or average weight average molecular weight range of about 5 kDa to about 144 kDa, wherein the article is flame resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the recombinant silk based proteins or fragments comprise recombinant silk and a copolymer, wherein the article is flame resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is natural fiber or yarn selected from the group consisting of cotton, alpaca fleece, alpaca wool, lama fleece, lama wool, cotton, cashmere, sheep fleece, sheep wool, and combinations thereof, wherein the article is flame resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the fiber or yarn is selected from the group consisting of natural fiber or yarn, synthetic fiber or yarn, or combinations thereof, wherein the fiber or yarn is synthetic fiber or yarn selected from the group consisting of polyester, nylon, polyester-polyurethane copolymer, and combinations thereof, wherein the article is flame resistant. In some embodiments, recombinant silk is as provided in any of the compositions #1001-2450, #3001-4450, and #5001-6595.

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, wherein the fabric is flame resistant. In some embodiments, recombinant silk is as provided in any of the compositions #1001-2450, #3001-4450, and #5001-6595. In an embodiment, the foregoing flame resistant property of the fabric is determined after a period of machine washing cycles selected from the group consisting of 5 cycles, 10 cycles, 25 cycles, and 50 cycles.

In an embodiment, a textile or leather of the present disclosure is flame resistant. In an embodiment, the foregoing flame resistant property of the textile is determined after a period of machine washing cycles selected from the group consisting of 5 cycles, 10 cycles, 25 cycles, and 50 cycles.

In an embodiment, the disclosure provides a leather coated with coating, wherein the coating comprises recombinant silk based proteins or fragments thereof having a weight average molecular weight, or average weight average molecular weight range of about 5 kDa to about 144 kDa, wherein the leather exhibits an property selected from the group consisting of an improved color retention property, improved mildew resistance, improved resistance to freeze-thaw cycle damage, improved resistance to abrasion, improved blocking of ultraviolet (UV) radiation, improved regulation of the body temperature of a wearer, improved tear resistance, improved elasticity, improved rebound dampening, improved anti-itch properties, improved insulation, improved wrinkle resistance, improved stain resistance, and improved stickiness. In some embodiments, recombinant silk is as provided in any of the compositions #1001-2450, #3001-4450, and #5001-6595. In an embodiment, the disclosure provides a leather coated with coating, wherein the coating comprises recombinant silk based proteins or fragments thereof having a weight average molecular weight, or average weight average molecular weight range of about 5 kDa to about 144 kDa, wherein the coating is transparent.

In any of the foregoing embodiments, at least one property of the article is improved, wherein the property that is improved is selected from the group consisting of color retention, resistance to microbial growth, resistance to bacterial growth, resistance to fungal growth, resistance to the buildup of static electrical charge, resistance to the growth of mildew, transparency of the coating, resistance to freeze-thaw cycle damage, resistance from abrasion, blocking of ultraviolet (UV) radiation, regulation of the body temperature of a wearer, resistance to tearing, elasticity of the article, rebound dampening, tendency to cause itching in the wearer, thermal insulation of the wearer, wrinkle resistance, stain resistance, stickiness to skin, and flame resistance, and wherein the property is improved by an amount relative to an uncoated article selected from the group consisting of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 200%, at least 300%, at least 400%, and at least 500%.

In any of the foregoing embodiments, the recombinant silk based proteins or protein fragments thereof have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof have a polydispersity of between 1 and about 5.0, or between about 1.5 and about 3.0, and optionally wherein the proteins or protein fragments, prior to coating the fabric, do not spontaneously or gradually gelate and do not visibly change in color or turbidity when in a solution for at least 10 days. In some embodiments, recombinant silk is as provided in any of the compositions #1001-2450, #3001-4450, and #5001-6595.

Additional Agents for Use with Textiles Coated with Recombinant Silk-Based Protein Fragments

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is pretreated with a wetting agent. In some embodiments, recombinant silk is as provided in any of the compositions #1001-2450, #3001-4450, and #5001-6595. In an embodiment, the wetting agent improves one or more coating properties. Suitable wetting agents are known to those of skill in the art. Exemplary, non-limiting examples of wetting agents from a representative supplier, Lamberti SPA, are given in the following table.

| | |
|---|---|
| Imbitex ® NDT | Non silicone low foaming with high |
| | wetting in both hot and cold conditions, |
| | with good detergency and good stability |
| | to alkalis. |
| Imbitex ® TBL | Wetting and de-aerating agent. |
| Imbitex ® MRC | Wetting and penetrating agent for |
| | mercerizing of cotton. |
| Tensolam ™ | Low foam, special wetting and |
| Na liq. | dispersing agent for non-woven wet |
| | treatments. |
| Imbitex ® | Wetting agent for water-and oil |
| NRW3 | repellent finishing. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is pretreated with a detergent. In some embodiments, recombinant silk is as provided in any of the compositions #1001-2450, #3001-4450, and #5001-6595. In an embodiment, the detergent improves one or more coating properties. Suitable detergents are known to those of skill in the art. Exemplary, non-limiting examples of detergents from a representative supplier, Lamberti SPA, are given in the following table.

| | |
|---|---|
| Biorol ™ | Wetting and detergent agent with alkaline stability in |
| CPNN | NaOH up to 10° C.. Recommended for continuous |
| | scouring, bleaching, and Jigger applications. |
| Biorol ™ | Wetting and detergent agent with extremely low foam |
| JK new | properties, recommended for high bath turbulence |
| | machine (e.g., jet, overflow, etc.). |
| Biorol ™ | General-purpose wetting and detergent agent suitable for |
| OW 60 | desizing, scouring, and bleaching processes. |
| Biorol ™ | Detergent/wetting agent, low foaming, high concentration, |
| OWK | recommended for over-flow. Useful for removal of |
| | silicone oil on Lycra blends. |
| Cesapon ™ | Specific scouring, de-gumming agent for silk. |
| Silk liq. | |
| Cesapon ™ | High detergent power product containing solvent. |
| Extra | |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is pretreated with a sequestering or dispersing agent. In some embodiments, recombinant silk is as provided in any of the compositions #1001-2450, #3001-4450, and #5001-6595. Suitable sequestering or dispersing agents are known to those of skill in the art. Exemplary, non-limiting examples of sequestering or dispersing agents from a representative supplier, Lamberti SPA, are given in the following table.

| | |
|---|---|
| Lamegal ™ | Dispersing and anti-redepositing agent useful for preparation |
| DSP | dyeing and after soaping of dyed and printed materials with |
| | reactive and vat dyes. This product is also useful as an anti- |
| | oligomer agent in reduction clearing of polyester, dyed or |
| | printed with disperse dyes. |
| Chelam ™ | Multi-purpose sequestring and dispersing agent for a wide |
| TLW/T | variety of textile processes. No shade variation on dyestuff |
| | containing metals. |
| Lamegal ™ | Multi-purpose sequestring and dispersing agent for a wide |
| TL5 | variety of textile processes. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is pretreated with an enzyme. In some embodiments, recombinant silk is as provided in any of the compositions #1001-2450, #3001-4450, and #5001-6595. Suitable enzymes are known to those of skill in the art. Exemplary, non-limiting examples of enzymes from a representative supplier, Lamberti SPA, are given in the following table.

| | |
|---|---|
| Lazim ™ | Thermo-stable amylase for rapid high temperature |
| HT | desizing. |
| Lazim ™ | Specific enzyme for bioscouring; provides optimal |
| PE | wettability, it improves dyeing and color fastness without |
| | causing depolymerization and fabric strength loss. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is pretreated with a bleaching agent. Suitable bleaching agents are known to those of skill in the art. Exemplary, non-limiting examples of bleaching agents from a representative supplier, Lamberti SPA, are given in the following table.

| | |
|---|---|
| Stabilox OTN | Highly concentrated stabilizer for alkaline bleaching with |
| conc. | hydrogen peroxide. Suitable for a wide variety of |
| | processes. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is pretreated with an antifoaming agent. In some embodiments, recombinant silk is as provided in any of the compositions #1001-2450, #3001-4450, and #5001-6595. Suitable antifoaming agents are known to those of skill in the art. Exemplary, non-limiting examples of antifoaming agents from a representative supplier, Lamberti SPA, are given in the following table.

| Antifoam ™ | General purpose defoaming agent. |
|---|---|
| SE 47 | |
| Defomex ™ | Silicone defoamer effective up to 130° C.. |
| JET | Recommended for HT and JET dyeing systems. |
| Defomex ™ | Non-silicone defoamer. |
| 2033 | |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is pretreated with an anti-creasing agent. In some embodiments, recombinant silk is as provided in any of the compositions #1001-2450, #3001-4450, and #5001-6595. Suitable anti-creasing agents are known to those of skill in the art. Exemplary, non-limiting examples of anti-creasing agents from a representative supplier, Lamberti SPA, are given in the following table.

| | |
|---|---|
| Lubisol ™ | Lubricating and anti-creasing agent for rope wet operation |
| AM | on all kind of fibers and machines. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is treated with a dye dispersing agent. In some embodiments, recombinant silk is as provided in any of the compositions #1001-2450, #3001-4450, and #5001-6595. Suitable dye dispersing agents are known to those of skill in the art. Exemplary, non-limiting examples of dye dispersing agents from a representative supplier, Lamberti SPA, are given in the following table.

| | |
|---|---|
| Lamegal ™ BO | Liquid dispersing agent (non-ionic), suitable for |
| | direct, reactive, disperse dyeing and PES |
| | stripping. |
| Lamegal ™ DSP | Dispersing and anti back-staining agent in |
| | preparation, dyeing and soaping of dyed and |
| | printed materials. Antioligomer agent. |
| Lamegal ™ 619 | Effective low foam dispersing leveling agent for |
| | dyeing of PES. |
| Lamegal ™ TL5 | Multi-purpose sequestering and dispersing agent |
| | for a variety of textile processes. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is treated with a dye leveling agent. In some embodiments, recombinant silk is as provided in any of the compositions #1001-2450, #3001-4450, and #5001-6595. Suitable dye leveling agents are known to those of skill in the art. Exemplary, non-limiting examples of dye leveling agents from a representative supplier, Lamberti SPA, are given in the following table.

| | |
|---|---|
| Lamegal ™ A 12 | Leveling agent for dyeing on wool, polyamide |
| | and its blends with acid or metal complex dyes. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is treated with a dye fixing agent. In some embodiments, recombinant silk is as provided in any of the compositions #1001-2450, #3001-4450, and #5001-6595. Suitable dye fixing agents are known to those of skill in the art. Exemplary, non-limiting examples of dye fixing agents from a representative supplier, Lamberti SPA, are given in the following table.

| | |
|---|---|
| Lamfix ™ L | Fixing agent for direct and reactive dyestuffs, |
| | containing formaldehyde. |
| Lamfix ™ LU conc. | Formaldehyde free cationic fixing agent for |
| | direct and reactive dyes. It does not affect the |
| | shade and light fastness. |
| Lamfix ™ PA/TR | Fixing agent to improve the wet fastness of acid |
| | dyes on polyamide fabrics, dyed or printed and |
| | polyamide yarns. Retarding agent in dyeing of |
| | Polyamide/cellulosic blends with direct dyes. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is treated with a dye special resin agent. In some embodiments, recombinant silk is as provided in any of the compositions #1001-2450, #3001-4450, and #5001-6595. Suitable dye special resin agents are known to those of skill in the art. Exemplary, non-limiting examples of dye special resin agents from a representative supplier, Lamberti SPA, are given in the following table.

| | |
|---|---|
| Denifast ™ TC | Special resin for cationization of cellulose fibers |
| | to obtain special effects (“DENIFAST system” |
| | and ”DENISOL system”). |
| Cobral ™ DD/50 | Special resin for cationization of cellulose fibers |
| | to obtain special effect (“DENIFAST system” |
| | and “DENISOL system”). |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is treated with a dye anti-reducing agent. In some embodiments, recombinant silk is as provided in any of the compositions #1001-2450, #3001-4450, and #5001-6595. Suitable dye anti-reducing agents are known to those of skill in the art. Exemplary, non-limiting examples of dye anti-reducing agents from a representative supplier, Lamberti SPA, are given in the following table.

| | |
|---|---|
| Lamberti Redox ™ | Anti-reducing agent in grain form. 100% active |
| L2S gra | content. |
| Lamberti Redox ™ | Anti-reducing agent in liquid form for automatic |
| L2S liq. | dosage. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is treated with a pigment dye system anti-migrating agent. In some embodiments, recombinant silk is as provided in any of the compositions #1001-2450, #3001-4450, and #5001-6595. Suitable pigment dye system anti-migrating agents are known to those of skill in the art. Exemplary, non-limiting examples of pigment dye system anti-migrating agents from a representative supplier, Lamberti SPA, are given in the following table.

| | |
|---|---|
| Neopat Compound | Compound, developed as migration inhibitor for |
| 96/m conc. | continuous dyeing process with pigments (pad- |
| | dry process). |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is treated with a pigment dye system binder. In some embodiments, recombinant silk is as provided in any of the compositions #1001-2450, #3001-4450, and #5001-6595. Suitable pigment dye system binders are known to those of skill in the art. Exemplary, non-limiting examples of pigment dye system binders from a representative supplier, Lamberti SPA, are given in the following table.

| | |
|---|---|
| Neopat Binder PM/S | Concentrated version of a specific binder used to |
| conc. | prepare pad-liquor for dyeing with pigments |
| | (pad-dry process). |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is treated with a pigment dye system binder and anti-migrating agent combination. In some embodiments, recombinant silk is as provided in any of the compositions #1001-2450, #3001-4450, and #5001-6595. Suitable pigment dye system binder and anti-migrating agent combinations are known to those of skill in the art. Exemplary, non-limiting examples of pigment dye system binder and anti-migrating agent combinations from a representative supplier, Lamherti SPA, are given in the following table.

| | |
|---|---|
| Neopat Compound | Highly concentrated all-in-one product |
| PK1 | specifically developed as migration inhibitor |
| | with specific binder for continuous dyeing |
| | process with pigments (pad-dry process). |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is treated with a delave agent. In some embodiments, recombinant silk is as provided in any of the compositions #1001-2450, #3001-4450, and #5001-6595. Suitable delave agents are known to those of skill in the art. Exemplary, non-limiting examples of delave agents from a representative supplier, Lamberti SPA, are given in the following table.

| | |
|---|---|
| Neopat compound | Highly concentrated compound of surfactants |
| FTN | and polymers specifically developed for pigment |
| | dyeing and pigment-reactive dyeing process; |
| | especially for medium/dark shades for wash off |
| | effect. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is traditionally finished with a wrinkle free treatment. In some embodiments, recombinant silk is as provided in any of the compositions #1001-2450, #3001-4450, and #5001-6595. Suitable wrinkle free treatments are known to those of skill in the art. Exemplary, non-limiting examples of wrinkle free treatments from a representative supplier, Lamberti SPA, are given in the following table.

| | |
|---|---|
| Cellofix ™ | Anti-crease modified glyoxalic resin for finishing of |
| ULF conc. | cottons, cellulosics and blends with synthetics fibers. |
| Poliflex ™ | Polyethilenic resin for waxy, full and slippy handle |
| PO 40 | by foulard applications. |
| Rolflex ™ | Aliphatic waterborne Nano-PU dispersion used as |
| WF | extender for wrinkle free treatments. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is traditionally finished with a softener. In some embodiments, recombinant silk is as provided in any of the compositions #1001-2450, #3001-4450, and #5001-6595. Suitable softeners are known to those of skill in the art. Exemplary, non-limiting examples of softeners from a representative supplier, Lamberti SPA, are given in the following table.

| | |
|---|---|
| Texamina ™ | Cationic softening agent with a very soft handle |
| C/FPN | particularly recommended for application by |
| | exhaustion for all kind of fabrics. Suitable also for |
| | cone application. |
| Texamina ™ | 100% cationic softening agent in flakes form for all |
| C SAL flakes | type of fabrics. Dispersible at room temperature. |
| Texamina ™ CL | Amphoteric softening agent for all types of fabrics. |
| LIQ. | Not yellowing. |
| Texamina ™ | Amphoteric softening agent for woven and knitted |
| HVO | fabrics of cotton, other cellulosics and blends. |
| | Provides a soft, smooth and dry handle. Applied by |
| | padding. |
| Texamina ™ | Nonionic silicon dispersion in water. Excellent |
| SIL | softening, lubricating and anti-static properties for |
| | all fiber types by padding. |
| Texamina ™ | Special cationic softener with silk protein inside. |
| SILK | Provides a “swollen touch” particularly suitable for |
| | cellulosic, wool, silk. |
| Lamfinish ™ | All-in compound based on special polymeric |
| LW | hydrophilic softeners; by coating, foulard, and |
| | exhaustion. |
| Elastolam ® | General purpose mono-component silicone |
| E50 | elastomeric softener for textile finishing. |
| Elastolam ® | Modified polysiloxane micro-emulsion which gives |
| EC 100 | a permanent finishing, with extremely soft and |
| | silky handle. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is traditionally finished with a handle modifier. In some embodiments, recombinant silk is as provided in any of the compositions #1001-2450, #3001-4450, and #5001-6595. Suitable handle modifiers are known to those of skill in the art. Exemplary, non-limiting examples of handle modifiers from a representative supplier, Lamberti SPA, are given in the following table.

| | |
|---|---|
| Poliflex ™ CSW | Cationic anti-slipping agent. |
| Poliflex ™ R 75 | Parafine finishing agent to give waxy handle. |
| Poliflex ™ s | Compound specifically developed for special |
| | writing effects. |
| Poliflex ™ m | Compound for special dry-waxy handle. |
| Lamsoft ™ SW 24 | Compound for special slippy handle specifically |
| | developed for coating application. |
| Lamfinish ™ | All-in-one compound to get a slippy touch; by |
| SLIPPY | coating. |
| Lamfinish ™ | All-in-one compound to get a gummy touch; |
| GUMMY | by coating. |
| Lamfinish ™ | All-in-one compound to get dry-sandy touch |
| OLDRY | especially suitable for vintage effects; by coating. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is traditionally finished with a waterborne polyurethane (PU) dispersion. In some embodiments, recombinant silk is as provided in any of the compositions #1001-2450, #3001-4450, and #5001-6595. Suitable waterborne polyurethane dispersions for traditional finishing are known to those of skill in the art. Exemplary, non-limiting examples of waterborne polyurethane dispersions for traditional finishing from a representative supplier, Lamberti SPA, are given in the following table.

| | |
|---|---|
| Rolflex ™ LB 2 | Aliphatic waterborne PU dispersion particularly |
| | suggested for the formulation of textile coatings |
| | where bright and rigid top finish is required. |
| | It is particularly suitable as a finishing agent |
| | for organza touch on silk fabrics. Transparent |
| | and shiny. |
| Rolflex ™ HP 51 | Aliphatic waterborne PU dispersion particularly |
| | suggested for the formulation of textile coatings |
| | for outwear, luggage, technical articles especially |
| | where hard and flexible touch is required. |
| | Transparent and shiny. |
| Rolflex ™ PU 879 | Aliphatic waterborne PU dispersion particularly |
| | suggested for the formulation of textile coatings |
| | for outwear, luggage, technical articles where a |
| | medium-hard and flexible touch is required. |
| Rolflex ™ ALM | Aliphatic waterborne PU dispersion particularly |
| | suggested for the formulation of textile coatings |
| | for outwear, luggage, technical articles where a |
| | soft and flexible touch is required. Can be also |
| | suitable for printing application. |
| Rolflex ™ AP | Aliphatic waterborne PU dispersion particularly |
| | suggested for the formulation of textile coatings |
| | for outwear, fashion where a soft and gummy |
| | touch is required. |
| Rolflex ™ W4 | Aliphatic waterborne PU dispersion particularly |
| | suggested for the formulation of textile coatings |
| | for clothing, outwear where a full, soft and non |
| | sticky touch is required. |
| Rolflex ™ ZB7 | Aliphatic waterborne PU dispersion particularly |
| | suggested for the formulation of textile coatings |
| | for clothing, outwear, sportswear, fashion and |
| | technical articles for industrial applications. |
| | The product has a very high charge digestion |
| | properties, electrolytes stability and excellent |
| | mechanical and tear resistance. Can be also |
| | suitable for foam coating and printing |
| | application. |
| Rolflex ™ BZ 78 | Aliphatic waterborne PU dispersion particularly |
| | suggested for the formulation of textile coatings |
| | for clothing, outwear, sportswear, fashion and |
| | technical articles for industrial applications. |
| | The product has an excellent hydrolysis |
| | resistance, a very high charge digestion and |
| | electrolytes stability and an excellent |
| | mechanical and tear resistance. Can be also |
| | suitable for foam coating and printing |
| | application. |
| Rolflex ™ K 110 | Gives to the coated fabric a full, soft, and |
| | slightly sticky handle with excellent fastness |
| | on all types of fabrics. |
| Rolflex ™ OP 80 | Aliphatic waterborne PU dispersion particularly |
| | suggested for the formulation of textile coatings |
| | for outwear, luggage and fashion finishes where |
| | an opaque non writing effect is desired. |
| Rolflex ™ NBC | Aliphatic waterborne PU dispersion generally |
| | used by padding application as a filling and zero |
| | formaldehyde sizing agent. Can be used for |
| | outwear and fashion finishing where a full, |
| | elastic and non-sticky touch is required. |
| Rolflex ™ PAD | Aliphatic waterborne PU dispersion specifically |
| | designed for padding application for outwear, |
| | sportswear and fashion applications where a full, |
| | elastic and non sticky touch is required. |
| | Excellent washing and dry cleaning fastness as |
| | well as good bath stability. |
| Rolflex ™ PN | Aliphatic waterborne PU dispersion generally |
| | applied by padding application for outerwear |
| | and fashion high quality applications where |
| | strong, elastic non sticky finishes are required. |
| Elafix ™ PV 4 | Aliphatic blocked isocyanate nano-dispersion |
| | used in order to give anti-felting and anti- |
| | pilling properties to pure wool fabrics and his |
| | blend. |
| Rolflex ™ SW3 | Aliphatic waterborne PU dispersion particularly |
| | suggested to be used by padding application |
| | for the finishing of outwear, sportswear and |
| | fashion where a slippery and elastic touch is |
| | required. It is also a good anti-pilling agent. |
| | Excellent in wool application. |
| Rolflex ™ C 86 | Aliphatic cationic waterborne PU dispersion |
| | particularly suggested for the formulation of |
| | textile coatings for clothing, outwear, fashion |
| | where medium-soft and pleasant full touch is |
| | required. Fabrics treated with the product can |
| | be dyed with a selection of dyes, to get |
| | double-color effects of different intensity. |
| Rolflex ™ CN 29 | Aliphatic cationic waterborne PU dispersion |
| | particularly suggested for the formulation |
| | of textile coatings for clothing, outwear, |
| | fashion where soft and pleasant full touch is |
| | required. Fabrics treated with the product |
| | can be dyed with a selection of dyes, to get |
| | double-color effects of different intensity. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is traditionally finished with a finishing resin. In some embodiments, recombinant silk is as provided in any of the compositions #1001-2450, #3001-4450, and #5001-6595. Suitable finishing resins are known to those of skill in the art. Exemplary, non-limiting examples of finishing resins from a representative supplier, Lamberti SPA, are given in the following table.

| | |
|---|---|
| Textol ™ 110 | Handle modifier with very soft handle for |
| | coating finishes |
| Textol ™ RGD | Water emulsion of acrylic copolymer for |
| | textile coating, with very rigid handle. |
| Textol ™ SB 21 | Butadienic resin for finishing and binder |
| | for textile printing |
| Appretto ™ PV/CC | Vinylacetate water dispersion for rigid |
| | stiffening |
| Amisolo ™ B | CMS water dispersion for textile |
| | finishing as stiffening agent |
| Lamovil ™ RP | PVOH stabilized solution as stiffening agent |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is technically finished with a waterborne polyurethane dispersion. In some embodiments, recombinant silk is as provided in any of the compositions #1001-2450, #3001-4450, and #5001-6595. Suitable waterborne polyurethane dispersions for technical finishing are known to those of skill in the art. Exemplary, non-limiting examples of waterborne polyurethane dispersions for technical finishing from a representative supplier, Lamberti SPA, are given in the following table

| | |
|---|---|
| Rolflex ™ AFP | Aliphatic polyether polyurethane dispersion |
| | in water. The product has high hydrolysis |
| | resistance, good breaking load resistance |
| | and excellent tear resistance. |
| Rolflex ™ ACF | Aliphatic polycarbonate polyurethane |
| | dispersion in water. The product shows |
| | good PU and PVC bonding properties, |
| | excellent abrasion resistance as well as |
| | chemical resistance, included alcohol. |
| Rolflex ™ V 13 | Aliphatic polyether/acrylic copolymer |
| | polyurethane dispersion in water. The |
| | product has good thermoadhesive |
| | properties and good adhesion properties |
| | on PVC. |
| Rolflex ™ K 80 | Aliphatic polyether/acrylic copolymer |
| | polyurethane dispersion in water. |
| | ROLFLEX K 80 is specifically designed |
| | as a high performing adhesive for textile |
| | lamination. The product has excellent |
| | perchloroethylene and water fastness. |
| Rolflex ™ ABC | Aliphatic polyether polyurethane |
| | dispersion in water. Particularly, the |
| | product presents very high water column, |
| | excellent electrolyte resistance, high |
| | LOI index, high resistance to multiple |
| | bending. |
| Rolflex ™ ADH | Aliphatic polyether polyurethane dispersion |
| | in water. The product has a very high water |
| | column resistance. |
| Rolflex ™ W4 | Aliphatic waterborne PU dispersion |
| | particularly suggested for the formulation of |
| | textile coatings for clothing, outwear where |
| | a full, soft and non-sticky touch is required. |
| Rolflex ™ ZB7 | Aliphatic waterborne PU dispersion |
| | particularly suggested for the formulation |
| | of textile coatings for clothing, outwear, |
| | sportswear, fashion and technical articles |
| | for industrial applications. The product |
| | has a very high charge digestion properties, |
| | electrolytes stability and excellent |
| | mechanical and tear resistance. Can be also |
| | suitable for foam coating and printing |
| | application. |
| Rolflex ™ BZ 78 | Aliphatic waterbomed PU dispersion |
| | particularly suggested for the |
| | formulation of textile coatings for clothing, |
| | outwear, sportswear, fashion and technical |
| | articles for industrial applications. The |
| | product has an excellent hydrolysis |
| | resistance, a very high charge digestion |
| | and electrolites stability and an excellent |
| | mechanical and tear resistance. Can be also |
| | suitable for foam coating and printing |
| | application. |
| Rolflex ™ PU 147 | Aliphatic polyether polyurethane dispersion |
| | in water. This product shows good film |
| | forming properties at room temperature. |
| | It has high fastness to light and ultraviolet |
| | radiation and good resistance to water, |
| | solvent and chemical agents, as well as |
| | mechanical resistance. |
| Rolflex ™ SG | Aliphatic polyether polyurethane dispersion |
| | in water. Due to its thermoplastic properties |
| | it is suggested to formulate heat activated |
| | adhesives at low temperatures. |
| Elafix ™ PV 4 | Aliphatic blocked isocyanate nano- |
| | dispersion used in order to give antifelting |
| | and antipilling properties to pure wool |
| | fabrics and his blend. |
| Rolflex ™ C 86 | Aliphatic cationic waterborne PU |
| | dispersion particularly suggested for the |
| | formulation of textile coatings for clothing, |
| | outwear, fashion where medium-soft and |
| | pleasant full touch is required. Fabrics |
| | treated with the product can be dyed with a |
| | selection of dyes, to get double-color |
| | effects of different intensity. |
| Rolflex ™ CN 29 | Aliphatic cationic waterborne PU |
| | dispersion particularly suggested for the |
| | formulation of textile coatings for clothing, |
| | outwear, fashion where soft and pleasant |
| | full touch is required. Fabrics treated with |
| | the product can be dyed with a selection |
| | of dyes, to get double-color effects of |
| | different intensity. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is technically finished with an oil or water repellant. In some embodiments, recombinant silk is as provided in any of the compositions #1001-2450, #3001-4450, and #5001-6595. Suitable oil or water repellants for technical finishing are known to those of skill in the art. Exemplary, non-limiting examples of oil or water repellants for technical finishing from a representative supplier, Lamberti SPA, are given in the following table.

| | |
|---|---|
| Lamgard ™ FT 60 | General purpose fluorocarbon resin for |
| | water and oil repellency; by padding |
| | application. |
| Lamgard ™ 48 | High performance fluorocarbon resin for |
| | water and oil repellency; by padding |
| | application. High rubbing fastness. |
| Imbitex ™ NRW3 | Wetting agent for water-and oil repellent |
| | finishing. |
| Lamgard ™ EXT | Crosslinker for fluorocarbon resins to |
| | improve washing fastness. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is technically finished with a flame retardant. In some embodiments, recombinant silk is as provided in any of the compositions #1001-2450, #3001-4450, and #5001-6595. Suitable flame retardants for technical finishing are known to those of skill in the art. Exemplary, non-limiting examples of flame retardants for technical finishing from a representative supplier, Lamberti SPA, are given in the following table.

| | |
|---|---|
| Piroflam ™ 712 | Non-permanent flame retardant compound |
| | for padding and spray application. |
| Piroflam ™ ECO | Alogen free flame retardant compound |
| | for back coating application for all kind |
| | of fibers. |
| Piroflam ™ UBC | Flame retardant compound for back |
| | coating application for all kind of fibers. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is technically finished with a crosslinker. In some embodiments, recombinant silk is as provided in any of the compositions #1001-2450, #3001-4450, and #5001-6595. Suitable crosslinkers for technical finishing are known to those of skill in the art. Exemplary, non-limiting examples of crosslinkers for technical finishing from a representative supplier, Lamberti SPA, are given in the following table.

| | |
|---|---|
| Rolflex ™ BK8 | Aromatic blocked polyisocyanate in water |
| | dispersion. It is suggested as a cross-linking |
| | agent in coating pastes based of polyurethane |
| | resins to improve washing fastness. |
| Fissativo ™ 05 | Water dispersible aliphatic polyisocyanate |
| | suitable as crosslinking agent for acrylic |
| | and polyurethane dispersions to improve |
| | adhesion and wet and dry scrub resistance. |
| Resina ™ MEL | Melammine-formaldheyde resin. |
| Cellofix ™ VLF | Low formaldehyde melamine resin. |

In an embodiment, the disclosure provides an article comprising a fiber or yarn having a coating, wherein the coating comprises recombinant silk based proteins or fragments thereof having a weight average molecular weight, or average weight average molecular weight range of about 5 kDa to about 144 kDa, wherein the article is a fabric, and wherein the fabric is technically finished with a thickener for technical finishing. In some embodiments, recombinant silk is as provided in any of the compositions #1001-2450, #3001-4450, and #5001-6595. Suitable thickeners for technical finishing are known to those of skill in the art. Exemplary, non-limiting examples of thickeners for technical finishing from a representative supplier, Lamberti SPA, are given in the following table.

| | |
|---|---|
| Lambicol ™ CL 60 | Fully neutralized synthetic thickener for |
| | pigment printing in oil/water emulsion; |
| | medium viscosity type |
| Viscolam ™ PU conc. | Nonionic polyurethane based thickener |
| | with pseudoplastic behavior. |
| Viscolam ™ 115 new | Acrylic thickener; not neutralized. |
| Viscolam ™ PS 202 | Nonionic polyurethane based thickener |
| | with newtonian behavior. |
| Viscolam ™ 1022 | Nonionic polyurethane based thickener |
| | with moderate pseudoplastic behavior. |

In any of the foregoing textile or leather embodiments, the coating comprises recombinant silk based proteins or fragments thereof having a weight average molecular weight, or average weight average molecular weight range of about 5 kDa to about 144 kDa. In any of the foregoing textile or leather embodiments, the coating comprises recombinant silk based proteins or fragments thereof having a weight average molecular weight, or average weight average molecular weight range of about 6 kDa to about 17 kDa. In any of the foregoing textile or leather embodiments, the coating comprises recombinant silk based proteins or fragments thereof having a weight average molecular weight, or average weight average molecular weight range of about 17 kDa to about 39 kDa. In any of the foregoing textile or leather embodiments, the coating comprises recombinant silk based proteins or fragments thereof having a weight average molecular weight, or average weight average molecular weight range of about 39 kDa to about 80 kDa. In some embodiments, recombinant silk is as provided in any of the compositions #1001-2450, #3001-4450, and #5001-6595.

In any of the foregoing textile or leather embodiments, the recombinant silk based proteins or protein fragments thereof have an average weight average molecular weight, or average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof have a polydispersity of between 1 and about 5.0, or between about 1.5 and about 3.0, and optionally wherein the proteins or protein fragments, prior to coating the fabric, do not spontaneously or gradually gelate and do not visibly change in color or turbidity when in a solution for at least 10 days. In some embodiments, recombinant silk is as provided in any of the compositions #1001-2450, #3001-4450, and #5001-6595.

Other Materials Coated with Recombinant Silk-Based Protein Fragments

In an embodiment, the disclosure provides a material coated with recombinant silk-based proteins or fragments thereof. The material may be any material suitable for coating, including plastics (e.g., vinyl), foams (e.g., for use in padding and cushioning), and various natural or synthetic products.

In an embodiment, the disclosure provides an automobile component coated with recombinant silk-based proteins or fragments thereof having a weight average molecular weight, or average weight average molecular weight range of about 5 kDa to about 144 kDa. In an embodiment, the disclosure provides an automobile component coated with recombinant silk-based proteins or fragments thereof having a weight average molecular weight, or average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof have a polydispersity of between about 1.0 and about 5.0, and optionally wherein the proteins or protein fragments, prior to coating the fabric, do not spontaneously or gradually gelate and do not visibly change in color or turbidity when in a solution for at least 10 days. In some embodiments, recombinant silk is as provided in any of the compositions #1001-2450, #3001-4450, and #5001-6595. In an embodiment, the disclosure provides an automobile component coated with recombinant silk-based proteins or fragments thereof, wherein the automobile component exhibits an improved property relative to an uncoated automobile component. In an embodiment, the disclosure provides an automobile component coated with recombinant silk-based proteins or fragments thereof, wherein the automobile component exhibits an improved property relative to an uncoated automobile component, and wherein the automobile component is selected from the group consisting of an upholstery fabric, a headliner, a seat, a headrest, a transmission control, a floor mat, a carpet fabric, a dashboard, a steering wheel, a trim, a wiring harness, an airbag cover, an airbag, a sunvisor, a seat belt, a headrest, an armrest, and a children's car seat. In an embodiment, the disclosure provides an electrical component insulated with a coating comprising recombinant silk-based proteins or fragments thereof.

In an embodiment, the disclosure provides a foam coated with recombinant silk-based proteins or fragments thereof having a weight average molecular weight, or average weight average molecular weight range of about 5 kDa to about 144 kDa. In some embodiments, recombinant silk is as provided in any of the compositions #1001-2450, #3001-4450, and #5001-6595. In an embodiment, the disclosure provides a foam coated with recombinant silk-based proteins or fragments thereof having a weight average molecular weight, or average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof have a polydispersity of between 1 and about 5.0, or between about 1.5 and about 3.0, and optionally wherein the proteins or protein fragments, prior to coating the fabric, do not spontaneously or gradually gelate and do not visibly change in color or turbidity when in a solution for at least 10 days. In some embodiments, recombinant silk is as provided in any of the compositions #1001-2450, #3001-4450, and #5001-6595. In an embodiment, the disclosure provides a foam coated with recombinant silk-based proteins or fragments thereof, wherein the foam exhibits an improved property relative to an uncoated foam, and wherein the foam is selected from the group consisting of a polyurethane foam, an ethylene-vinyl acetate copolymer foam, a low density polyethylene foam, a low density polyethylene foam, a high density polyethylene foam, a polypropylene copolymer foam, a linear low density polyethylene foam, a natural rubber foam, a latex foam, and combinations thereof.

In any of the foregoing embodiments, the material coating comprises recombinant silk based proteins or fragments thereof having a weight average molecular weight, or average weight average molecular weight range of about 5 kDa to about 144 kDa. In any of the foregoing embodiments, the material coating comprises recombinant silk based proteins or fragments thereof having a weight average molecular weight, or average weight average molecular weight range of about 6 kDa to about 17 kDa. In any of the foregoing embodiments, the material coating comprises recombinant silk based proteins or fragments thereof having a weight average molecular weight, or average weight average molecular weight range of about 17 kDa to about 39 kDa. In any of the foregoing embodiments, the material coating comprises recombinant silk based proteins or fragments thereof having a weight average molecular weight, or average weight average molecular weight range of about 39 kDa to about 80 kDa. In some embodiments, recombinant silk is as provided in any of the compositions #1001-2450, #3001-4450, and #5001-6595.

In any of the foregoing embodiments, the recombinant silk based proteins or protein fragments thereof have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof have a polydispersity of between about 1.0 and about 5.0, and wherein the proteins or protein fragments, prior to coating the fabric, do not spontaneously or gradually gelate and do not visibly change in color or turbidity when in a solution for at least 10 days. In some embodiments, recombinant silk is as provided in any of the compositions #1001-2450, #3001-4450, and #5001-6595.

Processes for Coating Textiles and Leathers with Recombinant Silk-Based Protein Fragments

In an embodiment, a method for recombinant silk coating a textile, leather, or other material (such as a foam) includes immersion of the textile, leather, or other material in any of the aqueous solutions of recombinant silk-based protein fragments of the present disclosure. In an embodiment, a method for coating a textile, leather, or other material (such as a foam) includes spraying. In an embodiment, a method for coating a textile, leather, or other material (such as a foam) includes chemical vapor deposition. In an embodiment, a method for recombinant silk coating a textile, leather, or other material (such as a foam) includes electrochemical coating. In an embodiment, a method for recombinant silk coating a textile, leather, or other material (such as a foam) includes knife coating to spread any of the aqueous solutions of recombinant silk-based protein fragments of the present disclosure onto the fabric. The coated article may then be air dried, dried under heat/air flow, or cross-linked to the fabric surface. In an embodiment, a drying process includes curing with additives, irradiation (e.g., using UV light), heat (e.g., microwave or radiofrequency irradiation), and/or drying at ambient condition. In an embodiment, the disclosure provides a method of coating a textile, leather, or other material (such as a foam) comprising the step of applying a coating, wherein the coating comprises a solution of recombinant silk based proteins or fragments thereof having a weight average molecular weight, or average weight average molecular weight range of about 5 kDa to about 144 kDa, wherein the coating is applied to at least one side of the textile, leather, or other material using a method selected from the group consisting of a bath coating process, a spray coating process, a stencil (i.e., screen) process, a recombinant silk-foam based process, a roller-based process, a magnetic roller process, a knife process, a transfer process, a foam process, a lacquering process, a supercritical fluid impregnation process, and a printing process. In some embodiments, recombinant silk is as provided in any of the compositions #1001-2450, #3001-4450, and #5001-6595. In an embodiment, the disclosure provides a method of coating a textile or leather comprising a step selected from the group consisting of providing an unwinding device used to unroll the fabric supply in a roll configuration, providing a feeding system used to control the feed rate of fabric, providing a material compensator used to maintain consistent the fabric tension, providing a coating machine to apply the recombinant silk solution (i.e., recombinant silk-based protein fragments) in different state (liquid or foam) to the fabric, providing a measuring system used to control the amount of recombinant silk solution applied, providing a dryer used to cure or dry the recombinant silk solution on the fabric, providing a cooling station used to bring the fabric temperature close to room value, providing a steering frame used to guide the fabric to the rewinding device and maintain straight edges, providing a rewinding step used to collect the coated fabric in roll, providing UV irradiation for curing of recombinant silk and/or other fabric additives (e.g., in a chemical cross-linking step), providing radiofrequency (RF) irradiation (e.g., using microwave irradiation) for drying and chemical cross-linking, and combinations thereof. Chemical and enzymatic cross-linking steps suitable for use with the compositions, articles, and methods of the disclosure include any method known to those of skill in the art, including but not limited to N-hydroxysuccinimide ester crosslinking, imidoester crosslinking, carbodiimide crosslinking, dicyclohexyl carbodiimide crosslinking, maleimide crosslinking, haloacetyl crosslinking, pyridyl disulfide crosslinking, hydrazide crosslinking, alkoxyamine crosslinking, reductive amination crossling, aryl azide crosslinking, diazirine crosslinking, azide-phosphine crosslinking, transferase crosslinking, hydrolase crosslinking, transglutaminase crosslinking, peptidase crosslinking (e.g., sortase SrtA from Staphylococcus aureus), oxidoreductase crosslinking, tyrosinase crosslinking, laccase crosslinking, peroxidase crosslinking (e.g., horseradish peroxidase), lysyl oxidase crosslinking, and combinations thereof.

In an embodiment, the disclosure provides a method of coating a textile or leather comprising the step of applying a coating, wherein the coating comprises a solution of recombinant silk based proteins or fragments thereof having a weight average molecular weight, or average weight average molecular weight range of about 5 kDa to about 144 kDa, and wherein the coating is applied to at least one side of the textile or leather using a supercritical fluid impregnation process. In some embodiments, recombinant silk is as provided in any of the compositions #1001-2450, #3001-4450, and #5001-6595. The supercritical fluid impregnation process may use CO₂ as the supercritical fluid to solubilize and impregnate recombinant silk based proteins or fragments thereof into a textile or leather, wherein the supercritical CO₂ may include optional organic modifiers known in the art (e.g., methanol) and may further include additional agents described herein, such as dyes.

In an embodiment, the disclosure provides a method of coating a textile or leather comprising the step of applying a coating, wherein the coating comprises a solution of recombinant silk based proteins or fragments thereof having a weight average molecular weight, or average weight average molecular weight range of about 5 kDa to about 144 kDa, using a handheld aerosol spray suitable for consumer use or an aerosol spray system suitable for use by a professional cleaner (e.g., a dry cleaner). In some embodiments, recombinant silk is as provided in any of the compositions #1001-2450, #3001-4450, and #5001-6595.

In an embodiment, the disclosure provides a method of coating a textile or leather comprising the step of applying a coating, wherein the coating comprises a solution of recombinant silk based proteins or fragments thereof having a weight average molecular weight, or average weight average molecular weight range of about 5 kDa to about 144 kDa, using a home washing machine. In some embodiments, recombinant silk is as provided in any of the compositions #1001-2450, #3001-4450, and #5001-6595.

In an embodiment, the disclosure provides a method of coating a fabric comprising the steps of:
(a) applying a pretreatment selected from the group consisting of a wetting agent, a detergent, a sequestering or dispersing agent, an enzyme, a bleaching agent, an antifoaming agent, an anti-creasing agent, a dye dispersing agent, a dye leveling agent, a dye fixing agent, a dye special resin agent, a dye anti-reducing agent, a pigment dye system anti-migrating agent, a pigment dye system binder, a delave agent, a wrinkle free treatment, a softener, a handle modifier, a waterborne polyurethane dispersion, a finishing resin, an oil or water repellant, a flame retardant, a crosslinker, a thickener for technical finishing, or any combination thereof; (b) applying a coating comprising a solution of recombinant silk based proteins or fragments thereof having a weight average molecular weight, or average weight average molecular weight range of about 5 kDa to about 144 kDa, using a spray, screen, or stencil coating process; and (c) drying and optionally curing the coating. In some embodiments, recombinant silk is as provided in any of the compositions #1001-2450, #3001-4450, and #5001-6595.

In any of the foregoing embodiments of methods, the recombinant silk based proteins or protein fragments thereof may have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the recombinant silk based proteins or fragments thereof have a polydispersity of between about 1.0 and about 5.0, and optionally wherein the proteins or protein fragments, prior to coating the fabric, do not spontaneously or gradually gelate and do not visibly change in color or turbidity when in a solution for at least 10 days. In some embodiments, recombinant silk is as provided in any of the compositions #1001-2450, #3001-4450, and #5001-6595.

Additives for Recombinant Silk-Based Protein Fragments and Solutions Thereof

In an embodiment, a solution of the present disclosure is contacted with an additive, such as a therapeutic agent and/or a molecule. In an embodiment, molecules include, but are not limited to, antioxidants and enzymes. In an embodiment, molecules include, but are not limited to, ceramics, ceramic particles, metals, metal particles, polymer particles, aldehydes, luminescent molecules, phosphorescent molecules, fluorescent molecules, inorganic particles, organic particles, selenium, ubiquinone derivatives, thiol-based antioxidants, saccharide-containing antioxidants, polyphenols, botanical extracts, caffeic acid, apigenin, pycnogenol, resveratrol, folic acid, vitamin B12, vitamin B6, vitamin B3, vitamin E, vitamin C and derivatives thereof, vitamin D, vitamin A, astaxathin, Lutein, lycopene, essential fatty acids (omegas 3 and 6), iron, zinc, magnesium, flavonoids (soy, Curcumin, Silymarin, Pycnongeol), growth factors, aloe, hyaluronic acid, extracellular matrix proteins, cells, nucleic acids, biomarkers, biological reagents, zinc oxide, benzoyl peroxide, retinoids, titanium, allergens in a known dose (for sensitization treatment), essential oils including, but not limited to, lemongrass or rosemary oil, and fragrances. Therapeutic agents include, but are not limited to, small molecules, drugs, proteins, peptides and nucleic acids. In an embodiment, a solution of the present disclosure is contacted with an allergen of known quantity prior to forming the article. Allergens include but are not limited to milk, eggs, peanuts, tree nuts, fish, shellfish, soy and wheat. Known doses of allergen loaded within a recombinant silk article can be released at a known rate for controlled exposure allergy study, tests and sensitization treatment.

In an embodiment, recombinant silk-based protein fragments and solutions thereof may be combined with other soluble and insoluble additives coated onto textiles and leather as described herein, wherein the recombinant silk-based protein fragments and solutions functions as a binder or a dispersion medium for the additives. Additives described herein and those known of ordinary skill in the art for use with coating textiles and leather may be used. The combinations of recombinant silk-based protein fragments and solutions thereof with other soluble and insoluble additives may exhibit improved properties as described herein. The property that is improved may be selected from the group consisting of color retention, resistance to microbial growth, resistance to bacterial growth, resistance to fungal growth, resistance to the buildup of static electrical charge, resistance to the growth of mildew, transparency of the coating, resistance to freeze-thaw cycle damage, resistance from abrasion, blocking of ultraviolet (UV) radiation, regulation of the body temperature of a wearer, resistance to tearing, elasticity of the article, rebound dampening, tendency to cause itching in the wearer, thermal insulation of the wearer, wrinkle resistance, stain resistance, stickiness to skin, flame resistance, and combinations thereof. For example, recombinant silk-based protein fragments and solutions thereof may be combined with insoluble ceramic particles as a suspension, and subsequently coated onto a textile using any of the methods described herein to provide further thermal insulation for the wearer and/or to provide improved flame resistance, or to provide other improved properties.

In an embodiment, a solution of the present disclosure is used to create an article with microneedles by standard methods known to one in the art for controlled delivery of molecules or therapeutic agents to or through the skin.

Processes for Production of Recombinant Silk-Based Protein Fragments and Solutions Thereof

As used herein, the term “recombinant silk” refers to recombinant spider and/or silkworm silk protein or fragments thereof. In an embodiment, the spider silk protein is selected from the group consisting of swathing silk (Achniform gland silk), egg sac silk (Cylindriform gland silk), egg case silk (Tubuliform silk), non-sticky dragline silk (Ampullate gland silk), attaching thread silk (Pyriform gland silk), sticky silk core fibers (Flagelliform gland silk), and sticky silk outer fibers (Aggregate gland silk). For example, recombinant spider silk protein, as described herein, includes the proteins described in U.S. Patent Application No. 2016/0222174 and U.S. Pat. Nos. 9,051,453, 9,617,315, 9,689,089, 8,173,772, and 8,642,734. In some embodiments, recombinant silk is as provided in any of the compositions #1001-2450, #3001-4450, and #5001-6595.

Some organisms make multiple silk fibers with unique sequences, structural elements, and mechanical properties. For example, orb weaving spiders have six unique types of glands that produce different silk polypeptide sequences that are polymerized into fibers tailored to fit an environmental or lifecycle niche. The fibers are named for the gland they originate from and the polypeptides are labeled with the gland abbreviation (e.g. “Ma”) and “Sp” for spidroin (short for spider fibroin). In orb weavers, these types include Major Ampullate (MaSp, also called dragline), Minor Ampullate (MiSp), Flagelliform (Flag), Aciniform (AcSp), Tubuliform (TuSp), and Pyriform (PySp). This combination of polypeptide sequences across fiber types, domains, and variation amongst different genus and species of organisms leads to a vast array of potential properties that can be harnessed by commercial production of the recombinant fibers. To date, the vast majority of the work with recombinant silks has focused on the Major Ampullate Spidroins (MaSp).

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSp1 and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1 of U.S. Published Application No. 2016/0222174, the entirety of which is incorporated herein, lists examples of block sequences from selected species and silk types, with further examples presented in Rising, A. et al., Spider silk proteins: recent advances in recombinant production, structure-function relationships and biomedical applications, Cell Mol. Life Sci., 68:2, μg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements.

The construction of certain spider silk block copolymer polypeptides from the blocks and/or macro-repeat domains, according to certain embodiments of the disclosure, is illustrated in U.S. Published Patent Application No. 2016/0222174.

The recombinant block copolymer polypeptides based on spider silk sequences produced by gene expression in a recombinant prokaryotic or eukaryotic system can be purified according to methods known in the art. In a preferred embodiment, a commercially available expression/secretion system can be used, whereby the recombinant polypeptide is expressed and thereafter secreted from the host cell, to be easily purified from the surrounding medium. If expression/secretion vectors are not used, an alternative approach involves purifying the recombinant block copolymer polypeptide from cell lysates (remains of cells following disruption of cellular integrity) derived from prokaryotic or eukaryotic cells in which a polypeptide was expressed. Methods for generation of such cell lysates are known to those of skill in the art. In some embodiments, recombinant block copolymer polypeptides are isolated from cell culture supernatant.

Recombinant block copolymer polypeptide may be purified by affinity separation, such as by immunological interaction with antibodies that bind specifically to the recombinant polypeptide or nickel columns for isolation of recombinant polypeptides tagged with 6-8 histidine residues at their N-terminus or C-terminus Alternative tags may comprise the FLAG epitope or the hemagglutinin epitope. Such methods are commonly used by skilled practitioners.

A solution of such polypeptides (i.e., recombinant silk protein) may then be prepared and used as described herein.

In another embodiment, recombinant silk protein may be prepared according to the methods described in U.S. Pat. No. 8,642,734, the entirety of which is incorporated herein, and used as described herein.

In an embodiment, a recombinant spider silk protein is provided. The spider silk protein typically consists of from 170 to 760 amino acid residues, such as from 170 to 600 amino acid residues, preferably from 280 to 600 amino acid residues, such as from 300 to 400 amino acid residues, more preferably from 340 to 380 amino acid residues. The small size is advantageous because longer spider silk proteins tend to form amorphous aggregates, which require use of harsh solvents for solubilization and polymerization. The recombinant spider silk protein may contain more than 760 residues, in particular in cases where the spider silk protein contains more than two fragments derived from the N-terminal part of a spider silk protein, The spider silk protein comprises an N-terminal fragment consisting of at least one fragment (NT) derived from the corresponding part of a spider silk protein, and a repetitive fragment (REP) derived from the corresponding internal fragment of a spider silk protein. Optionally, the spider silk protein comprises a C-terminal fragment (CT) derived from the corresponding fragment of a spider silk protein. The spider silk protein comprises typically a single fragment (NT) derived from the N-terminal part of a spider silk protein, but in preferred embodiments, the N-terminal fragment include at least two, such as two fragments (NT) derived from the N-terminal part of a spider silk protein. Thus, the spidroin can schematically be represented by the formula NTₘ-REP, and alternatively NTₘ-REP-CT, where m is an integer that is 1 or higher, such as 2 or higher, preferably in the ranges of 1-2, 1-4, 1-6, 2-4 or 2-6. Preferred spidroins can schematically be represented by the formulas NT₂-REP or NT-REP, and alternatively NT₂-REP-CT or NT-REP-CT. The protein fragments are covalently coupled, typically via a peptide bond. In one embodiment, the spider silk protein consists of the NT fragment(s) coupled to the REP fragment, which REP fragment is optionally coupled to the CT fragment.

In one embodiment, the first step of the method of producing polymers of an isolated spider silk protein involves expression of a polynucleic acid molecule which encodes the spider silk protein in a suitable host, such as Escherichia coli. The thus obtained protein is isolated using standard procedures. Optionally, lipopolysaccharides and other pyrogens are actively removed at this stage.

In the second step of the method of producing polymers of an isolated spider silk protein, a solution of the spider silk protein in a liquid medium is provided. By the terms “soluble” and “in solution” is meant that the protein is not visibly aggregated and does not precipitate from the solvent at 60,000×g. The liquid medium can be any suitable medium, such as an aqueous medium, preferably a physiological medium, typically a buffered aqueous medium, such as a 10-50 mM Tris-HCl buffer or phosphate buffer. The liquid medium has a pH of 6.4 or higher and/or an ion composition that prevents polymerization of the spider silk protein. That is, the liquid medium has either a pH of 6.4 or higher or an ion composition that prevents polymerization of the spider silk protein, or both.

Ion compositions that prevent polymerization of the spider silk protein can readily be prepared by the skilled person utilizing the methods disclosed herein. A preferred ion composition that prevents polymerization of the spider silk protein has an ionic strength of more than 300 mM. Specific examples of ion compositions that prevent polymerization of the spider silk protein include above 300 mM NaCl, 100 mM phosphate and combinations of these ions having desired preventive effect on the polymerization of the spider silk protein, e.g. a combination of 10 mM phosphate and 300 mM NaCl.

The presence of an NT fragment improves the stability of the solution and prevents polymer formation under these conditions. This can be advantageous when immediate polymerization may be undesirable, e.g. during protein purification, in preparation of large batches, or when other conditions need to be optimized. It is preferred that the pH of the liquid medium is adjusted to 6.7 or higher, such as 7.0 or higher, or even 8.0 or higher, such as up to 10.5, to achieve high solubility of the spider silk protein. It can also be advantageous that the pH of the liquid medium is adjusted to the range of 6.4-6.8, which provides sufficient solubility of the spider silk protein but facilitates subsequent pH adjustment to 6.3 or lower.

In the third step, the properties of the liquid medium are adjusted to a pH of 6.3 or lower and ion composition that allows polymerization. That is, if the liquid medium wherein the spider silk protein is dissolved has a pH of 6.4 or higher, the pH is decreased to 6.3 or lower. The skilled person is well aware of various ways of achieving this, typically involving addition of a strong or weak acid. If the liquid medium wherein the spider silk protein is dissolved has an ion composition that prevents polymerization, the ion composition is changed so as to allow polymerization. The skilled person is well aware of various ways of achieving this, e.g. dilution, dialysis or gel filtration. If required, this step involves both decreasing the pH of the liquid medium to 6.3 or lower and changing the ion composition so as to allow polymerization. It is preferred that the pH of the liquid medium is adjusted to 6.2 or lower, such as 6.0 or lower. In particular, it may be advantageous from a practical point of view to limit the pH drop from 6.4 or 6.4-6.8 in the preceding step to 6.3 or 6.0-6.3, e.g. 6.2 in this step. In a preferred embodiment, the pH of the liquid medium of this step is 3 or higher, such as 4.2 or higher. The resulting pH range, e.g. 4.2-6.3 promotes rapid polymerization,

In the fourth step, the spider silk protein is allowed to polymerize in the liquid medium having pH of 6.3 or lower and an ion composition that allows polymerization of the spider silk protein. Although the presence of the NT fragment improves solubility of the spider silk protein at a pH of 6.4 or higher and/or an ion composition that prevents polymerization of the spider silk protein, it accelerates polymer formation at a pH of 6.3 or lower when the ion composition allows polymerization of the spider silk protein. The resulting polymers are preferably solid and macroscopic, and they are formed in the liquid medium having a pH of 6.3 or lower and an ion composition that allows polymerization of the spider silk protein. In a preferred embodiment, the pH of the liquid medium of this step is 3 or higher, such as 4.2 or higher. The resulting pH range, e.g. 4.2-6.3 promotes rapid polymerization, Resulting polymer may be provided at the molecular weights described herein and prepared as a solution form that may be used as necessary for article coatings.

Ion compositions that allow polymerization of the spider silk protein can readily be prepared by the skilled person utilizing the methods disclosed herein. A preferred ion composition that allows polymerization of the spider silk protein has an ionic strength of less than 300 mM. Specific examples of ion compositions that allow polymerization of the spider silk protein include 150 mM NaCl, 10 mM phosphate, 20 mM phosphate and combinations of these ions lacking preventive effect on the polymerization of the spider silk protein, e.g. a combination of 10 mM phosphate or 20 mM phosphate and 150 mM NaCl. It is preferred that the ionic strength of this liquid medium is adjusted to the range of 1-250 mM.

Without desiring to be limited to any specific theory, it is envisaged that the NT fragments have oppositely charged poles, and that environmental changes in pH affects the charge balance on the surface of the protein followed by polymerization, whereas salt inhibits the same event.

At neutral pH, the energetic cost of burying the excess negative charge of the acidic pole may be expected to prevent polymerization. However, as the dimer approaches its isoelectric point at lower pH, attractive electrostatic forces will eventually become dominant, explaining the observed salt and pH-dependent polymerization behavior of NT and NT-containing minispidroins. It is proposed that, in some embodiments, pH-induced NT polymerization, and increased efficiency of fiber assembly of NT-minispidroins, are due to surface electrostatic potential changes, and that clustering of acidic residues at one pole of NT shifts its charge balance such that the polymerization transition occurs at pH values of 6.3 or lower.

In a fifth step, the resulting, preferably solid spider silk protein polymers are isolated from said liquid medium. Optionally, this step involves actively removing lipopolysaccharides and other pyrogens from the spidroin polymers.

Without desiring to be limited to any specific theory, it has been observed that formation of spidroin polymers progresses via formation of water-soluble spidroin dimers. The present disclosure thus also provides a method of producing dimers of an isolated spider silk protein, wherein the first two method steps are as described above. The spider silk proteins are present as dimers in a liquid medium at a pH of 6.4 or higher and/or an ion composition that prevents polymerization of said spider silk protein. The third step involves isolating the dimers obtained in the second step, and optionally removal of lipopolysaccharides and other pyrogens. In a preferred embodiment, the spider silk protein polymer of the disclosure consists of polymerized protein dimers. The present disclosure thus provides a novel use of a spider silk protein, preferably those disclosed herein, for producing dimers of the spider silk protein.

According to another aspect, the disclosure provides a polymer of a spider silk protein as disclosed herein. In an embodiment, the polymer of this protein is obtainable by any one of the methods therefor according to the disclosure. Thus, the disclosure provides various uses of recombinant spider silk protein, preferably those disclosed herein, for producing polymers of the spider silk protein as recombinant silk based coatings. According to one embodiment, the present disclosure provides a novel use of a dimer of a spider silk protein, preferably those disclosed herein, for producing polymers of the isolated spider silk protein as recombinant silk based coatings. In these uses, it is preferred that the polymers are produced in a liquid medium having a pH of 6.3 or lower and an ion composition that allows polymerisation of said spider silk protein. In an embodiment, the pH of the liquid medium is 3 or higher, such as 4.2 or higher. The resulting pH range, e.g. 4.2-6.3 promotes rapid polymerization,

Using the method(s) of the present disclosure, it is possible to control the polymerization process, and this allows for optimization of parameters for obtaining silk polymers with desirable properties and shapes.

In an embodiment, the recombinant silk proteins described herein, include those described in U.S. Pat. No. 8,642,734, the entirety of which is incorporated by reference.

In another embodiment, the recombinant silk proteins described herein may be prepared according to the methods described in U.S. Pat. No. 9,051,453, the entirety of which is incorporated herein by reference.

In an embodiment, a silk protein may include a polypeptide derived from natural spider silk proteins. The polypeptide is not limited particularly as long as it is derived from natural spider silk proteins, and examples of the polypeptide include natural spider silk proteins and recombinant spider silk proteins such as variants, analogs, derivatives or the like of the natural spider silk proteins. In terms of excellent tenacity, the polypeptide may be derived from major dragline silk proteins produced in major ampullate glands of spiders. Examples of the major dragline silk proteins include major ampullate spidroin MaSp1 and MaSp2 from Nephila clavipes, and ADF3 and ADF4 from Araneus diadematus, etc. Examples of the polypeptide derived from major dragline silk proteins include variants, analogs, derivatives or the like of the major dragline silk proteins. Further, the polypeptide may be derived from flagelliform silk proteins produced in flagelliform glands of spiders. Examples of the flagelliform silk proteins include flagelliform silk proteins derived from Nephila clavipes, etc.

Examples of the polypeptide derived from major dragline silk proteins include a polypeptide containing two or more units of an amino acid sequence represented by the formula 1: REP1-REP2 (1), preferably a polypeptide containing five or more units thereof, and more preferably a polypeptide containing ten or more units thereof. Alternatively, the polypeptide derived from major dragline silk proteins may be a polypeptide that contains units of the amino acid sequence represented by the formula 1: REP1-REP2 (1) and that has, at a C-terminal, an amino acid sequence represented by any of SEQ ID NOS: 1 to 3 of U.S. Pat. No. 9,051,453 or an amino acid sequence having a homology of 90% or more with the amino acid sequence represented by any of SEQ ID NOS: 1 to 3 of U.S. Pat. No. 9,051,453. In the polypeptide derived from major dragline silk proteins, units of the amino acid sequence represented by the formula 1: REP1-REP2 (1) may be the same or may be different from each other. In the case of producing a recombinant protein using a microbe such as Escherichia coli as a host, the molecular weight of the polypeptide derived from major dragline silk proteins is preferably 500 kDa or less, more preferably 300 kDa or less, and further preferably 200 kDa or less, in terms of productivity.

In the formula (1), the REP1 indicates polyalanine. In the REP1, the number of alanine residues arranged in succession is preferably 2 or more, more preferably 3 or more, further preferably 4 or more, and particularly preferably 5 or more. Further, in the REP1, the number of alanine residues arranged in succession is preferably 20 or less, more preferably 16 or less, further preferably 12 or less, and particularly preferably 10 or less. In the formula (1), the REP2 is an amino acid sequence composed of 10 to 200 amino acid residues. The total number of glycine, serine, glutamine and alanine residues contained in the amino acid sequence is 40% or more, preferably 60% or more, and more preferably 70% or more with respect to the total number of amino acid residues contained therein.

In the major dragline silk, the REP1 corresponds to a crystal region in a fiber where a crystal 13 sheet is formed, and the REP2 corresponds to an amorphous region in a fiber where most of the parts lack regular configurations and that has more flexibility. Further, the [REP1-REP2] corresponds to a repetitious region (repetitive sequence) composed of the crystal region and the amorphous region, which is a characteristic sequence of dragline silk proteins.

An amino acid sequence represented by SEQ ID NO: 1 of U.S. Pat. No. 9,051,453 is identical to an amino acid sequence that is composed of 50 amino acid residues of an amino acid sequence of ADF3 at the C-terminal (NCBI Accession No.: AAC47010, GI: 1263287). An amino acid sequence represented by SEQ ID NO: 2 of U.S. Pat. No. 9,051,453 is identical to an amino acid sequence represented by SEQ ID NO: 1 of U.S. Pat. No. 9,051,453 from which 20 residues have been removed from the C-terminal. An amino acid sequence represented by SEQ ID NO: 3 of U.S. Pat. No. 9,051,453 is identical to an amino acid sequence represented by SEQ ID NO: 1 from which 29 residues have been removed from the C-terminal.

An example of the polypeptide that contains units of the amino acid sequence represented by the formula 1: REP1-REP2 (1) and that has, at a C-terminal, an amino acid sequence represented by any of SEQ ID NOS: 1 to 3 or an amino acid sequence having a homology of 90% or more with the amino acid sequence represented by any of SEQ ID NOS: 1 to 3 of U.S. Pat. No. 9,051,453 is a polypeptide having an amino acid sequence represented by SEQ ID NO: 8 of U.S. Pat. No. 9,051,453. The polypeptide having the amino acid sequence represented by SEQ ID NO: 8 of U.S. Pat. No. 9,051,453 is obtained by the following mutation: in an amino acid sequence of ADF3 (NCBI Accession No.: AAC47010, GI: 1263287) to the N-terminal of which has been added an amino acid sequence (SEQ ID NO: 5 of U.S. Pat. No. 9,051,453) composed of a start codon, His 10 tags and an HRV3C Protease (Human rhinovirus 3C Protease) recognition site, 1^{st} to 13^{th} repetitive regions are about doubled and the translation ends at the 1154th amino acid residue. In the polypeptide having the amino acid sequence represented by SEQ ID NO: 8 of U.S. Pat. No. 9,051,453, the C-terminal sequence is identical to the amino acid sequence represented by SEQ ID NO: 3.

Further, the polypeptide that contains units of the amino acid sequence represented by the formula 1: REP1-REP2 (1) and that has, at a C-terminal, an amino acid sequence represented by any of SEQ ID NOS: 1 to 3 of U.S. Pat. No. 9,051,453 or an amino acid sequence having a homology of 90% or more with the amino acid sequence represented by any of SEQ ID NOS: 1 to 3 of U.S. Pat. No. 9,051,453 may be a protein that has an amino acid sequence represented by SEQ ID NO: 8 of U.S. Pat. No. 9,051,453 in which one or a plurality of amino acids have been substituted, deleted, inserted and/or added and that has a repetitious region composed of a crystal region and an amorphous region.

Further, an example of the polypeptide containing two or more units of the amino acid sequence represented by the formula 1: REP1-REP2 (1) is a recombinant protein derived from ADF4 having an amino acid sequence represented by SEQ ID NO: 15 of U.S. Pat. No. 9,051,453. The amino acid sequence represented by SEQ ID NO: 15 of U.S. Pat. No. 9,051,453 is an amino acid sequence obtained by adding the amino acid sequence (SEQ ID NO: 5 of U.S. Pat. No. 9,051,453) composed of a start codon, His 10 tags and an HRV3C Protease (Human rhinovirus 3C Protease) recognition site, to the N-terminal of a partial amino acid sequence of ADF4 obtained from the NCBI database (NCBI Accession No.: AAC47011, GI: 1263289). Further, the polypeptide containing two or more units of the amino acid sequence represented by the formula 1: REP1-REP2 (1) may be a polypeptide that has an amino acid sequence represented by SEQ ID NO: 15 of U.S. Pat. No. 9,051,453 in which one or a plurality of amino acids have been substituted, deleted, inserted and/or added and that has a repetitious region composed of a crystal region and an amorphous region. Further, an example of the polypeptide containing two or more units of the amino acid sequence represented by the formula 1: REP1-REP2 (1) is a recombinant protein derived from MaSp2 that has an amino acid sequence represented by SEQ ID NO: 17 of U.S. Pat. No. 9,051,453. The amino acid sequence represented by SEQ ID NO: 17 of U.S. Pat. No. 9,051,453 is an amino acid sequence obtained by adding the amino acid sequence (SEQ ID NO: 5 of U.S. Pat. No. 9,051,453) composed of a start codon, His 10 tags and an HRV3C Protease (Human rhinovirus 3C Protease) recognition site, to the N-terminal of a partial sequence of MaSp2 obtained from the NCBI web database (NCBI Accession No.: AAT75313, GI: 50363147). Furthermore, the polypeptide containing two or more units of the amino acid sequence represented by the formula 1: REP1-REP2 (1) may be a polypeptide that has an amino acid sequence represented by SEQ ID NO: 17 of U.S. Pat. No. 9,051,453 in which one or a plurality of amino acids have been substituted, deleted, inserted and/or added and that has a repetitious region composed of a crystal region and an amorphous region.

Examples of the polypeptide derived from flagelliform silk proteins include a polypeptide containing 10 or more units of an amino acid sequence represented by the formula 2: REP3 (2), preferably a polypeptide containing 20 or more units thereof, and more preferably a polypeptide containing 30 or more units thereof. In the case of producing a recombinant protein using a microbe such as Escherichia coli as a host, the molecular weight of the polypeptide derived from flagelliform silk proteins is preferably 500 kDa or less, more preferably 300 kDa or less, and further preferably 200 kDa or less, in terms of productivity.

In the formula (2), the REP 3 indicates an amino acid sequence composed of Gly-Pro-Gly-Gly-X, where X indicates an amino acid selected from the group consisting of Ala, Ser, Tyr and Val.

A major characteristic of the spider silk is that the flagelliform silk does not have a crystal region, but has a repetitious region composed of an amorphous region. Since the major dragline silk and the like have a repetitious region composed of a crystal region and an amorphous region, they are expected to have both high stress and stretchability. Meanwhile, as to the flagelliform silk, although the stress is inferior to that of the major dragline silk, the stretchability is high. The reason for this is considered to be that most of the flagelliform silk is composed of amorphous regions.

An example of the polypeptide containing 10 or more units of the amino acid sequence represented by the formula 2: REP3 (2) is a recombinant protein derived from flagelliform silk proteins having an amino acid sequence represented by SEQ ID NO: 19 of U.S. Pat. No. 9,051,453. The amino acid sequence represented by SEQ ID NO: 19 of U.S. Pat. No. 9,051,453 is an amino acid sequence obtained by combining a partial sequence of flagelliform silk protein of Nephila clavipes obtained from the NCBI database (NCBI Accession No.: AAF36090, GI: 7106224), specifically, an amino acid sequence thereof from the 1220th residue to the 1659th residue from the N-terminal that corresponds to repetitive sections and motifs (referred to as a PR1 sequence), with a partial sequence of flagelliform silk protein of Nephila clavipes obtained from the NCBI database (NCBI Accession No.: AAC38847, GI: 2833649), specifically, a C-terminal amino acid sequence thereof from the 816th residue to the 907th residue from the C-terminal, and thereafter adding the amino acid sequence (SEQ ID NO: 5 of U.S. Pat. No. 9,051,453) composed of a start codon, His 10 tags and an HRV3C Protease recognition site, to the N-terminal of the combined sequence. Further, the polypeptide containing 10 or more units of the amino acid sequence represented by the formula 2: REP3 (2) may be a polypeptide that has an amino acid sequence represented by SEQ ID NO: 19 of U.S. Pat. No. 9,051,453 in which one or a plurality of amino acids have been substituted, deleted, inserted and/or added and that has a repetitious region composed of an amorphous region.

The polypeptide can be produced using a host that has been transformed by an expression vector containing a gene encoding a polypeptide. A method for producing a gene is not limited particularly, and it may be produced by amplifying a gene encoding a natural spider silk protein from a cell derived from spiders by a polymerase chain reaction (PCR), etc., and cloning it, or may be synthesized chemically. Also, a method for chemically synthesizing a gene is not limited particularly, and it can be synthesized as follows, for example: based on information of amino acid sequences of natural spider silk proteins obtained from the NCBI web database, etc., oligonucleotides that have been synthesized automatically with AKTA oligopilot plus 10/100 (GE Healthcare Japan Corporation) are linked by PCR, etc. At this time, in order to facilitate the purification and observation of protein, it is possible to synthesize a gene that encodes a protein having an amino acid sequence of the above-described amino acid sequence to the N-terminal of which has been added an amino acid sequence composed of a start codon and His 10 tags.

Examples of the expression vector include a plasmid, a phage, a virus, and the like that can express protein based on a DNA sequence. The plasmid-type expression vector is not limited particularly as long as it allows a target gene to be expressed in a host cell and it can amplify itself. For example, in the case of using Escherichia coli Rosetta (DE3) as a host, a pET22b(+) plasmid vector, a pCold plasmid vector, and the like can be used. Among these, in terms of productivity of protein, it is preferable to use the pET22b(+) plasmid vector. Examples of the host include animal cells, plant cells, microbes, etc.

The polypeptide used in the present disclosure is preferably a polypeptide derived from ADF3, which is one of two principal dragline silk proteins of Araneus diadematus. This polypeptide has advantages of basically having high strength-elongation and toughness and of being synthesized easily.

Accordingly, the recombinant silk protein (e.g., the recombinant spider silk-based protein) used in accordance with the embodiments, articles, and/or methods described herein, may include one or more recombinant silk proteins described above or recited in U.S. Pat. Nos. 8,173,772, 8,278,416, 8,618,255, 8,642,734, 8,691,581, 8,729,235, 9,115,204, 9,157,070, 9,309,299, 9,644,012, 9,708,376, 9,051,453, 9,617,315, 9,968,682, 9,689,089, 9,732,125, 9,856,308, 9,926,348, 10,065,997, 10,316,069, and 10,329,332; and U.S. Patent Publication Nos. 2009/0226969, 2011/0281273, 2012/0041177, 2013/0065278, 2013/0115698, 2013/0316376, 2014/0058066, 2014/0079674, 2014/0245923, 2015/0087046, 2015/0119554, 2015/0141618, 2015/0291673, 2015/0291674, 2015/0239587, 2015/0344542, 2015/0361144, 2015/0374833, 2015/0376247, 2016/0024464, 2017/0066804, 2017/0066805, 2015/0293076, 2016/0222174, 2017/0283474, 2017/0088675, 2019/0135880, 2015/0329587, 2019/0040109, 2019/0135881, 2019/0177363, 2019/0225646, 2019/0233481, 2019/0031842, 2018/0355120, 2019/0186050, 2019/0002644, 2020/0031887, 2018/0273590, 20191/094403, 2019/0031843, 2018/0251501, 2017/0066805, 2018/0127553, 2019/03290, 2020/0031886, 2018/0080147, 2019/0352349, 2020/0043085, 2019/0144819, 2019/0228449, 2019/0340666, 2020/0000091, 2019/0194710, 2019/0151505, 2018/0265555, 2019/0352330, 2019/0248847, and 2019/0378191, the entirety of which are incorporated herein by reference.

In an embodiment, when producing a recombinant silk gel, an acid is used to help facilitate gelation. In an embodiment, when producing a recombinant silk gel that includes a neutral or a basic molecule and/or therapeutic agent, an acid can be added to facilitate gelation. In an embodiment, when producing a recombinant silk gel, increasing the pH (making the gel more basic) increases the shelf stability of the gel.

In an embodiment, when producing a silk gel, increasing the pH (making the gel more basic) allows for a greater quantity of an acidic molecule to be loaded into the gel.

In an embodiment, natural additives may be added to the recombinant silk gel to further stabilize additives. For example, trace elements such as selenium or magnesium or L-methionine can be used. Further, light-block containers can be added to further increase stability.

In some embodiments, a composition of the present disclosure can further include skin penetration enhancers, including, but not limited to, sulfoxides (such as dimethylsulfoxide), pyrrolidones (such as 2-pyrrolidone), alcohols (such as ethanol or decanol), azones (such as laurocapram and 1-dodecylazacycloheptan-2-one), surfactants (including alkyl carboxylates and their corresponding acids such as oleic acid, fluoroalkylcarboxylates and their corresponding acids, alkyl sulfates, alkyl ether sulfates, docusates such as dioctyl sodium sulfosuccinate, alkyl benzene sulfonates, alkyl ether phosphates, and alkyl aryl ether phosphates), glycols (such as propylene glycol), terpenes (such as limonene, p-cymene, geraniol, farnesol, eugenol, menthol, terpineol, carveol, carvone, fenchone, and verbenone), and dimethyl isosorbide.

Following are non-limiting examples of suitable ranges for various parameters in and for preparation of the silk solutions of the present disclosure. The silk solutions of the present disclosure may include one or more, but not necessarily all, of these parameters and may be prepared using various combinations of ranges of such parameters.

In an embodiment, the percent recombinant silk in the solution is less than 30%. In an embodiment, the percent recombinant silk in the solution is less than 25%. In an embodiment, the percent recombinant silk in in the solution is less than 20%. In an embodiment, the percent recombinant silk in the solution is less than 19%. In an embodiment, the percent recombinant silk in the solution is less than 18%. In an embodiment, the percent recombinant silk in the solution is less than 17%. In an embodiment, the percent recombinant silk in the solution is less than 16%. In an embodiment, the percent recombinant silk in the solution is less than 15%. In an embodiment, the percent recombinant silk in the solution is less than 14%. In an embodiment, the percent recombinant silk in the solution is less than 13%. In an embodiment, the percent recombinant silk in the solution is less than 12%. In an embodiment, the percent recombinant silk in the solution is less than 11%. In an embodiment, the percent recombinant silk in the solution is less than 10%. In an embodiment, the percent recombinant silk in the solution is less than 9%. In an embodiment, the percent recombinant silk in the solution is less than 8%. In an embodiment, the percent recombinant silk in the solution is less than 7%. In an embodiment, the percent recombinant silk in the solution is less than 6%. In an embodiment, the percent recombinant silk in the solution is less than 5%. In an embodiment, the percent recombinant silk in the solution is less than 4%. In an embodiment, the percent recombinant silk in the solution is less than 3%. In an embodiment, the percent recombinant silk in the solution is less than 2%. In an embodiment, the percent recombinant silk in the solution is less than 1%. In an embodiment, the percent recombinant silk in the solution is less than 0.9%. In an embodiment, the percent recombinant silk in the solution is less than 0.8%. In an embodiment, the percent recombinant silk in the solution is less than 0.7%. In an embodiment, the percent recombinant silk in the solution is less than 0.6%. In an embodiment, the percent recombinant silk in the solution is less than 0.5%. In an embodiment, the percent recombinant silk in the solution is less than 0.4%. In an embodiment, the percent recombinant silk in the solution is less than 0.3%. In an embodiment, the percent recombinant silk in the solution is less than 0.2%. In an embodiment, the percent recombinant silk in the solution is less than 0.1%. In an embodiment, the percent recombinant silk in the solution is greater than 0.1%. In an embodiment, the percent recombinant silk in the solution is greater than 0.2%. In an embodiment, the percent recombinant silk in the solution is greater than 0.3%. In an embodiment, the percent recombinant silk in the solution is greater than 0.4%. In an embodiment, the percent recombinant silk in the solution is greater than 0.5%. In an embodiment, the percent recombinant silk in the solution is greater than 0.6%. In an embodiment, the percent recombinant silk in the solution is greater than 0.7%. In an embodiment, the percent recombinant silk in the solution is greater than 0.8%. In an embodiment, the percent recombinant silk in the solution is greater than 0.9%. In an embodiment, the percent recombinant silk in the solution is greater than 1%. In an embodiment, the percent recombinant silk in the solution is greater than 2%. In an embodiment, the percent recombinant silk in the solution is greater than 3%. In an embodiment, the percent recombinant silk in the solution is greater than 4%. In an embodiment, the percent recombinant silk in the solution is greater than 5%. In an embodiment, the percent recombinant silk in the solution is greater than 6%. In an embodiment, the percent recombinant silk in the solution is greater than 7%. In an embodiment, the percent recombinant silk in the solution is greater than 8%. In an embodiment, the percent recombinant silk in the solution is greater than 9%. In an embodiment, the percent recombinant silk in the solution is greater than 10%. In an embodiment, the percent recombinant silk in the solution is greater than 11%. In an embodiment, the percent recombinant silk in the solution is greater than 12%. In an embodiment, the percent recombinant silk in the solution is greater than 13%. In an embodiment, the percent recombinant silk in the solution is greater than 14%. In an embodiment, the percent recombinant silk in the solution is greater than 15%. In an embodiment, the percent recombinant silk in the solution is greater than 16%. In an embodiment, the percent recombinant silk in the solution is greater than 17%. In an embodiment, the percent recombinant silk in the solution is greater than 18%. In an embodiment, the percent recombinant silk in the solution is greater than 19%. In an embodiment, the percent recombinant silk in the solution is greater than 20%. In an embodiment, the percent recombinant silk in the solution is greater than 25%. In an embodiment, the percent recombinant silk in the solution is between 0.1% and 30%. In an embodiment, the percent recombinant silk in the solution is between 0.1% and 25%. In an embodiment, the percent recombinant silk in the solution is between 0.1% and 20%. In an embodiment, the percent recombinant silk in the solution is between 0.1% and 15%. In an embodiment, the percent recombinant silk in the solution is between 0.1% and 10%. In an embodiment, the percent recombinant silk in the solution is between 0.1% and 9%. In an embodiment, the percent recombinant silk in the solution is between 0.1% and 8%. In an embodiment, the percent recombinant silk in the solution is between 0.1% and 7%. In an embodiment, the percent recombinant silk in the solution is between 0.1% and 6.5%. In an embodiment, the percent recombinant silk in the solution is between 0.1% and 6%. In an embodiment, the percent recombinant silk in the solution is between 0.1% and 5.5%. In an embodiment, the percent recombinant silk in the solution is between 0.1% and 5%. In an embodiment, the percent recombinant silk in the solution is between 0.1% and 4.5%. In an embodiment, the percent recombinant silk in the solution is between 0.1% and 4%. In an embodiment, the percent recombinant silk in the solution is between 0.1% and 3.5%. In an embodiment, the percent recombinant silk in the solution is between 0.1% and 3%. In an embodiment, the percent recombinant silk in the solution is between 0.1% and 2.5%. In an embodiment, the percent recombinant silk in the solution is between 0.1% and 2.0%. In an embodiment, the percent recombinant silk in the solution is between 0.1% and 2.4%. In an embodiment, the percent recombinant silk in the solution is between 0.5% and 5%. In an embodiment, the percent recombinant silk in the solution is between 0.5% and 4.5%. In an embodiment, the percent recombinant silk in the solution is between 0.5% and 4%. In an embodiment, the percent recombinant silk in the solution is between 0.5% and 3.5%. In an embodiment, the percent recombinant silk in the solution is between 0.5% and 3%. In an embodiment, the percent recombinant silk in the solution is between 0.5% and 2.5%. In an embodiment, the percent recombinant silk in the solution is between 1 and 4%. In an embodiment, the percent recombinant silk in the solution is between 1 and 3.5%. In an embodiment, the percent recombinant silk in the solution is between 1 and 3%. In an embodiment, the percent recombinant silk in the solution is between 1 and 2.5%. In an embodiment, the percent recombinant silk in the solution is between 1 and 2.4%. In an embodiment, the percent recombinant silk in the solution is between 1 and 2%. In an embodiment, the percent recombinant silk in the solution is between 20% and 30%. In an embodiment, the percent recombinant silk in the solution is between 0.1% and 6%. In an embodiment, the percent recombinant silk in the solution is between 6% and 10%. In an embodiment, the percent recombinant silk in the solution is between 6% and 8%. In an embodiment, the percent recombinant silk in the solution is between 6% and 9%. In an embodiment, the percent recombinant silk in the solution is between 10% and 20%. In an embodiment, the percent recombinant silk in the solution is between 11% and 19%. In an embodiment, the percent recombinant silk in the solution is between 12% and 18%. In an embodiment, the percent recombinant silk in the solution is between 13% and 17%. In an embodiment, the percent recombinant silk in the solution is between 14% and 16%. In an embodiment, the percent recombinant silk in the solution is 2.4%. In an embodiment, the percent recombinant silk in the solution is 2.0%. In some embodiments, recombinant silk is as provided in any of the compositions #1001-2450, #3001-4450, and #5001-6595.

In an embodiment, the stability of a composition of the present disclosure is about 1 day. In an embodiment, the stability of a composition of the present disclosure is about 2 days. In an embodiment, the stability of a composition of the present disclosure is about 3 days. In an embodiment, the stability of a composition of the present disclosure is about 4 days. In an embodiment, the stability of a composition of the present disclosure is about 5 days. In an embodiment, the stability of a composition of the present disclosure is about 6 days. In an embodiment, the stability of a composition of the present disclosure is about 7 days. In an embodiment, the stability of a composition of the present disclosure is about 8 days. In an embodiment, the stability of a composition of the present disclosure is about 9 days. In an embodiment, the stability of a composition of the present disclosure is about 10 days.

In an embodiment, the stability of a composition of the present disclosure is about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a composition of the present disclosure includes recombinant silk-based protein fragments having an average weight average molecular weight ranging from 6 kDa to 17 kDa. In an embodiment, a composition of the present disclosure includes recombinant silk-based protein fragments having a weight average molecular weight ranging from 17 kDa to 39 kDa. In an embodiment, a composition of the present disclosure includes recombinant silk-based protein fragments having an average weight average molecular weight ranging from 17 kDa to 39 kDa. In an embodiment, a composition of the present disclosure includes recombinant silk-based protein fragments having an average weight average molecular weight ranging from 39 kDa to 80 kDa. In some embodiments, recombinant silk is as provided in any of the compositions #1001-2450, #3001-4450, and #5001-6595.

In an embodiment, a composition of the present disclosure includes recombinant silk-based protein fragments having an average weight average molecular weight ranging from 1 to about 5 kDa. In an embodiment, a composition of the present disclosure includes recombinant silk-based protein fragments having an average weight average molecular weight ranging from about 5 to about 10 kDa. In an embodiment, a composition of the present disclosure includes recombinant silk-based protein fragments having an average weight average molecular weight ranging from about 10 to about 15 kDa. In an embodiment, a composition of the present disclosure includes recombinant silk-based protein fragments having an average weight average molecular weight ranging from about 15 to about 20 kDa. In an embodiment, a composition of the present disclosure includes recombinant silk-based protein fragments having an average weight average molecular weight ranging from about 20 to about 25 kDa. In an embodiment, a composition of the present disclosure includes recombinant silk-based protein fragments having an average weight average molecular weight ranging from about 25 to about 30 kDa. In an embodiment, a composition of the present disclosure includes recombinant silk-based protein fragments having an average weight average molecular weight ranging from about 30 to about 35 kDa. In an embodiment, a composition of the present disclosure includes recombinant silk-based protein fragments having an average weight average molecular weight ranging from about 35 to about 40 kDa. In an embodiment, a composition of the present disclosure includes recombinant silk-based protein fragments having an average weight average molecular weight ranging from about 40 to about 45 kDa. In an embodiment, a composition of the present disclosure includes recombinant silk-based protein fragments having an average weight average molecular weight ranging from about 45 to about 50 kDa. In an embodiment, a composition of the present disclosure includes recombinant silk-based protein fragments having an average weight average molecular weight ranging from about 50 to about 55 kDa. In an embodiment, a composition of the present disclosure includes recombinant silk-based protein fragments having an average weight average molecular weight ranging from about 55 to about 60 kDa. In an embodiment, a composition of the present disclosure includes recombinant silk-based protein fragments having an average weight average molecular weight ranging from about 60 to about 65 kDa. In an embodiment, a composition of the present disclosure includes recombinant silk-based protein fragments having an average weight average molecular weight ranging from about 65 to about 70 kDa. In an embodiment, a composition of the present disclosure includes recombinant silk-based protein fragments having an average weight average molecular weight ranging from about 70 to about 75 kDa. In an embodiment, a composition of the present disclosure includes recombinant silk-based protein fragments having an average weight average molecular weight ranging from about 75 to about 80 kDa. In an embodiment, a composition of the present disclosure includes recombinant silk-based protein fragments having an average weight average molecular weight ranging from about 80 to about 85 kDa. In an embodiment, a composition of the present disclosure includes recombinant silk-based protein fragments having an average weight average molecular weight ranging from about 85 to about 90 kDa. In an embodiment, a composition of the present disclosure includes recombinant silk-based protein fragments having an average weight average molecular weight ranging from about 90 to about 95 kDa. In an embodiment, a composition of the present disclosure includes recombinant silk-based protein fragments having an average weight average molecular weight ranging from about 95 to about 100 kDa. In an embodiment, a composition of the present disclosure includes recombinant silk-based protein fragments having an average weight average molecular weight ranging from about 100 to about 105 kDa. In an embodiment, a composition of the present disclosure includes recombinant silk-based protein fragments having an average weight average molecular weight ranging from about 105 to about 110 kDa. In an embodiment, a composition of the present disclosure includes recombinant silk-based protein fragments having an average weight average molecular weight ranging from about 110 to about 115 kDa. In an embodiment, a composition of the present disclosure includes recombinant silk-based protein fragments having an average weight average molecular weight ranging from about 115 to about 120 kDa. In an embodiment, a composition of the present disclosure includes recombinant silk-based protein fragments having an average weight average molecular weight ranging from about 120 to about 125 kDa. In an embodiment, a composition of the present disclosure includes recombinant silk-based protein fragments having an average weight average molecular weight ranging from about 125 to about 130 kDa. In an embodiment, a composition of the present disclosure includes recombinant silk-based protein fragments having an average weight average molecular weight ranging from about 130 to about 135 kDa. In an embodiment, a composition of the present disclosure includes recombinant silk-based protein fragments having an average weight average molecular weight ranging from about 135 to about 140 kDa. In an embodiment, a composition of the present disclosure includes recombinant silk-based protein fragments having an average weight average molecular weight ranging from about 140 to about 145 kDa. In an embodiment, a composition of the present disclosure includes recombinant silk-based protein fragments having an average weight average molecular weight ranging from about 145 to about 150 kDa. In an embodiment, a composition of the present disclosure includes recombinant silk-based protein fragments having an average weight average molecular weight ranging from about 150 to about 155 kDa. In an embodiment, a composition of the present disclosure includes recombinant silk-based protein fragments having an average weight average molecular weight ranging from about 155 to about 160 kDa. In an embodiment, a composition of the present disclosure includes recombinant silk-based protein fragments having an average weight average molecular weight ranging from about 160 to about 165 kDa. In an embodiment, a composition of the present disclosure includes recombinant silk-based protein fragments having an average weight average molecular weight ranging from about 165 to about 170 kDa. In an embodiment, a composition of the present disclosure includes recombinant silk-based protein fragments having an average weight average molecular weight ranging from about 170 to about 175 kDa. In an embodiment, a composition of the present disclosure includes recombinant silk-based protein fragments having an average weight average molecular weight ranging from about 175 to about 180 kDa. In an embodiment, a composition of the present disclosure includes recombinant silk-based protein fragments having an average weight average molecular weight ranging from about 180 to about 185 kDa. In an embodiment, a composition of the present disclosure includes recombinant silk-based protein fragments having an average weight average molecular weight ranging from about 185 to about 190 kDa. In an embodiment, a composition of the present disclosure includes recombinant silk-based protein fragments having an average weight average molecular weight ranging from about 190 to about 195 kDa. In an embodiment, a composition of the present disclosure includes recombinant silk-based protein fragments having an average weight average molecular weight ranging from about 195 to about 200 kDa. In an embodiment, a composition of the present disclosure includes recombinant silk-based protein fragments having an average weight average molecular weight ranging from about 200 to about 205 kDa. In an embodiment, a composition of the present disclosure includes recombinant silk-based protein fragments having an average weight average molecular weight ranging from about 205 to about 210 kDa. In an embodiment, a composition of the present disclosure includes recombinant silk-based protein fragments having an average weight average molecular weight ranging from about 210 to about 215 kDa. In an embodiment, a composition of the present disclosure includes recombinant silk-based protein fragments having an average weight average molecular weight ranging from about 215 to about 220 kDa. In an embodiment, a composition of the present disclosure includes recombinant silk-based protein fragments having an average weight average molecular weight ranging from about 220 to about 225 kDa. In an embodiment, a composition of the present disclosure includes recombinant silk-based protein fragments having an average weight average molecular weight ranging from about 225 to about 230 kDa. In an embodiment, a composition of the present disclosure includes recombinant silk-based protein fragments having an average weight average molecular weight ranging from about 230 to about 235 kDa. In an embodiment, a composition of the present disclosure includes recombinant silk-based protein fragments having an average weight average molecular weight ranging from about 235 to about 240 kDa. In an embodiment, a composition of the present disclosure includes recombinant silk-based protein fragments having an average weight average molecular weight ranging from about 240 to about 245 kDa. In an embodiment, a composition of the present disclosure includes recombinant silk-based protein fragments having an average weight average molecular weight ranging from about 245 to about 250 kDa. In an embodiment, a composition of the present disclosure includes recombinant silk-based protein fragments having an average weight average molecular weight ranging from about 250 to about 255 kDa. In an embodiment, a composition of the present disclosure includes recombinant silk-based protein fragments having an average weight average molecular weight ranging from about 255 to about 260 kDa. In an embodiment, a composition of the present disclosure includes recombinant silk-based protein fragments having an average weight average molecular weight ranging from about 260 to about 265 kDa. In an embodiment, a composition of the present disclosure includes recombinant silk-based protein fragments having an average weight average molecular weight ranging from about 265 to about 270 kDa. In an embodiment, a composition of the present disclosure includes recombinant silk-based protein fragments having an average weight average molecular weight ranging from about 270 to about 275 kDa. In an embodiment, a composition of the present disclosure includes recombinant silk-based protein fragments having an average weight average molecular weight ranging from about 275 to about 280 kDa. In an embodiment, a composition of the present disclosure includes recombinant silk-based protein fragments having an average weight average molecular weight ranging from about 280 to about 285 kDa. In an embodiment, a composition of the present disclosure includes recombinant silk-based protein fragments having an average weight average molecular weight ranging from about 285 to about 290 kDa. In an embodiment, a composition of the present disclosure includes recombinant silk-based protein fragments having an average weight average molecular weight ranging from about 290 to about 295 kDa. In an embodiment, a composition of the present disclosure includes recombinant silk-based protein fragments having an average weight average molecular weight ranging from about 295 to about 300 kDa. In an embodiment, a composition of the present disclosure includes recombinant silk-based protein fragments having an average weight average molecular weight ranging from about 300 to about 305 kDa. In an embodiment, a composition of the present disclosure includes recombinant silk-based protein fragments having an average weight average molecular weight ranging from about 305 to about 310 kDa. In an embodiment, a composition of the present disclosure includes recombinant silk-based protein fragments having an average weight average molecular weight ranging from about 310 to about 315 kDa. In an embodiment, a composition of the present disclosure includes recombinant silk-based protein fragments having an average weight average molecular weight ranging from about 315 to about 320 kDa. In an embodiment, a composition of the present disclosure includes recombinant silk-based protein fragments having an average weight average molecular weight ranging from about 320 to about 325 kDa. In an embodiment, a composition of the present disclosure includes recombinant silk-based protein fragments having an average weight average molecular weight ranging from about 325 to about 330 kDa. In an embodiment, a composition of the present disclosure includes recombinant silk-based protein fragments having an average weight average molecular weight ranging from about 330 to about 335 kDa. In an embodiment, a composition of the present disclosure includes recombinant silk-based protein fragments having an average weight average molecular weight ranging from about 335 to about 340 kDa. In an embodiment, a composition of the present disclosure includes recombinant silk-based protein fragments having an average weight average molecular weight ranging from about 340 to about 345 kDa. In an embodiment, a composition of the present disclosure includes recombinant silk-based protein fragments having an average weight average molecular weight ranging from about 345 to about 350 kDa. In some embodiments, recombinant silk is as provided in any of the compositions #1001-2450, #3001-4450, and #5001-6595.

In an embodiment, a composition of the present disclosure includes recombinant silk protein fragments having an average weight average molecular weight of about 1 kDa to about 350 kDa, or about 1 kDa to about 300 kDa, or about 1 kDa to about 250 kDa, or about 1 kDa to about 200 kDa, or about 1 kDa to about 150 kDa, or about 1 kDa to about 100 kDa, or about 1 kDa to about 50 kDa, or about 1 kDa to about 25 kDa.

In an embodiment, a composition of the present disclosure includes recombinant silk protein fragments having an average weight average molecular weight ranging from about 1 to about 5 kDa. In an embodiment, a composition of the present disclosure includes recombinant silk protein fragments having an average weight average molecular weight ranging from about 5 to about 10 kDa. In an embodiment, a composition of the present disclosure includes recombinant silk protein fragments having an average weight average molecular weight ranging from about 10 to about 15 kDa. In an embodiment, a composition of the present disclosure includes recombinant silk protein fragments having an average weight average molecular weight ranging from about 15 to about 20 kDa. In an embodiment, a composition of the present disclosure includes recombinant silk protein fragments having an average weight average molecular weight ranging from about 20 to about 25 kDa. In an embodiment, a composition of the present disclosure includes recombinant silk protein fragments having an average weight average molecular weight ranging from about 25 to about 30 kDa. In an embodiment, a composition of the present disclosure includes recombinant silk protein fragments having an average weight average molecular weight ranging from about 30 to about 35 kDa. In an embodiment, a composition of the present disclosure includes recombinant silk protein fragments having an average weight average molecular weight ranging from about 35 to about 40 kDa. In an embodiment, a composition of the present disclosure includes recombinant silk protein fragments having an average weight average molecular weight ranging from about 40 to about 45 kDa. In an embodiment, a composition of the present disclosure includes recombinant silk protein fragments having an average weight average molecular weight ranging from about 45 to about 50 kDa. In an embodiment, a composition of the present disclosure includes recombinant silk protein fragments having an average weight average molecular weight ranging from about 50 to about 55 kDa. In an embodiment, a composition of the present disclosure includes recombinant silk protein fragments having an average weight average molecular weight ranging from about 55 to about 60 kDa. In an embodiment, a composition of the present disclosure includes recombinant silk protein fragments having an average weight average molecular weight ranging from about 60 to about 65 kDa. In an embodiment, a composition of the present disclosure includes recombinant silk protein fragments having an average weight average molecular weight ranging from about 65 to about 70 kDa. In an embodiment, a composition of the present disclosure includes recombinant silk protein fragments having an average weight average molecular weight ranging from about 70 to about 75 kDa. In an embodiment, a composition of the present disclosure includes recombinant silk protein fragments having an average weight average molecular weight ranging from about 75 to about 80 kDa. In an embodiment, a composition of the present disclosure includes recombinant silk protein fragments having an average weight average molecular weight ranging from about 80 to about 85 kDa. In an embodiment, a composition of the present disclosure includes recombinant silk protein fragments having an average weight average molecular weight ranging from about 85 to about 90 kDa. In an embodiment, a composition of the present disclosure includes recombinant silk protein fragments having an average weight average molecular weight ranging from about 90 to about 95 kDa. In an embodiment, a composition of the present disclosure includes recombinant silk protein fragments having an average weight average molecular weight ranging from about 95 to about 100 kDa. In an embodiment, a composition of the present disclosure includes recombinant silk protein fragments having an average weight average molecular weight ranging from about 100 to about 105 kDa. In an embodiment, a composition of the present disclosure includes recombinant silk protein fragments having an average weight average molecular weight ranging from about 105 to about 110 kDa. In an embodiment, a composition of the present disclosure includes recombinant silk protein fragments having an average weight average molecular weight ranging from about 110 to about 115 kDa. In an embodiment, a composition of the present disclosure includes recombinant silk protein fragments having an average weight average molecular weight ranging from about 115 to about 120 kDa. In an embodiment, a composition of the present disclosure includes recombinant silk protein fragments having an average weight average molecular weight ranging from about 120 to about 125 kDa. In an embodiment, a composition of the present disclosure includes recombinant silk protein fragments having an average weight average molecular weight ranging from about 125 to about 130 kDa. In an embodiment, a composition of the present disclosure includes recombinant silk protein fragments having an average weight average molecular weight ranging from about 130 to about 135 kDa. In an embodiment, a composition of the present disclosure includes recombinant silk protein fragments having an average weight average molecular weight ranging from about 135 to about 140 kDa. In an embodiment, a composition of the present disclosure includes recombinant silk protein fragments having an average weight average molecular weight ranging from about 140 to about 145 kDa. In an embodiment, a composition of the present disclosure includes recombinant silk protein fragments having an average weight average molecular weight ranging from about 145 to about 150 kDa. In an embodiment, a composition of the present disclosure includes recombinant silk protein fragments having an average weight average molecular weight ranging from about 150 to about 155 kDa. In an embodiment, a composition of the present disclosure includes recombinant silk protein fragments having an average weight average molecular weight ranging from about 155 to about 160 kDa. In an embodiment, a composition of the present disclosure includes recombinant silk protein fragments having an average weight average molecular weight ranging from about 160 to about 165 kDa. In an embodiment, a composition of the present disclosure includes recombinant silk protein fragments having an average weight average molecular weight ranging from about 165 to about 170 kDa. In an embodiment, a composition of the present disclosure includes recombinant silk protein fragments having an average weight average molecular weight ranging from about 170 to about 175 kDa. In an embodiment, a composition of the present disclosure includes recombinant silk protein fragments having an average weight average molecular weight ranging from about 175 to about 180 kDa. In an embodiment, a composition of the present disclosure includes recombinant silk protein fragments having an average weight average molecular weight ranging from about 180 to about 185 kDa. In an embodiment, a composition of the present disclosure includes recombinant silk protein fragments having an average weight average molecular weight ranging from about 185 to about 190 kDa. In an embodiment, a composition of the present disclosure includes recombinant silk protein fragments having an average weight average molecular weight ranging from about 190 to about 195 kDa. In an embodiment, a composition of the present disclosure includes recombinant silk protein fragments having an average weight average molecular weight ranging from about 195 to about 200 kDa. In an embodiment, a composition of the present disclosure includes recombinant silk protein fragments having an average weight average molecular weight ranging from about 200 to about 205 kDa. In an embodiment, a composition of the present disclosure includes recombinant silk protein fragments having an average weight average molecular weight ranging from about 205 to about 210 kDa. In an embodiment, a composition of the present disclosure includes recombinant silk protein fragments having an average weight average molecular weight ranging from about 210 to about 215 kDa. In an embodiment, a composition of the present disclosure includes recombinant silk protein fragments having an average weight average molecular weight ranging from about 215 to about 220 kDa. In an embodiment, a composition of the present disclosure includes recombinant silk protein fragments having an average weight average molecular weight ranging from about 220 to about 225 kDa. In an embodiment, a composition of the present disclosure includes recombinant silk protein fragments having an average weight average molecular weight ranging from about 225 to about 230 kDa. In an embodiment, a composition of the present disclosure includes recombinant silk protein fragments having an average weight average molecular weight ranging from about 230 to about 235 kDa. In an embodiment, a composition of the present disclosure includes recombinant silk protein fragments having an average weight average molecular weight ranging from about 235 to about 240 kDa. In an embodiment, a composition of the present disclosure includes recombinant silk protein fragments having an average weight average molecular weight ranging from about 240 to about 245 kDa. In an embodiment, a composition of the present disclosure includes recombinant silk protein fragments having an average weight average molecular weight ranging from about 245 to about 250 kDa. In an embodiment, a composition of the present disclosure includes recombinant silk protein fragments having an average weight average molecular weight ranging from about 250 to about 255 kDa. In an embodiment, a composition of the present disclosure includes recombinant silk protein fragments having an average weight average molecular weight ranging from about 255 to about 260 kDa. In an embodiment, a composition of the present disclosure includes recombinant silk protein fragments having an average weight average molecular weight ranging from about 260 to about 265 kDa. In an embodiment, a composition of the present disclosure includes recombinant silk protein fragments having an average weight average molecular weight ranging from about 265 to about 270 kDa. In an embodiment, a composition of the present disclosure includes recombinant silk protein fragments having an average weight average molecular weight ranging from about 270 to about 275 kDa. In an embodiment, a composition of the present disclosure includes recombinant silk protein fragments having an average weight average molecular weight ranging from about 275 to about 280 kDa. In an embodiment, a composition of the present disclosure includes recombinant silk protein fragments having an average weight average molecular weight ranging from about 280 to about 285 kDa. In an embodiment, a composition of the present disclosure includes recombinant silk protein fragments having an average weight average molecular weight ranging from about 285 to about 290 kDa. In an embodiment, a composition of the present disclosure includes recombinant silk protein fragments having an average weight average molecular weight ranging from about 290 to about 295 kDa. In an embodiment, a composition of the present disclosure includes recombinant silk protein fragments having an average weight average molecular weight ranging from about 295 to about 300 kDa. In an embodiment, a composition of the present disclosure includes recombinant silk protein fragments having an average weight average molecular weight ranging from about 300 to about 305 kDa. In an embodiment, a composition of the present disclosure includes recombinant silk protein fragments having an average weight average molecular weight ranging from about 305 to about 310 kDa. In an embodiment, a composition of the present disclosure includes recombinant silk protein fragments having an average weight average molecular weight ranging from about 310 to about 315 kDa. In an embodiment, a composition of the present disclosure includes recombinant silk protein fragments having an average weight average molecular weight ranging from about 315 to about 320 kDa. In an embodiment, a composition of the present disclosure includes recombinant silk protein fragments having an average weight average molecular weight ranging from about 320 to about 325 kDa. In an embodiment, a composition of the present disclosure includes recombinant silk protein fragments having an average weight average molecular weight ranging from about 325 to about 330 kDa. In an embodiment, a composition of the present disclosure includes recombinant silk protein fragments having an average weight average molecular weight ranging from about 330 to about 335 kDa. In an embodiment, a composition of the present disclosure includes recombinant silk protein fragments having an average weight average molecular weight ranging from about 335 to about 340 kDa. In an embodiment, a composition of the present disclosure includes recombinant silk protein fragments having an average weight average molecular weight ranging from about 340 to about 345 kDa. In an embodiment, a composition of the present disclosure includes recombinant silk protein fragments having an average weight average molecular weight ranging from about 345 to about 350 kDa. In some embodiments, recombinant silk is as provided in any of the compositions #1001-2450, #3001-4450, and #5001-6595.

In an embodiment, a composition of the present disclosure includes recombinant silk-based protein fragments having one or more of low molecular weight, medium molecular weight, and high molecular weight. In an embodiment, a composition of the present disclosure includes recombinant silk-based protein fragments having low molecular weight and recombinant silk-based protein fragments having medium molecular weight. In an embodiment, a composition of the present disclosure includes recombinant silk-based protein fragments having low molecular weight and recombinant silk-based protein fragments having high molecular weight. In an embodiment, a composition of the present disclosure includes recombinant silk-based protein fragments having medium molecular weight and recombinant silk-based protein fragments having high molecular weight. In an embodiment, a composition of the present disclosure includes recombinant silk-based protein fragments having low molecular weight, recombinant silk-based protein fragments having medium molecular weight, and recombinant silk-based protein fragments having high molecular weight.

In an embodiment, a composition of the present disclosure includes recombinant silk-based protein fragments having low molecular weight and recombinant silk-based protein fragments having medium molecular weight. In some embodiments, the w/w ratio between low molecular weight recombinant silk-based protein fragments and medium molecular weight recombinant silk-based protein fragments is between about 99:1 to about 1:99, between about 95:5 to about 5:95, between about 90:10 to about 10:90, between about 75:25 to about 25:75, between about 65:35 to about 35:65, or between about 55:45 to about 45:55. In some embodiments, the w/w ratio between low molecular weight recombinant silk-based protein fragments and medium molecular weight recombinant silk-based protein fragments is between about 99:1 to about 55:45, between about 95:5 to about 45:55, between about 90:10 to about 35:65, between about 75:25 to about 15:85, between about 65:35 to about 10:90, or between about 55:45 to about 1:99. In an embodiment, the w/w ratio between low molecular weight recombinant silk-based protein fragments and medium molecular weight recombinant silk-based protein fragments is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99. In an embodiment, the w/w ratio between low molecular weight recombinant silk-based protein fragments and medium molecular weight recombinant silk-based protein fragments is about 3:1. In an embodiment, the w/w ratio between low molecular weight recombinant silk-based protein fragments and medium molecular weight recombinant silk-based protein fragments is about 1:3.

In an embodiment, a composition of the present disclosure includes recombinant silk-based protein fragments having low molecular weight and recombinant silk-based protein fragments having high molecular weight. In some embodiments, the w/w ratio between low molecular weight recombinant silk-based protein fragments and high molecular weight recombinant silk-based protein fragments is between about 99:1 to about 1:99, between about 95:5 to about 5:95, between about 90:10 to about 10:90, between about 75:25 to about 25:75, between about 65:35 to about 35:65, or between about 55:45 to about 45:55. In some embodiments, the w/w ratio between low molecular weight recombinant silk-based protein fragments and high molecular weight recombinant silk-based protein fragments is between about 99:1 to about 55:45, between about 95:5 to about 45:55, between about 90:10 to about 35:65, between about 75:25 to about 15:85, between about 65:35 to about 10:90, or between about 55:45 to about 1:99. In an embodiment, the w/w ratio between low molecular weight recombinant silk-based protein fragments and high molecular weight recombinant silk-based protein fragments is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

In an embodiment, a composition of the present disclosure includes recombinant silk-based protein fragments having medium molecular weight and recombinant silk-based protein fragments having high molecular weight. In some embodiments, the w/w ratio between medium molecular weight recombinant silk-based protein fragments and high molecular weight recombinant silk-based protein fragments is between about 99:1 to about 1:99, between about 95:5 to about 5:95, between about 90:10 to about 10:90, between about 75:25 to about 25:75, between about 65:35 to about 35:65, or between about 55:45 to about 45:55. In some embodiments, the w/w ratio between medium molecular weight recombinant silk-based protein fragments and high molecular weight recombinant silk-based protein fragments is between about 99:1 to about 55:45, between about 95:5 to about 45:55, between about 90:10 to about 35:65, between about 75:25 to about 15:85, between about 65:35 to about 10:90, or between about 55:45 to about 1:99. In an embodiment, the w/w ratio between medium molecular weight recombinant silk-based protein fragments and high molecular weight recombinant silk-based protein fragments is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

In an embodiment, a composition of the present disclosure includes recombinant silk-based protein fragments having low molecular weight, recombinant silk-based protein fragments having medium molecular weight, and recombinant silk-based protein fragments having high molecular weight. In an embodiment, the w/w ratio between low molecular weight recombinant silk-based protein fragments, medium molecular weight recombinant silk-based protein fragments, and high molecular weight recombinant silk-based protein fragments is about 1:1:8, about 1:2:7, about 1:3:6, about 1:4:5, about 1:5:4, about 1:6:3, about 1:7:2, about 1:8:1, about 2:1:7, about 2:2:6, about 2:3:5, about 2:4:4, about 2:5:3, about 2:6:2, about 2:7:1, about 3:1:6, about 3:2:5, about 3:3:4, about 3:4:3, about 3:5:2, about 3:6:1, about 4:1:5, about 4:2:4, about 4:3:3, about 4:4:2, about 4:5:1, about 5:1:4, about 5:2:3, about 5:3:2, about 5:4:1, about 6:1:3, about 6:2:2, about 6:3:1, about 7:1:2, about 7:2:1, or about 8:1:1. In an embodiment, the w/w ratio between low molecular weight recombinant silk-based protein fragments, medium molecular weight recombinant silk-based protein fragments, and high molecular weight recombinant silk-based protein fragments is about 3:0.1:0.9, about 3:0.2:0.8, about 3:0.3:0.7, about 3:0.4:0.6, about 3:0.5:0.5, about 3:0.6:0.4, about 3:0.7:0.3, about 3:0.8:0.2, or about 3:0.9:0.1.

In some embodiments, the silk compositions provided herein may be applied as mixtures to an article to be coated or in stepwise processes to form coating layers on the article. Methods of using silk fragments for coating an article are described for example in U.S. Pat. Nos. 10,287,728 and 10,301,768, and U.S. Patent Application Publications 2019/0309467, 2019/0211498, and 2019/0003113, and International Application Publication WO 2019/067745, all incorporated herein by reference in their entirety. For example, a recombinant silk composition that includes low molecular weight recombinant silk and medium molecular weight recombinant silk may be applied to an article to be coated. Alternatively, a low molecular weight recombinant silk composition may be applied to an article to be coated, as provided by the processes described herein, and then a medium or high molecular weight recombinant silk may then be applied to the article. The low, medium, and high molecular weight recombinant silk compositions may be added in any order or any combination (e.g., low/med, low/high, med/high, low/med/high).

In some embodiments, where multiple layers of recombinant silk compositions are applied to an article to be coated, they may have at least one layer, or 1 layer to 1 million layers, or 1 layer to 100,000 layers, or 1 layer to 10,000 layers, or 1 layer to 1,000 layers of such recombinant silk compositions, wherein the layers may have the same or different thicknesses. For example, in some embodiments, the layers may have a thickness of from about 1 nm to about 1 mm, or about 1 nm to about 1 μm, or about 1 nm to about 500 nm, or about 1 nm to about 400 nm, or about 1 nm to about 300 nm, or about 1 nm to about 200 nm, or about 1 nm to about 100 nm, or about 1 nm to about 75 nm, or about 1 nm to about 50 nm, or about 1 nm to about 25 nm, or about 1 nm to about 20 nm, or about 1 nm to about 15 nm, or about 1 nm to about 10 nm, or about 1 nm to about 5 nm.

In an embodiment, a composition of the present disclosure having recombinant silk-based protein fragments has a polydispersity ranging from 1 to about 5.0. In an embodiment, a composition of the present disclosure having recombinant silk-based protein fragments has a polydispersity ranging from about 1.5 to about 3.0. In an embodiment, a composition of the present disclosure having recombinant silk-based protein fragments has a polydispersity ranging from 1 to about 1.5. In an embodiment, a composition of the present disclosure having recombinant silk-based protein fragments has a polydispersity ranging from about 1.5 to about 2.0. In an embodiment, a composition of the present disclosure having recombinant silk-based protein fragments has a polydispersity ranging from about 2.0 to about 2.5. In an embodiment, a composition of the present disclosure having recombinant silk-based protein fragments, has a polydispersity ranging from about is 2.0 to about 3.0. In an embodiment, a composition of the present disclosure having recombinant silk-based protein fragments, has a polydispersity ranging from about is 2.5 to about 3.0.

In an embodiment, a composition of the present disclosure having recombinant silk protein fragments has a polydispersity ranging from 1 to about 5.0. In an embodiment, a composition of the present disclosure having recombinant silk protein fragments has a polydispersity ranging from about 1.5 to about 3.0. In an embodiment, a composition of the present disclosure having recombinant silk protein fragments has a polydispersity ranging from 1 to about 1.5. In an embodiment, a composition of the present disclosure having recombinant silk protein fragments has a polydispersity ranging from about 1.5 to about 2.0. In an embodiment, a composition of the present disclosure having recombinant silk protein fragments has a polydispersity ranging from about 2.0 to about 2.5. In an embodiment, a composition of the present disclosure having recombinant silk protein fragments, has a polydispersity ranging from about is 2.0 to about 3.0. In an embodiment, a composition of the present disclosure having recombinant silk protein fragments, has a polydispersity ranging from about is 2.5 to about 3.0.

In some embodiments the polydispersity of low molecular weight recombinant silk protein fragments may be from 1 to about 5.0, or from about 1.5 to about 3.0, or from 1 to about 1.5, or from about 1.5 to about 2.0, or from about 2.0 to about 2.5, or from about 2.5 to about 3.0.

In some embodiments the polydispersity of medium molecular weight recombinant silk protein fragments may be from 1 to about 5.0, or from about 1.5 to about 3.0, or from 1 to about 1.5, or from about 1.5 to about 2.0, or from about 2.0 to about 2.5, or from about 2.5 to about 3.0.

In some embodiments the polydispersity of high molecular weight recombinant silk protein fragments may be from 1 to about 5.0, or from about 1.5 to about 3.0, or from 1 to about 1.5, or from about 1.5 to about 2.0, or from about 2.0 to about 2.5, or from about 2.5 to about 3.0.

In some embodiments, in compositions described herein having combinations of low, medium, and/or high molecular weight recombinant silk protein fragments, such low, medium, and/or high molecular weight recombinant silk proteins may have the same or different polydispersities.

In an embodiment, the water solubility of recombinant silk-based protein fragments of the present disclosure is 50 to 100%. In an embodiment, the water solubility of recombinant silk-based protein fragments of the present disclosure is 60 to 100%. In an embodiment, the water solubility of recombinant silk-based protein fragments of the present disclosure is 70 to 100%. In an embodiment, the water solubility of recombinant silk-based protein fragments of the present disclosure is 80 to 100%. In an embodiment, the water solubility is 90 to 100%. In an embodiment, the recombinant silk-based fragments of the present disclosure are non-soluble in aqueous solutions.

In an embodiment, the solubility of recombinant silk-based protein fragments of the present disclosure in organic solutions is 50 to 100%. In an embodiment, the solubility of recombinant silk-based protein fragments of the present disclosure in organic solutions is 60 to 100%. In an embodiment, the solubility of recombinant silk-based protein fragments of the present disclosure in organic solutions is 70 to 100%. In an embodiment, the solubility of recombinant silk-based protein fragments of the present disclosure in organic solutions is 80 to 100%. In an embodiment, the solubility of recombinant silk-based protein fragments of the present disclosure in organic solutions is 90 to 100%. In an embodiment, the recombinant silk-based fragments of the present disclosure are non-soluble in organic solutions.

### Compositions and Processes Including Recombinant Silk-Based Coatings

In an embodiment, the disclosure may include textiles, such as fibers, yarns, fabrics, or other materials and combinations thereof, that may be coated with an RSPF mixture solution (i.e., recombinant silk solution (RSS)) as described herein to produce a coated article. In some embodiments, recombinant silk is as provided in any of the compositions #1001-2450, #3001-4450, and #5001-6595. In an embodiment, the coated articles described herein may be treated with additional chemical agents that may enhance the properties of the coated article. In an embodiment, the RSS may include one or more chemical agents that may enhance the properties of the coated article. Silk coated materials are known and have been described for example in patents and patent application publications WO 2016/090055, WO 20170/11679, US 2016/0222579, US 2016/0281294, US 2019/0003113, U.S. Pat. Nos. 10,287,728, 10,301,768, US 2019/0309467, US 2019/0211498, US 2019/0003113, and WO 2019/067745, all incorporated herein by reference in their entirety.

In an embodiment, textiles may be flexible materials (woven or non-woven) that include a network of natural and/or man-made fibers, thread, yarn, or a combination thereof. RSS may be applied at any stage of textile processing from individual fibers, to yarn, to fabric, to thread, or a combination thereof.

In an embodiment, fibers may be natural fibers that may include a natural fiber cellulose base, wherein the natural fiber cellulose base may include one or more of: (1) a baste such as flax, hemp, kenaf, jute, linen, and/or ramie; (2) a leaf such as flax, hemp, sisal, abaca, banana, henequen, ramie, sunn, and/or coir; and (3) seed hair such as cotton and/or kapok. In an embodiment, fibers may be natural fibers that may include a natural fiber protein base, wherein the natural fiber protein base may include one or more of: (1) hair such as alpaca, camel, cashmere, llama, mohair, and/or vicuna; (2) wool such as sheep; (3) filament such as silk. In an embodiment, fibers may be natural fibers that may include a natural fiber mineral base, including asbestos. In an embodiment, fibers may be man-made fibers that may include a man-made fiber organic natural polymer base, which may include one or more of: (1) a cellulose base such as bamboo, rayon, lyocell, acetate, and/or triacetate; (2) a protein base such as azlon; (3) an alginate; and (4) rubber. In an embodiment, fibers may be man-made fibers that may include a man-made fiber organic synthetic base, which may include one or more of acrylic, anidex, aramid, fluorocarbon, modacrylic, novoloid, nylon, nytril, olefin, PBI, polycarbonate, polyester, rubber, saran, spandex, vinal vinvon. In an embodiment, fibers may be man-made fibers that may include a man-made fiber inorganic base, which may include one or more of a glass material, metallic material, and carbon material.

In an embodiment, yarn may include natural fibers that may include a natural fiber cellulose base, wherein the natural fiber cellulose base may be from: (1) a baste such as flax, hemp, kenaf, jute, linen, and/or ramie; (2) a leaf such as flax, hemp, sisal, abaca, banana, henequen, ramie, sunn, and/or coir; or (3) seed hair such as cotton and/or kapok. In an embodiment, yarn may include natural fibers that may include a natural fiber protein base, wherein the natural fiber protein base may be from: (1) hair such as alpaca, camel, cashmere, llama, mohair, and/or vicuna; (2) wool such as sheep; or (3) filament such as silk. In an embodiment, yarn may include natural fibers that may include a natural fiber mineral base, including asbestos. In an embodiment, yarn may include man-made fibers that may include a man-made fiber organic natural polymer base, which may include: (1) a cellulose base such as bamboo, rayon, lyocell, acetate, and/or triacetate; (2) a protein base such as azlon; (3) an alginate; or (4) rubber. In an embodiment, yarn may include man-made fibers that may include a man-made fiber organic synthetic base, which may include acrylic, anidex, aramid, fluorocarbon, modacrylic, novoloid, nylon, nytril, olefin, PBI, polycarbonate, polyester, rubber, saran, spandex, vinal and/or vinvon. In an embodiment, yarn may include man-made fibers that may include a man-made fiber inorganic base, which may include a glass material, metallic material, carbon material, and/or specialty material.

In an embodiment, fabrics may include natural fibers and/or yarn that may include a natural fiber cellulose base, wherein the natural fiber cellulose base may be from: (1) a baste such as flax, hemp, kenaf, jute, linen, and/or ramie; (2) a leaf such as flax, hemp, sisal, abaca, banana, henequen, ramie, sunn, and/or coir; or (3) seed hair such as cotton and/or kapok. In an embodiment, fabric may include natural fibers and/or yarn that may include a natural fiber protein base, wherein the natural fiber protein base may be from: (1) hair such as alpaca, camel, cashmere, llama, mohair, and/or vicuna; (2) wool such as sheep; or (3) filament such as silk. In an embodiment, fabric may include natural fibers and/or yarn that may include a natural fiber mineral base, including asbestos. In an embodiment, fabric may include man-made fibers and/or yarn that may include a man-made fiber organic natural polymer base, which may include: (1) a cellulose base such as bamboo, rayon, lyocell, acetate, and/or triacetate; (2) a protein base such as azlon; (3) an alginate; or (4) rubber. In an embodiment, fabric may include man-made fibers and/or yarn that may include a man-made fiber organic synthetic base, which may include acrylic, anidex, aramid, fluorocarbon, modacrylic, novoloid, nylon, nytril, olefin, PBI, polycarbonate, polyester, rubber, saran, spandex, vinal and/or vinvon. In an embodiment, fabric may include man-made fibers and/or yarn that may include a man-made fiber inorganic base, which may include a glass material, metallic material, carbon material, and/or specialty material.

In an embodiment, textiles may be manufactured via one or more of the following processes weaving processes, knitting processes, and non-woven processes. In an embodiment, weaving processes may include plain weaving, twill weaving, and/or satin weaving. In an embodiment, knitting processes may include weft knitting (e.g., circular, flat bed, and/or full fashioned) and/or warp knitting (e.g., tricot, Raschel, and/or crochet). In an embodiment, non-woven processes may include stable fiber (e.g., dry laid and/or wet laid) and/or continuous filament (e.g., spun laid and/or melt blown).

In some embodiments, RSS may be applied to fibers and/or yarn having a diameter of less than about 100 nm, or less than about 200 nm, or less than about 300 nm, or less than about 400 nm, or less than about 500 nm, or less than about 600 nm, or less than about 700 nm, or less than about 800 nm, or less than about 900 nm, or less than about 1000 nm, or less than about 2 μm, or less than about 5 μm, or less than about 10 μm, or less than about 20 μm, or less than about 30 μm, or less than about 40 μm, or less than about 50 μm, or less than about 60 μm, or less than about 70 μm, or less than about 80 μm, or less than about 90 μm, or less than about 100 μm, or less than about 200 μm, or less than about 300 μm, or less than about 400 μm, or less than about 500 μm, or less than about 600 μm, or less than about 700 μm, or less than about 800 μm, or less than about 900 μm, or less than about 1000 μm, or less than about 2 mm, or less than about 3 mm, or less than about 4 mm, or less than about 5 mm, 6 mm, or less than about 7 mm, or less than about 8 mm, or less than about 9 mm, or less than about 10 mm, or less than about 20 mm, or less than about 30 mm, or less than about 40 mm, or less than about 50 mm, or less than about 60 mm, or less than about 70 mm, or less than about 80 mm, or less than about 90 mm, or less than about 100 mm, or less than about 200 mm, or less than about 300 mm, or less than about 400 mm, or less than about 500 mm, or less than about 600 mm, or less than about 700 mm, or less than about 800 mm, or less than about 900 mm, or less than about 1000 mm.

In some embodiments, RSS may be applied to fibers and/or yarn having a diameter of greater than about 100 nm, or greater than about 200 nm, or greater than about 300 nm, or greater than about 400 nm, or greater than about 500 nm, or greater than about 600 nm, or greater than about 700 nm, or greater than about 800 nm, or greater than about 900 nm, or greater than about 1000 nm, or greater than about 2 μm, or greater than about 5 μm, or greater than about 10 μm, or greater than about 20 μm, or greater than about 30 μm, or greater than about 40 μm, or greater than about 50 μm, or greater than about 60 μm, or greater than about 70 μm, or greater than about 80 μm, or greater than about 90 μm, or greater than about 100 μm, or greater than about 200 μm, or greater than about 300 μm, or greater than about 400 μm, or greater than about 500 μm, or greater than about 600 μm, or greater than about 700 μm, or greater than about 800 μm, or greater than about 900 μm, or greater than about 1000 μm, or greater than about 2 mm, or greater than about 3 mm, or greater than about 4 mm, or greater than about 5 mm, 6 mm, or greater than about 7 mm, or greater than about 8 mm, or greater than about 9 mm, or greater than about 10 mm, or greater than about 20 mm, or greater than about 30 mm, or greater than about 40 mm, or greater than about 50 mm, or greater than about 60 mm, or greater than about 70 mm, or greater than about 80 mm, or greater than about 90 mm, or greater than about 100 mm, or greater than about 200 mm, or greater than about 300 mm, or greater than about 400 mm, or greater than about 500 mm, or greater than about 600 mm, or greater than about 700 mm, or greater than about 800 mm, or greater than about 900 mm, or greater than about 1000 mm.

In some embodiments, RSS may be applied to fibers and/or yarn having a length of less than about 100 nm, or less than about 200 nm, or less than about 300 nm, or less than about 400 nm, or less than about 500 nm, or less than about 600 nm, or less than about 700 nm, or less than about 800 nm, or less than about 900 nm, or less than about 1000 nm, or less than about 2 μm, or less than about 5 μm, or less than about 10 μm, or less than about 20 μm, or less than about 30 μm, or less than about 40 μm, or less than about 50 μm, or less than about 60 μm, or less than about 70 μm, or less than about 80 μm, or less than about 90 μm, or less than about 100 μm, or less than about 200 μm, or less than about 300 μm, or less than about 400 μm, or less than about 500 μm, or less than about 600 μm, or less than about 700 μm, or less than about 800 μm, or less than about 900 μm, or less than about 1000 μm, or less than about 2 mm, or less than about 3 mm, or less than about 4 mm, or less than about 5 mm, 6 mm, or less than about 7 mm, or less than about 8 mm, or less than about 9 mm, or less than about 10 mm, or less than about 20 mm, or less than about 30 mm, or less than about 40 mm, or less than about 50 mm, or less than about 60 mm, or less than about 70 mm, or less than about 80 mm, or less than about 90 mm, or less than about 100 mm, or less than about 200 mm, or less than about 300 mm, or less than about 400 mm, or less than about 500 mm, or less than about 600 mm, or less than about 700 mm, or less than about 800 mm, or less than about 900 mm, or less than about 1000 mm.

In some embodiments, RSS may be applied to fibers and/or yarn having a length of greater than about 100 nm, or greater than about 200 nm, or greater than about 300 nm, or greater than about 400 nm, or greater than about 500 nm, or greater than about 600 nm, or greater than about 700 nm, or greater than about 800 nm, or greater than about 900 nm, or greater than about 1000 nm, or greater than about 2 μm, or greater than about 5 μm, or greater than about 10 μm, or greater than about 20 μm, or greater than about 30 μm, or greater than about 40 μm, or greater than about 50 μm, or greater than about 60 μm, or greater than about 70 μm, or greater than about 80 μm, or greater than about 90 μm, or greater than about 100 μm, or greater than about 200 μm, or greater than about 300 μm, or greater than about 400 μm, or greater than about 500 μm, or greater than about 600 μm, or greater than about 700 μm, or greater than about 800 μm, or greater than about 900 μm, or greater than about 1000 μm, or greater than about 2 mm, or greater than about 3 mm, or greater than about 4 mm, or greater than about 5 mm, 6 mm, or greater than about 7 mm, or greater than about 8 mm, or greater than about 9 mm, or greater than about 10 mm, or greater than about 20 mm, or greater than about 30 mm, or greater than about 40 mm, or greater than about 50 mm, or greater than about 60 mm, or greater than about 70 mm, or greater than about 80 mm, or greater than about 90 mm, or greater than about 100 mm, or greater than about 200 mm, or greater than about 300 mm, or greater than about 400 mm, or greater than about 500 mm, or greater than about 600 mm, or greater than about 700 mm, or greater than about 800 mm, or greater than about 900 mm, or greater than about 1000 mm.

In some embodiments, RSS may be applied to fibers and/or yarn having a weight (g/m²) of less than about 1 g/m², or less than about 2 g/m², or less than about 3 g/m², or less than about 4 g/m², or less than about 5 g/m², or less than about 6 g/m², or less than about 7 g/m², or less than about 8 g/m², or less than about 9 g/m², or less than about 10 g/m², or less than about 20 g/m², or less than about 30 g/m², or less than about 40 g/m², or less than about 50 g/m², or less than about 60 g/m², or less than about 70 g/m², or less than about 80 g/m², or less than about 90 g/m², or less than about 100 g/m², or less than about 200 g/m², or less than about 300 g/m², or less than about 400 g/m², or less than about 500 g/m².

In some embodiments, RSS may be applied to fibers and/or yarn having a weight (g/m²) of at greater than about 1 g/m², or greater than about 2 g/m², or greater than about 3 g/m², or greater than about 4 g/m², or greater than about 5 g/m², or greater than about 6 g/m², or greater than about 7 g/m², or greater than about 8 g/m², or greater than about 9 g/m², or greater than about 10 g/m², or greater than about 20 g/m², or greater than about 30 g/m², or greater than about 40 g/m², or greater than about 50 g/m², or greater than about 60 g/m², or greater than about 70 g/m², or greater than about 80 g/m², or greater than about 90 g/m², or greater than about 100 g/m², or greater than about 200 g/m², or greater than about 300 g/m², or greater than about 400 g/m², or greater than about 500 g/m².

In some embodiments, RSS may be applied to fabric having a thickness of less than about 100 nm, or less than about 200 nm, or less than about 300 nm, or less than about 400 nm, or less than about 500 nm, or less than about 600 nm, or less than about 700 nm, or less than about 800 nm, or less than about 900 nm, or less than about 1000 nm, or less than about 2 μm, or less than about 5 μm, or less than about 10 μm, or less than about 20 μm, or less than about 30 μm, or less than about 40 μm, or less than about 50 μm, or less than about 60 μm, or less than about 70 μm, or less than about 80 μm, or less than about 90 μm, or less than about 100 μm, or less than about 200 μm, or less than about 300 μm, or less than about 400 μm, or less than about 500 μm, or less than about 600 μm, or less than about 700 μm, or less than about 800 μm, or less than about 900 μm, or less than about 1000 μm, or less than about 2 mm, or less than about 3 mm, or less than about 4 mm, or less than about 5 mm, 6 mm, or less than about 7 mm, or less than about 8 mm, or less than about 9 mm, or less than about 10 mm.

In some embodiments, RSS may be applied to fabric having a thickness of greater than about 100 nm, or greater than about 200 nm, or greater than about 300 nm, or greater than about 400 nm, or greater than about 500 nm, or greater than about 600 nm, or greater than about 700 nm, or greater than about 800 nm, or greater than about 900 nm, or greater than about 1000 nm, or greater than about 2 μm, or greater than about 5 μm, or greater than about 10 μm, or greater than about 20 μm, or greater than about 30 μm, or greater than about 40 μm, or greater than about 50 μm, or greater than about 60 μm, or greater than about 70 μm, or greater than about 80 μm, or greater than about 90 μm, or greater than about 100 μm, or greater than about 200 μm, or greater than about 300 μm, or greater than about 400 μm, or greater than about 500 μm, or greater than about 600 μm, or greater than about 700 μm, or greater than about 800 μm, or greater than about 900 μm, or greater than about 1000 μm, or greater than about 2 mm, or greater than about 3 mm, or greater than about 4 mm, or greater than about 5 mm, 6 mm, or greater than about 7 mm, or greater than about 8 mm, or greater than about 9 mm, or greater than about 10 mm.

In some embodiments, RSS may be applied to fabric having a width of less than about 100 nm, or less than about 200 nm, or less than about 300 nm, or less than about 400 nm, or less than about 500 nm, or less than about 600 nm, or less than about 700 nm, or less than about 800 nm, or less than about 900 nm, or less than about 1000 nm, or less than about 2 μm, or less than about 5 μm, or less than about 10 μm, or less than about 20 μm, or less than about 30 μm, or less than about 40 μm, or less than about 50 μm, or less than about 60 μm, or less than about 70 μm, or less than about 80 μm, or less than about 90 μm, or less than about 100 μm, or less than about 200 μm, or less than about 300 μm, or less than about 400 μm, or less than about 500 μm, or less than about 600 μm, or less than about 700 μm, or less than about 800 μm, or less than about 900 μm, or less than about 1000 μm, or less than about 2 mm, or less than about 3 mm, or less than about 4 mm, or less than about 5 mm, 6 mm, or less than about 7 mm, or less than about 8 mm, or less than about 9 mm, or less than about 10 mm, or less than about 20 mm, or less than about 30 mm, or less than about 40 mm, or less than about 50 mm, or less than about 60 mm, or less than about 70 mm, or less than about 80 mm, or less than about 90 mm, or less than about 100 mm, or less than about 200 mm, or less than about 300 mm, or less than about 400 mm, or less than about 500 mm, or less than about 600 mm, or less than about 700 mm, or less than about 800 mm, or less than about 900 mm, or less than about 1000 mm, or less than about 2 m, or less than about 3 m, or less than about 4 m, or less than about 5 m.

In some embodiments, RSS may be applied to fabric having a width of greater than about 100 nm, or greater than about 200 nm, or greater than about 300 nm, or greater than about 400 nm, or greater than about 500 nm, or greater than about 600 nm, or greater than about 700 nm, or greater than about 800 nm, or greater than about 900 nm, or greater than about 1000 nm, or greater than about 2 μm, or greater than about 5 μm, or greater than about 10 μm, or greater than about 20 μm, or greater than about 30 μm, or greater than about 40 μm, or greater than about 50 μm, or greater than about 60 μm, or greater than about 70 μm, or greater than about 80 μm, or greater than about 90 μm, or greater than about 100 μm, or greater than about 200 μm, or greater than about 300 μm, or greater than about 400 μm, or greater than about 500 μm, or greater than about 600 μm, or greater than about 700 μm, or greater than about 800 μm, or greater than about 900 μm, or greater than about 1000 μm, or greater than about 2 mm, or greater than about 3 mm, or greater than about 4 mm, or greater than about 5 mm, 6 mm, or greater than about 7 mm, or greater than about 8 mm, or greater than about 9 mm, or greater than about 10 mm, or greater than about 20 mm, or greater than about 30 mm, or greater than about 40 mm, or greater than about 50 mm, or greater than about 60 mm, or greater than about 70 mm, or greater than about 80 mm, or greater than about 90 mm, or greater than about 100 mm, or greater than about 200 mm, or greater than about 300 mm, or greater than about 400 mm, or greater than about 500 mm, or greater than about 600 mm, or greater than about 700 mm, or greater than about 800 mm, or greater than about 900 mm, or greater than about 1000 mm, or greater than about 2 m, or greater than about 3 m, or greater than about 4 m, or greater than about 5 m.

In some embodiments, RSS may be applied to fabric having a length of less than about 100 nm, or less than about 200 nm, or less than about 300 nm, or less than about 400 nm, or less than about 500 nm, or less than about 600 nm, or less than about 700 nm, or less than about 800 nm, or less than about 900 nm, or less than about 1000 nm, or less than about 2 μm, or less than about 5 μm, or less than about 10 μm, or less than about 20 μm, or less than about 30 μm, or less than about 40 μm, or less than about 50 μm, or less than about 60 μm, or less than about 70 μm, or less than about 80 μm, or less than about 90 μm, or less than about 100 μm, or less than about 200 μm, or less than about 300 μm, or less than about 400 μm, or less than about 500 μm, or less than about 600 μm, or less than about 700 μm, or less than about 800 μm, or less than about 900 μm, or less than about 1000 μm, or less than about 2 mm, or less than about 3 mm, or less than about 4 mm, or less than about 5 mm, 6 mm, or less than about 7 mm, or less than about 8 mm, or less than about 9 mm, or less than about 10 mm, or less than about 20 mm, or less than about 30 mm, or less than about 40 mm, or less than about 50 mm, or less than about 60 mm, or less than about 70 mm, or less than about 80 mm, or less than about 90 mm, or less than about 100 mm, or less than about 200 mm, or less than about 300 mm, or less than about 400 mm, or less than about 500 mm, or less than about 600 mm, or less than about 700 mm, or less than about 800 mm, or less than about 900 mm, or less than about 1000 mm.

In some embodiments, RSS may be applied to fabric having a length of greater than about 100 nm, or greater than about 200 nm, or greater than about 300 nm, or greater than about 400 nm, or greater than about 500 nm, or greater than about 600 nm, or greater than about 700 nm, or greater than about 800 nm, or greater than about 900 nm, or greater than about 1000 nm, or greater than about 2 μm, or greater than about 5 μm, or greater than about 10 μm, or greater than about 20 μm, or greater than about 30 μm, or greater than about 40 μm, or greater than about 50 μm, or greater than about 60 μm, or greater than about 70 μm, or greater than about 80 μm, or greater than about 90 μm, or greater than about 100 μm, or greater than about 200 μm, or greater than about 300 μm, or greater than about 400 μm, or greater than about 500 μm, or greater than about 600 μm, or greater than about 700 μm, or greater than about 800 μm, or greater than about 900 μm, or greater than about 1000 μm, or greater than about 2 mm, or greater than about 3 mm, or greater than about 4 mm, or greater than about 5 mm, 6 mm, or greater than about 7 mm, or greater than about 8 mm, or greater than about 9 mm, or greater than about 10 mm, or greater than about 20 mm, or greater than about 30 mm, or greater than about 40 mm, or greater than about 50 mm, or greater than about 60 mm, or greater than about 70 mm, or greater than about 80 mm, or greater than about 90 mm, or greater than about 100 mm, or greater than about 200 mm, or greater than about 300 mm, or greater than about 400 mm, or greater than about 500 mm, or greater than about 600 mm, or greater than about 700 mm, or greater than about 800 mm, or greater than about 900 mm, or greater than about 1000 mm.

In some embodiments, RSS may be applied to fabric having a stretch percentage of less than about 1%, or less than about 2%, or less than about 3%, or less than about 4%, or less than about 5%, or less than about 6%, or less than about 7%, or less than about 8%, or less than about 9%, or less than about 10%, or less than about 20%, or less than about 30%, or less than about 40%, or less than about 50%, or less than about 60%, or less than about 70%, or less than about 80%, or less than about 90%, or less than about 100, or less than about 110%, or less than about 120%, or less than about 130%, or less than about 140%, or less than about 150%, or less than about 160%, or less than about 170%, or less than about 180%, or less than about 190%, or less than about 200%. Stretch percentage may be determined for a fabric having an unstretched width and stretching the fabric to a stretched width, then subtracting the unstretched width from the stretched width to yield the net stretched width, then dividing the net stretched width and multiplying the quotient by 100 to find the stretch percentage (%)

$$( Stretch Percentage = \frac{( Stretched Width - Unstretched Width )}{Unstretched Width} * 100 ) .$$

In some embodiments, RSS may be applied to fabric having a stretch percentage of greater than about 1%, or greater than about 2%, or greater than about 3%, or greater than about 4%, or greater than about 5%, or greater than about 6%, or greater than about 7%, or greater than about 8%, or greater than about 9%, or greater than about 10%, or greater than about 20%, or greater than about 30%, or greater than about 40%, or greater than about 50%, or greater than about 60%, or greater than about 70%, or greater than about 80%, or greater than about 90%, or greater than about 100, or greater than about 110%, or greater than about 120%, or greater than about 130%, or greater than about 140%, or greater than about 150%, or greater than about 160%, or greater than about 170%, or greater than about 180%, or greater than about 190%, or greater than about 200%.

In some embodiments, RSS may be applied to fabric having a tensile energy (N/cm²) of less than about 1 cN/cm², or less than about 2 cN/cm², or less than about 3 cN/cm², or less than about 4 cN/cm², or less than about 5 cN/cm², or less than about 5 cN/cm², or less than about 6 cN/cm², or less than about 7 cN/cm², or less than about 8 cN/cm², or less than about 9 cN/cm², or less than about 10 cN/cm², or less than about 20 cN/cm², or less than about 30 cN/cm², or less than about 40 cN/cm², or less than about 50 cN/cm², or less than about 60 cN/cm², or less than about 70 cN/cm², or less than about 80 cN/cm², or less than about 90 cN/cm², or less than about 100 cN/cm², or less than about 2 N/cm², or less than about 3 N/cm², or less than about 4 N/cm², or less than about 5 N/cm², or less than about 6 N/cm², or less than about 7 N/cm², or less than about 8 N/cm², or less than about 9 N/cm², or less than about 10 N/cm², or less than about 20 N/cm², or less than about 30 N/cm², or less than about 40 N/cm², or less than about 50 N/cm², or less than about 60 N/cm², or less than about 70 N/cm², or less than about 80 N/cm², or less than about 90 N/cm², or less than about 100 N/cm², or less than about 150 N/cm², or less than about 200 N/cm².

In some embodiments, RSS may be applied to fabric having a tensile energy (N/cm²) of greater than about 1 cN/cm², or greater than about 2 cN/cm², or greater than about 3 cN/cm², or greater than about 4 cN/cm², or greater than about 5 cN/cm², or greater than about 5 cN/cm², or greater than about 6 cN/cm², or greater than about 7 cN/cm², or greater than about 8 cN/cm², or greater than about 9 cN/cm², or greater than about 10 cN/cm², or greater than about 20 cN/cm², or greater than about 30 cN/cm², or greater than about 40 cN/cm², or greater than about 50 cN/cm², or greater than about 60 cN/cm², or greater than about 70 cN/cm², or greater than about 80 cN/cm², or greater than about 90 cN/cm², or greater than about 100 cN/cm², or greater than about 2 N/cm², or greater than about 3 N/cm², or greater than about 4 N/cm², or greater than about 5 N/cm², or greater than about 6 N/cm², or greater than about 7 N/cm², or greater than about 8 N/cm², or greater than about 9 N/cm², or greater than about 10 N/cm², or greater than about 20 N/cm², or greater than about 30 N/cm², or greater than about 40 N/cm², or greater than about 50 N/cm², or greater than about 60 N/cm², or greater than about 70 N/cm², or greater than about 80 N/cm², or greater than about 90 N/cm², or greater than about 100 N/cm², or greater than about 150 N/cm², or greater than about 200 N/cm².

In some embodiments, RSS may be applied to fabric having a shear rigidity (N/cm-degree) of less than about 1 cN/cm-degree, or less than about 2 cN/cm-degree, or less than about 3 cN/cm-degree, or less than about 4 cN/cm-degree, or less than about 5 cN/cm-degree, or less than about 5 cN/cm-degree, or less than about 6 cN/cm-degree, or less than about 7 cN/cm-degree, or less than about 8 cN/cm-degree, or less than about 9 cN/cm-degree, or less than about 10 cN/cm-degree, or less than about 20 cN/cm-degree, or less than about 30 cN/cm-degree, or less than about 40 cN/cm-degree, or less than about 50 cN/cm-degree, or less than about 60 cN/cm-degree, or less than about 70 cN/cm-degree, or less than about 80 cN/cm-degree, or less than about 90 cN/cm-degree, or less than about 100 cN/cm-degree, or less than about 2 N/cm-degree, or less than about 3 N/cm-degree, or less than about 4 N/cm-degree, or less than about 5 N/cm-degree, or less than about 6 N/cm-degree, or less than about 7 N/cm-degree, or less than about 8 N/cm-degree, or less than about 9 N/cm-degree, or less than about 10 N/cm-degree, or less than about 20 N/cm-degree, or less than about 30 N/cm-degree, or less than about 40 N/cm-degree, or less than about 50 N/cm-degree, or less than about 60 N/cm-degree, or less than about 70 N/cm-degree, or less than about 80 N/cm-degree, or less than about 90 N/cm-degree, or less than about 100 N/cm-degree, or less than about 150 N/cm-degree, or less than about 200 N/cm-degree.

In some embodiments, RSS may be applied to fabric having a shear rigidity (N/cm-degree) of greater than about 1 cN/cm-degree, or greater than about 2 cN/cm-degree, or greater than about 3 cN/cm-degree, or greater than about 4 cN/cm-degree, or greater than about 5 cN/cm-degree, or greater than about 5 cN/cm-degree, or greater than about 6 cN/cm-degree, or greater than about 7 cN/cm-degree, or greater than about 8 cN/cm-degree, or greater than about 9 cN/cm-degree, or greater than about 10 cN/cm-degree, or greater than about 20 cN/cm-degree, or greater than about 30 cN/cm-degree, or greater than about 40 cN/cm-degree, or greater than about 50 cN/cm-degree, or greater than about 60 cN/cm-degree, or greater than about 70 cN/cm-degree, or greater than about 80 cN/cm-degree, or greater than about 90 cN/cm-degree, or greater than about 100 cN/cm-degree, or greater than about 2 N/cm-degree, or greater than about 3 N/cm-degree, or greater than about 4 N/cm-degree, or greater than about 5 N/cm-degree, or greater than about 6 N/cm-degree, or greater than about 7 N/cm-degree, or greater than about 8 N/cm-degree, or greater than about 9 N/cm-degree, or greater than about 10 N/cm-degree, or greater than about 20 N/cm-degree, or greater than about 30 N/cm-degree, or greater than about 40 N/cm-degree, or greater than about 50 N/cm-degree, or greater than about 60 N/cm-degree, or greater than about 70 N/cm-degree, or greater than about 80 N/cm-degree, or greater than about 90 N/cm-degree, or greater than about 100 N/cm-degree, or greater than about 150 N/cm-degree, or greater than about 200 N/cm-degree.

In some embodiments, RSS may be applied to fabric having a bending rigidity (N·cm²/cm) of less than about 1 cN·cm²/cm, or less than about 2 cN·cm²/cm, or less than about 3 cN·cm²/cm, or less than about 4 cN·cm²/cm, or less than about 5 cN·cm²/cm, or less than about 5 cN·cm²/cm, or less than about 6 cN·cm²/cm, or less than about 7 cN·cm²/cm, or less than about 8 cN·cm²/cm, or less than about 9 cN·cm²/cm, or less than about 10 cN·cm²/cm, or less than about 20 cN·cm²/cm, or less than about 30 cN·cm²/cm, or less than about 40 cN·cm²/cm, or less than about 50 cN·cm²/cm, or less than about 60 cN·cm²/cm, or less than about 70 cN·cm²/cm, or less than about 80 cN·cm²/cm, or less than about 90 cN·cm²/cm, or less than about 100 cN·cm²/cm, or less than about 2 N·cm²/cm, or less than about 3 N·cm²/cm, or less than about 4 N·cm²/cm, or less than about 5 N·cm²/cm, or less than about 6 N·cm²/cm, or less than about 7 N·cm²/cm, or less than about 8 N·cm²/cm, or less than about 9 N·cm²/cm, or less than about 10 N·cm²/cm, or less than about 20 N·cm²/cm, or less than about 30 N·cm²/cm, or less than about 40 N·cm²/cm, or less than about 50 N·cm²/cm, or less than about 60 N·cm²/cm, or less than about 70 N·cm²/cm, or less than about 80 N·cm²/cm, or less than about 90 N·cm²/cm, or less than about 100 N·cm²/cm, or less than about 150 N·cm²/cm, or less than about 200 N·cm²/cm.

In some embodiments, RSS may be applied to fabric having a bending rigidity (N·cm²/cm) of greater than about 1 cN·cm²/cm, or greater than about 2 cN·cm²/cm, or greater than about 3 cN·cm²/cm, or greater than about 4 cN·cm²/cm, or greater than about 5 cN·cm²/cm, or greater than about 5 cN·cm²/cm, or greater than about 6 cN·cm²/cm, or greater than about 7 cN·cm²/cm, or greater than about 8 cN·cm²/cm, or greater than about 9 cN·cm²/cm, or greater than about 10 cN·cm²/cm, or greater than about 20 cN·cm²/cm, or greater than about 30 cN·cm²/cm, or greater than about 40 cN·cm²/cm, or greater than about 50 cN·cm²/cm, or greater than about 60 cN·cm²/cm, or greater than about 70 cN·cm²/cm, or greater than about 80 cN·cm²/cm, or greater than about 90 cN·cm²/cm, or greater than about 100 cN·cm²/cm, or greater than about 2 N·cm²/cm, or greater than about 3 N·cm²/cm, or greater than about 4 N·cm²/cm, or greater than about 5 N·cm²/cm, or greater than about 6 N·cm²/cm, or greater than about 7 N·cm²/cm, or greater than about 8 N·cm²/cm, or greater than about 9 N·cm²/cm, or greater than about 10 N·cm²/cm, or greater than about 20 N·cm²/cm, or greater than about 30 N·cm²/cm, or greater than about 40 N·cm²/cm, or greater than about 50 N·cm²/cm, or greater than about 60 N·cm²/cm, or greater than about 70 N·cm²/cm, or greater than about 80 N·cm²/cm, or greater than about 90 N·cm²/cm, or greater than about 100 N·cm²/cm, or greater than about 150 N·cm²/cm, or greater than about 200 N·cm²/cm.

In some embodiments, RSS may be applied to fabric having a compression energy (N·cm/cm²) of less than about 1 cN·cm/cm², or less than about 2 cN·cm/cm², or less than about 3 cN·cm/cm², or less than about 4 cN·cm/cm², or less than about 5 c N·cm/cm², or less than about 5 cN·cm/cm², or less than about 6 cN·cm/cm², or less than about 7 cN·cm/cm², or less than about 8 cN·cm/cm², or less than about 9 cN·cm/cm², or less than about 10 cN·cm/cm², or less than about 20 cN·cm/cm², or less than about 30 cN·cm/cm², or less than about 40 cN·cm/cm², or less than about 50 cN·cm/cm², or less than about 60 cN·cm/cm², or less than about 70 cN·cm/cm², or less than about 80 cN·cm/cm², or less than about 90 cN·cm/cm², or less than about 100 cN·cm/cm², or less than about 2 N·cm/cm², or less than about 3 N·cm/cm², or less than about 4 N·cm/cm², or less than about 5 N·cm/cm², or less than about 6 N·cm/cm², or less than about 7 N·cm/cm², or less than about 8 N·cm/cm², or less than about 9 N·cm/cm², or less than about 10 N·cm/cm², or less than about 20 N·cm/cm², or less than about 30 N·cm/cm², or less than about 40 N·cm/cm², or less than about 50 N·cm/cm², or less than about 60 N·cm/cm², or less than about 70 N·cm/cm², or less than about 80 N·cm/cm², or less than about 90 N·cm/cm², or less than about 100 N·cm/cm², or less than about 150 N·cm/cm², or less than about 200 N·cm/cm².

In some embodiments, RSS may be applied to fabric having a compression energy (N·cm/cm²) of greater than about 1 cN·cm/cm², or greater than about 2 cN·cm/cm², or greater than about 3 cN·cm/cm², or greater than about 4 cN·cm/cm², or greater than about 5 cN·cm/cm², or greater than about 5 cN·cm/cm², or greater than about 6 cN·cm/cm², or greater than about 7 cN·cm/cm², or greater than about 8 cN·cm/cm², or greater than about 9 cN·cm/cm², or greater than about 10 cN·cm/cm², or greater than about 20 cN·cm/cm², or greater than about 30 cN·cm/cm², or greater than about 40 cN·cm/cm², or greater than about 50 cN·cm/cm², or greater than about 60 cN·cm/cm², or greater than about 70 cN·cm/cm², or greater than about 80 cN·cm/cm², or greater than about 90 cN·cm/cm², or greater than about 100 cN·cm/cm², or greater than about 2 N·cm/cm², or greater than about 3 N·cm/cm², or greater than about 4 N·cm/cm², or greater than about 5 N·cm/cm², or greater than about 6 N·cm/cm², or greater than about 7 N·cm/cm², or greater than about 8 N·cm/cm², or greater than about 9 N·cm/cm², or greater than about 10 N·cm/cm², or greater than about 20 N·cm/cm², or greater than about 30 N·cm/cm², or greater than about 40 N·cm/cm², or greater than about 50 N·cm/cm², or greater than about 60 N·cm/cm², or greater than about 70 N·cm/cm², or greater than about 80 N·cm/cm², or greater than about 90 N·cm/cm², or greater than about 100 N·cm/cm², or greater than about 150 N·cm/cm², or greater than about 200 N·cm/cm².

In some embodiments, RSS may be applied to fabric having a coefficient of friction of less than about 0.04, or less than about 0.05, or less than about 0.06, or less than about 0.07, or less than about 0.08, or less than about 0.09, or less than about 0.10, or less than about 0.10, or less than about 0.15, or less than about 0.20, or less than about 0.25, or less than about 0.30, or less than about 0.35, or less than about 0.40, or less than about 0.45, or less than about 0.50, or less than about 0.55, or less than about 0.60, or less than about 0.65, or less than about 0.70, or less than about 0.75, or less than about 0.80, or less than about 0.85, or less than about 0.90, or less than about 0.95, or less than about 1.00, or less than about 1.05.

In some embodiments, RSS may be applied to fabric having a coefficient of friction of greater than about 0.04, or greater than about 0.05, or greater than about 0.06, or greater than about 0.07, or greater than about 0.08, or greater than about 0.09, or greater than about 0.10, or greater than about 0.10, or greater than about 0.15, or greater than about 0.20, or greater than about 0.25, or greater than about 0.30, or greater than about 0.35, or greater than about 0.40, or greater than about 0.45, or greater than about 0.50, or greater than about 0.55, or greater than about 0.60, or greater than about 0.65, or greater than about 0.70, or greater than about 0.75, or greater than about 0.80, or greater than about 0.85, or greater than about 0.90, or greater than about 0.95, or greater than about 1.00, or greater than about 1.05.

In some embodiments, chemical finishes may be applied to textiles before or after such textiles are coated with RSS. In an embodiment, chemical finishing may be intended as the application of chemical agents and/or RSS to textiles, including fibers, yarn, and fabric, or to garments that are prepared by such fibers, yarn, and fabric to modify the original textile's or garment's properties and achieve properties in the textile or garment that would be otherwise absent. With chemical finishes, textiles treated with such chemical finishes may act as surface treatments and/or the treatments may modify the elemental analysis of treated textile base polymers.

In an embodiment, a type of chemical finishing may include the application of certain recombinant silk based solutions to textiles. For example, RSS may be applied to a fabric after it is dyed, but there are also scenarios that may require the application of RSS during processing, during dyeing, or after a garment is assembled from a selected textile or fabric, thread, or yarn. In some embodiments, after its application, RSS may be dried with the use of heat. RSS may then be fixed to the surface of the textile in a processing step called curing.

In some embodiments, RSS may be supplied in a concentrated form suspended in water. In some embodiments, RSS may have a concentration by weight (% w/w or % w/v) or by volume (v/v) of less than about 50%, or less than about 45%, or less than about 40%, or less than about 35%, or less than about 30%, or less than about 25%, or less than about 20%, or less than about 15%, or less than about 10%, or less than about 5%, or less than about 4%, or less than about 3%, or less than about 2%, or less than about 1%, or less than about 0.1%, or less than about 0.01%, or less than about 0.001%, or less than about 0.0001%, or less than about 0.00001%. In some embodiments, RSS may have a concentration by weight (% w/w or % w/v) or by volume (v/v) of greater than about 50%, or greater than about 45%, or greater than about 40%, or greater than about 35%, or greater than about 30%, or greater than about 25%, or greater than about 20%, or greater than about 15%, or greater than about 10%, or greater than about 5%, or greater than about 4%, or greater than about 3%, or greater than about 2%, or greater than about 1%, or greater than about 0.1%, or greater than about 0.01%, or greater than about 0.001%, or greater than about 0.0001%, or greater than about 0.00001%.

In some embodiments, the solution concentration and the wet pick of the material determines the amount of recombinant silk solution (RSS), which may include recombinant silk-based proteins or fragments thereof, that may be fixed or otherwise adhered to the textile being coated. The wet pick up may be expressed by the following formula:

$$wet pick up ( \% ) = \frac{weight of SFS applied \times 100}{weight of dry textile material} .$$

The total amount of RSS added to the textile material may be expressed by the following formula:

$$SFS added ( \% ) = \frac{weight of dry SFS coated textile material \times 100}{weight of dry textile material before coating} .$$

Regarding methods for applying RSS to textiles more broadly, RSS may be applied to textiles through a pad or roller application on process, a saturation and removal process, and/or a topical application process. Moreover, the methods of silk application (i.e., RSS application or coating) may include bath coating, kiss rolling, spray coating, and/or two-sided rolling. In some embodiments, the coating processes (e.g., bath coating, kiss rolling, spray coating, two-sided rolling, roller application, saturation and removal application, and/or topical application), drying processes, and curing processes may be varied as described herein to modify one or more selected textile (e.g., fabric) properties of the resulting coated textile wherein such properties include, but are not limited to wetting time, absorption rate, spreading speed, accumulative one-way transport, and/or overall moisture management capability. In some embodiments, the aforementioned selected properties may be enhanced by varying one or more of the coating processes, drying processes, and curing processes as described herein.

In some embodiments, the silk compositions provided herein may be applied in a wet process or a dry process, such as by applying the silk compositions to a wet textile or a dry textile.

In an embodiment, the padder application may be used on dry or wet textile. For example, it may be applied on fabric after the dyeing process. The fabric may be fed into a water bath solution and may reach saturation. The fabric to be coated may then pass through a set of rollers that, based on multiple variables, extract the bath solution in excess to the desired wet pick up %. The variables that affect the wet pick up % are the roller pressure and materials, the fabric composition and construction, and the RSS viscosity.

In an embodiment, the padder application on wet textile may be used to reduce the cost of drying the fabric post dyeing. The fabric exiting the pad rollers may maintain a higher weight % than the incoming fabric to maintain a RSS deposit on the fabric; and the RSS solution may need to account for any dilution taking place due to water present on the incoming fabric.

In an embodiment, the saturation and removal application is a low wet pick up method that may, for example, solve some of the issues associated with removing large amounts of water during drying processes. Since fabric may dry in an oven from the outside surface towards the inside, water may move from the inside to the outside resulting in a higher coating concentration on the outside surface. With less water content, migration may be reduced due to a higher viscosity in the solution. However, decreased wet pick up may result in an uneven solution deposit.

In an embodiment, vacuum extraction may be used as a method for low wet pick up. Saturated fabric may be subject to a vacuum that pulls solution out of the fabric and returns it to an application loop. Air jet ejection may be a method for providing low wet pick up. The saturated fabric may be subjected to high pressure steam that removes solution out of the fabric and returns it to an application loop. In an embodiment, a porous bowl method may be used for low wet pick up.

Solid pad rollers may be substituted with rubber coated fiber rollers. Saturated fabric may be subjected to the pressure of the roller since the porosity of the rollers may allow for more solution to be squeezed from the fabric.

In an embodiment, a transfer padding method may be used for low wet pick up. Saturated fabric may be passed through two continuous dry non-woven fabrics and may be pressed at low pressure. The non-woven fabrics may extract excess solution from the fabric being treated.

In an embodiment, topical application may be used as a low wet pick up method of application that deposits the desired amount of RSS to the fabric without removing any excess material. The methods described above may be used for one-sided coating applications, but there are variations that may allow for two-sided coating.

In an embodiment, kiss rolling may be used as a topical method of application that transfers the RSS from a roller (i.e., a kiss roller) to one side of the fabric. The solution viscosity, roller surface finish, speed of the roller, speed of the fabric, contact angle of the fabric on the roller and properties of the fabric are parameters that control the amount of solution deposited on the fabric.

In an embodiment, a variation to the kiss roller technique may be the Triatex MA system that uses two moisture content sensors to determine the solution pick up at the kiss roller and adjust the kiss roller controllable variable to maintain consistent the solution deposit onto the fabric.

In an embodiment, a loop transfer application may be used as a topical method of application that transfers the RSS from a saturated loop fabric to the fabric to be coated between low pressure pad rollers. There is a two rollers version that may allow for minimum contact with the fabric and a three rollers version that allows for greater contact with the fabric.

In an embodiment, an engrave roller application may be used as a topical method of application that may transfer a metered amount of RSS onto the fabric. This may be achieved by engraving a pattern on the surface of the roller with precise depth and design that contains a controlled amount of RSS. A blade may be used to remove any solution that is deposited on the surface of the roller in order to maintain a consistent transfer of solution to the fabric to be coated.

In an embodiment, rotary screen printing may be used as a topical method of application that may deposit RSS onto the fabric by seeping the solution through a roller screen. The solution may be contained in the screen print roller core at a set level while a blade may be used to remove any excess solution from the interior roller wall, providing a clean surface for the next revolution of the screen printer roller.

In an embodiment, magnetic roller coating may be used as a topical method of application that may deposit RSS from a kiss roller onto the fabric to be coated. The kiss roller is semi-submersed in a bath solution while a magnetic field created in the fabric driving roller determines the amount of pressure applied by the kiss roller, controlling the solution pick up rate.

In an embodiment, spraying may be used as a topical method of application that may transfer RSS onto the fabric by nebulizing the solution. The spray pattern may be controlled by the nozzle pattern, size, and the air flow. Spray application may be used for one side application or also two sided applications.

In an embodiment, foam application may be used a topical method of application that may transfer RSS onto the fabric. Foam may be made by substituting part of the water in the solution with air therefore reducing the amount of water to be applied to the fabric. Foam application may be used for one-sided application or two-sided application where the same foam may be deposited through a squeeze roller or different foam solutions may be provided through transfer rolls or through a slot applicator.

In an embodiment, the application of RSS may take place after a garment is assembled. In an embodiment, the process may take place in a washing and dyeing machine or in a spray booth. For example, a washing and dyeing machine may be similar in shape to a household front loader washing machine, it allows the process to take place at exhaustion post dyeing or with an independent processing cycle. In an embodiment, a spray booth machine may include a manual or a fully automated process. For example, a garment may be held by a mannequin while an operator or an anthropomorphic robot may spray the solution onto the fabric.

In an embodiment, RSS may be a water based solution that, after its application to the textile, may require thermal vaporization to infuse the RSS onto the textile. Thermal vaporization may be applied by heat transfer through radiation with equipment such as infrared or radio frequency dryer.

In an embodiment, thermal vaporization may be applied by convection through heated air circulating in an oven to the required temperature, while the fabric is clamped and is transported by a conveyor. This allows full control on fabric width dimension.

In an embodiment, thermal vaporization may be applied by conduction through contacting the textile with heated cylinder or calendar cylinder. Since the fabric is not clamp there is minimal control on fabric width.

In an embodiment, curing of the RSS on the textile may be completed with the same equipment used for the thermal vaporization in a continuous cycle or in a separate cycle.

In an embodiment, curing time temperature may be dependent the textile polymer content and the binding method of preference for the RSS with the specific polymer. The curing process may not start until the thermal vaporization is completed.

In some embodiments, sensor may be used to monitor RSS deposition on the textile and the drying and curing steps.

In some embodiments, for monitoring the deposition of RSS, a contactless sensor, like the one supplied by Pleva model AF120 based on microwave absorption of water, may be used. Measurement of the material moisture may be based on microwave absorption by water. A semiconductor oscillator transmits microwave energy through the web. The non-absorbed part of the energy may be received on the opposite side by a microwave receiver. The amount of absorption is a measurement of the absolute moisture content. The microwave sensor is capable of detecting and measuring water content from a minimum of 0 up to 2000 g H₂O/m².

In some embodiments, for wide fabric processing multiple sensor may be paired side by side, delivering the data analysis to a centralized control system loop capable to add more solution in the area of the fabric that is low.

In some embodiments, another sensor may be used that is based on microwave technology, such as Aqualot by Mahlo. The sensor may evaluate the shift in the resonant frequency of the two standing waves with respect to each other rather than the attenuation of the microwaves by the quantity of water molecules in the measuring gap.

In some embodiments, another contactless sensor for RSS may be the IR-3000 by MoistTech based on near infrared sensing technology. The sensor measures the amount of near infrared energy reflected at a given wavelength that is inversely proportional to the quantity of absorbing molecules in the fabric.

In some embodiments, the residual moister at the end of the curing process may be measured to further confirm the drying and curing process. In addition to the above sensor, a contact sensor such as the Textometer RMS by Mahlo may be used for measuring moister through conductivity.

In some embodiments, monitoring the end of the drying process phase may be achieved by measuring the fabric temperature with a contactless temperature sensor. When wet product enters the dryer, it first heats up to the cooling limit temperature. In some embodiments, when the water content drops to residual moisture levels, the product temperature may begin to rise again. The closer the product temperature approaches the circulation air temperature in the dryer, the slower the temperature continues to rise. In some embodiments, at a certain temperature threshold (called the fixing temperature) the temperature necessary for processing, fixing, or condensing is reached.

In some embodiments, to determine the dwell time for a desired product temperature, the surface temperature of the product may be measured without contact at several locations in the dryer using high-temperature resistant infrared pyrometers. Mahlo Permaset VMT is an infrarem Pyrometer that may be assembled in multiple units to monitors temperature through the dryer. Setex is another manufacturer offering fabric temperature sensors for use in dryers and oven like the models WTM V11, V21, and V41.

In some embodiments, RSS may be applied to a textile during exhaust dyeing. In some embodiments, the process may involve loading fabric into a bath, originally known as a batch, and allowing it to come into equilibrium with the solution. Exhaust dyeing may be the ability of the recombinant silk molecules to move from the solution onto the fibers or thread of a textile (substantivity). The substantivity of the recombinant silk may be influenced by temperature or additives, such as salt.

In some embodiments, an exhaust dyeing process may take anywhere from a few minutes to a few hours. When the fabric has been absorbed, or fixed, as much recombinant silk as it can, the bath may be emptied and the fabric may be rinsed to remove any excess solution.

In some embodiments, an important parameter in exhaust dyeing may be what is known as the specific liquor ratio. This describes the ratio of the mass of the fabric to the volume of the RSS bath and determines the amount of recombinant silk deposited on a textile.

In some embodiments, RSS can be applied to a textile during jet dyeing processes. A jet dyeing machine may be formed by closed tubular system where the fabric is placed. For transporting the fabric through the tube, a jet of dye liquor is supplied through a venturi. The jet may create turbulence. This may help in RSS penetration along with preventing the fabric from touching the walls of the tube. For example, as the fabric is often exposed to comparatively higher concentrations of liquor within the transport tube, a small RSS bath is needed in the bottom of the vessel. This arrangement may be enough for the smooth movement from rear to front of the vessel.

In some embodiments, RSS may be applied during Paddle dyeing. Paddle dyeing machines may be generally used to many forms of textiles but the method best suits to garments. Heat may be generated through steam injection directly into the coating bath. In an embodiment, a paddle dyeing machine operates through a paddle that circulates both the bath and garments in a perforated central island. It is here that the RSS, water, and steam for heat are added. The overhead paddle machine may be described as a vat with a paddle that has blades of full width. The blades may generally dip a few centimeters into the vat. This action may stir the bath and push garments to be died down, thus keeping them submerged in the dye liquor.

In some embodiments, the processing methods set forth herein may be used to apply RSS to textiles with one or more of the following parameters including, but not limited to, fabric speed, solution viscosity, solution added to fabric, fabric range width, drying temperature, drying time, curing time, fabric tension, padder pressure, padder roller shore hardness, stenter temperature, and common drying and curing temperatures. In an embodiment, the processing method parameters may also include a condensation temperature, which may vary depending upon the chemical recipe used to apply the RSS to the textiles.

In an embodiment, the fabric speed for the processes of the disclosure may be less than about 0.1 m/min, or less than about 0.2 m/min, or less than about 0.3 m/min, or less than about 0.4 m/min, or less than about 0.5 m/min, or less than about 0.6 m/min, or less than about 0.7 m/min, or less than about 0.8 m/min, or less than about 0.9 m/min, or less than about 1 m/min, or less than about 2 m/min, or less than about 3 m/min, or less than about 4 m/min, or less than about 5 m/min, or less than about 6 m/min, or less than about 7 m/min, or less than about 8 m/min, or less than about 9 m/min, or less than about 10 m/min, or less than about 20 m/min, or less than about 30 m/min, or less than about 40 m/min, or less than about 50 m/min, or less than about 60 m/min.

In an embodiment, the fabric speed for the processes of the disclosure may be greater than about 0.1 m/min, or greater than about 0.2 m/min, or greater than about 0.3 m/min, or greater than about 0.4 m/min, or greater than about 0.5 m/min, or greater than about 0.6 m/min, or greater than about 0.7 m/min, or greater than about 0.8 m/min, or greater than about 0.9 m/min, or greater than about 1 m/min, or greater than about 2 m/min, or greater than about 3 m/min, or greater than about 4 m/min, or greater than about 5 m/min, or greater than about 6 m/min, or greater than about 7 m/min, or greater than about 8 m/min, or greater than about 9 m/min, or greater than about 10 m/min, or greater than about 20 m/min, or greater than about 30 m/min, or greater than about 40 m/min, or greater than about 50 m/min, or greater than about 60 m/min.

In an embodiment, the solution viscosity for the processes of the disclosure may be less than about 1000 mPas, or less than about 1500 mPas, or less than about 2000 mPas, or less than about 2500, or less than about 3000 mPas, or less than about 4000 mPas, or less than about 4500 mPas, or less than about 5000 mPas, or less than about 5500 mPas, or less than about 6000 mPas, or less than about 6500 mPas, or less than about 7000 mPas, or less than about 7500 mPas, or less than about 8000 mPas, or less than about 8500 mPas, or less than about 9000 mPas, or less than about 9500 mPas, or less than about 10000 mPas, or less than about 10500 mPas, or less than about 11000 mPas, or less than about 11500 mPas, or less than about 12000 mPas.

In an embodiment, the solution viscosity for the processes of the disclosure may be greater than about 1000 mPas, or greater than about 1500 mPas, or greater than about 2000 mPas, or greater than about 2500, or greater than about 3000 mPas, or greater than about 4000 mPas, or greater than about 4500 mPas, or greater than about 5000 mPas, or greater than about 5500 mPas, or greater than about 6000 mPas, or greater than about 6500 mPas, or greater than about 7000 mPas, or greater than about 7500 mPas, or greater than about 8000 mPas, or greater than about 8500 mPas, or greater than about 9000 mPas, or greater than about 9500 mPas, or greater than about 10000 mPas, or greater than about 10500 mPas, or greater than about 11000 mPas, or greater than about 11500 mPas, or greater than about 12000 mPas.

In an embodiment, the solution may be added to a textile (e.g., fabric) for the processes of the disclosure in less than about 0.01 g/m², or less than about 0.02 g/m², or less than about 0.03 g/m², or less than about 0.04 g/m², or less than about 0.05 g/m², or less than about 0.06 g/m², or less than about 0.07 g/m², or less than about 0.08 g/m², or less than about 0.09 g/m², or less than about 0.10 g/m², or less than about 0.2 g/m², or less than about 0.3 g/m², or less than about 0.4 g/m², or less than about 0.5 g/m², or less than about 0.6 g/m², or less than about 0.7 g/m², or less than about 0.8 g/m², or less than about 0.9 g/m², or less than about 1 g/m², or less than about 2 g/m², or less than about 3 g/m², or less than about 4 g/m², or less than about 5 g/m², or less than about 6 g/m², or less than about 7 g/m², or less than about 8 g/m², or less than about 9 g/m², or less than about 10 g/m², or less than about 20 g/m², or less than about 30 g/m², or less than about 40 g/m², or less than about 50 g/m², or less than about 60 g/m², or less than about 70 g/m², or less than about 80 g/m², or less than about 90 g/m², or less than about 100 g/m².

In an embodiment, the solution may be added to a textile (e.g., fabric) for the processes of the disclosure in greater than about 0.01 g/m², or greater than about 0.02 g/m², or greater than about 0.03 g/m², or greater than about 0.04 g/m², or greater than about 0.05 g/m², or greater than about 0.06 g/m², or greater than about 0.07 g/m², or greater than about 0.08 g/m², or greater than about 0.09 g/m², or greater than about 0.10 g/m², or greater than about 0.2 g/m², or greater than about 0.3 g/m², or greater than about 0.4 g/m², or greater than about 0.5 g/m², or greater than about 0.6 g/m², or greater than about 0.7 g/m², or greater than about 0.8 g/m², or greater than about 0.9 g/m², or greater than about 1 g/m², or greater than about 2 g/m², or greater than about 3 g/m², or greater than about 4 g/m², or greater than about 5 g/m², or greater than about 6 g/m², or greater than about 7 g/m², or greater than about 8 g/m², or greater than about 9 g/m², or greater than about 10 g/m², or greater than about 20 g/m², or greater than about 30 g/m², or greater than about 40 g/m², or greater than about 50 g/m², or greater than about 60 g/m², or greater than about 70 g/m², or greater than about 80 g/m², or greater than about 90 g/m², or greater than about 100 g/m².

In an embodiment, the fabric range width for the processes of the disclosure may be less than about 1 mm, or less than about 2 mm, or less than about 3 mm, or less than about 4 mm, or less than about 5 mm, or less than about 6 mm, or less than about 7 mm, or less than about 8 mm, or less than about 9, or less than about 10 mm, or less than about 20 mm, or less than about 30 mm, or less than about 40 mm, or less than about 50 mm, or less than about 60 mm, or less than about 70 mm, or less than about 80 mm, or less than about 90 mm, or less than about 100 mm, or less than about 200, or less than about 300 mm, or less than about 400 mm, or less than about 500 mm, or less than about 600 mm, or less than about 700 mm, or less than about 800 mm, or less than about 900 mm, or less than about 1000 mm, or less than about 2000 mm, or less than about 2000 mm, or less than about 3000 mm, or less than about 4000 mm, or less than about 5000 mm.

In an embodiment, the fabric range width for the processes of the disclosure may be greater than about 1 mm, or greater than about 2 mm, or greater than about 3 mm, or greater than about 4 mm, or greater than about 5 mm, or greater than about 6 mm, or greater than about 7 mm, or greater than about 8 mm, or greater than about 9, or greater than about 10 mm, or greater than about 20 mm, or greater than about 30 mm, or greater than about 40 mm, or greater than about 50 mm, or greater than about 60 mm, or greater than about 70 mm, or greater than about 80 mm, or greater than about 90 mm, or greater than about 100 mm, or greater than about 200, or greater than about 300 mm, or greater than about 400 mm, or greater than about 500 mm, or greater than about 600 mm, or greater than about 700 mm, or greater than about 800 mm, or greater than about 900 mm, or greater than about 1000 mm, or greater than about 2000 mm, or greater than about 2000 mm, or greater than about 3000 mm, or greater than about 4000 mm, or greater than about 5000 mm.

In an embodiment, the drying and/or curing temperature for the processes of the disclosure may be less than about 70° C., or less than about 75° C., or less than about 80° C., or less than about 85° C., or less than about 90° C., or less than about 95° C., or less than about 100° C., or less than about 110° C., or less than about 120° C., or less than about 130° C., or less than about 140° C., or less than about 150° C., or less than about 160° C., or less than about 170° C., or less than about 180° C., or less than about 190° C., or less than about 200° C., or less than about 210° C., or less than about 220° C., or less than about 230° C.

In an embodiment, the drying and/or curing temperature for the processes of the disclosure may be greater than about 70° C., or greater than about 75° C., or greater than about 80° C., or greater than about 85° C., or greater than about 90° C., or greater than about 95° C., or greater than about 100° C., or greater than about 110° C., or greater than about 120° C., or greater than about 130° C., or greater than about 140° C., or greater than about 150° C., or greater than about 160° C., or greater than about 170° C., or greater than about 180° C., or greater than about 190° C., or greater than about 200° C., or greater than about 210° C., or greater than about 220° C., or greater than about 230° C.

In an embodiment, the drying time for the processes of the disclosure may be less than about 10 seconds, or less than about 20 seconds, or less than about 30 seconds, or less than about 40 seconds, or less than about 50 seconds, or less than about 60 seconds, or less than about 2 minutes, or less than about, 3 minutes, or less than about 4 minutes, or less than about 5 minutes, or less than about 6 minutes, or less than about 7 minutes, or less than about 8 minutes, or less than about 9 minutes, or less than about 10 minutes, or less than about 20 minutes, or less than about 30 minutes, or less than about 40 minutes, or less than about 50 minutes, or less than about 60 minutes.

In an embodiment, the drying time for the processes of the disclosure may be greater than about 10 seconds, or greater than about 20 seconds, or greater than about 30 seconds, or greater than about 40 seconds, or greater than about 50 seconds, or greater than about 60 seconds, or greater than about 2 minutes, or greater than about, 3 minutes, or greater than about 4 minutes, or greater than about 5 minutes, or greater than about 6 minutes, or greater than about 7 minutes, or greater than about 8 minutes, or greater than about 9 minutes, or greater than about 10 minutes, or greater than about 20 minutes, or greater than about 30 minutes, or greater than about 40 minutes, or greater than about 50 minutes, or greater than about 60 minutes.

In an embodiment, the curing time for the processes of the disclosure may be less than about 1 second, or less than about 2 seconds, or less than about 3 seconds, or less than about 4 seconds, or less than about 5 seconds, or less than about 6 seconds, or less than about 7 seconds, or less than about 8 seconds, or less than about 9 seconds, or less than about 10 seconds, or less than about 20 seconds, or less than about 30 seconds, or less than about 40 seconds, or less than about 50 seconds, or less than about 60 seconds, or less than about 2 minutes, or less than about 3 minutes, or less than about 4 minutes, or less than about 5 minutes, or less than about 6 minutes, or less than about 7 minutes, or less than about 8 minutes, or less than about 9 minutes, or less than about 10 minutes, or less than about 20 minutes, or less than about 30 minutes, or less than about 40 minutes, or less than about 50 minutes, or less than about 60 minutes.

In an embodiment, the curing time for the processes of the disclosure may be greater than about 1 second, or greater than about 2 seconds, or greater than about 3 seconds, or greater than about 4 seconds, or greater than about 5 seconds, or greater than about 6 seconds, or greater than about 7 seconds, or greater than about 8 seconds, or greater than about 9 seconds, or greater than about 10 seconds, or greater than about 20 seconds, or greater than about 30 seconds, or greater than about 40 seconds, or greater than about 50 seconds, or greater than about 60 seconds, or greater than about 2 minutes, or greater than about 3 minutes, or greater than about 4 minutes, or greater than about 5 minutes, or greater than about 6 minutes, or greater than about 7 minutes, or greater than about 8 minutes, or greater than about 9 minutes, or greater than about 10 minutes, or greater than about 20 minutes, or greater than about 30 minutes, or greater than about 40 minutes, or greater than about 50 minutes, or greater than about 60 minutes.

In an embodiment, the fabric tension for the processes of the disclosure may be less than about 1 N, or less than about 2 N, or less than about 3 N, or less than about 4 N, or less than about 5 N, or less than about 6 N, or less than about 7 N, or less than about 8 N, or less than about 9 N, or less than about 10 N, or less than about 20 N, or less than about 30 N, or less than about 40 N, or less than about 50 N, or less than about 60 N, or less than about 70 N, or less than about 80 N, or less than about 90 N, or less than about 100 N, or less than about 150 N, or less than about 200 N, or less than about 250 N, or less than about 300 N.

In an embodiment, the fabric tension for the processes of the disclosure may be greater than about 1 N, or greater than about 2 N, or greater than about 3 N, or greater than about 4 N, or greater than about 5 N, or greater than about 6 N, or greater than about 7 N, or greater than about 8 N, or greater than about 9 N, or greater than about 10 N, or greater than about 20 N, or greater than about 30 N, or greater than about 40 N, or greater than about 50 N, or greater than about 60 N, or greater than about 70 N, or greater than about 80 N, or greater than about 90 N, or greater than about 100 N, or greater than about 150 N, or greater than about 200 N, or greater than about 250 N, or greater than about 300 N.

In an embodiment, the padder pressure for the processes of the disclosure may be less than about 1 N/mm, or less than about 2 N/mm, or less than about 3 N/mm, or less than about 4 N/mm, or less than about 4 N/mm, or less than about 5 N/mm, or less than about 6 N/mm, or less than about 7 N/mm, or less than about 8 N/mm, or less than about 9 N/mm, or less than about 10 N/mm, or less than about 20 N/mm, or less than about 30 N/mm, or less than about 40 N/mm, or less than about 50 N/mm, or less than about 60 N/mm, or less than about 70 N/mm, or less than about 80 N/mm, or less than about 90 N/mm.

In an embodiment, the padder pressure for the processes of the disclosure may be greater than about 1 N/mm, or greater than about 2 N/mm, or greater than about 3 N/mm, or greater than about 4 N/mm, or greater than about 4 N/mm, or greater than about 5 N/mm, or greater than about 6 N/mm, or greater than about 7 N/mm, or greater than about 8 N/mm, or greater than about 9 N/mm, or greater than about 10 N/mm, or greater than about 20 N/mm, or greater than about 30 N/mm, or greater than about 40 N/mm, or greater than about 50 N/mm, or greater than about 60 N/mm, or greater than about 70 N/mm, or greater than about 80 N/mm, or greater than about 90 N/mm.

In an embodiment, the padder roller shore hardness for the processes of the disclosure may be less than about 70 shore A, or less than about 75 shore A, or less than about 80 shore A, or less than about 85 shore A, or less than about 90 shore A, or less than about 95 shore A, or less than about 100 shore A.

In an embodiment, the padder roller shore hardness for the processes of the disclosure may be greater than about 70 shore A, or greater than about 75 shore A, or greater than about 80 shore A, or greater than about 85 shore A, or greater than about 90 shore A, or greater than about 95 shore A, or greater than about 100 shore A.

In an embodiment, the stenter temperature for the processes of the disclosure may be less than about 70° C., or less than about 75° C., or less than about 80° C., or less than about 85° C., or less than about 90° C., or less than about 95° C., or less than about 100° C., or less than about 110° C., or less than about 120° C., or less than about 130° C., or less than about 140° C., or less than about 150° C., or less than about 160° C., or less than about 170° C., or less than about 180° C., or less than about 190° C., or less than about 200° C., or less than about 210° C., or less than about 220° C., or less than about 230° C.

In an embodiment, the stenter temperature for the processes of the disclosure may be greater than about 70° C., or greater than about 75° C., or greater than about 80° C., or greater than about 85° C., or greater than about 90° C., or greater than about 95° C., or greater than about 100° C., or greater than about 110° C., or greater than about 120° C., or greater than about 130° C., or greater than about 140° C., or greater than about 150° C., or greater than about 160° C., or greater than about 170° C., or greater than about 180° C., or greater than about 190° C., or greater than about 200° C., or greater than about 210° C., or greater than about 220° C., or greater than about 230° C.

In an embodiment, the common drying temperatures for the processes of the disclosure may be less than about 110° C., or less than about 115° C., or less than about 120° C., or less than about 125° C., or less than about 130° C., or less than about 135° C., or less than about 140° C., or less than about 145° C., or less than about 150° C. In an embodiment, the common drying temperatures for the processes of the disclosure may be greater than about 110° C., or greater than about 115° C., or greater than about 120° C., or greater than about 125° C., or greater than about 130° C., or greater than about 135° C., or greater than about 140° C., or greater than about 145° C., or greater than about 150° C.

In some embodiments, a recombinant silk coated material (e.g., fabric) may be heat resistant to a selected temperature where the selected temperature is chosen for drying, curing, and/or heat setting a dye that may be applied to the material (e.g., LYCRA). As used herein, a “heat resistant” may refer to a property of the recombinant silk coating deposited on the material where the recombinant silk coating and/or recombinant silk protein does not exhibit a substantial modification (i.e., “substantially modifying”) in recombinant silk coating performance as compared to a control material having a comparable recombinant silk coating that was not subjected to the selected temperature for drying, curing, wash cycling, and/or heat setting purposes. In some embodiments, the selected temperature is the glass transition temperature (Tg) for the material upon which the recombinant silk coating is applied. In some embodiments, the selected temperature is greater than about 65° C., or greater than about 70° C., or greater than about 80° C., or greater than about 90° C., or greater than about 100° C., or greater than about 110° C., or greater than about 120° C., or greater than about 130° C., or greater than about 140° C., or greater than about 150° C., or greater than about 160° C., or greater than about 170° C., or greater than about 180° C., or greater than about 190° C., or greater than about 200° C., or greater than about 210° C., or greater than about 220° C. In some embodiments, the selected temperature is less than about 65° C., or less than about 70° C., or less than about 80° C., or less than about 90° C., or less than about 100° C., or less than about 110° C., or less than about 120° C., or less than about 130° C., or less than about 140° C., or less than about 150° C., or less than about 160° C., or less than about 170° C., or less than about 180° C., or less than about 190° C., or less than about 200° C., or less than about 210° C., or less than about 220° C.

In an embodiment, “substantially modifying” recombinant silk coating performance may be a decrease in a selected property of recombinant silk coating, such as wetting time, absorption rate, spreading speed, accumulative one-way transport, or overall moisture management capability as compared to a control recombinant silk coating that was not subjected to the selected temperature for drying, curing, wash cycling, and/or heat setting purposes, where such decrease is less than about a 1% decrease, or less than about a 2% decrease, or less than about a 3% decrease, or less than about a 4% decrease, or less than about a 5% decrease, or less than about a 6% decrease, or less than about a 7% decrease, or less than about an 8% decrease, or less than about a 9% decrease, or less than about a 10% decrease, or less than about a 15% decrease, or less than about a 20% decrease, or less than about a 25% decrease, or less than about a 30% decrease, or less than about a 35% decrease, or less than about a 40% decrease, or less than about a 45% decrease, or less than about a 50% decrease, or less than about a 60% decrease, or less than about a 70% decrease, or less than about a 80% decrease, or less than about a 90% decrease, or less than about 100% decrease in wetting time, absorption rate, spreading speed, accumulative one-way transport, or overall moisture management capability as compared to a control recombinant silk coating that was not subjected to the selected temperature for drying, curing, wash cycling, and/or heat setting purposes. In some embodiments, “wash cycling” may refer to at least one wash cycle, or at least two wash cycles, or at least three wash cycles, or at least four wash cycles, or at least five wash cycles.

In an embodiment, “substantially modifying” recombinant silk coating performance may be an increase in a selected property of recombinant silk coating, such as wetting time, absorption rate, spreading speed, accumulative one-way transport, or overall moisture management capability as compared to a control recombinant silk coating that was not subjected to the selected temperature for drying, curing, wash cycling, and/or heat setting purposes, where such increase is less than about a 1% increase, or less than about a 2% increase, or less than about a 3% increase, or less than about a 4% increase, or less than about a 5% increase, or less than about a 6% increase, or less than about a 7% increase, or less than about an 8% increase, or less than about a 9% increase, or less than about a 10% increase, or less than about a 15% increase, or less than about a 20% increase, or less than about a 25% increase, or less than about a 30% increase, or less than about a 35% increase, or less than about a 40% increase, or less than about a 45% increase, or less than about a 50% increase, or less than about a 60% increase, or less than about a 70% increase, or less than about a 80% increase, or less than about a 90% increase, or less than about 100% increase in wetting time, absorption rate, spreading speed, accumulative one-way transport, or overall moisture management capability as compared to a control recombinant silk coating that was not subjected to the selected temperature for drying, curing, wash cycling, and/or heat setting purposes. In some embodiments, “wash cycling” may refer to at least one wash cycle, or at least two wash cycles, or at least three wash cycles, or at least four wash cycles, or at least five wash cycles.

In some embodiments, the RSS coated article may be subjected to heat setting in order to set one or more dyes that may be applied to the RSS coated article in order to permanently set the one or more dyes on the RSS coated article. In some embodiments, the RSS coated article may be heat setting resistant, wherein the RSS coating on the RSS coated article may resist a heat setting temperature of greater than about 100° C., or greater than about 110° C., or greater than about 120° C., or greater than about 130° C., or greater than about 140° C., or greater than about 150° C., or greater than about 160° C., or greater than about 170° C., or greater than about 180° C., or greater than about 190° C., or greater than about 200° C., or greater than about 210° C., or greater than about 220° C. In some embodiments, the selected temperature is less than about 100° C., or less than about 110° C., or less than about 120° C., or less than about 130° C., or less than about 140° C., or less than about 150° C., or less than about 160° C., or less than about 170° C., or less than about 180° C., or less than about 190° C., or less than about 200° C., or less than about 210° C., or less than about 220° C.

In an embodiment, a material coated by the recombinant silk coating as described herein may partially dissolved or otherwise partially incorporated within a portion of the material after the recombinant silk coated material is subjected to heating and/or curing as described herein. Without being limited to any one theory of the disclosure, where the recombinant silk coated material is heated to greater than about the glass transition temperature (Tg) for the material that is coated, the recombinant silk coating may become partially dissolved or otherwise partially incorporated within a portion of the material.

In some embodiments, a material coated by the recombinant silk coating as described herein may be sterile or may be sterilized to provide a sterilized recombinant silk coated material. Alternatively, or in addition thereto, the methods described herein may include a sterile RSS prepared from sterile recombinant silk. In some embodiments, the fabric constructions that are compatible with the processes of the disclosure include woven fabrics, knitted fabrics, and non-woven fabrics.

In some embodiments, the coating pattern provided by the processes of the disclosure include one side coating, two side coating, and/or throughout coating. In some embodiments, the equipment manufacturers that are capable of producing equipment configured to continuously coat RSS on textiles include, but are not limited to, Aigle, Amba Projex, Bombi, Bruckner, Cavitec, Crosta, Dienes Apparatebau, Eastsign, Europlasma, Fermor, Fontanet, Gaston Systems, Hansa Mixer, Harish, Has Group, Icomatex, Idealtech, Interspare, Isotex, Klieverik, KTP, M P, Mageba, Mahr Feinpruef, Matex, Mathis, Menzel LP, Meyer, Monforts, Morrison Textile, Mtex, Muller Frick, Muratex Textile, Reliant Machinery, Rollmac, Salvade, Sandvik Tps, Santex, Chmitt-Machinen, Schott & Meissner, Sellers, Sicam, Siltex, Starlinger, Swatik Group India, Techfull, TMT Manenti, Unitech Textile Machinery, Weko, Willy, Wumag Texroll, Yamuna, Zappa, and Zimmer Austria.

In some embodiments, the equipment manufactures that are capable of producing equipment configured to dry RSS coated on textiles include, but are not limited to, Alea, Alkan Makina, Anglada, Atac Makina, Bianco, Bruckner, Campen, CHTC, CTMTC, Dilmenler, Elteksmak, Erbatech, Fontanet, Harish, Icomatex, Ilsung, Inspiron, Interspare, Master, Mathis, Monfongs, Monforts, Salvade, Schmitt-Maschinen, Sellers, Sicam, Siltex, Swastik Group India, Tacome, Tubetex, Turbang, Unitech Textile Machinery, and Yamuna.

In some embodiments, RSS may be used in combination with chemical agents. In some embodiments, RSS may include a chemical agent. In some embodiments, a chemical agent may be applied to a textile to be coated prior to providing an RSS coating. In some embodiments, a chemical agent may be applied to a textile after such textile has been coated with an RSS coating. One or more chemical agents may be applied, as set forth above, and may include a first chemical agent, second chemical agent, third chemical agent, and the like, where the chemical agents may be the same or a combination of two or more of the chemical agents described herein. In some embodiments, chemical agents may provide selected properties to coated textile (e.g., fabric) including, but not limited to, an antimicrobial property, an antiodor property, a water repellant property, an oil repellant property, a coloring property, a flame retardant property, a fabric softening property, a pH adjusting property, an anticrocking property, an antipilling property, and/or an antifelting property. In some embodiments, chemical agents may include, but are not limited to, an antimicrobial agent, acidic agents (e.g., Brønsted acids, citric acid, acetic acid, etc.), a softener, a water repellant agent, an oil repellant agent, a dye, a flame retardant, a fabric softener, a pH adjusting agent (e.g., an acidic agent), an anticrocking agent, an antipilling agent, and/or an antifelting agent. Such chemical agents may include, but are not limited to, softeners (e.g., chemical fabric softeners), acidic agents, antimicrobials, dyes, finishing agents including monomers (e.g., melted polyester), and combinations thereof.

In an embodiment, a selected property of the RSS coated articles that may be enhanced as compared to non-coated articles may include one or more of dimensional stability to laundering, dimensional stability to dry cleaning, appearance after laundering, appearance after dry cleaning, colorfastness to laundering, colorfastness to dry cleaning, colorfastness to non-chlorine bleach, seam torque/spirality (on knits), colorfastness to crocking, colorfastness to rubbing, colorfastness to water, colorfastness to light, colorfastness to perspiration, colorfastness to chlorinated pool water, colorfastness to sea water, tensile strength, seam slippage, tearing strength, seam breaking strength, abrasion resistance, pilling resistance, stretch recovery, bursting strength, colorfastness to die transfer in storage (labels), colorfastness to ozone, pile retention, bowing and skewing, colorfastness to saliva, snagging resistance, wrinkle resistance (e.g., appearance of apparel, retention of creases in fabrics, smooth appearance of fabrics), water repellency, water resistance, stain repellant (e.g., water repellency, oil repellency, water/alcohol repellency), vertical wicking, water absorption, dry rate, soil release, air permeability, wicking, antimicrobial properties, ultraviolet protection, resistance to torque, malodor resistant, biocompatibility, wetting time, absorption rate, spreading speed, accumulative one-way transport, flame retardant properties, coloring properties, fabric softening properties, a pH adjusting property, an antifelting property, and overall moisture management capability.

In some embodiments, RSS may be used in an RSS coating, where such coating includes one or more chemical agents (e.g., a silicone). RSS may be provided in such an RSS coating at a concentration by weight (% w/w or % w/v) or by volume (v/v) of less than about 25%, or less than about 20%, or less than about 15%, or less than about 10%, or less than about 9%, or less than about 8%, or less than about 7%, or less than about 6%, or less than about 5%, or less than about 4%, or less than about 3%, or less than about 2%, or less than about 1%, or less than about 0.9%, or less than about 0.8%, or less than about 0.7%, or less than about 0.6%, or less than about 0.5%, or less than about 0.4%, or less than about 0.3%, or less than about 0.2%, or less than about 0.1%, or less than about 0.01%, or less than about 0.001%. In some embodiments, RSS may be provided in such an RSS coating at a concentration by weight (% w/w or % w/v) or by volume (v/v) of greater than about 25%, or greater than about 20%, or greater than about 15%, or greater than about 10%, or greater than about 9%, or greater than about 8%, or greater than about 7%, or greater than about 6%, or greater than about 5%, or greater than about 4%, or greater than about 3%, or greater than about 2%, or greater than about 1%, or greater than about 0.9%, or greater than about 0.8%, or greater than about 0.7%, or greater than about 0.6%, or greater than about 0.5%, or greater than about 0.4%, or greater than about 0.3%, or greater than about 0.2%, or greater than about 0.1%, or greater than about 0.01%, or greater than about 0.001%.

In some embodiments, chemical fabric softeners may include silicones as described herein.

In some embodiments, the chemical agents may include the following, which are supplied by CHT Bezema and are associated with certain selected textile (e.g., fabric) properties, which may be used to strengthen RSS binding on coated surfaces and/or RSS may be used for enhancing the following chemical agents' properties:

ALPAPRINT CLEAR
Silicone printing and coating
Component B is mentioned in the technical leaflet
Dry handle
Good rubbing fastness
Good washfastness

ALPAPRINT ELASTIC ADD
Silicone printing and coating
Component B is mentioned in the technical leaflet
Good rubbing fastness
Good washfastness
Suited for yardage printing

ALPAPRINT WHITE
Silicone printing and coating
Component B is mentioned in the technical leaflet
Dry handle
Good rubbing fastness
Good washfastness

ALPATEC 30142 A
Textile finishing
Coating
Silicone printing and coating
Component B is mentioned in the technical leaflet
Suitable for narrow ribbon coating
Good rubbing fastness
Good washfastness

ALPATEC 30143 A
Silicone printing and coating
Component B is mentioned in the technical leaflet
Good rubbing fastness
Good washfastness
Suited for yardage printing

ALPATEC 30191 A
Silicone printing and coating
Component B is mentioned in the technical leaflet
Suitable for narrow ribbon coating
High transparency
Coating

ALPATEC 30203 A
Silicone printing and coating
Component B is mentioned in the technical leaflet
Suitable for narrow ribbon coating
High transparency
Coating

ALPATEC 3040 LSR KOMP. A
Functional coatings, Silicone printing and coating
Component B is mentioned in the technical leaflet
High abrasion resistance
High transparency
Coating

ALPATEC 3060 LSR KOMP. A
Functional coatings, Silicone printing and coating
Component B is mentioned in the technical leaflet
High abrasion resistance
High transparency
Coating

ALPATEC 530
Silicone printing and coating
Suitable for narrow ribbon coating
High transparency
Coating
One component system

ALPATEC 540
Silicone printing and coating
Suitable for narrow ribbon coating
High transparency
Coating
One component system

ALPATEC 545
Silicone printing and coating
Suitable for narrow ribbon coating
High transparency
Coating
One component system

ALPATEC 550
Silicone printing and coating
Suitable for narrow ribbon coating
High transparency
Coating
One component system

ALPATEC 730
Silicone printing and coating
Suitable for narrow ribbon coating
Good washfastness
High abrasion resistance
High transparency

ALPATEC 740
Silicone printing and coating
Suitable for narrow ribbon coating
Good washfastness
High abrasion resistance
High transparency

ALPATEC 745
Silicone printing and coating
Suitable for narrow ribbon coating
Good washfastness
High abrasion resistance
High transparency

ALPATEC 750
Silicone printing and coating
Suitable for narrow ribbon coating
Good washfastness
High abrasion resistance
High transparency

ALPATEC BANDAGE A
Silicone printing and coating
Component B is mentioned in the technical leaflet
Suitable for narrow ribbon coating
Coating
Two component system

APYROL BASE2 E
Flame retardants
Liquid
Soft handle
For BS 5852/1+2
Suited for paste coating
APYROL FCR-2
Water repellency/oil repellency
Cationic
High effectiveness
Water-based
Liquid

APYROL FFD E
Flame retardants
Liquid
Suited for polyester
Suited for polyamide
Flame inhibiting filler

APYROL FR CONC E
Flame retardants, Functional coatings
Liquid
Suited for polyester
Suited for polyamide
Flame inhibiting filler

APYROL GBO-E
Flame retardants, Functional coatings
Suited for polyester
Black-out coating
For DIN 4102/B1
Containing halogen

APYROL LV 21
Flame retardants, Functional coatings
For DIN 4102/B1
Suited for paste coating
Suited for backcoating of black-out vertical blinds and roller blinds
Containing halogen

APYROL PP 31
Flame retardants
Liquid
Free from antimony
Flame inhibiting filler
For BS 5852/1+2

APYROL PP 46
Flame retardants
Powder
Free from antimony
Flame inhibiting filler
Suited for paste coating

APYROL PREM E
Flame retardants
Soft handle
For BS 5852/1+2
Containing halogen
Semi-permanent

APYROL PREM2 E
Flame retardants
Soft handle
For BS 5852/1+2
Containing halogen
Semi-permanent

COLORDUR 005 WHITE
Flock adhesives, Functional coatings, Silicone printing and coating
Based on silicone
Dyestuff pigment suspension

COLORDUR 105 LEMON
Flock adhesives, Functional coatings, Silicone printing and coating
Based on silicone
Dyestuff pigment suspension

COLORDUR 115 GOLDEN YELLOW

Flock adhesives, Functional coatings, Silicone printing and coating
Based on silicone
Dyestuff pigment suspension

COLORDUR 185 ORANGE
Flock adhesives, Functional coatings, Silicone printing and coating
Based on silicone
Dyestuff pigment suspension

COLORDUR 215 RED
Flock adhesives, Functional coatings, Silicone printing and coating
Based on silicone
Dyestuff pigment suspension

COLORDUR 225 DARK RED
Flock adhesives, Functional coatings, Silicone printing and coating
Based on silicone
Dyestuff pigment suspension

COLORDUR 285 VIOLET
Flock adhesives, Functional coatings, Silicone printing and coating
Based on silicone
Dyestuff pigment suspension

COLORDUR 305 BLUE
Flock adhesives, Functional coatings, Silicone printing and coating
Based on silicone
Dyestuff pigment suspension

COLORDUR 355 MARINE
Flock adhesives, Functional coatings, Silicone printing and coating
Based on silicone
Dyestuff pigment suspension

COLORDUR 405 GREEN
Flock adhesives, Functional coatings, Silicone printing and coating
Based on silicone
Dyestuff pigment suspension

COLORDUR 465 OLIVE GREEN
Flock adhesives, Functional coatings, Silicone printing and coating
Based on silicone
Dyestuff pigment suspension

COLORDUR 705 BLACK
Flock adhesives, Functional coatings, Silicone printing and coating
Based on silicone
Dyestuff pigment suspension

COLORDUR AM ADDITIVE
Flock adhesives, Silicone printing and coating
Based on silicone
Migration prevention
Dyestuff pigment suspension

COLORDUR FL 1015 YELLOW
Flock adhesives, Functional coatings, Silicone printing and coating
Based on silicone
Dyestuff pigment suspension

COLORDUR FL 1815 ORANGE
Flock adhesives, Functional coatings, Silicone printing and coating
Based on silicone
Dyestuff pigment suspension

COLORDUR FL 2415 PINK
Flock adhesives, Functional coatings, Silicone printing and coating
Based on silicone
Dyestuff pigment suspension

COLORDUR FL 4015 GREEN
Flock adhesives, Functional coatings, Silicone printing and coating
Based on silicone
Dyestuff pigment suspension

ECOPERL 1
Water repellency/oil repellency
Washfast
Sprayable
Based on special functionalised polymers/waxes
Cationic

ECOPERL ACTIVE
Water repellency/oil repellency
Washfast
Based on special functionalised polymers/waxes
Cationic
High effectiveness

LAMETHAN 1 ET 25 BR 160
Functional coatings, Lamination
Washfast
Transparent
25 μm strong
Film based on polyester urethane

LAMETHAN ADH-1
Functional coatings, Lamination
Breathable
Suited for dry laminating
Good stability to washing at 40° C.
Stable foam adhesive

LAMETHAN ADH-L
Functional coatings, Lamination
Washfast
Transparent
Suited for paste coating
Suited for wet laminating

### LAMETHAN ALF-K

Functional coatings, Lamination
Adhesive additive for bondings
Suited for dry laminating
Stable foam adhesive
Suited for stable foam coating

### LAMETHAN LB 15-T BR 152DK

Functional coatings, Lamination
Transparent
15 nm strong
Breathable
Suited for dry laminating

LAMETHAN LB 25 BR 155
Functional coatings, Lamination
Transparent
25 nm strong
Suited for dry laminating
Good stability to washing at 40° C.

LAMETHAN LB 25 W BR 152
Lamination
25 nm strong
Breathable
Suited for dry laminating
Good stability to washing at 40° C.

LAMETHAN TAPE DE 80
Functional coatings, Lamination
Polymer base: polyurethane
Transparent
Good stability to washing at 40° C.
Tape for seam sealing

LAMETHAN TAPE ME 160
Functional coatings, Lamination
Polymer base: polyurethane
Transparent
Good stability to washing at 40° C.
Tape for seam sealing

LAMETHAN VL-H920 0 BR150
Functional coatings, Lamination
Two coats with membrane and PES charmeuse
Breathable
Suited for dry laminating
Good stability to washing at 40° C.

LAMETHAN VL-H920 S BR 150
Functional coatings, Lamination
Two coats with membrane and PES charmeuse
Breathable
Suited for dry laminating
Good stability to washing at 40° C.

LAMETHAN VL-H920 W BR150
Functional coatings, Lamination
Two coats with membrane and PES charmeuse
Breathable
Suited for dry laminating
Good stability to washing at 40° C.

TUBICOAT A 12 E
Binders, Functional coatings
Anionic
Liquid
Formaldehyde-free
Polymer base: polyacrylate

TUBICOAT A 17
Binders, Functional coatings
Suitable for tablecloth coating
Anionic
Liquid
Self-crosslinking

TUBICOAT A 19
Binders, Functional coatings
Washfast
Anionic
Formaldehyde-free
Good stability to washing

TUBICOAT A 22
Binders, Functional coatings
Washfast
Medium-hard film
Anionic
Liquid

TUBICOAT A 23
Binders
Medium-hard film
Anionic
Liquid
Application for varying the handle

TUBICOAT A 28
Binders, Functional coatings
Anionic
Liquid
Formaldehyde-free
Good stability to washing

TUBICOAT A 36
Binders, Functional coatings
Washfast
Anionic
Liquid
Low formaldehyde

TUBICOAT A 37
Binders, Functional coatings
Washfast
Suitable for tablecloth coating
Anionic
Liquid

TUBICOAT A 41
Binders, Functional coatings
Anionic
Liquid
Self-crosslinking
Good fastnesses

TUBICOAT A 61
Binders, Functional coatings
Suitable for tablecloth coating
Liquid
Non-ionic
Self-crosslinking

TUBICOAT A 94
Binders, Functional coatings
Anionic
Liquid
Self-crosslinking
Good fastnesses

TUBICOAT AIB 20
Fashion coatings
Transparent
Suited for foam coating
Pearl Gloss Finish

TUBICOAT AOS
Foaming auxiliaries
Non-ionic
Foaming
Suited for the fluorocarbon finishing

TUBICOAT ASK
Functional coatings, Lamination
Adhesive additive for bondings
Transparent
Suited for paste coating
Suited for dry laminating

TUBICOAT B-H
Binders, Functional coatings
Polymer base: Styrene butadiene
Anionic
Liquid
Formaldehyde-free

TUBICOAT B 45
Binders, Functional coatings
Washfast
Polymer base: Styrene butadiene
Anionic
Liquid

TUBICOAT BO-NB
Functional coatings
Medium hard
Suited for black-out coating
Good flexibility at low temperatures
Suited for stable foam coating

TUBICOAT BO-W
Functional coatings
Suited for black-out coating
Impermeable for light
Suited for stable foam coating
Water vapour permeable

TUBICOAT BOS
Foaming auxiliaries
Anionic
Foaming
Foam stabilizer

TUBICOAT DW-FI
Functional coatings, Special products
Anionic
Suited for coating pastes
Suited for stable foam
Foamable

TUBICOAT E 4
Binders
Anionic
Self-crosslinking
Low formaldehyde
Polymer base: polyethylene vinyl acetate

TUBICOAT ELC
Functional coatings
Suited for paste coating
Black
Electrically conductive
Soft

TUBICOAT EMULGATOR HF
Functional coatings, Special products
Anionic
Dispersing
Suited for coating pastes
Suited for stable foam

TUBICOAT ENTSCHAUMER N
Defoamers and deaerators
Liquid
Non-ionic
Silicone-free
Suited for coating pastes

TUBICOAT FIX FC
Fixing agents
Cationic
Water-based
Liquid
Formaldehyde-free

TUBICOAT FIX ICB CONC.
Fixing agents
Liquid
Non-ionic
Formaldehyde-free
Suited for crosslinking

TUBICOAT FIXIERER AZ
Fixing agents
Liquid
Suited for crosslinking
Based on polyaziridin
Unblocked

TUBICOAT FIXIERER FA
Fixing agents
Anionic
Water-based
Liquid
Low formaldehyde

TUBICOAT FIXIERER H 24
Fixing agents
Anionic
Water-based
Liquid
Formaldehyde-free

TUBICOAT FIXIERER HT
Fixing agents
Water-based
Liquid
Non-ionic
Suited for crosslinking

TUBICOAT FOAMER NY
Foaming auxiliaries
Non-ionic
Foaming
Suited for the fluorocarbon finishing
Non-yellowing

TUBICOAT GC PU
Fashion coatings
Washfast
Soft handle
Polymer base: polyurethane
Transparent

TUBICOAT GRIP
Functional coatings
Slip resistant
Suited for stable foam coating
Soft

TUBICOAT HEC
Thickeners
Powder
Non-ionic
Stable to electrolytes
Stable to shear forces

TUBICOAT HOP-S
Special products
Anionic
Suited for coating pastes
Coating
Adhesion promoter

TUBICOAT HS 8
Binders
Anionic
Liquid
Formaldehyde-free
Hard film

TUBICOAT HWS-1
Functional coatings
Suited for paste coating
Water-proof
Suited for giant umbrellas and tents

TUBICOAT KL-TOP F
Fashion coatings, Functional coatings
Washfast
Polymer base: polyurethane
Transparent
Suited for paste coating

TUBICOAT KLS-M
Fashion coatings, Functional coatings
Washfast
Soft handle
Polymer base: polyurethane
Breathable

TUBICOAT MAF
Fashion coatings
Washfast
Matrix effect
Improves the rubbing fastnesses
Soft handle

TUBICOAT MD TC 70
Fashion coatings
Vintage wax
Suited for foam coating
Suited for topcoats

TUBICOAT MEA
Functional coatings
Washfast
Polymer base: polyurethane
Suited for paste coating
Suited for topcoat coatings

TUBICOAT MG-R
Fashion coatings
Washfast
Soft handle
Suited for paste coating
Duo Leather Finish

TUBICOAT MOP NEU
Functional coatings, Special products
Washfast
Anionic
Foamable
Finish

TUBICOAT MP-D
Fashion coatings, Functional coatings
Washfast
Soft handle
Medium hard
Breathable

TUBICOAT MP-W
Functional coatings
Washfast
Polymer base: polyurethane
Breathable
Water-proof

TUBICOAT NTC-SG
Functional coatings
Washfast
Transparent
Suited for paste coating
Medium hard

TUBICOAT PERL A22-20
Fashion coatings
Suited for paste coating
Suited for foam coating
Pearl Gloss Finish

TUBICOAT PERL HS-1
Functional coatings
Suited for paste coating
Suited for black-out coating
Suited for pearlescent coating
Suited for topcoat coatings

TUBICOAT PERL PU SOFT
Fashion coatings
Washfast
Scarabaeus effect
Soft handle
Polymer base: polyurethane

TUBICOAT PERL VC CONC.
Fashion coatings, Functional coatings
Soft handle
Polymer base: polyurethane
Suited for paste coating
Suited for black-out coating

TUBICOAT PHV
Functional coatings
Medium hard
Suited for three-dimensional dot coating

TUBICOAT PSA 1731
Functional coatings, Lamination
Transparent
Suited for paste coating
Suited for dry laminating
Non-breathable

TUBICOAT PU-UV
Binders
Anionic
Liquid
Formaldehyde-free
Good fastnesses

TUBICOAT PU 60
Binders
Anionic
Liquid
Application for varying the handle
Formaldehyde-free

TUBICOAT PU 80
Binders, Functional coatings
Washfast
Anionic
Liquid
Can be washed off

TUBICOAT PUH-BI
Binders
Anionic
Liquid
Formaldehyde-free
Hard film

TUBICOAT PUL
Functional coatings
Polymer base: polyurethane
Suited for paste coating
Suited for three-dimensional dot coating
Slip resistant

TUBICOAT PUS
Binders, Functional coatings
Anionic
Liquid
Formaldehyde-free
Polymer base: polyurethane

TUBICOAT PUW-M
Binders
Medium-hard film
Anionic
Liquid
Formaldehyde-free

TUBICOAT PUW-S
Binders
Anionic
Liquid
Formaldehyde-free
Good stability to washing

TUBICOAT PW 14
Binders, Functional coatings
Anionic
Formaldehyde-free
Heat-sealable
Not wetting

TUBICOAT SA-M
Functional coatings
Washfast
Suited for paste coating
Suited for three-dimensional dot coating

TUBICOAT SCHAUMER HP
Foaming auxiliaries, Functional coatings
Non-ionic
Foaming
Suited for the fluorocarbon finishing

TUBICOAT SF-BASE
Fashion coatings
Washfast
Soft handle
Suited for foam coating
Silk gloss effect

TUBICOAT SHM
Foaming auxiliaries
Anionic
Foam stabilizer

TUBICOAT SI 55
Special products
Pseudo-cationic
Suited for coating pastes
Foamable
Coating

TUBICOAT STABILISATOR RP
Foaming auxiliaries
Anionic
Foam stabilizer

TUBICOAT STC 100
Fashion coatings, Functional coatings
Transparent
Breathable
Suited for stable foam coating

TUBICOAT STC 150
Fashion coatings, Functional coatings
Washfast
Soft handle
Transparent
Breathable

TUBICOAT STL
Functional coatings
Washfast
Slip resistant
Suited for stable foam coating
Soft

TUBICOAT TCT
Fashion coatings, Functional coatings
Washfast
Polymer base: polyurethane
Transparent
Suited for paste coating

TUBICOAT VA 10
Binders
Anionic
Liquid
Formaldehyde-free
Hard film

TUBICOAT VCP
Functional coatings
Suited for paste coating
Medium hard
Suited for black-out coating

TUBICOAT VERDICKER 17
Thickeners
Anionic
High efficiency
Synthetic

TUBICOAT VERDICKER ASD
Thickeners
Anionic
Quick swelling
Stable to shear forces
Pseudoplastic

TUBICOAT VERDICKER LP
Thickeners
Anionic
Stable to shear forces
Pseudoplastic
Dispersible

TUBICOAT VERDICKER PRA
Thickeners
Anionic
Liquid
Stable to electrolytes
Rheological additive

TUBICOAT WBH 36
Special products
Finish
Application for preventing roller deposits

TUBICOAT WBV
Special products
Non-ionic
Finish
Application for preventing roller deposits

TUBICOAT WEISS EU
Functional coatings, Special products
Suited for coating pastes
Suited for stable foam
Suited for topcoat coatings
Titanium dioxide paste

TUBICOAT WLI-LT KONZ
Functional coatings
Washfast
Suited for paste coating
Slip resistant
Soft

TUBICOAT WLI
Fashion coatings, Functional coatings
Washfast
Scarabaeus effect

Soft handle
Suited for paste coating
TUBICOAT WOT
Fashion coatings
Washfast
Soft handle
Suited for paste coating
Wash-out effect

TUBICOAT WX-TCA 70
Fashion coatings, Functional coatings
Vintage wax
Suited for paste coating
Suited for topcoat coatings

TUBICOAT WX BASE
Fashion coatings
Vintage wax
Soft handle
Suited for paste coating
Application in the prime coat

TUBICOAT ZP NEU
Water repellency/oil repellency
Zircon-paraffine base
Suited for aqueous systems
Cationic
Foamable

TUBIGUARD 10-F
Water repellency/oil repellency
Washfast
Sprayable
Cationic
Liquid

TUBIGUARD 21
Water repellency/oil repellency
Washfast
Cationic
High effectiveness
Water-based

TUBIGUARD 25-F
Water repellency/oil repellency
Washfast
Sprayable
Cationic
High effectiveness

TUBIGUARD 270
Functional coatings, Water repellency/oil repellency
Washfast
Cationic
High effectiveness
Liquid

TUBIGUARD 30-F
Water repellency/oil repellency
Washfast
Sprayable
Cationic
High effectiveness

TUBIGUARD 44 N
Water repellency/oil repellency
Washfast
Sprayable
Suited for aqueous systems
Liquid

TUBIGUARD 44N-F
Water repellency/oil repellency
Suited for aqueous systems
Non-ionic
Suited for polyester
Foamable

TUBIGUARD 66
Water repellency/oil repellency
Washfast
Sprayable
High effectiveness
Liquid

TUBIGUARD 90-F
Water repellency/oil repellency
Washfast
Cationic
High effectiveness
Liquid

TUBIGUARD AN-F
Water repellency/oil repellency
Washfast
Sprayable
Cationic
High effectiveness

TUBIGUARD FA2-F
Water repellency/oil repellency
Sprayable
Cationic
Suited for polyester
Foamable

TUBIGUARD PC3-F
Functional coatings, Water repellency/oil repellency
Washfast
Cationic
Liquid
Paste

TUBIGUARD SR 2010-F W
Water repellency/oil repellency
Cationic
High effectiveness
Foamable
Based on C6 fluorocarbon

In some embodiments, the chemical agents may include the following, which are supplied by CHT Bezema and are associated with certain selected textile (e.g., fabric) properties, which may be used to strengthen RSS binding to inkjet printing dye:

CHT-ALGINAT MVU
Ink jet printing preparation, Thickeners
Cationic
Powder
Anionic
High colour brilliance

PRISULON CR-F 50
Ink jet printing preparation, Thickeners
Liquid
Good outlines
High surface levelness
Good penetration

TUBIJET DU 01
Ink jet printing preparation
Antimigrant
Anionic
Liquid
Formaldehyde-free

TUBIJET NWA
Ink jet printing preparation
Liquid
Non-ionic
Without impact on the handle
Formaldehyde-free

TUBIJET PUS
Ink jet printing preparation
Film forming
Anionic
Liquid
Formaldehyde-free

TUBIJET VDK
Ink jet printing preparation
Liquid
Formaldehyde-free
Halogen-free
Flame protection effect

TUBIJET WET
Ink jet printing preparation
Anionic
Liquid
Without impact on the handle
Formaldehyde-free

In some embodiments, the chemical agents of the disclosure may include the following inkjet printing dyes, which are supplied by CHT Bezema and are associated with certain selected textile (e.g., fabric) properties, which may be used in combination with RSS:

BEZAFLUOR BLUE BB
Pigments
High Performance
BEZAFLUOR (fluorescent pigments)

BEZAFLUOR GREEN BT
Pigments
High Performance
BEZAFLUOR (fluorescent pigments)

BEZAFLUOR ORANGE R
Pigments
High Performance
BEZAFLUOR (fluorescent pigments)

BEZAFLUOR PINK BB
Pigments
High Performance
BEZAFLUOR (fluorescent pigments)
BEZAFLUOR RED R
Pigments
High Performance
BEZAFLUOR (fluorescent pigments)

BEZAFLUOR VIOLET BR
Pigments
High Performance
BEZAFLUOR (fluorescent pigments) BEZAFLUOR YELLOW BA
Pigments
High Performance
BEZAFLUOR (fluorescent pigments)

BEZAPRINT BLACK BDC
Pigments
Advanced
BEZAPRINT (classic pigments)

BEZAPRINT BLACK DT
Pigments
Advanced
BEZAPRINT (classic pigments)

BEZAPRINT BLACK DW
Pigments
Advanced
BEZAPRINT (classic pigments)

BEZAPRINT BLACK GOT
Pigments
High Performance
BEZAKTIV GOT (GOTS)

BEZAPRINT BLUE BN
Pigments
Advanced
BEZAPRINT (classic pigments)

BEZAPRINT BLUE BT
Pigments
Advanced
BEZAPRINT (classic pigments)

BEZAPRINT BLUE GOT
Pigments
High Performance
BEZAKTIV GOT (GOTS)

BEZAPRINT BLUE RR
Pigments
Advanced
BEZAPRINT (classic pigments)

BEZAPRINT BLUE RT
Pigments
Advanced
BEZAPRINT (classic pigments)

BEZAPRINT BLUE RTM
Pigments
Advanced
BEZAPRINT (classic pigments)

BEZAPRINT BLUE TB
Pigments
Advanced
BEZAPRINT (classic pigments)

BEZAPRINT BORDEAUX K2R
Pigments
Advanced
BEZAPRINT (classic pigments)

BEZAPRINT BROWN RP
Pigments
Advanced
BEZAPRINT (classic pigments)

BEZAPRINT BROWN TM
Pigments
Advanced
BEZAPRINT (classic pigments)

BEZAPRINT CITRON 10G
Pigments
Advanced
BEZAPRINT (classic pigments)

BEZAPRINT CITRON GOT
Pigments
High Performance
BEZAKTIV GOT (GOTS)

BEZAPRINT GREEN 2B
Pigments
Advanced
BEZAPRINT (classic pigments)

BEZAPRINT GREEN BS
Pigments
Advanced
BEZAPRINT (classic pigments)

BEZAPRINT GREEN BT
Pigments
Advanced
BEZAPRINT (classic pigments)

BEZAPRINT GREY BB
Pigments
Advanced
BEZAPRINT (classic pigments)

BEZAPRINT NAVY GOT
Pigments
High Performance
BEZAKTIV GOT (GOTS)

BEZAPRINT NAVY RRM
Pigments
Advanced
BEZAPRINT (classic pigments)

BEZAPRINT NAVY TR
Pigments
Advanced
BEZAPRINT (classic pigments)

BEZAPRINT OLIVE GREEN BT
Pigments
Advanced
BEZAPRINT (classic pigments)

BEZAPRINT ORANGE 2G
Pigments
Advanced
BEZAPRINT (classic pigments)

BEZAPRINT ORANGE GOT
Pigments
High Performance
BEZAKTIV GOT (GOTS)

BEZAPRINT ORANGE GT
Pigments
Advanced
BEZAPRINT (classic pigments)

BEZAPRINT ORANGE RG
Pigments
Advanced
BEZAPRINT (classic pigments)

BEZAPRINT PINK BW
Pigments
Advanced
BEZAPRINT (classic pigments)

BEZAPRINT RED 2BN
Pigments
Advanced
BEZAPRINT (classic pigments)

BEZAPRINT RED GOT
Pigments
High Performance
BEZAKTIV GOT (GOTS)

BEZAPRINT RED KF
Pigments
Advanced
BEZAPRINT (classic pigments)

BEZAPRINT RED KGC
Pigments
Advanced
BEZAPRINT (classic pigments)

BEZAPRINT SCARLET GRL
Pigments
Advanced
BEZAPRINT (classic pigments)

BEZAPRINT SCARLET RR
Pigments
Advanced
BEZAPRINT (classic pigments)

BEZAPRINT TURQUOISE GT
Pigments
Advanced
BEZAPRINT (classic pigments)

BEZAPRINT VIOLET FB
Pigments
Advanced
BEZAPRINT (classic pigments)

BEZAPRINT VIOLET KB
Pigments
Advanced
BEZAPRINT (classic pigments)

BEZAPRINT VIOLET R
Pigments
Advanced
BEZAPRINT (classic pigments)

BEZAPRINT VIOLET TN
Pigments
Advanced
BEZAPRINT (classic pigments)

BEZAPRINT YELLOW 2GN
Pigments
Advanced
BEZAPRINT (classic pigments)

BEZAPRINT YELLOW 3GT
Pigments
Advanced
BEZAPRINT (classic pigments)

BEZAPRINT YELLOW 4RM
Pigments
Advanced
BEZAPRINT (classic pigments)

BEZAPRINT YELLOW GOT
Pigments
High Performance
BEZAKTIV GOT (GOTS)

BEZAPRINT YELLOW RR
Pigments
Advanced
BEZAPRINT (classic pigments)

In some embodiments, the chemical agents of the disclosure may include the following, which are supplied by Lamberti SPA and are associated with certain selected textile (e.g., fabric) properties, which may be used to strengthen RSS binding on coated surfaces or RSS may be used for enhancing such chemical agent properties:

### Pre Treatment:

Waterborne Polyurethanes Dispersions
Rolflex AFP.
   Aliphatic polyether polyurethane dispersion in water. The product has high hydrolysis resistance, good breaking load resistance and excellent tear resistance.
Rolflex ACF.
   Aliphatic polycarbonate polyurethane dispersion in water. The product shows good PU and PVC bonding properties, excellent abrasion resistance as well as chemical resistance, included alcohol.
Rolflex V 13.
   Aliphatic polyether/acrylic copolymer polyurethane dispersion in water. The product has good thermoadhesive properties and good adhesion properties on PVC.
Rolflex K 80.
   Aliphatic polyether/acrylic copolymer polyurethane dispersion in water. ROLFLEX K 80 is specifically designed as a high performing adhesive for textile lamination. The product has excellent perchloroethylene and water fastness.
Rolflex ABC.
   Aliphatic polyether polyurethane dispersion in water. Particularly, the product presents very high water column, excellent electrolytes resistance, high LOI index, high resistance to multiple bending.
Rolflex ADH.
   Aliphatic polyether polyurethane dispersion in water. The product has a very high water column resistance.
Rolflex W4.
   Aliphatic waterborned PU dispersion particularly suggested for the formulation of textile coatings for clothing, outwear where a full, soft and non sticky touch is required.
Rolflex ZB7.
   Aliphatic waterborned PU dispersion particularly suggested for the formulation of textile coatings for clothing, outwear, sportswear, fashion and technical articles for industrial applications. The product has a very high charge digestion properties, electrolites stability and excellent mechanical and tear resistance. Can be also suitable for foam coating and printing application.
Rolflex BZ 78.
   Aliphatic waterborned PU dispersion particularly suggested for the formulation of textile coatings for clothing, outwear, sportswear, fashion and technical articles for industrial applications. The product has an excellent hydrolysis resistance, a very high charge digestion and electrolites stability and an excellent mechanical and tear resistance. Can be also suitable for foam coating and printing application.
Rolflex PU 147.
   Aliphatic polyether polyurethane dispersion in water. This product shows good film forming properties at room temperature. It has high fastness to light and ultraviolet radiation and good resistance to water, solvent and chemical agents, as well as mechanical resistance.
Rolflex SG.
   Aliphatic polyether polyurethane dispersion in water. Due to its thermoplastic properties it is suggested to formulate heat activated adhesives at low temperatures.
Elafix PV 4.
   Aliphatic blocked isocyanate Nano-dispersion used in order to give antifelting and antipilling properties to pure wool fabrics and his blend.
Rolflex C 86.
   Aliphatic cationic waterborned PU dispersion particularly suggested for the formulation of textile coatings for clothing, outwear, fashion where medium-soft and pleasant full touch is required. Fabrics treated with the product can be dyed with a selection of dyes, to get double-color effects of different intensity.
Rolflex CN 29.
   Aliphatic cationic waterborned PU dispersion particularly suggested for the formulation of textile coatings for clothing, outwear, fashion where soft and pleasant full touch is required. Fabrics treated with the product can be dyed with a selection of dyes, to get double-color effects of different intensity.

### Oil and Water Repellents

Lamgard FT 60.
   General purpose fluorocarbon resin for water and oil repellency; by padding application.
Lamgard 48.
   High performance fluorocarbon resin for water and oil repellency; by padding application. High rubbing fastness.
Imbitex NRW3
   Wetting agent for water- and oil repellent finishing.
Lamgard EXT.
   Crosslinker for fluorocarbon resins to improve washing fastness.

Flame Retardants
Piroflam 712.
   Non-permanent flame retardant compound for padding and spray application.
Piroflam ECO.
   Alogen free flame retardant compound for back coating application for all kind of fibers.
Piroflam UBC.
   Flame retardant compound for back coating application for all kind of fibers.

Crosslinkers
Rolflex BK8.
   Aromatic blocked polyisocyanate in water dispersion. It is suggested as a cross-linking agent in coating pastes based of polyurethane resins to improve washing fastness.
Fissativo 05.
   Water dispersible aliphatic polyisocyanate suitable as crosslinking agent for acrylic and polyurethane dispersions to improve adhesion and wet and dry scrub resistance.
Resina MEL.
   Melammine-formaldheyde resin.
Cellofix VLF.
   Low formaldehyde melamine resin.

Thickeners
Lambicol CL 60.
   Fully neutralised synthetic thickener for pigment printing in oil/water emulsion; medium viscosity type
Viscolam PU conc.
   Nonionic polyurethane based thickener with pseudoplastic behavior
Viscolam 115 new.
   Acrylic thickener not neutralised
Viscolam PS 202.
   Nonionic polyurethane based thickener with newtonian behavior
Viscolam 1022.
   Nonionic polyurethane based thickener with moderate pseudoplastic behavior.

### Dyeing

Dispersing agents
Lamegal BO.
   Liquid dispersing agent non ionic, suitable for direct, reactive, disperse dyeing and PES stripping
Lamegal DSP.
   Dispersing/anti back-staining agent in preparation, dyeing and soaping of dyed and printed materials. Antioligomer agent.
Lamegal 619.
   Effective low foam dispersing leveling agent for dyeing of PES
Lamegal TLS.
   Multi-purpose sequestring and dispersing agent for all kind of textile process

Levelling agents
Lamegal A 12.
   Leveling agent for dyeing on wool, polyamide and its blends with acid or metalcomplex dyes

Fixing Agents
Lamfix L.
   Fixing agent for direct and reactive dyestuffs, containing formaldheyde
Lamfix LU conc.
   Formaldehyde free cationic fixing agent for direct and reactive dyes. It does not affect the shade and light fastness.
Lamfix PA/TR.
   Fixing agent to improve the wet fastness of acid dyes on polyamide fabrics, dyed or printed and polyamide yarns. Retarding agent in dyeing of Polyamide/cellulosic blends with direct dyes.

Special Resins
Denifast TC.
   Special resin for cationization of cellulose fibers to obtain special effects (“DENIFAST system” and “DENISOL system”).
Cobral DD/50.
   Special resin for cationization of cellulose fibers to obtain special effect (“DENIFAST system” and “DENISOL system”).

Antireducing Agents
Lamberti Redox L2S gra.
   Anti-reducing agent in grain form. 100% active content
Lamberti Redox L2S liq.
   Anti-reducing agent in liquid form for automatic dosage.

Anticreasing agent
Lubisol AM.
   Lubricating and anti creasing agent for rope wet operation on all kind of fibers and machines.

### Pigment Dye

Antimigrating agent
Neopat Compound 96/m conc.
   Compound, developed as migration inhibitor for continuous dyeing process with pigments (pad-dry process).

Binding Agent
Neopat Binder PM/S conc.
   Concentrated version of a specific binder used to prepare pad-liquor for dyeing with pigments (pad-dry process).

All in One Agent
Neopat Compound PK1.
   High concentrated compound specifically developed as migration inhibitor with specific binder for continuous dyeing process with pigments (pad-dry process) all in one

Dela\grave{v}e Agent
Neopat compound FTN.
   High concentrated compound of surfactants and polymers specifically developed for pigment dyeing and pigment-reactive dyeing process; especially for medium/dark shades for wash off effect

### Traditional Finishing Agents

Wrinkle Free Treatment
Cellofix ULF conc.
   Anti-crease modified glyoxalic resin for finishing of cottons, cellulosics and blend with synthetics fibers.
Poliflex PO 40.
   Polyethilenic resin for waxy, full and slippy handle by foulard applications.
Rolflex WF.
   Aliphatic waterborned Nano-PU dispersion used as extender for wrinkle free treatments.

Softeners
Texamina C/FPN.
   Cationic softening agent with a very soft handle particularly recommended for application by exhaustion for all kind of fabrics. Suitable also for cone application.
Texamina C SAL flakes.
   100% cationic softening agent in flakes form for all type of fabrics. Dispersible at room temperature.
Texamina CL LIQ.
   Anphoteric softening agent for all types of fabrics. Not yellowing.
Texamina HVO.
   Anphoteric softening agent for woven and knitted fabrics of cotton, other cellulosics and blends. Gives a soft, smooth and dry handle. Applied by padding.
Texamina SIL.
   Nonionic silicon dispersion in water. Excellent softening, lubricating and anti-static properties for all fibre types by padding.
Texamina SILK.
   Special cationic softener with silk protein inside. Gives a “swollen touch” particularly suitable for cellulosic, wool, silk.
Lamfinish LW.
   All-in compound based on special polymeric hydrophilic softeners; by coating, foulard, and exhaustion.
Elastolam E50.
   General purpose mono-component silicone elastomeric softener for textile finishing.
Elastolam EC 100.
   Modified polysiloxane micro-emulsion which gives a permanent finishing, with extremely soft and silky handle.

Handle Modifier
Poliflex CSW.
   Cationic anti-slipping agent.
Poliflex R 75.
   Parafine finishing agent to give waxy handle.
Poliflex s.
   Compound specifically developed for special writing effects.
Poliflex m.
   Compound for special dry-waxy handle.
Lamsoft SW 24.
   Compound for special slippy handle specifically developed for coating application.
Lamfinish SLIPPY.
   All-in compound to get a slippy touch; by coating.
Lamfinish GUMMY.
   All-in compound to get a gummy touch; by coating.
Lamfinish OLDRY.
   All-in compound to get dry-sandy touch especially suitable for vintage effects; by coating

Waterborne Polyurethanes Dispersions
Rolflex LB 2.
   Aliphatic waterborned PU dispersion particularly suggested for the formulation of textile coatings where bright and rigid top finish is required. It is particularly suitable as a finishing agent for organza touch on silk fabrics. Transparent and shiny.
Rolflex HP 51.
   Aliphatic waterborned PU dispersion particularly suggested for the formulation of textile coatings for outwear, luggage, technical articles especially where hard and flexible touch is required. Transparent and shiny.
Rolflex PU 879.
   Aliphatic waterborned PU dispersion particularly suggested for the formulation of textile coatings for outwear, luggage, technical articles where a medium-hard and flexible touch is required.
Rolflex ALM.
   Aliphatic waterborned PU dispersion particularly suggested for the formulation of textile coatings for outwear, luggage, technical articles where a soft and flexible touch is required. Can be also suitable for printing application.
Rolflex AP.
   Aliphatic waterborned PU dispersion particularly suggested for the formulation of textile coatings for outwear, fashion where a soft and gummy touch is required.
Rolflex W4.
   Aliphatic waterborned PU dispersion particularly suggested for the formulation of textile coatings for clothing, outwear where a full, soft and non sticky touch is required.
Rolflex ZB7.
   Aliphatic waterborned PU dispersion particularly suggested for the formulation of textile coatings for clothing, outwear, sportswear, fashion and technical articles for industrial applications. The product has a very high charge digestion properties, electrolites stability and excellent mechanical and tear resistance. Can be also suitable for foam coating and printing application.
Rolflex BZ 78.
   Aliphatic waterborned PU dispersion particularly suggested for the formulation of textile coatings for clothing, outwear, sportswear, fashion and technical articles for industrial applications. The product has an excellent hydrolysis resistance, a very high charge digestion and electrolites stability and an excellent mechanical and tear resistance. Can be also suitable for foam coating and printing application.
Rolflex K 110.
   Gives to the coated fabric a full, soft, and slightly sticky handle with excellent fastness on all types of fabrics.
Rolflex OP 80.
   Aliphatic waterborned PU dispersion particularly suggested for the formulation of textile coatings for outwear, luggage and fashion finishes where an opaque non writing effect is desired.
Rolflex NBC.
   Aliphatic waterborned PU dispersion generally used by padding application as a filling and zero formaldheyde sizing agent. Can be used for outwear and fashion finishings where a full, elastic and non sticky touch is required.
Rolflex PAD.
   Aliphatic waterborned PU dispersion specifically designed for padding application for outwear, sportswear and fashion applications where a full, elastic and non sticky touch is required. Excellent washing and dry cleaning fastness as well as good bath stability.
Rolflex PN.
   Aliphatic waterborned PU dispersion generally applied by padding application for outerwear and fashion high quality applications where strong, elastic non sticky finishes are required.
Elafix PV 4.
   Aliphatic blocked isocyanate Nano-dispersion used in order to give antifelting and antipilling properties to pure wool fabrics and his blend.
Rolflex SW3.
   Aliphatic waterborned PU dispersion particularly suggested to be used by padding application for the finishing of outwear, sportswear and fashion where a slippery and elastic touch is required. It is also a good antipilling agent. Excellent in wool application.
Rolflex C 86.
   Aliphatic cationic waterborned PU dispersion particularly suggested for the formulation of textile coatings for clothing, outwear, fashion where medium-soft and pleasant full touch is required. Fabrics treated with the product can be dyed with a selection of dyes, to get double-color effects of different intensity.
Rolflex CN 29.
   Aliphatic cationic waterborned PU dispersion particularly suggested for the formulation of textile coatings for clothing, outwear, fashion where soft and pleasant full touch is required. Fabrics treated with the product can be dyed with a selection of Dyes, to Get Double-Color Effects of Different Intensity.

Other Resins
Textol 110.
   Handle modifier with very soft handle for coating finishes
Textol RGD.
   Water emulsion of acrylic copolymer for textile coating, with very rigid handle.
Textol SB 21.
   Butadienic resin for finishing and binder for textile printing
Appretto PV/CC.
   Vinylacetate water dispersion for rigid stiffening
Amisolo B.
   CMS water dispersion for textile finishing as stiffening agent
Lamovil RP.
   PVOH stabilized solution as stiffening agent

### Technical Finishing Agents

Waterborne Polyurethanes Dispersions
Rolflex AFP.
   Aliphatic polyether polyurethane dispersion in water. The product has high hydrolysis resistance, good breaking load resistance and excellent tear resistance.
Rolflex ACF.
   Aliphatic polycarbonate polyurethane dispersion in water. The product shows good PU and PVC bonding properties, excellent abrasion resistance as well as chemical resistance, included alcohol.
Rolflex V 13.
   Aliphatic polyether/acrylic copolymer polyurethane dispersion in water. The product has good thermoadhesive properties and good adhesion properties on PVC.
Rolflex K 80.
   Aliphatic polyether/acrylic copolymer polyurethane dispersion in water. ROLFLEX K 80 is specifically designed as a high performing adhesive for textile lamination. The product has excellent perchloroethylene and water fastness.
Rolflex ABC.
   Aliphatic polyether polyurethane dispersion in water. Particularly, the product presents very high water column, excellent electrolytes resistance, high LOI index, high resistance to multiple bending.
Rolflex ADH.
   Aliphatic polyether polyurethane dispersion in water. The product has a very high water column resistance.
Rolflex W4.
   Aliphatic waterborned PU dispersion particularly suggested for the formulation of textile coatings for clothing, outwear where a full, soft and non sticky touch is required.
Rolflex ZB7.
   Aliphatic waterborned PU dispersion particularly suggested for the formulation of textile coatings for clothing, outwear, sportswear, fashion and technical articles for industrial applications. The product has a very high charge digestion properties, electrolites stability and excellent mechanical and tear resistance. Can be also suitable for foam coating and printing application.
Rolflex BZ 78.
   Aliphatic waterborned PU dispersion particularly suggested for the formulation of textile coatings for clothing, outwear, sportswear, fashion and technical articles for industrial applications. The product has an excellent hydrolysis resistance, a very high charge digestion and electrolites stability and an excellent mechanical and tear resistance. Can be also suitable for foam coating and printing application.
Rolflex PU 147.
   Aliphatic polyether polyurethane dispersion in water. This product shows good film forming properties at room temperature. It has high fastness to light and ultraviolet radiation and good resistance to water, solvent and chemical agents, as well as mechanical resistance.
Rolflex SG.
   Aliphatic polyether polyurethane dispersion in water. Due to its thermoplastic properties it is suggested to formulate heat activated adhesives at low temperatures.
Elafix PV 4.
   Aliphatic blocked isocyanate Nano-dispersion used in order to give antifelting and antipilling properties to pure wool fabrics and his blend.
Rolflex C 86.
   Aliphatic cationic waterborned PU dispersion particularly suggested for the formulation of textile coatings for clothing, outwear, fashion where medium-soft and pleasant full touch is required. Fabrics treated with the product can be dyed with a selection of dyes, to get double-color effects of different intensity.
Rolflex CN 29.
   Aliphatic cationic waterborned PU dispersion particularly suggested for the formulation of textile coatings for clothing, outwear, fashion where soft and pleasant full touch is required. Fabrics treated with the product can be dyed with a selection of dyes, to get double-color effects of different intensity.

Oil and Water Repellents
Lamgard FT 60.
   General purpose fluorocarbon resin for water and oil repellency; by padding application.
Lamgard 48.
   High performance fluorocarbon resin for water and oil repellency; by padding application. High rubbing fastness.
Imbitex NRW3.
   Wetting agent for water- and oil repellent finishing.
Lamgard EXT.
   Crosslinker for fluorocarbon resins to improve washing fastness.

Flame Retardants
Piroflam 712.
   Non-permanent flame retardant compound for padding and spray application.
Piroflam ECO.
   Alogen free flame retardant compound for back coating application for all kind of fibers.
Piroflam UBC.
   Flame retardant compound for back coating application for all kind of fibers.

Crosslinkers
Rolflex BK8.
   Aromatic blocked polyisocyanate in water dispersion. It is suggested as a cross-linking agent in coating pastes based of polyurethane resins to improve washing fastness.
Fissativo 05.
   Water dispersible aliphatic polyisocyanate suitable as crosslinking agent for acrylic and polyurethane dispersions to improve adhesion and wet and dry scrub resistance.
Resina MEL.
   Melammine-formaldheyde resin.
Cellofix VLF.
   Low formaldehyde melamine resin.

Thickeners
Lambicol CL 60.
   Fully neutralized synthetic thickener for pigment printing in oil/water emulsion; medium viscosity type
Viscolam PU conc.
   Nonionic polyurethane based thickener with pseudoplastic behavior
Viscolam 115 new.
   Acrylic thickener not neutralized
Viscolam PS 202.
   Nonionic polyurethane based thickener with newtonian behavior
Viscolam 1022.
   Nonionic polyurethane based thickener with moderate pseudoplastic behavior.

In some embodiments, the chemical agent may include one or more of a silicone, an acidic agent, a dyeing agent, a pigment dye, a traditional finishing agent, and a technical finishing agent. The dyeing agent may include one or more of a dispersing agent, a levelling agent, a fixing agent, a special resin, an antireducing agent, and an anticreasing agent. The pigment dye may include one or more of an antimigrating agent, a binding agent, an all in one agent, and a delave agent. The traditional finishing agent may include one or more of a wrinkle free treatment, a softener, a handle modifier, a waterborne polyurethanes dispersion, and other resins. The technical finishing agent may include one or more of a waterborne polyurethanes dispersion, an oil repellant, a water repellant, a crosslinker, and a thickener.

In some embodiments, certain chemical agents of the disclosure may be provided by one or more of the following chemical suppliers: Adrasa, AcHitex Minerva, Akkim, Archroma, Asutex, Avocet dyes, BCC India, Bozzetto group, CHT, Clearity, Dilube, Dystar, Eksoy, Erca group, Genkim, Giovannelli e Figli, Graf Chemie, Huntsman, KDN Bio, Lamberti, LJ Specialties, Marlateks, Montegauno,

Protex, Pulcra Chemicals, Ran Chemicals, Fratelli Ricci, Ronkimya, Sarex, Setas, Silitex, Soko Chimica, Tanatex Chemicals, Zaitex, Zetaesseti, and Z Schimmer.

In some embodiments, the chemical agent may include an acidic agent. Accordingly, in some embodiments, RSS may include an acidic agent. In some embodiments, an acidic agent may be a Brønsted acid. In an embodiment, the acidic agent includes one or more of citric acid and acetic acid. In an embodiment, the acidic agent aids the deposition and coating of RSPF mixtures (i.e., RSS coating) on the textile to be coated as compared to the absence of such acidic agent. In an embodiment, the acidic agent improves crystallization of the RSPF mixtures at the textile to be coated.

In an embodiment, the acidic agent is added at a concentration by weight (% w/w or % w/v) or by volume (v/v) of greater than about 0.001%, or greater than about 0.002%, or greater than about 0.003%, or greater than about 0.004%, or greater than about 0.005%, or greater than about 0.006%, or greater than about 0.007%, or greater than about 0.008%, or greater than about 0.009%, or greater than about 0.01%, or greater than about 0.02%, or greater than about 0.03%, or greater than about 0.04%, or greater than about 0.05%, or greater than about 0.06%, or greater than about 0.07%, or greater than about 0.08%, or greater than about 0.09%, or greater than about 0.1%, or greater than about 0.2%, or greater than about 0.3%, or greater than about 0.4%, or greater than about 0.5%, or greater than about 0.6%, or greater than about 0.7%, or greater than about 0.8%, or greater than about 0.9%, or greater than about 1.0% or greater than about 2.0%, or greater than about 3.0%, or greater than about 4.0%, or greater than about 5.0%.

In an embodiment, the acidic agent is added at a concentration by weight (% w/w or % w/v) or by volume (v/v) of less than about 0.001%, or less than about 0.002%, or less than about 0.003%, or less than about 0.004%, or less than about 0.005%, or less than about 0.006%, or less than about 0.007%, or less than about 0.008%, or less than about 0.009%, or less than about 0.01%, or less than about 0.02%, or less than about 0.03%, or less than about 0.04%, or less than about 0.05%, or less than about 0.06%, or less than about 0.07%, or less than about 0.08%, or less than about 0.09%, or less than about 0.1%, or less than about 0.2%, or less than about 0.3%, or less than about 0.4%, or less than about 0.5%, or less than about 0.6%, or less than about 0.7%, or less than about 0.8%, or less than about 0.9%, or less than about 1.0% or less than about 2.0%, or less than about 3.0%, or less than about 4.0%, or less than about 5.0%.

In some embodiments, RSS may have a pH of less than about 9, or less than about 8.5, or less than about 8, or less than about 7.5, or less than about 7, or less than about 6.5, or less than about 6, or less than about 5.5, or less than about 5, or less than about 4.5, or less than about 4, or greater than about 3.5, or greater than about 4, or greater than about 4.5, or greater than about 5, or greater than about 5.5, or greater than about 6, or greater than about 6.5, or greater than about 7, or greater than about 7.5, or greater than about 8, or greater than about 8.5.

In some embodiments, RSS may include an acidic agent, and may have a pH of less than about 9, or less than about 8.5, or less than about 8, or less than about 7.5, or less than about 7, or less than about 6.5, or less than about 6, or less than about 5.5, or less than about 5, or less than about 4.5, or less than about 4, or greater than about 3.5, or greater than about 4, or greater than about 4.5, or greater than about 5, or greater than about 5.5, or greater than about 6, or greater than about 6.5, or greater than about 7, or greater than about 7.5, or greater than about 8, or greater than about 8.5.

In an embodiment, the chemical agent may include silicone. In some embodiments, a RSS may include silicone. In some embodiments, silicone may include a silicone emulsion. The term “silicone,” may generally refer to a broad family of synthetic polymers, mixtures of polymers, and/or emulsions thereof, that have a repeating silicon-oxygen backbone including, but not limited to, polysiloxanes. For example, a silicone may include ULTRATEX® CSP, which is a commercially available (Huntsman International LLC) silicone emulsion that may be used as a softening agent and which may also increase fabric resilience, elasticity of knitted fabrics, and fiber lubrication and also improve sewability. A silicone may also include ULTRATEX® CI, which is a commercially available silicone composition

(Huntsman International LLC) that may be used as a fabric softening agent. In some embodiments, a silicone may include any silicone species disclosed herein.

Describing the compositions and coatings more broadly, silicone may be used, for example to improve fabric hand, but may also increase the water repellency (or reduce water transport properties) of a fabric coated with silicone. Silicone may be used in combination with RSS to counteract the water repellant (water transport) properties of silicone.

In some embodiments, RSS may include silicone in a concentration by weight (% w/w or % w/v) or by volume (v/v) of less than about 25%, or less than about 20%, or less than about 15%, or less than about 10%, or less than about 9%, or less than about 8%, or less than about 7%, or less than about 6%, or less than about 5%, or less than about 4%, or less than about 3%, or less than about 2%, or less than about 1%, or less than about 0.9%, or less than about 0.8%, or less than about 0.7%, or less than about 0.6%, or less than about 0.5%, or less than about 0.4%, or less than about 0.3%, or less than about 0.2%, or less than about 0.1%, or less than about 0.01%, or less than about 0.001%.

In some embodiments, RSS may include silicone in a concentration by weight (% w/w or % w/v) or by volume (v/v) of greater than about 25%, or greater than about 20%, or greater than about 15%, or greater than about 10%, or greater than about 9%, or greater than about 8%, or greater than about 7%, or greater than about 6%, or greater than about 5%, or greater than about 4%, or greater than about 3%, or greater than about 2%, or greater than about 1%, or greater than about 0.9%, or greater than about 0.8%, or greater than about 0.7%, or greater than about 0.6%, or greater than about 0.5%, or greater than about 0.4%, or greater than about 0.3%, or greater than about 0.2%, or greater than about 0.1%, or greater than about 0.01%, or greater than about 0.001%.

In some embodiments, RSS may be supplied in a concentrated form suspended in water. In some embodiments, RSS may have a concentration by weight (% w/w or % w/v) or by volume (v/v) of less than about 50%, or less than about 45%, or less than about 40%, or less than about 35%, or less than about 30%, or less than about 25%, or less than about 20%, or less than about 15%, or less than about 10%, or less than about 5%, or less than about 4%, or less than about 3%, or less than about 2%, or less than about 1%, or less than about 0.1%, or less than about 0.01%, or less than about 0.001%, or less than about 0.0001%, or less than about 0.00001%. In some embodiments, RSS may have a concentration by weight (% w/w or % w/v) or by volume (v/v) of greater than about 50%, or greater than about 45%, or greater than about 40%, or greater than about 35%, or greater than about 30%, or greater than about 25%, or greater than about 20%, or greater than about 15%, or greater than about 10%, or greater than about 5%, or greater than about 4%, or greater than about 3%, or greater than about 2%, or greater than about 1%, or greater than about 0.1%, or greater than about 0.01%, or greater than about 0.001%, or greater than about 0.0001%, or greater than about 0.00001%.

In some embodiments, an RSS coating may include RSS, as described herein. In some embodiments, RSS may include a silicone and/or an acidic agent. In some embodiments, RSS may include a silicone and an acidic agent. In some embodiments, the RSS may include a silicone, an acidic agent, and/or an additional chemical agent, wherein the additional chemical agent may be one or more of the chemical agents described herein. In some embodiments, RSS may include a silicone emulsion and an acidic agent, such as acetic acid or citric acid.

In some embodiments, the coating processes of the disclosure may include a finishing step for the resulting coated textiles. In some embodiments, the finishing or final finishing of the textiles (e.g., fabrics) that are coated with RSS under the processes of the disclosure may include sueding, steaming, brushing, polishing, compacting, raising, tigering, shearing, heatsetting, waxing, air jet, calendaring, pressing, shrinking, treatment with polymerizer, coating, lamination, and/or laser etching. In some embodiments, finishing of the RSS coated textiles may include treatment of the textiles with an AIRO® 24 dryer that may be used for continuous and open-width tumbling treatments of woven, non-woven, and knitted fabrics.

In some embodiments, a coated textile (e.g., a fabric) may be prepared by unrolling a fabric roll to prepare a piece of fabric. The perimeter of such fabric may be processed. For example, fabric may have dimensions of 35 cm×35 cm (13.5 inch×13.5 inch) with a tolerance of +/−1 cm (+/−0.4 inch). In some embodiments, every fabric sample may be massed on analytical balance by folding the fabric sample multiple times until it may be contained by a weighing boat on a balance. Each measurement may be recorded. In some embodiments, a coating process may be initiated by preparing a curing oven by setting a selected temperature therein. A padder laboratory unit may be turned on and the speed of said padder unit may be set to a selected velocity and the roller pressure may be adjusted to a selected pressure by operating a cam lever system and locking it in place once the desired pressure is achieved. A silk solution (i.e., RSS) may be poured into a bath (e.g., a stainless steel bath). After a fabric sample is submerged in the bath, it may be allowed to reach saturation, and the fabric sample may then be removed from the bath and laid between two rollers of the padder unit. The fabric sample as it is transported through the rollers it may be squeezed of excessive fluid as determined by the rollers' pressure. The fabric sample may then exit to the opposite side of the rollers. The resulting fabric sample may then be placed on top of the curing frame and may then be gently pushed one edge at a time to engage the fabric edges with frame pins. The frame may be placed in the drying and curing oven, with the door of said oven secured and kept closed for the drying and curing time. A timer may be started to alert when the drying and curing time has elapsed. When the timer signals completion of the curing process, the oven door is opened and a temperature sensor (e.g., an IR temperature sensor) may be used to measure the fabric sample surface temperature. The frame bearing the fabric sample may then be removed from the oven and placed on a cooling rack. The sample fabric may then be removed from the frame and weighed.

In some embodiments, the RSS coated textiles (e.g., fabrics) described herein may meet or exceed requirements established by the following Test Methods:

| Test Description | Test Method | Requirements |
|---|---|---|
| Dimensional Stability to Laundering | AATCC 135 | Maximum, Length: −3%, |
| | | Width: −3% |
| | | Maximum, Length: −3%, |
| | | Width: −5%, for twoway |
| | | Stretch Fabrics |
| | | Maximum, Length: −5%, |
| | | Width: −5%, for fourway |
| | | Stretch Fabrics |
| | | No Growth |
| Dimensional Stability to Dry Cleaning | AATCC 158 | Maximum, Length: −3%, |
| | | Width: −3% |
| | | Maximum, Length: −3%, |
| | | Width: −5%, for twoway |
| | | Stretch Fabrics |
| | | Maximum, Length: −5%, |
| | | Width: −5%, for fourway |
| | | Stretch Fabrics |
| | | No Growth |
| Pilling Resistance | ASTM D 3512 | Minimum 3.0 |
| Abrasion Resistance | ASTM D 4966 | No rupture to 10,000 |
| | | cycles (plain fabrics up to |
| | | 7.5 oz/yd²; or no rupture to |
| | | 15,000 cycles (plain |
| | | fabrics over 7.5 oz/yd²) |
| Tearing Strength | ASTM D 1424 | Shorts, Pants, Jeans, |
| | | Jackets, All Plus Size |
| | | Styles: 2.5 Lbs Minimum; |
| | | or |
| | | Blouse, Skirt Dress, |
| | | Lining, excluding plus |
| | | size styles: 1.5 Lbs |
| | | Minimum; or |
| | | Intimate: |
| | | <3 oz/yd²: Minimum |
| | | 1.51 bs; |
| | | 3~6 oz/yd²: Minimum 2.0 |
| | | lbs |
| | | >6 oz/yd²: Minimum 2.5 |
| | | lbs |
| Colorfastness to Laundering/ | AATCC 61, 2A | Color Change: Minimum |
| Colorfastness to Washing | | 4.0 |
| | | Staining: Minimum 3.0 |
| Colorfastness to Dry Cleaning | AATCC 132 | Color Change: Minimum |
| | | 4.0 |
| | | Staining: Minimum 3.0 |
| Colorfastness to Crocking/ | AATCC 8 | All except below - Dry: |
| Colorfastness to Rubbing | | Minimum 4.0; Wet: |
| | | Minimum 3.0; or |
| | | Dark Shades (black, red, |
| | | navy) - Dry: Minimum |
| | | 4.0; Wet: Minimum 2.5; |
| | | or |
| | | Indigos - Dry: Minimum |
| | | 3.0; Wet: Minimum 2.0; |
| | | or |
| | | Pigments - Dry: Minimum |
| | | 3.5; Wet: Minimum 2.5. |
| Colorfastness to Water | AATCC 107 | Color Change: Minimum |
| | | 4.0; Staining: Minimum |
| | | 3.0 |
| Colorfastness to Perspiration | AATCC 15 | Color Change: Minimum |
| | | 4.0; Staining: Minimum 3 |
| Colorfastness to Light | AATCC 16/20 AFU | Color Change: Minimum |
| | AATCC 16/5 AFU | 4.0 |
| pH Value | AATCC 81 | 4.0~8.5 or 4.0~7.5 |
| | | (children <36 months) |
| Antimicrobial | AATCC 147 | Original: 0% Bacterial |
| | | Growth 20 Washes: 0% |
| | | Bacterial Growth |
| | AATCC 100 | Minimum 99.9% |
| | | Reduction |
| | ASTM E 2149 | Original: Minimum |
| | | 99.9% Reduction |
| | | 20 Washes: Minimum |
| | | 80% Reduction |
| Wicking | AATCC 79 | 1.0 second or less |
| Water Repellency - Spray Test | AATCC 22 | Original: 100 Rating |
| | | After 3× Washes: |
| | | Minimum 70 Rating |
| Water Resistance - Rain Test | AATCC 35 | Maximum 1 gram on |
| | | original and after 3× |
| | | washes |
| Dimensional Stability to | AATCC 150 | Maximum, Length = −3%, |
| Laundering (Yoga Garment) | | Width = −3% |
| | | Maximum, Length = −3%, |
| | | Width = −5% for two-way |
| | | Stretch Fabrics |
| | | Maximum, Length = −5%, |
| | | Width = −5% for four-way |
| | | Stretch fabrics |
| | | No Distortion Between |
| | | Components |
| | | No Growth |
| Dimensional Stability to | AATCC 158 | Maximum, Length = −3%, |
| Dry Cleaning (Yoga Garment) | | Width = −3% |
| | | Maximum, Length = −3%, |
| | | Width = −5%, for two-way |
| | | Stretch Fabrics |
| | | Maximum, Length = −5%, |
| | | Width = −5%, for four-way |
| | | Stretch Fabrics |
| | | No Distortion Between |
| | | Components |
| | | No Growth |
| Pilling Resistance (Yoga Garment) | ASM D 3512 | Minimum 3.0 |
| Colorfastness to | AATCC 61, 2A | Color Change: Minimum |
| Laundering/Colorfastness | | 4.0 |
| to Washing (Yoga Garment) | | Staining: Minimum 3.0 |
| Colorfastness | AATCC 8 | General: Dry: Minimum |
| Crocking/Colorfastness to | | 4.0; Wet: Minimum 3.0; |
| Rubbing (Yoga Garment) | | For Dark Colors (Black, |
| | | Red, Navy): Wet: |
| | | Minimum 2.5 |
| | | Pigment: Dry: Minimum |
| | | 3.5; Wet: Minimum 2.5 |
| | | Indigos: Dry: Minimum |
| | | 3.0; Wet: Minimum 2.0 |
| Colorfastness to Water | AATCC 107 | Color Change: Minimum |
| (Yoga Garment) | | 4.0 |
| | | Staining: Minimum 3.0 |
| Colorfastness to Perspiration | AATCC 15 | Color Change: 4.0 or |
| (Yoga Garment) | | better |
| | | Staining: 3.0 or better |
| Colorfastness to Light | AATCC 16, 20 AFU/5 | Minimum 4.0, All, Except |
| (Yoga Garment) | AFU | Silk/Minimum 4.0, Silk |
| pH Value (Yoga Garment) | AATCC 81 | Children (>36 months) & |
| | | Adults: 4.0~8.5 |
| | | Children (<36 months): |
| | | 4.0~7.5 |

In some embodiments, the RSS coated textiles (e.g., fabrics) described herein may meet requirements established by the foregoing Test Methods. In some embodiments, the RSS coated textiles (e.g., fabrics) described herein may exceed the requirements established by the foregoing Test Methods. In some embodiments, the RSS coated textiles (e.g., fabrics) may have antiodor activity due to the RSS coating.

In some embodiments, the RSS coated textiles (e.g., fabrics) may have antimicrobial activity (e.g., antifungal and/or antibacterial activity) due to the RSS coating. In an embodiment, antibacterial activity may be determined by the ability of bacteria on the RSS coated textile's surface to be washed away from the RSS coated textile surface following one or more wash cycles, or two or more wash cycles, or three or more wash cycles, or four or more wash cycles, or five or more wash cycles, where the bacteria do not adhere to the surface of the RSS coated textile. In an embodiment, antibacterial activity may be determined by the ability of the RSS coating to reduce the quantity of the bacteria deposited on a surface of the RSS coated textile, wherein the RSS coating may reduce the quantity of the bacteria by greater than about 1%, or greater than about 2%, or greater than about 3%, or greater than about 4%, or greater than about 5%, or greater than about 10%, or greater than about 20%, or greater than about 30%, or greater than about 40%, or greater than about 50%, or greater than about 60%, or greater than about 70%, or greater than about 80%, or greater than about 90%, or greater than about 95%, or greater than about 96%, or greater than about 97%, or greater than about 98%, or greater than about 99%, or by about 100%. In an embodiment, antibacterial activity of the RSS coating on the coated textile may be determined by fluorescent activity (see, e.g., U.S. Pat. Nos. 5,089,395 and 5,968,762, the entirety of which are incorporated herein by reference). In an embodiment, antibacterial activity for an SFS coating may be determined by the ability of the RSS coating on a coated textile to break up colonies of bacteria that may be deposited on a surface of the coated textile. In an embodiment, antibacterial activity for an RSS coating may be determined by the ability of the RSS coating on a coated textile to: (a) prevent the formation of a bacterial biofilm on the coated textile; and/or (b) reduce the size of a bacterial biofilm on the coated textile.

In some embodiments, RSS may be coated upon a textile or other material having antimicrobial (e.g., antibacterial and/or antifungal) properties without interfering with such properties or otherwise inhibiting such properties.

In an embodiment, a textile may be coated with RSS to provide an RSS coated article. In some embodiments, the textile may include one or more of polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, and LYCRA (polyester-polyurethane copolymer, also known as SPANDEX and elastomer). In some embodiments, the textile may include LYCRA.

In some embodiments, the RSS coated article may have a crocking value of greater than 4 as determined by AATCC 8. In some embodiments, the RSS coated article may have a crocking value of greater than 4 as determined by AATCC 8, wherein the RSS coated article includes one or more of a silicone and an acidic agent. In some embodiments, the RSS coated article may have a crocking value of greater than 4 as determined by AATCC 8, wherein the RSS coated article includes a silicone.

In some embodiments, the RSS coated article may have an overall moisture management capability (OMMC) of greater than 0.3. In some embodiments, the RSS coated article may have an overall moisture management capability (OMMC) of greater than 0.3, wherein the RSS coated article includes one or more of a silicone and an acidic agent. In some embodiments, the RSS coated article may have an overall moisture management capability (OMMC) of greater than 0.3, wherein the RSS coated article includes a silicone.

In some embodiments, the RSS coated article may contain no sites for bacterial adhesion. In some embodiments, the RSS coated article may contain no sites for bacterial adhesion after heat treatment. In some embodiments, the RSS coated article may contain no sites for bacterial adhesion following a wash cycle with non-chlorinated bleach. In some embodiments, the RSS coated article may contain no bacteria after washing.

### EXAMPLES

The following examples are put forth so as to provide those of ordinary skill in the art with a complete disclosure and description of how to make and use the described embodiments, and are not intended to limit the scope of what the inventors regard as their disclosure nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Fragmentation of Silk Fibroin Proteins, Recombinant Silk Fibroin Fragments, and Fibroin-Like Proteins

Provided herein are methods for producing pure and highly scalable silk fibroin-protein fragment mixture solutions that may be used across multiple industries for a variety of applications. Without wishing to be bound by any particular theory, it is believed that these methods are equally applicable to fragmentation of recombinant silk proteins, and fragmentation of silk-like or fibroin-like proteins.

As used herein, the term “fibroin” includes silk worm fibroin and insect or spider silk protein. In an embodiment, fibroin is obtained from Bombyx mori. Raw silk from Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. Conversion of these insoluble silk fibroin fibrils into water-soluble silk fibroin protein fragments requires the addition of a concentrated neutral salt (e.g., 8-10 M lithium bromide), which interferes with inter- and intramolecular ionic and hydrogen bonding that would otherwise render the fibroin protein insoluble in water. Methods of making silk fibroin protein fragments, and/or compositions thereof, are known and are described for example in U.S. Pat. Nos. 9,187,538, 9,511,012, 9,517,191, 9,522,107, 9,522,108, 9,545,369, and 10,166,177.

In some embodiments, silk fibroin-protein based fragment (SPF) mixture solutions are obtained by dissolving raw unscoured, partially scoured, or scoured silkworm fibers with a neutral lithium bromide salt. The raw silkworm silks are processed under selected temperature and other conditions in order to remove any sericin and achieve the desired weight average molecular weight (Mw) and polydispersity (PD) of the fragment mixture. Selection of process parameters may be altered to achieve distinct final silk protein fragment characteristics depending upon the intended use. The resulting final fragment solution is silk fibroin protein fragments and water with parts per million (ppm) to non-detectable levels of process contaminants, levels acceptable in the pharmaceutical, medical and consumer eye care markets. The concentration, size and polydispersity of silk fibroin protein fragments in the solution may further be altered depending upon the desired use and performance requirements.

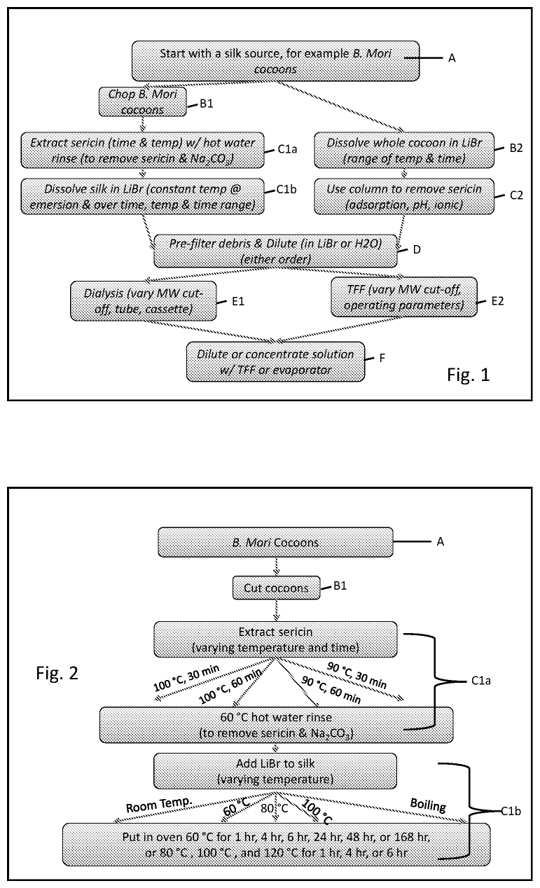

FIG. 1 is a flow chart showing various embodiments for producing pure silk fibroin protein fragments (SPFs) of the present disclosure. It should be understood that not all of the steps illustrated are necessarily required to fabricate all silk solutions of the present disclosure. As illustrated in FIG. 1, step A, cocoons (heat-treated or non-heat-treated), silk fibers, silk powder, spider silk or recombinant spider silk can be used as the silk source. If starting from raw silk cocoons from Bombyx mori, the cocoons can be cut into small pieces, for example pieces of approximately equal size, step B1. The raw silk is then extracted and rinsed to remove any sericin, step C1a. This results in substantially sericin free raw silk. In an embodiment, water is heated to a temperature between 84° C. and 100° C. (ideally boiling) and then Na₂CO₃ (sodium carbonate) is added to the boiling water until the Na₂CO₃ is completely dissolved. The raw silk is added to the boiling water/Na₂CO₃ (100° C.) and submerged for approximately 15-90 minutes, where boiling for a longer time results in smaller silk protein fragments. In an embodiment, the water volume equals about 0.4×raw silk weight and the Na₂CO₃ volume equals about 0.848×raw silk weight. In an embodiment, the water volume equals 0.1×raw silk weight and the Na₂CO₃ volume is maintained at 2.12 g/L.

Subsequently, the water dissolved Na₂CO₃ solution is drained and excess water/Na₂CO₃ is removed from the silk fibroin fibers (e.g., ring out the fibroin extract by hand, spin cycle using a machine, etc.). The resulting silk fibroin extract is rinsed with warm to hot water to remove any remaining adsorbed sericin or contaminate, typically at a temperature range of about 40° C. to about 80° C., changing the volume of water at least once (repeated for as many times as required). The resulting silk fibroin extract is a substantially sericin-depleted silk fibroin. In an embodiment, the resulting silk fibroin extract is rinsed with water at a temperature of about 60° C. In an embodiment, the volume of rinse water for each cycle equals 0.1 L to 0.2 L×raw silk weight. It may be advantageous to agitate, turn or circulate the rinse water to maximize the rinse effect. After rinsing, excess water is removed from the extracted silk fibroin fibers (e.g., ring out fibroin extract by hand or using a machine). Alternatively, methods known to one skilled in the art such as pressure, temperature, or other reagents or combinations thereof may be used for the purpose of sericin extraction. Alternatively, the silk gland (100% sericin free silk protein) can be removed directly from a worm. This would result in liquid silk protein, without any alteration of the protein structure, free of sericin.

The extracted fibroin fibers are then allowed to dry completely. Once dry, the extracted silk fibroin is dissolved using a solvent added to the silk fibroin at a temperature between ambient and boiling, step Clb. In an embodiment, the solvent is a solution of Lithium bromide (LiBr) (boiling for LiBr is 140° C.). Alternatively, the extracted fibroin fibers are not dried but wet and placed in the solvent; solvent concentration can then be varied to achieve similar concentrations as to when adding dried silk to the solvent. The final concentration of LiBr solvent can range from 0.1 M to 9.3 M. Complete dissolution of the extracted fibroin fibers can be achieved by varying the treatment time and temperature along with the concentration of dissolving solvent. Other solvents may be used including, but not limited to, phosphate phosphoric acid, calcium nitrate, calcium chloride solution or other concentrated aqueous solutions of inorganic salts. To ensure complete dissolution, the silk fibers should be fully immersed within the already heated solvent solution and then maintained at a temperature ranging from about 60° C. to about 140° C. for 1-168 hrs. In an embodiment, the silk fibers should be fully immersed within the solvent solution and then placed into a dry oven at a temperature of about 100° C. for about 1 hour.

The temperature at which the silk fibroin extract is added to the LiBr solution (or vice versa) has an effect on the time required to completely dissolve the fibroin and on the resulting molecular weight and polydispersity of the final SPF mixture solution. In an embodiment, silk solvent solution concentration is less than or equal to 20% w/v. In addition, agitation during introduction or dissolution may be used to facilitate dissolution at varying temperatures and concentrations. The temperature of the LiBr solution will provide control over the silk protein fragment mixture molecular weight and polydispersity created. In an embodiment, a higher temperature will more quickly dissolve the silk offering enhanced process scalability and mass production of silk solution. In an embodiment, using a LiBr solution heated to a temperature between 80° C.-140° C. reduces the time required in an oven in order to achieve full dissolution. Varying time and temperature at or above 60° C. of the dissolution solvent will alter and control the MW and polydispersity of the SPF mixture solutions formed from the original molecular weight of the native silk fibroin protein.

Alternatively, whole cocoons may be placed directly into a solvent, such as LiBr, bypassing extraction, step B2. This requires subsequent filtration of silk worm particles from the silk and solvent solution and sericin removal using methods know in the art for separating hydrophobic and hydrophilic proteins such as a column separation and/or chromatography, ion exchange, chemical precipitation with salt and/or pH, and or enzymatic digestion and filtration or extraction, all methods are common examples and without limitation for standard protein separation methods, step C2. Non-heat treated cocoons with the silkworm removed, may alternatively be placed into a solvent such as LiBr, bypassing extraction. The methods described above may be used for sericin separation, with the advantage that non-heat treated cocoons will contain significantly less worm debris.

Dialysis may be used to remove the dissolution solvent from the resulting dissolved fibroin protein fragment solution by dialyzing the solution against a volume of water, step E1. Pre-filtration prior to dialysis is helpful to remove any debris (i.e., silk worm remnants) from the silk and LiBr solution, step D. In one example, a 3 μm or 5 μm filter is used with a flow-rate of 200-300 mL/min to filter a 0.1% to 1.0% silk-LiBr solution prior to dialysis and potential concentration if desired. A method disclosed herein, as described above, is to use time and/or temperature to decrease the concentration from 9.3 M LiBr to a range from 0.1 M to 9.3 M to facilitate filtration and downstream dialysis, particularly when considering creating a scalable process method. Alternatively, without the use of additional time or temperate, a 9.3 M LiBr-silk protein fragment solution may be diluted with water to facilitate debris filtration and dialysis. The result of dissolution at the desired time and temperate filtration is a translucent particle-free room temperature shelf-stable silk protein fragment-LiBr solution of a known MW and polydispersity. It is advantageous to change the dialysis water regularly until the solvent has been removed (e.g., change water after 1 hour, 4 hours, and then every 12 hours for a total of 6 water changes). The total number of water volume changes may be varied based on the resulting concentration of solvent used for silk protein dissolution and fragmentation. After dialysis, the final silk solution maybe further filtered to remove any remaining debris (i.e., silk worm remnants).

Alternatively, Tangential Flow Filtration (TFF), which is a rapid and efficient method for the separation and purification of biomolecules, may be used to remove the solvent from the resulting dissolved fibroin solution, step E2. TFF offers a highly pure aqueous silk protein fragment solution and enables scalability of the process in order to produce large volumes of the solution in a controlled and repeatable manner. The silk and LiBr solution may be diluted prior to TFF (20% down to 0.1% silk in either water or LiBr). Pre-filtration as described above prior to TFF processing may maintain filter efficiency and potentially avoids the creation of silk gel boundary layers on the filter's surface as the result of the presence of debris particles. Pre-filtration prior to TFF is also helpful to remove any remaining debris (i.e., silk worm remnants) from the silk and LiBr solution that may cause spontaneous or long-term gelation of the resulting water only solution, step D. TFF, recirculating or single pass, may be used for the creation of water-silk protein fragment solutions ranging from 0.1% silk to 30.0% silk (more preferably, 0.1%-6.0% silk). Different cutoff size TFF membranes may be required based upon the desired concentration, molecular weight and polydispersity of the silk protein fragment mixture in solution. Membranes ranging from 1-100 kDa may be necessary for varying molecular weight silk solutions created for example by varying the length of extraction boil time or the time and temperate in dissolution solvent (e.g., LiBr). In an embodiment, a TFF 5 or 10 kDa membrane is used to purify the silk protein fragment mixture solution and to create the final desired silk-to-water ratio. As well, TFF single pass, TFF, and other methods known in the art, such as a falling film evaporator, may be used to concentrate the solution following removal of the dissolution solvent (e.g., LiBr) (with resulting desired concentration ranging from 0.1% to 30% silk). This can be used as an alternative to standard HFIP concentration methods known in the art to create a water-based solution. A larger pore membrane could also be utilized to filter out small silk protein fragments and to create a solution of higher molecular weight silk with and/or without tighter polydispersity values.

An assay for LiBr and Na₂CO₃ detection was performed using an HPLC system equipped with evaporative light scattering detector (ELSD). The calculation was performed by linear regression of the resulting peak areas for the analyte plotted against concentration. More than one sample of a number of formulations of the present disclosure was used for sample preparation and analysis. Generally, four samples of different formulations were weighed directly in a 10 mL volumetric flask. The samples were suspended in 5 mL of 20 mM ammonium formate (pH 3.0) and kept at 2-8° C. for 2 hours with occasional shaking to extract analytes from the film. After 2 hours the solution was diluted with 20 mM ammonium formate (pH 3.0). The sample solution from the volumetric flask was transferred into HPLC vials and injected into the HPLC-ELSD system for the estimation of sodium carbonate and lithium bromide.

The analytical method developed for the quantitation of Na₂CO₃ and LiBr in silk protein formulations was found to be linear in the range 10-165 pg/mL, with RSD for injection precision as 2% and 1% for area and 0.38% and 0.19% for retention time for sodium carbonate and lithium bromide respectively. The analytical method can be applied for the quantitative determination of sodium carbonate and lithium bromide in silk protein formulations.

In an embodiment, a SPF composition of the present disclosure is not soluble in an aqueous solution due to the crystallinity of the protein. In an embodiment, a SPF composition of the present disclosure is soluble in an aqueous solution. In an embodiment, the SPFs of a composition of the present disclosure include a crystalline portion of about two-thirds and an amorphous region of about one-third. In an embodiment, the SPFs of a composition of the present disclosure include a crystalline portion of about one-half and an amorphous region of about one-half. In an embodiment, the SPFs of a composition of the present disclosure include a 99% crystalline portion and a 1% amorphous region. In an embodiment, the SPFs of a composition of the present disclosure include a 95% crystalline portion and a 5% amorphous region. In an embodiment, the SPFs of a composition of the present disclosure include a 90% crystalline portion and a 10% amorphous region. In an embodiment, the SPFs of a composition of the present disclosure include a 85% crystalline portion and a 15% amorphous region. In an embodiment, the SPFs of a composition of the present disclosure include a 80% crystalline portion and a 20% amorphous region. In an embodiment, the SPFs of a composition of the present disclosure include a 75% crystalline portion and a 25% amorphous region. In an embodiment, the SPFs of a composition of the present disclosure include a 70% crystalline portion and a 30% amorphous region. In an embodiment, the SPFs of a composition of the present disclosure include a 65% crystalline portion and a 35% amorphous region. In an embodiment, the SPFs of a composition of the present disclosure include a 60% crystalline portion and a 40% amorphous region. In an embodiment, the SPFs of a composition of the present disclosure include a 50% crystalline portion and a 50% amorphous region. In an embodiment, the SPFs of a composition of the present disclosure include a 40% crystalline portion and a 60% amorphous region. In an embodiment, the SPFs of a composition of the present disclosure include a 35% crystalline portion and a 65% amorphous region. In an embodiment, the SPFs of a composition of the present disclosure include a 30% crystalline portion and a 70% amorphous region. In an embodiment, the SPFs of a composition of the present disclosure include a 25% crystalline portion and a 75% amorphous region. In an embodiment, the SPFs of a composition of the present disclosure include a 20% crystalline portion and a 80% amorphous region. In an embodiment, the SPFs of a composition of the present disclosure include a 15% crystalline portion and a 85% amorphous region. In an embodiment, the SPFs of a composition of the present disclosure include a 10% crystalline portion and a 90% amorphous region. In an embodiment, the SPFs of a composition of the present disclosure include a 5% crystalline portion and a 90% amorphous region. In an embodiment, the SPFs of a composition of the present disclosure include a 1% crystalline portion and a 99% amorphous region.

FIG. 2 is a flow chart showing various parameters that can be modified during the process of producing a silk protein fragment solution of the present disclosure during the extraction and the dissolution steps. Select method parameters may be altered to achieve distinct final solution characteristics depending upon the intended use, e.g., molecular weight and polydispersity. It should be understood that not all of the steps illustrated are necessarily required to fabricate all silk solutions of the present disclosure.

In an embodiment, silk protein fragment solutions useful for a wide variety of applications are prepared according to the following steps: forming pieces of silk cocoons from the Bombyx mori silkworm; extracting the pieces at about 100° C. in a Na₂CO₃ water solution for about 60 minutes, wherein a volume of the water equals about 0.4×raw silk weight and the amount of Na₂CO₃ is about 0.848×the weight of the pieces to form a silk fibroin extract; triple rinsing the silk fibroin extract at about 60° C. for about 20 minutes per rinse in a volume of rinse water, wherein the rinse water for each cycle equals about 0.2 L×the weight of the pieces; removing excess water from the silk fibroin extract; drying the silk fibroin extract; dissolving the dry silk fibroin extract in a LiBr solution, wherein the LiBr solution is first heated to about 100° C. to create a silk and LiBr solution and maintained; placing the silk and LiBr solution in a dry oven at about 100° C. for about 60 minutes to achieve complete dissolution and further fragmentation of the native silk protein structure into mixture with desired molecular weight and polydispersity; filtering the solution to remove any remaining debris from the silkworm; diluting the solution with water to result in a 1.0 wt. % silk solution; and removing solvent from the solution using Tangential Flow Filtration (TFF). In an embodiment, a 10 kDa membrane is utilized to purify the silk solution and create the final desired silk-to-water ratio. TFF can then be used to further concentrate the silk solution to a concentration of 2.0 wt. % silk in water.

Without wishing to be bound by any particular theory, varying extraction (i.e., time and temperature), LiBr (i.e., temperature of LiBr solution when added to silk fibroin extract or vice versa) and dissolution (i.e., time and temperature) parameters results in solvent and silk solutions with different viscosities, homogeneities, and colors. Also without wishing to be bound by any particular theory, increasing the temperature for extraction, lengthening the extraction time, using a higher temperature LiBr solution at emersion and over time when dissolving the silk and increasing the time at temperature (e.g., in an oven as shown here, or an alternative heat source) all resulted in less viscous and more homogeneous solvent and silk solutions.

The extraction step could be completed in a larger vessel, for example an industrial washing machine where temperatures at or in between 60° C. to 100° C. can be maintained. The rinsing step could also be completed in the industrial washing machine, eliminating the manual rinse cycles. Dissolution of the silk in LiBr solution could occur in a vessel other than a convection oven, for example a stirred tank reactor. Dialyzing the silk through a series of water changes is a manual and time intensive process, which could be accelerated by changing certain parameters, for example diluting the silk solution prior to dialysis. The dialysis process could be scaled for manufacturing by using semi-automated equipment, for example a tangential flow filtration system.

Varying extraction (i.e., time and temperature), LiBr (i.e., temperature of LiBr solution when added to silk fibroin extract or vice versa) and dissolution (i.e., time and temperature) parameters results in solvent and silk solutions with different viscosities, homogeneities, and colors. Increasing the temperature for extraction, lengthening the extraction time, using a higher temperature LiBr solution at emersion and over time when dissolving the silk and increasing the time at temperature (e.g., in an oven as shown here, or an alternative heat source) all resulted in less viscous and more homogeneous solvent and silk solutions. While almost all parameters resulted in a viable silk solution, methods that allow complete dissolution to be achieved in fewer than 4 to 6 hours are preferred for process scalability.

In an embodiment, solutions of silk fibroin protein fragments having a weight average ranging from about 6 kDa to about 17 kDa are prepared according to following steps: degumming a silk source by adding the silk source to a boiling (100° C.) aqueous solution of sodium carbonate for a treatment time of between about 30 minutes to about 60 minutes; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 60° C. to about 140° C.; maintaining the solution of silk fibroin-lithium bromide in an oven having a temperature of about 140° C. for a period of at most 1 hour; removing the lithium bromide from the silk fibroin extract; and producing an aqueous solution of silk protein fragments, the aqueous solution comprising: fragments having a weight average molecular weight ranging from about 6 kDa to about 17 kDa, and a polydispersity of between 1 and about 5, or between about 1.5 and about 3.0. The method may further comprise drying the silk fibroin extract prior to the dissolving step. The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay. The aqueous solution of silk fibroin protein fragments may be lyophilized. In some embodiments, the silk fibroin protein fragment solution may be further processed into various forms including gel, powder, and nanofiber.

In an embodiment, solutions of silk fibroin protein fragments having a weight average molecular weight ranging from about 17 kDa to about 39 kDa are prepared according to the following steps: adding a silk source to a boiling (100° C.) aqueous solution of sodium carbonate for a treatment time of between about 30 minutes to about 60 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at most 1 hour; removing the lithium bromide from the silk fibroin extract; and producing an aqueous solution of silk fibroin protein fragments, wherein the aqueous solution of silk fibroin protein fragments comprises lithium bromide residuals of between about 10 ppm and about 300 ppm, wherein the aqueous solution of silk protein fragments comprises sodium carbonate residuals of between about 10 ppm and about 100 ppm, wherein the aqueous solution of silk fibroin protein fragments comprises fragments having a weight average molecular weight ranging from about 17 kDa to about 39 kDa, and a polydispersity of between 1 and about 5, or between about 1.5 and about 3.0. The method may further comprise drying the silk fibroin extract prior to the dissolving step. The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay.

In some embodiments, a method for preparing an aqueous solution of silk fibroin protein fragments having an average weight average molecular weight ranging from about 6 kDa to about 16 kDa includes the steps of: degumming a silk source by adding the silk source to a boiling (100° C.) aqueous solution of sodium carbonate for a treatment time of between about 30 minutes to about 60 minutes; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 60° C. to about 140° C.; maintaining the solution of silk fibroin-lithium bromide in an oven having a temperature of about 140° C. for a period of at least 1 hour; removing the lithium bromide from the silk fibroin extract; and producing an aqueous solution of silk protein fragments, the aqueous solution comprising: fragments having an average weight average molecular weight ranging from about 6 kDa to about 16 kDa, and a polydispersity of between 1 and about 5, or between about 1.5 and about 3.0. The method may further comprise drying the silk fibroin extract prior to the dissolving step. The aqueous solution of pure silk fibroin protein fragments may comprise lithium bromide residuals of less than 300 ppm as measured using a high-performance liquid chromatography lithium bromide assay. The aqueous solution of pure silk fibroin protein fragments may comprise sodium carbonate residuals of less than 100 ppm as measured using a high-performance liquid chromatography sodium carbonate assay. The method may further comprise adding a therapeutic agent to the aqueous solution of pure silk fibroin protein fragments. The method may further comprise adding a molecule selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. The method may further comprise adding at least one of zinc oxide or titanium dioxide. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2% and a vitamin content of at least 20%.

In some embodiments, a method for preparing an aqueous solution of silk fibroin protein fragments having an average weight average molecular weight ranging from about 17 kDa to about 38 kDa includes the steps of: adding a silk source to a boiling (100° C.) aqueous solution of sodium carbonate for a treatment time of between about 30 minutes to about 60 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at least 1 hour; removing the lithium bromide from the silk fibroin extract; and producing an aqueous solution of pure silk fibroin protein fragments, wherein the aqueous solution of pure silk fibroin protein fragments comprises lithium bromide residuals of between about 10 ppm and about 300 ppm, wherein the aqueous solution of silk protein fragments comprises sodium carbonate residuals of between about 10 ppm and about 100 ppm, wherein the aqueous solution of pure silk fibroin protein fragments comprises fragments having an average weight average molecular weight ranging from about 17 kDa to about 38 kDa, and a polydispersity of between 1 and about 5, or between about 1.5 and about 3.0. The method may further comprise drying the silk fibroin extract prior to the dissolving step. The aqueous solution of pure silk fibroin protein fragments may comprise lithium bromide residuals of less than 300 ppm as measured using a high-performance liquid chromatography lithium bromide assay. The aqueous solution of pure silk fibroin protein fragments may comprise sodium carbonate residuals of less than 100 ppm as measured using a high-performance liquid chromatography sodium carbonate assay. The method may further comprise adding a therapeutic agent to the aqueous solution of pure silk fibroin protein fragments. The method may further comprise adding a molecule selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. The method may further comprise adding at least one of zinc oxide or titanium dioxide. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2% and a vitamin content of at least 20%.

In an embodiment, solutions of silk fibroin protein fragments having a weight average molecular weight ranging from about 39 kDa to about 80 kDa are prepared according to the following steps: adding a silk source to a boiling (100° C.) aqueous solution of sodium carbonate for a treatment time of about 30 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at most 1 hour; removing the lithium bromide from the silk fibroin extract; and producing an aqueous solution of silk fibroin protein fragments, wherein the aqueous solution of silk fibroin protein fragments comprises lithium bromide residuals of between about 10 ppm and about 300 ppm, sodium carbonate residuals of between about 10 ppm and about 100 ppm, fragments having a weight average molecular weight ranging from about 39 kDa to about 80 kDa, and a polydispersity of between 1 and about 5, or between about 1.5 and about 3.0. The method may further comprise drying the silk fibroin extract prior to the dissolving step. The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay. In some embodiments, the method may further comprise adding an active agent (e.g., therapeutic agent) to the aqueous solution of pure silk fibroin protein fragments. The method may further comprise adding an active agent selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha-hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2 wt. % and a vitamin content of at least 20 wt. %.

In an embodiment, the silk fibroin protein fragments in the solution are substantially devoid of sericin, have a weight average molecular weight ranging from about 6 kDa to about 17 kDa, and have a polydispersity ranging from about 1.5 and about 3.0. In an embodiment, the silk fibroin protein fragments in the solution are substantially devoid of sericin, have a weight average molecular weight ranging from about 17 kDa to about 39 kDa, and have a polydispersity ranging from 1 and to 5, or between about 1.5 and about 3.0. In an embodiment, the silk fibroin protein fragments in the solution are substantially devoid of sericin, have a weight average molecular weight ranging from about 39 kDa to about 80 kDa, and have a polydispersity ranging from 1 to about 5, or between about 1.5 and about 3.0.

As used herein, the term “substantially free of inorganic residuals” means that the composition exhibits residuals of 0.1% (w/w) or less. In an embodiment, substantially free of inorganic residuals refers to a composition that exhibits residuals of 0.05% (w/w) or less. In an embodiment, substantially free of inorganic residuals refers to a composition that exhibits residuals of 0.01% (w/w) or less. In an embodiment, the amount of inorganic residuals is between 0 ppm (“non-detectable” or “ND”) and 1000 ppm. In an embodiment, the amount of inorganic residuals is ND to about 500 ppm. In an embodiment, the amount of inorganic residuals is ND to about 400 ppm. In an embodiment, the amount of inorganic residuals is ND to about 300 ppm. In an embodiment, the amount of inorganic residuals is ND to about 200 ppm. In an embodiment, the amount of inorganic residuals is ND to about 100 ppm. In an embodiment, the amount of inorganic residuals is between 10 ppm and 1000 ppm. As used herein, the term “substantially free of organic residuals” means that the composition exhibits residuals of 0.1% (w/w) or less, in an embodiment, substantially free of organic residuals refers to a composition that exhibits residuals of 0.05% (w/w) or less. In an embodiment, substantially free of organic residuals refers to a composition that exhibits residuals of 0.01% (w/w) or less. In an embodiment, the amount of organic residuals is between 0 ppm (“non-detectable” or “ND”) and 1000 ppm. In an embodiment, the amount of organic residuals is ND to about 500 ppm. In an embodiment, the amount of organic residuals is ND to about 400 ppm. In an embodiment, the amount of organic residuals is ND to about 300 ppm. In an embodiment, the amount of organic residuals is ND to about 200 ppm. In an embodiment, the amount of organic residuals is ND to about 100 ppm. In an embodiment, the amount of organic residuals is between 10 ppm and 1000 ppm.

Compositions of the present disclosure exhibit “biocompatibility” meaning that the compositions are compatible with living tissue or a living system by not being toxic, injurious, or physiologically reactive and not causing immunological rejection. Such biocompatibility can be evidenced by participants topically applying compositions of the present disclosure on their skin for an extended period of time. In an embodiment, the extended period of time is about 3 days. In an embodiment, the extended period of time is about 7 days, in an embodiment, the extended period of time is about 14 days, in an embodiment, the extended period of time is about 21 days. In an embodiment, the extended period of time is about 30 days. In an embodiment, the extended period of time is selected from the group consisting of about I month, about 2 months, about 3 months, about 4 months, about 5 months, about 6 months, about 7 months, about 8 months, about 9 months, about 10 months, about 11 months, about 12 months, and indefinitely.

Compositions of the present disclosure are “hypoallergenic” meaning that they are relatively unlikely to cause an allergic reaction. Such hypoallergenicity can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely.

Following are non-limiting examples of suitable ranges for various parameters in and for preparation of the silk solutions of the present disclosure. The silk solutions of the present disclosure may include one or more, but not necessarily all, of these parameters and may be prepared using various combinations of ranges of such parameters.

In an embodiment, the percent SPF in the solution is less than 30.0 wt. %. In an embodiment, the percent SPF in the solution is less than 25.0 wt. %. In an embodiment, the percent SPF in the solution is less than 20.0 wt. %. In an embodiment, the percent SPF in the solution is less than 19.0 wt. %. In an embodiment, the percent SPF in the solution is less than 18.0 wt. %. In an embodiment, the percent SPF in the solution is less than 17.0 wt. %. In an embodiment, the percent SPF in the solution is less than 16.0 wt. %. In an embodiment, the percent SPF in the solution is less than 15.0 wt. %. In an embodiment, the percent SPF in the solution is less than 14.0 wt. %. In an embodiment, the percent SPF in the solution is less than 13.0 wt. %. In an embodiment, the percent SPF in the solution is less than 12.0 wt. %. In an embodiment, the percent SPF in the solution is less than 11.0 wt. %. In an embodiment, the percent SPF in the solution is less than 10.0 wt. %. In an embodiment, the percent SPF in the solution is less than 9.0 wt. %. In an embodiment, the percent SPF in the solution is less than 8.0 wt. %. In an embodiment, the percent SPF in the solution is less than 7.0 wt. %. In an embodiment, the percent SPF in the solution is less than 6.0 wt. %. In an embodiment, the percent SPF in the solution is less than 5.0 wt. %. In an embodiment, the percent SPF in the solution is less than 4.0 wt. %. In an embodiment, the percent SPF in the solution is less than 3.0 wt. %. In an embodiment, the percent SPF in the solution is less than 2.0 wt. %. In an embodiment, the percent SPF in the solution is less than 1.0 wt. %. In an embodiment, the percent SPF in the solution is less than 0.9 wt. %. In an embodiment, the percent SPF in the solution is less than 0.8 wt. %. In an embodiment, the percent SPF in the solution is less than 0.7 wt. %. In an embodiment, the percent SPF in the solution is less than 0.6 wt. %. In an embodiment, the percent SPF in the solution is less than 0.5 wt. %. In an embodiment, the percent SPF in the solution is less than 0.4 wt. %. In an embodiment, the percent SPF in the solution is less than 0.3 wt. %. In an embodiment, the percent SPF in the solution is less than 0.2 wt. %. In an embodiment, the percent SPF in the solution is less than 0.1 wt. %.

In an embodiment, the percent SPF in the solution is greater than 0.1 wt. %. In an embodiment, the percent SPF in the solution is greater than 0.2 wt. %. In an embodiment, the percent SPF in the solution is greater than 0.3 wt. %. In an embodiment, the percent SPF in the solution is greater than 0.4 wt. %. In an embodiment, the percent SPF in the solution is greater than 0.5 wt. %. In an embodiment, the percent SPF in the solution is greater than 0.6 wt. %. In an embodiment, the percent SPF in the solution is greater than 0.7 wt. %. In an embodiment, the percent SPF in the solution is greater than 0.8 wt. %. In an embodiment, the percent SPF in the solution is greater than 0.9 wt. %. In an embodiment, the percent SPF in the solution is greater than 1.0 wt. %. In an embodiment, the percent SPF in the solution is greater than 2.0 wt. %. In an embodiment, the percent SPF in the solution is greater than 3.0 wt. %. In an embodiment, the percent SPF in the solution is greater than 4.0 wt. %. In an embodiment, the percent SPF in the solution is greater than 5.0 wt. %. In an embodiment, the percent SPF in the solution is greater than 6.0 wt. %. In an embodiment, the percent SPF in the solution is greater than 7.0 wt. %. In an embodiment, the percent SPF in the solution is greater than 8.0 wt. %. In an embodiment, the percent SPF in the solution is greater than 9.0 wt. %. In an embodiment, the percent SPF in the solution is greater than 10.0 wt. %. In an embodiment, the percent SPF in the solution is greater than 11.0 wt. %. In an embodiment, the percent SPF in the solution is greater than 12.0 wt. %. In an embodiment, the percent SPF in the solution is greater than 13.0 wt. %. In an embodiment, the percent SPF in the solution is greater than 14.0 wt. %. In an embodiment, the percent SPF in the solution is greater than 15.0 wt. %. In an embodiment, the percent SPF in the solution is greater than 16.0 wt. %. In an embodiment, the percent SPF in the solution is greater than 17.0 wt. %. In an embodiment, the percent SPF in the solution is greater than 18.0 wt. %. In an embodiment, the percent SPF in the solution is greater than 19.0 wt. %. In an embodiment, the percent SPF in the solution is greater than 20.0 wt. %. In an embodiment, the percent SPF in the solution is greater than 25.0 wt. %.

In an embodiment, the percent SPF in the solution ranges from about 0.1 wt. % to about 30.0 wt. %. In an embodiment, the percent SPF in the solution ranges from about 0.1 wt. % to about 25.0 wt. %. In an embodiment, the percent SPF in the solution ranges from about 0.1 wt. % to about 20.0 wt. %. In an embodiment, the percent SPF in the solution ranges from about 0.1 wt. % to about 15.0 wt. %. In an embodiment, the percent SPF in the solution ranges from about 0.1 wt. % to about 10.0 wt. %. In an embodiment, the percent SPF in the solution ranges from about 0.1 wt. % to about 9.0 wt. %. In an embodiment, the percent SPF in the solution ranges from about 0.1 wt. % to about 8.0 wt. %. In an embodiment, the percent SPF in the solution ranges from about 0.1 wt. % to about 7.0 wt. %. In an embodiment, the percent SPF in the solution ranges from about 0.1 wt. % to about 6.5 wt. %. In an embodiment, the percent SPF in the solution ranges from about 0.1 wt. % to about 6.0 wt. %. In an embodiment, the percent SPF in the solution ranges from about 0.1 wt. % to about 5.5 wt. %. In an embodiment, the percent SPF in the solution ranges from about 0.1 wt. % to about 5.0 wt. %. In an embodiment, the percent SPF in the solution ranges from about 0.1 wt. % to about 4.5 wt. %. In an embodiment, the percent SPF in the solution ranges from about 0.1 wt. % to about 4.0 wt. %. In an embodiment, the percent SPF in the solution ranges from about 0.1 wt. % to about 3.5 wt. %. In an embodiment, the percent SPF in the solution ranges from about 0.1 wt. % to about 3.0 wt. %. In an embodiment, the percent SPF in the solution ranges from about 0.1 wt. % to about 2.5 wt. %. In an embodiment, the percent SPF in the solution ranges from about 0.1 wt. % to about 2.0 wt. %. In an embodiment, the percent SPF in the solution ranges from about 0.1 wt. % to about 2.4 wt. %. In an embodiment, the percent SPF in the solution ranges from about 0.5 wt. % to about 5.0 wt. %. In an embodiment, the percent SPF in the solution ranges from about 0.5 wt. % to about 4.5 wt. %. In an embodiment, the percent SPF in the solution ranges from about 0.5 wt. % to about 4.0 wt. %. In an embodiment, the percent SPF in the solution ranges from about 0.5 wt. % to about 3.5 wt. %. In an embodiment, the percent SPF in the solution ranges from about 0.5 wt. % to about 3.0 wt. %. In an embodiment, the percent SPF in the solution ranges from about 0.5 wt. % to about 2.5 wt. %. In an embodiment, the percent SPF in the solution ranges from about 1.0 wt. % to about 4.0 wt. %. In an embodiment, the percent SPF in the solution ranges from about 1.0 wt. % to about 3.5 wt. %. In an embodiment, the percent SPF in the solution ranges from about 1.0 wt. % to about 3.0 wt. %. In an embodiment, the percent SPF in the solution ranges from about 1.0 wt. % to about 2.5 wt. %. In an embodiment, the percent SPF in the solution ranges from about 1.0 wt. % to about 2.4 wt. %. In an embodiment, the percent SPF in the solution ranges from about 1.0 wt. % to about 2.0 wt. %.

In an embodiment, the percent SPF in the solution ranges from about 20.0 wt. % to about 30.0 wt. %. In an embodiment, the percent SPF in the solution ranges from about 0.1 wt. % to about 10.0 wt. %. In an embodiment, the percent SPF in the solution ranges from about 1.0 wt. % to about 10.0 wt. %. In an embodiment, the percent SPF in the solution ranges from about 2 wt. % to about 10.0 wt. %. In an embodiment, the percent SPF in the solution ranges from about 0.1 wt. % to about 6.0 wt. %. In an embodiment, the percent SPF in the solution ranges from about 6.0 wt. % to about 10.0 wt. %. In an embodiment, the percent SPF in the solution ranges from about 6.0 wt. % to about 8.0 wt. %. In an embodiment, the percent SPF in the solution ranges from about 6.0 wt. % to about 9.0 wt. %. In an embodiment, the percent SPF in the solution ranges from about 10.0 wt. % to about 20.0 wt. %. In an embodiment, the percent SPF in the solution ranges from about 11.0 wt. % to about 19.0 wt. %. In an embodiment, the percent SPF in the solution ranges from about 12.0 wt. % to about 18.0 wt. %. In an embodiment, the percent SPF in the solution ranges from about 13.0 wt. % to about 17.0 wt. %. In an embodiment, the percent SPF in the solution ranges from about 14.0 wt. % to about 16.0 wt. %. In an embodiment, the percent SPF in the solution is about 1.0 wt. %. In an embodiment, the percent SPF in the solution is about 1.5 wt. %. In an embodiment, the percent SPF in the solution is about 2.0 wt. %. In an embodiment, the percent SPF in the solution is about 2.4 wt. %. In an embodiment, the percent SPF in the solution is 3.0 wt. %. In an embodiment, the percent SPF in the solution is 3.5 wt. %. In an embodiment, the percent SPF in the solution is about 4.0 wt. %. In an embodiment, the percent SPF in the solution is about 4.5 wt. %. In an embodiment, the percent SPF in the solution is about 5.0 wt. %. In an embodiment, the percent SPF in the solution is about 5.5 wt. %. In an embodiment the percent SPF in the solution is about 6.0 wt. %. In an embodiment, the percent SPF in the solution is about 6.5 wt. %. In an embodiment, the percent SPF in the solution is about 7.0 wt. %. In an embodiment, the percent SPF in the solution is about 7.5 wt. %. In an embodiment, the percent SPF in the solution is about 8.0 wt. %. In an embodiment, the percent SPF in the solution is about 8.5 wt. %. In an embodiment, the percent SPF in the solution is about 9.0 wt. %. In an embodiment, the percent SPF in the solution is about 9.5 wt. %. In an embodiment, the percent SPF in the solution is about 10.0 wt. %.

In an embodiment, the percent sericin in the solution is non-detectable to 25.0 wt. %. In an embodiment, the percent sericin in the solution is non-detectable to 5.0 wt. %. In an embodiment, the percent sericin in the solution is 1.0 wt. %. In an embodiment, the percent sericin in the solution is 2.0 wt. %. In an embodiment, the percent sericin in the solution is 3.0 wt. %. In an embodiment, the percent sericin in the solution is 4.0 wt. %. In an embodiment, the percent sericin in the solution is 5.0 wt. %. In an embodiment, the percent sericin in the solution is 10.0 wt. %. In an embodiment, the percent sericin in the solution is 25.0 wt. %.

In some embodiments, the silk fibroin protein fragments of the present disclosure are shelf stable (they will not slowly or spontaneously gel when stored in an aqueous solution and there is no aggregation of fragments and therefore no increase in molecular weight over time), from 10 days to 3 years depending on storage conditions, percent SPF, and number of shipments and shipment conditions. Additionally, pH may be altered to extend shelf life and/or support shipping conditions by preventing premature folding and aggregation of the silk. In an embodiment, the stability of the LiBr-silk fragment solution is 0 to 1 year. In an embodiment, the stability of the LiBr-silk fragment solution is 0 to 2 years. In an embodiment, the stability of the LiBr-silk fragment solution is 0 to 3 years. In an embodiment, the stability of the LiBr-silk fragment solution is 0 to 4 years. In an embodiment, the stability of the LiBr-silk fragment solution is 0 to 5 years. In an embodiment, the stability of the LiBr-silk fragment solution is 1 to 2 years. In an embodiment, the stability of the LiBr-silk fragment solution is 1 to 3 years. In an embodiment, the stability of the LiBr-silk fragment solution is 1 to 4 years. In an embodiment, the stability of the LiBr-silk fragment solution is 1 to 5 years. In an embodiment, the stability of the LiBr-silk fragment solution is 2 to 3 years. In an embodiment, the stability of the LiBr-silk fragment solution is 2 to 4 years. In an embodiment, the stability of the LiBr-silk fragment solution is 2 to 5 years. In an embodiment, the stability of the LiBr-silk fragment solution is 3 to 4 years. In an embodiment, the stability of the LiBr-silk fragment solution is 3 to 5 years. In an embodiment, the stability of the LiBr-silk fragment solution is 4 to 5 years.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a composition of the present disclosure having pure silk fibroin protein fragments has non-detectable levels of LiBr residuals. In an embodiment, the amount of the LiBr residuals in a composition of the present disclosure is between 10 ppm and 1000 ppm. In an embodiment, the amount of the LiBr residuals in a composition of the present disclosure is between 10 ppm and 300 ppm. In an embodiment, the amount of the LiBr residuals in a composition of the present disclosure is less than 25 ppm. In an embodiment, the amount of the Li Br residuals in a composition of the present disclosure is less than 50 ppm. In an embodiment, the amount of the LiBr residuals in a composition of the present disclosure is less than 75 ppm. In an embodiment, the amount of the LiBr residuals in a composition of the present disclosure is less than 100 ppm. In an embodiment, the amount of the LiBr residuals in a composition of the present disclosure is less than 200 ppm. In an embodiment, the amount of the LiBr residuals in a composition of the present disclosure is less than 300 ppm. In an embodiment, the amount of the LiBr residuals in a composition of the present disclosure is less than 400 ppm. In an embodiment, the amount of the LiBr residuals in a composition of the present disclosure is less than 500 ppm. In an embodiment, the amount of the LiBr residuals in a composition of the present disclosure is less than 600 ppm. In an embodiment, the amount of the LiBr residuals in a composition of the present disclosure is less than 700 ppm. In an embodiment, the amount of the LiBr residuals in a composition of the present disclosure is less than 800 ppm. In an embodiment, the amount of the LiBr residuals in a composition of the present disclosure is less than 900 ppm. In an embodiment, the amount of the LiBr residuals in a composition of the present disclosure is less than 1000 ppm. In an embodiment, the amount of the LiBr residuals in a composition of the present disclosure is non-detectable to 500 ppm. In an embodiment, the amount of the LiBr residuals in a composition of the present disclosure is non-detectable to 450 ppm. In an embodiment, the amount of the LiBr residue in a composition of the present disclosure is non-detectable to 400 ppm. In an embodiment, the amount of the LiBr residuals in a composition of the present disclosure is non-detectable to 350 ppm. In an embodiment, the amount of the LiBr residuals in a composition of the present disclosure is non-detectable to 300 ppm. In an embodiment, the amount of the LiBr residuals in a composition of the present disclosure is non-detectable to 250 ppm. In an embodiment, the amount of the LiBr residuals in a composition of the present disclosure is non-detectable to 200 ppm. In an embodiment, the amount of the LiBr residuals in a composition of the present disclosure is non-detectable to 150 ppm. In an embodiment, the amount of the LiBr residuals in a composition of the present disclosure is non-detectable to 100 ppm. In an embodiment, the amount of the LiBr residuals in a composition of the present disclosure is 100 ppm to 200 ppm. In an embodiment, the amount of the LiBr residuals in a composition of the present disclosure is 200 ppm to 300 ppm. In an embodiment, the amount of the LiBr residuals in a composition of the present disclosure is 300 ppm to 400 ppm. In an embodiment, the amount of the LiBr residuals in a composition of the present disclosure is 400 ppm to 500 ppm.

In an embodiment, a composition of the present disclosure having pure silk fibroin protein fragments, has non-detectable levels of Na₂CO₃ residuals. In an embodiment, the amount of the Na₂CO₃ residuals in a composition of the present disclosure is less than 100 ppm. In an embodiment, the amount of the Na₂CO₃ residuals in a composition of the present disclosure is less than 200 ppm. In an embodiment, the amount of the Na₂CO₃ residuals in a composition of the present disclosure is less than 300 ppm. In an embodiment, the amount of the Na₂CO₃ residuals in a composition of the present disclosure is less than 400 ppm. In an embodiment, the amount of the Na₂CO₃ residuals in a composition of the present disclosure is less than 500 ppm. In an embodiment, the amount of the Na₂CO₃ residuals in a composition of the present disclosure is less than 600 ppm. In an embodiment, the amount of the Na₂CO₃ residuals in a composition of the present disclosure is less than 700 ppm. In an embodiment, the amount of the Na₂CO₃ residuals in a composition of the present disclosure is less than 800 ppm. In an embodiment, the amount of the Na₂CO₃ residuals in a composition of the present disclosure is less than 900 ppm. In an embodiment, the amount of the Na₂CO₃ residuals in a composition of the present disclosure is less than 1000 ppm. In an embodiment, the amount of the Na₂CO₃ residuals in a composition of the present disclosure is non-detectable to 500 ppm. In an embodiment, the amount of the Na₂CO₃ residuals in a composition of the present disclosure is non-detectable to 450 ppm. In an embodiment, the amount of the Na₂CO₃ residuals in a composition of the present disclosure is non-detectable to 400 ppm. In an embodiment, the amount of the Na₂CO₃ residuals in a composition of the present disclosure is non-detectable to 350 ppm. In an embodiment, the amount of the Na₂CO₃ residuals in a composition of the present disclosure is non-detectable to 300 ppm. In an embodiment, the amount of the Na₂CO₃ residuals in a composition of the present disclosure is non-detectable to 250 ppm. In an embodiment, the amount of the Na₂CO₃ residuals in a composition of the present disclosure is non-detectable to 200 ppm. In an embodiment, the amount of the Na₂CO₃ residuals in a composition of the present disclosure is non-detectable to 150 ppm. In an embodiment, the amount of the Na₂CO₃ residuals in a composition of the present disclosure is non-detectable to 100 ppm. In an embodiment, the amount of the Na₂CO₃ residuals in a composition of the present disclosure is 100 ppm to 200 ppm. In an embodiment, the amount of the Na₂CO₃ residuals in a composition of the present disclosure is 200 ppm to 300 ppm. In an embodiment, the amount of the Na₂CO₃ residuals in a composition of the present disclosure is 300 ppm to 400 ppm. In an embodiment, the amount of the Na₂CO₃ residuals in a composition of the present disclosure is 400 ppm to 500 ppm.

A unique feature of the SPF compositions of the present disclosure are shelf stability (they will not slowly or spontaneously gel when stored in an aqueous solution and there is no aggregation of fragments and therefore no increase in molecular weight over time), from 10 days to 3 years depending on storage conditions, percent silk, and number of shipments and shipment conditions. Additionally pH may be altered to extend shelf-life and/or support shipping conditions by preventing premature folding and aggregation of the silk. In an embodiment, a SPF solution composition of the present disclosure has a shelf stability for up to 2 weeks at room temperature (RT). In an embodiment, a SPF solution composition of the present disclosure has a shelf stability for up to 4 weeks at RT. In an embodiment, a SPF solution composition of the present disclosure has a shelf stability for up to 6 weeks at RT. In an embodiment, a SPF solution composition of the present disclosure has a shelf stability for up to 8 weeks at RT. In an embodiment, a SPF solution composition of the present disclosure has a shelf stability for up to 10 weeks at RT. In an embodiment, a SPF solution composition of the present disclosure has a shelf stability for up to 12 weeks at RT. In an embodiment, a SPF solution composition of the present disclosure has a shelf stability ranging from about 4 weeks to about 52 weeks at RT. Table A below shows shelf stability test results for embodiments of SPF compositions of the present disclosure.

TABLE A
Shelf Stability of SPF Compositions of the Present Disclosure

| % Silk | Temperature | Time to Gelation |
|---|---|---|
| 2 | RT | 4 weeks |
| 2 | 4 ° C. | >9 weeks |
| 4 | RT | 4 weeks |
| 4 | 4° C. | >9 weeks |
| 6 | RT | 2 weeks |
| 6 | 4° C. | >9 weeks |

Silk fibroin film has been reported, e.g. ultrathin films in WO 2007/016524, thick films, conformal coatings in WO 2005/000483 and WO 2005/123114. Japanese Patent Laid-Open Publication No. 1-118,545 describes various uses of silk films such as artificial skins, wigs, and sweat clothes. These types of silk film have excellent vapor permeability, improved transparency and mechanical strength, and desirable affinity to the human body.

This disclosure exploits the potential of silk film or gels derived from the hydrophobic silk fibroin protein for manufacturing silk products in a wide variety of fields. In some embodiments, this disclosure provides a silk film comprising the silk fibroin protein fragments as described above. In some embodiments, the silk film further comprises an active agent (e.g., cosmetic active agent, therapeutic agent) as described above to form a drug loaded silk film. In some embodiments, the silk film has a water content less than 10 wt. % by the total weight of the silk film. In some embodiments, the silk film has a water content less than 9 wt. % by the total weight of the silk film. In some embodiments, the silk film has a water content less than 8 wt. % by the total weight of the silk film. In some embodiments, the silk film has a water content less than 7 wt. % by the total weight of the silk film. In some embodiments, the silk film has a water content less than 6 wt. % by the total weight of the silk film. In some embodiments, the silk film has a water content less than 5 wt. % by the total weight of the silk film. In some embodiments, the silk film has a water content less than 4 wt. % by the total weight of the silk film. In some embodiments, the silk film has a water content less than 3 wt. % by the total weight of the silk film. In some embodiments, the silk film has a water content less than 2 wt. % by the total weight of the silk film. In some embodiments, the silk film has a water content less than 1 wt. % by the total weight of the silk film.

In some embodiments, this disclosure provides a silk gel comprising the silk fibroin protein fragments as described above. In some embodiments, the silk gel further comprises an active agent to form a drug loaded silk gel. In some embodiments, the silk gel comprises silk aqueous gel formed by gelling the silk solution described below via sol-gel process. In some embodiments, the gelation of silk fibroin protein fragment solutions may be induced by sonication, vortex, heating, solvent treatment (e.g. methanol, ethanol), electrogelation, ultrasonication, chemicals (e.g. vitamin C), or the like.

In some embodiments, the silk film comprises amorphous silk films fabricated from the silk solutions as described below. In some embodiments, the silk film comprises crystalline silk film fabricated from the silk solutions as described below. In an embodiment, this disclosure provides a method for producing a silk film or gel from a silk solution described below comprising the following steps: step A, a silk solution of the present disclosure is chosen, and then at least one therapeutic agent is added directly to the silk solution prior to gel or film processing step B. When producing a silk film, the silk solution with an active agent may be cast directly onto a shaped mold to achieve a unique film shape (e.g., silicone mold) or the silk solution may be cast as a sheet and then subsequently cut or punched into a variety of shapes, with a variety of cutting techniques, including, but not limited to cutting with a rotary blade or laser cutting for example, depending upon the desired application, step C. If cast on a mold, for example silicone, the silicone mold may be heated on a laser-etched/patterned surface to create an impression that will be transferred to the final film. For example, the product logo could be transferred to the film, visible, but not palpable by hand, and used to show authenticity of the product. The concentration and/or mass of the final silk protein fragment film can be varied to control the film's degree of flexibility and conformity to the different anatomical topographies of the body organ. Altering the drying method for a silk film will also result in different final film characteristics. Applying airflow and/or heat impacts the properties of the film (e.g., brittleness, number of bubbles, curling, solubility, surface appearance), step D. Additionally, the percent moisture within the film at the time of packaging will impact stability over time with too much moisture resulting in yellowing of the films with time. In some embodiments, films ideally may have between about 2 to about 20% water content at completion of drying. It was observed that greater moisture content than 20% in the films will decrease shelf life. If films are not dry enough (that is they have greater than 20% water content) before packaging, they will yellow over time (2+weeks). It is advised that films are dried in an incubator until the relative humidity in the incubator is less than the relative humidity in the surrounding area and no greater than 36%. Ambient humidity will have an effect on the ability to remove moisture and therefore, a tactile/audio test can be used to determine whether films are ready for packaging. In an embodiment, the test includes removal of a film from the drying system, slightly bending one end of the film and releasing it. If the film feels and sounds similar to a piece of paper or thin plastic, it is considered dry. If the film has not completed drying, it will be pliable and will make no noise upon bending and release. In an embodiment, the film is flexible without the need for process additives such as glycerin, such that a film that is 2.5 cm wide by 10 cm long can be bent in half so that opposite ends of the film can touch one another without the film breaking or cracking A film of this same size can be bent in half along the length of the film to create a 45-degree angle without breaking or cracking the film. The final silk protein fragment-film is pure with undetectable levels of particulate debris and/or process contaminants, including LiBr and Na₂CO₃. Alternatively, the final the silk fibroin protein fragment solutions has less than 500 ppm process contaminants.

In some embodiments, at least one active agent is physically entrapped into a silk solution of the present disclosure during processing into aqueous gels. An aqueous silk gel of the present disclosure can be used to release at least one active agent. In some embodiments, the silk solutions described below may be used to generate silk gels of varying gel and liquid consistencies by varying water content/concentration.

In some embodiments, when producing a silk gel, an acid is used to help facilitate gelation. In an embodiment, when producing a silk gel that includes a neutral or a basic molecule and/or therapeutic agent, an acid can be added to facilitate gelation. In an embodiment, when producing a silk gel, increasing the pH (making the gel more basic) increases the shelf stability of the gel. In an embodiment, when producing a silk gel, increasing the pH (making the gel more basic) allows for a greater quantity of an acidic molecule to be loaded into the gel.

In some embodiments, the water solubility of the silk film derived from silk fibroin protein fragments as described herein can be modified by solvent annealing (water annealing or methanol annealing), chemical crosslinking, enzyme crosslinking and heat treatment.

In some embodiments, the process of annealing may involve inducing beta-sheet formation in the silk fibroin protein fragment solutions used as a coating material. Techniques of annealing (e.g., increase crystallinity) or otherwise promoting “molecular packing” of silk fibroin-protein based fragments have been described. In some embodiments, the amorphous silk film is annealed to introduce beta-sheet in the presence of a solvent selected from the group of water or organic solvent. In some embodiments, the amorphous silk film is annealed to introduce beta-sheet in the presence of water (water annealing process). In some embodiments, the amorphous silk fibroin protein fragment film is annealed to introduce beta-sheet in the presence of methanol. In some embodiments, annealing (e.g., the beta sheet formation) is induced by addition of an organic solvent. Suitable organic solvents include, but are not limited to methanol, ethanol, acetone, isopropanol, or combination thereof. In some embodiments, annealing is carried out by so-called “water-annealing” or “water vapor annealing” in which water vapor is used as an intermediate plasticizing agent or catalyst to promote the packing of beta-sheets. In some embodiments, the process of water annealing may be performed under vacuum. Suitable such methods have been described in Jin H-J et al. (2005), Water-stable Silk Films with Reduced Beta-Sheet Content, Advanced Functional Materials, 15: 1241-1247; Xiao H. et al. (2011), Regulation of Silk Material Structure by Temperature-Controlled Water Vapor Annealing, Biomacromolecules, 12(5): 1686-1696.

The important feature of the water annealing process is to drive the formation of crystalline beta-sheet in the silk fibroin protein fragment peptide chain to allow the silk fibroin self-assembling into a continuous film. In some embodiments, the crystallinity of the silk fibroin protein fragment film is controlled by controlling the temperature of water vapor and duration of the annealing. In some embodiments, the annealing is performed at a temperature ranging from about 65° C. to about 110° C. In some embodiments, the temperature of the water is maintained at about 80° C. In some embodiments, annealing is performed at a temperature selected from the group of about 65° C., about 70° C., about 75° C., about 80° C., about 85° C., about 90° C., about 95° C., about 100° C., about 105° C., and about 110° C.

In some embodiments, the annealing process lasts a period of time selected from the group of about 1 minute to about 40 minutes, about 1 minute to about 50 minutes, about 1 minute to about 60 minutes, about 1 minute to about 70 minutes, about 1 minute to about 80 minutes, about 1 minute to about 90 minutes, about 1 minute to about 100 minutes, about 1 minute to about 110 minutes, about 1 minute to about 120 minutes, about 1 minute to about 130 minutes, about 5 minutes to about 40 minutes, about 5 minutes to about 50 minutes, about 5 minutes to about 60 minutes, about 5 minutes to about 70 minutes, about 5 minutes to about 80 minutes, about 5 minutes to about 90 minutes, about 5 minutes to about 100 minutes, about 5 minutes to about 110 minutes, about 5 minutes to about 120 minutes, about 5 minutes to about 130 minutes, about 10 minutes to about 40 minutes, about 10 minutes to about 50 minutes, about 10 minutes to about 60 minutes, about 10 minutes to about 70 minutes, about 10 minutes to about 80 minutes, about 10 minutes to about 90 minutes, about 10 minutes to about 100 minutes, about 10 minutes to about 110 minutes, about 10 minutes to about 120 minutes, about 10 minutes to about 130 minutes, about 15 minutes to about 40 minutes, about 15 minutes to about 50 minutes, about 15 minutes to about 60 minutes, about 15 minutes to about 70 minutes, about 15 minutes to about 80 minutes, about 15 minutes to about 90 minutes, about 15 minutes to about 100 minutes, about 15 minutes to about 110 minutes, about 15 minutes to about 120 minutes, about 15 minutes to about 130 minutes, about 20 minutes to about 40 minutes, about 20 minutes to about 50 minutes, about 20 minutes to about 60 minutes, about 20 minutes to about 70 minutes, about 20 minutes to about 80 minutes, about 20 minutes to about 90 minutes, about 20 minutes to about 100 minutes, about 20 minutes to about 110 minutes, about 20 minutes to about 120 minutes, about 20 minutes to about 130 minutes, about 25 minutes to about 40 minutes, about 25 minutes to about 50 minutes, about 25 minutes to about 60 minutes, about 25 minutes to about 70 minutes, about 25 minutes to about 80 minutes, about 25 minutes to about 90 minutes, about 25 minutes to about 100 minutes, about 25 minutes to about 110 minutes, about 25 minutes to about 120 minutes, about 25 minutes to about 130 minutes, about 30 minutes to about 40 minutes, about 30 minutes to about 50 minutes, about 30 minutes to about 60 minutes, about 30 minutes to about 70 minutes, about 30 minutes to about 80 minutes, about 30 minutes to about 90 minutes, about 30 minutes to about 100 minutes, about 30 minutes to about 110 minutes, about 30 minutes to about 120 minutes, about 30 minutes to about 130 minutes, about 35 minutes to about 40 minutes, about 35 minutes to about 50 minutes, about 35 minutes to about 60 minutes, about 35 minutes to about 70 minutes, about 35 minutes to about 80 minutes, about 35 minutes to about 90 minutes, about 35 minutes to about 100 minutes, about 35 minutes to about 110 minutes, about 35 minutes to about 120 minutes, about 35 minutes to about 130 minutes, about 40 minutes to about 50 minutes, about 40 minutes to about 60 minutes, about 40 minutes to about 70 minutes, about 40 minutes to about 80 minutes, about 40 minutes to about 90 minutes, about 40 minutes to about 100 minutes, about 40 minutes to about 110 minutes, about 40 minutes to about 120 minutes, about 40 minutes to about 130 minutes, about 45 minutes to about 50 minutes, about 45 minutes to about 60 minutes, about 45 minutes to about 70 minutes, about 45 minutes to about 80 minutes, about 45 minutes to about 90 minutes, about 45 minutes to about 100 minutes, about 45 minutes to about 110 minutes, about 45 minutes to about 120 minutes, and about 45 minutes to about 130 minutes. In some embodiments, the annealing process lasts a period of time ranging from about 1 minute to about 60 minutes. In some embodiments, the annealing process lasts a period of time ranging from about 45 minutes to about 60 minutes. The longer water annealing post-processing corresponded an increased crystallinity of silk fibroin protein fragments.

In some embodiments, the annealed silk fibroin protein fragment film is immersing the wet silk fibroin protein fragment film in 100% methanol for 60 minutes at room temperature. The methanol annealing changed the composition of silk fibroin protein fragment film from predominantly amorphous random coil to crystalline antiparallel beta-sheet structure.

In some embodiments, the silk fibroin protein fragments are present in the silk film at a weight amount ranging from about 85.0 wt. % to about 99.0 wt. % by the total weight of the silk film. In some embodiments, the silk fibroin protein fragments are present in the silk film at a weight amount ranging from about 90.0 wt. % to about 99.0 wt. % by the total weight of the silk film. In some embodiments, the silk fibroin protein fragments are present in the silk film at a weight amount ranging from about 90.0 wt. % to about 95.0 wt. % by the total weight of the silk film. In some embodiments, the silk fibroin protein fragments are present in the silk film at a weight amount ranging from about 91.0 wt. % to about 96.0 wt. % by the total weight of the silk film. In some embodiments, the silk fibroin protein fragments are present in the silk film at a weight amount ranging from about 92.0 wt. % to about 97.0 wt. % by the total weight of the silk film. In some embodiments, the silk fibroin protein fragments are present in the silk film at a weight amount ranging from about 93.0 wt. % to about 98.0 wt. % by the total weight of the silk film. In some embodiments, the silk fibroin protein fragments are present in the silk film at a weight amount ranging from about 94.0 wt. % to about 99.0 wt. % by the total weight of the silk film.

In some embodiments, the silk fibroin protein fragments are present in the silk gel at a weight amount ranging from about 0.001 wt. % to about 10.0 wt. % by the total weight of the silk gel. In some embodiments, the silk fibroin protein fragments are present in the silk gel at a weight amount ranging from about 0.001 wt. % to about 5.0 wt. % by the total weight of the silk gel. In some embodiments, the silk fibroin protein fragments are present in the silk gel at a weight amount ranging from about 0.001 wt. % to about 1.0 wt. % by the total weight of the silk gel. In some embodiments, the silk fibroin protein fragments are present in the silk gel at a weight amount ranging from about 10 wt. % by the total weight of the silk gel.

In some embodiments, the silk film or silk gel is transparent. In some embodiments, the silk film is translucent. Transparency (also called pellucidity or diaphaneity) as used herein refers to the physical property of allowing light to pass through a material, whereas translucency (also called translucence or translucidity) only allows light to pass through diffusely. The opposite property is opacity. Transparent materials are clear, while translucent ones cannot be seen through clearly.

In an embodiment, the percent water content in gels of the present disclosure is 20% to 99.9%. In an embodiment, the percent water content in gels of the present disclosure is 20% to 25%. In an embodiment, the percent water content in gels of the present disclosure is 25% to 30%. In an embodiment, the percent water content in gels of the present disclosure is 30% to 35%. In an embodiment, the percent water content in gels of the present disclosure is 35% to 40%. In an embodiment, the percent water content in gels of the present disclosure is 40% to 45%. In an embodiment, the percent water content in gels of the present disclosure is 45% to 50%. in an embodiment, the percent water content in gels of the present disclosure is 50% to 55%. In an embodiment, the percent water content in gels of the present disclosure is 55% to 60%. In an embodiment, the percent water content in gels of the present disclosure is 60% to 65%. In an embodiment, the percent water content in gels of the present disclosure is 65% to 70%. In an embodiment, the percent water content in gels of the present disclosure is 70% to 75%. In an embodiment, the percent water content in gels of the present disclosure is 75% to 80%. In an embodiment, the percent water content in gels of the present disclosure is 80% to 85%. In an embodiment, the percent water content in gels of the present disclosure is 85% to 90%. In an embodiment, the percent water content in gels of the present disclosure is 90% to 95%. In an embodiment, the percent water content in gels of the present disclosure is 95% to 99%.

In an embodiment, the percent water content in films of the present disclosure is 20%. In an embodiment, the percent water content in films of the present disclosure is less than 20%). In an embodiment, the percent water content in films of the present disclosure is less than 18%. In an embodiment, the percent water content in films of the present disclosure is less than 16%. In an embodiment, the percent water content in films of the present disclosure is less than 14%. In an embodiment, the percent water content in films of the present disclosure is less than 12%. In an embodiment, the percent water content in films of the present disclosure is less than 10%). In an embodiment, the percent water content in films of the present disclosure is between about 2% and about 20%.

In some embodiments, the fibroin protein fragment solution can be freeze dried to form lyophilized silk powder. In some embodiments, lyophilized silk powder can be resuspended in water, hexafluoroisopropanol (HFIP), or organic solution following storage to create silk solutions of varying concentrations, including higher concentration solutions than those produced initially.

In some embodiments, the silk solution as described above can be casted on a substrate to form a silk film containing silk fibroin protein fragments after drying. The silk film is then pulverized to form fine powders.

In some embodiments, the silk fibroin protein fragments are dried using a rototherm evaporator or other methods known in the art for creating a dry protein form containing less than 10.0% water by mass. In an embodiment, the solubility of silk fibroin protein fragments of the present disclosure in organic solutions ranges from about 50.0% to about 100%. In an embodiment, the solubility of silk fibroin protein fragments of the present disclosure in organic solutions ranges from about 60.0% to about 100%. In an embodiment, the solubility of silk fibroin protein fragments of the present disclosure in organic solutions ranges from about 70.0% to about 100%. In an embodiment, the solubility of silk fibroin protein fragments of the present disclosure in organic solutions ranges from about 80.0% to about 100%. In an embodiment, the solubility of silk fibroin protein fragments of the present disclosure in organic solutions ranges from about 90.0% to about 100%. In some embodiments, the silk fibroin fragments of the present disclosure are non-soluble in organic solutions.

In some embodiments, the silk solution as described above can be dried by subjecting to thin film evaporation process (also known as Rototherm) followed by milling. The silk solution is placed in a thin film evaporator under reduced pressure, gentle heating and water is continuously removed from the aqueous solution to result in a solid of variable particle size. The particle size can be varied by controlling the evaporation process parameters including pressure, temperature, rotational speed of the cylinder, thickness of the liquid film in the evaporator. The dry protein powder resulted from the rototherm evaporation contains less than 10.0 wt. % moisture content.

In some embodiments, the silk solution as described above can be used to prepare SPF microparticles by precipitation with methanol. Alternative flash drying, fluid-bed drying, spray drying or vacuum drying can be applied to remove water from the silk solution. The SPF powder can then be stored and handled without refrigeration or other special handling procedures. In some embodiments, the SPF powders comprise low molecular weight silk fibroin protein fragments. In some embodiments, the SPF powders comprise mid-molecular weight silk fibroin protein fragments. In some embodiments, the SPF powders comprise a mixture of low molecular weight silk fibroin protein fragments and mid-molecular weight silk fibroin protein fragment.

In some embodiments, the SPF powders comprise low molecular weight silk fibroin protein fragments having a weight average molecular weight ranging from about 5 kDa to about 20 kDa. In some embodiments, the SPF powders comprise low molecular weight silk fibroin protein fragments having a weight average molecular weight selected from the group consisting of from about 5 kDa to 10 kDa, about 10 kDa to about 20 kDa, and about 20 kDa to about 25 kDa. In some embodiments, the SPF powders comprise low molecular weight silk fibroin protein fragments having a weight average molecular weight ranging from about 10 kDa to about 20 kDa. In some embodiments, the SPF powders comprise mid-molecular weight silk fibroin protein fragments having an average weight average molecular weight selected from the group consisting of from about 25 kDa to about 30 kDa, about 30 kDa to about 35 kDa, from about 35 kDa to about 40 kDa, from about 17 kDa to about 39 kDa, from about 45 kDa to about 50 kDa, from about 50 kDa to about 55 kDa, from about 55 kDa to about 60 kDa, from about 60 kDa to about 65 kDa, from about 40 kDa to about 65 kDa, from 65 kDa to about 70 kDa, from about 70 kDa to about 75 kDa, from about 75 kDa to about 80 kDa, from about 39 kDa to about 80 kDa, from about 80 kDa to about 85 kDa, from about 85 kDa to about 90 kDa, from about 90 kDa to about 95 kDa, from about 95 kDa to about 100 kDa, from about 100 kDa to about 105 kDa, from about 105 kDa to about 110 kDa, from about 60 kDa to about 100 kDa, and from about 80 kDa to about 144 kDa. In some embodiments, the SPF powders comprise mid-molecular weight silk fibroin protein fragments having a weight average molecular weight ranging from about 17 kDa to about 39 kDa. In some embodiments, the SPF powders comprise mid-molecular weight silk fibroin protein fragments having a weight average molecular weight ranging from about 40 kDa to about 65 kDa. In some embodiments, the SPF powders comprise mid-molecular weight silk fibroin protein fragments having a weight average molecular weight ranging from about 39 kDa to about 80 kDa. In some embodiments, the SPF powders comprise mid-molecular weight silk fibroin protein fragments having a weight average molecular weight ranging from about 80 kDa to about 144 kDa. In some embodiments, the SPF powders comprise low molecular weight silk fibroin fragments (low-MW silk) having a weight average molecular weight (Mw) 6 kDa and about 17 kDa and a polydispersity between about 1.5 and about 3.0. In some embodiments, the SPF powders comprise mid-molecular weight silk fibroin fragments (Med-MW silk) having a weight average molecular weight ranging from about 17 kDa and about 39 kDa and a polydispersity between about 1.5 and about 3.0. In some embodiments, the SPF powders comprise mid-molecular weight silk fibroin fragments (high-MW silk) having a weight average molecular weight ranging from about 39 kDa to about 80 kDa and a polydispersity between about 1.5 and about 3.0.

In some embodiments, the moisture content in the SPF powder ranges from 0.1 wt. % to 20 wt. % by the total weight of the SPF powder. In some embodiments, the moisture content in the SPF powder ranges from 1.0 wt. % to 10 wt. % by the total weight of the SPF powder. In some embodiments, the moisture content in the SPF powder is less than 1.0 wt. % by the total weight of the SPF powder. In some embodiments, the moisture content in the SPF powder is less than 5.0 wt. % by the total weight of the SPF powder. In some embodiments, the moisture content in the SPF powder is less than 10.0 wt. % by the total weight of the SPF powder. In some embodiments, the moisture content in the SPF powder is selected from the group consisting of less than 1.0 wt. %, less than 1.5 wt. %, less than 2.0 wt. %, less than 2.5 wt. %, less than 3.0 wt. %, less than 3.5 wt. %, less than 4.0 wt. %, less than 4.5 wt. %, less than 5.0 wt. %, less than 5.5 wt. %, less than 6.0 wt. %, less than 6.5 wt. %, less than 7.0 wt. %, less than 7.5 wt. %, less than 8.0 wt. %, less than 8.5 wt. %, less than 9.0 wt. %, less than 9.5 wt. % and less than 10.0 wt. % by the total weight of the SPF powder.

In some embodiments, the SPF powder are solid particles having median particle size ranging from 1.0 μm to 1000 μm. In some embodiments, the SPF powder are microparticles having median particle size ranging from 1.0 μm to 500 μm. In some embodiments, the SPF powder are microparticles having median particle size ranging from 1.0 μm to 300 μm. In some embodiments, the SPF powder are microparticles having median particle size ranging from 1.0 μm to 250 μm. In some embodiments, the SPF powder are microparticles having median particle size ranging from 1.0 μm to 200 μm. In some embodiments, the SPF powder are microparticles having median particle size ranging from 1.0 μm to 100 μm. In some embodiments, the SPF powder are microparticles having median particle size ranging from 1.0 μm to 50.0 μm. In some embodiments, the SPF powder are microparticles having median particle size ranging from 1.0 μm to 25.0 μm. In some embodiments, the SPF powder are microparticles having median particle size ranging from 1.0 μm to 10.0 μm. In some embodiments, the SPF powder are microparticles having median particle size selected from the group consisting of about 1.0 μm, about 2.0 μm, about 3.0 μm, about 4.0 μm, about 5.0 μm, about 6.0 μm, about 7.0 μm, about 8.0 μm, about 9.0 μm, about 10.0 μm, about 11.0 μm, about 12.0 μm, about 13.0 μm, about 14.0 μm, about 15.0 μm, about 16.0 μm, about 17.0 μm, about 18.0 μm, about 19.0 μm, about 20.0 μm, about 21.0 μm, about 22.0 μm, about 23.0 μm, about 24.0 μm, about 25.0 μm, about 26.0 μm, about 27.0 μm, about 28.0 μm, about 29.0 μm, about 30.0 μm, about 31.0 μm, about 32.0 μm, about 33.0 μm, about 34.0 μm, about 35.0 μm, about 36.0 μm, about 37.0 μm, about 38.0 μm, about 39.0 μm, about 40.0 μm, about 41.0 μm, about 42.0 μm, about 43.0 μm, about 44.0 μm, about 45.0 μm, about 46.0 μm, about 47.0 μm, about 48.0 μm, about 49.0 μm, about 50.0 μm, about 51.0 μm, about 52.0 μm, about 53.0 μm, about 54.0 μm, about 55.0 μm, about 56.0 μm, about 57.0 μm, about 58.0 μm, about 59.0 μm, about 60.0 μm, about 61.0 μm, about 62.0 μm, about 63.0 μm, about 64.0 μm, about 65.0 μm, about 66.0 μm, about 67.0 μm, about 68.0 μm, about 69.0 μm, about 70.0 μm, about 71.0 μm, about 72.0 μm, about 73.0 μm, about 74.0 μm, about 75.0 μm, about 76.0 μm, about 77.0 μm, about 78.0 μm, about 79.0 μm, about 80.0 μm, about 81.0 μm, about 82.0 μm, about 83.0 μm, about 84.0 μm, about 85.0 μm, about 86.0 μm, about 87.0 μm, about 88.0 μm, about 89.0 μm, about 90.0 μm, about 91.0 μm, about 92.0 μm, about 93.0 μm, about 94.0 μm, about 95.0 μm, about 96.0 μm, about 97.0 μm, about 98.0 μm, about 99.0 μm, about 100.0 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, about 500 μm, about 510 μm, about 520 μm, about 530 μm, about 540 μm, about 550 μm, about 560 μm, about 570 μm, about 580 μm, about 590 μm, about 600 μm, about 610 μm, about 620 μm, about 630 μm, about 640 μm, about 650 μm, about 660 μm, about 670 μm, about 680 μm, about 690 μm, about 700 μm, about 710 μm, about 720 μm, about 730 μm, about 740 μm, about 750 μm, about 760 μm, about 770 μm, about 780 μm, about 790 μm, about 800 μm, about 810 μm, about 820 μm, about 830 μm, about 840 μm, about 850 μm, about 860 μm, about 870 μm, about 880 μm, about 890 μm, about 900 μm, about 910 μm, about 920 μm, about 930 μm, about 940 μm, about 950 μm, about 960 μm, about 970 μm, about 980 μm, about 990 μm, and about 1000 μM.

In some embodiments, the SPF powder are microparticles having median particle size less than 500 μm. In some embodiments, the SPF powder are microparticles having median particle size less than 325 μm. In some embodiments, the SPF powder are microparticles having median particle size less than 250 μm. In some embodiments, the SPF powder are microparticles having median particle size less than 100 μm. In some embodiments, the SPF powder are microparticles having median particle size less than 50 μm. In some embodiments, the SPF powder are microparticles having median particle size less than 10 μm.

In an embodiment, the water solubility of pure silk fibroin protein fragments of the present disclosure is 50 to 100%. In an embodiment, the water solubility of pure silk fibroin protein fragments of the present disclosure is 60 to 100%. In an embodiment, the water solubility of pure silk fibroin protein fragments of the present disclosure is 70 to 100%. In an embodiment, the water solubility of pure silk fibroin protein fragments of the present disclosure is 80 to 100%. In an embodiment, the water solubility is 90 to 100%. In an embodiment, the silk fibroin fragments of the present disclosure are non-soluble in aqueous solutions.

In an embodiment, the solubility of pure silk fibroin protein fragments of the present disclosure in organic solutions is 50 to 100%. In an embodiment, the solubility of pure silk fibroin protein fragments of the present disclosure in organic solutions is 60 to 100%. In an embodiment, the solubility of pure silk fibroin protein fragments of the present disclosure in organic solutions is 70 to 100%. In an embodiment, the solubility of pure silk fibroin protein fragments of the present disclosure in organic solutions is 80 to 100%. In an embodiment, the solubility of pure silk fibroin protein fragments of the present disclosure in organic solutions is 90 to 100%. In an embodiment, the silk fibroin fragments of the present disclosure are non-soluble in organic solutions.

In some embodiments, the silk fibroin protein fragments comprise cationic quatemized amino acid residue (cationic quatemized silk fibroin) with fatty alkyl groups, wherein the silk fibroin protein fragments having an average weight average molecular weight selected from the group of 5 kDa, 10 kDa, 15 kDa, 20 kDa, 25 kDa, 30 KDa, 35 kDa, 40 kDa, 45 kDa, 50 KDa, 55 kDa, 60 kDa, 65 kDa, 70 kDa, 75 kDa, 80 kDa, 85 kDa, 90 kDa, 95 kDa, and 100 kDa, and a polydispersity of about 1.5 to about 3.0. In some embodiments, the fatty alkyl group for quaternization of amine groups of the silk fibroin protein fragment is selected from the group of cocodimonium hydroxypropyl, hydroxypropyltrimonium, lauryidimonium hydroxypropyl, steardimonium hydroxypropyl, quaternium-79, and combinations thereof.

The silk fibroin-protein based fragments as described herein may find application in cosmetics, personal care, eye care, house care, food and textile industry. In some embodiments, the silk fibroin-protein based fragments described herein may find applications as active agent for personal care product, for example, as micro-exfoliators or micro-exfoliates, as delivery systems for scents/volatile molecule (e.g., perfume encapsulated silk microparticles), as delivery systems for oral care active agents, as delivery systems for eye care active agents, as mucoadhesive delivery systems for systemic delivery of therapeutic agent, as mucoadhesive delivery systems for local delivery of therapeutic drug (e.g., to oral cavity, stomach, ocular cavity). In some embodiments, the silk microparticles described herein may find applications as delivery systems for therapeutically active agent, e.g., delivery systems for sustained release of drugs.

Silk solutions of various molecular weights and/or combinations of molecular weights can be optimized for specific applications. The following provides an example of this process but it not intended to be limiting in application or formulation. Methods of making silk fibroin or silk fibroin protein fragments and their applications in various fields are known and are described for example in U.S. Pat. Nos. 9,187,538, 9,511,012, 9,517,191, 9,522,107, 9,522,108, 9,545,369, and 10,166,177, 10,287,728 and 10,301,768, all of which are incorporated herein in their entireties. The raw silk cocoons from the silkworm Bombyx mori was cut into pieces. The pieces silk cocoons were processed in an aqueous solution of Na₂CO₃ at about 100° C. for about 60 minutes to remove sericin (degumming). The volume of the water used equals about 0.4×raw silk weight and the amount of Na₂CO₃ is about 0.848×the weight of the raw silk cocoon pieces. The resulting degummed silk cocoon pieces were rinsed with deionized water three times at about 60° C. (20 minutes per rinse). The volume of rinse water for each cycle was 0.2 L×the weight of the raw silk cocoon pieces. The excess water from the degummed silk cocoon pieces was removed. After the DI water washing step, the wet degummed silk cocoon pieces were dried at room temperature. The degummed silk cocoon pieces were mixed with a LiBr solution, and the mixture was heated to about 100° C. The warmed mixture was placed in a dry oven and was heated at about 100° C. for about 60 minutes to achieve complete dissolution of the native silk protein. The resulting silk fibroin solution was filtered and dialyzed using Tangential Flow Filtration (TFF) and a 10 kDa membrane against deionized water for 72 hours. The resulting silk fibroin aqueous solution has a concentration of about 8.5 wt. %. Then, 8.5% silk solution was diluted with water to result in a 1.0% w/v silk solution. TFF can then be used to further concentrate the pure silk solution to a concentration of 20.0% w/w silk to water.

Dialyzing the silk through a series of water changes is a manual and time intensive process, which could be accelerated by changing certain parameters, for example diluting the silk solution prior to dialysis. The dialysis process could be scaled for manufacturing by using semi-automated equipment, for example a tangential flow filtration system.

In some embodiments, the silk solutions are prepared under various preparation condition parameters such as: 90° C. 30 min, 90° C. 60 min, 100° C. 30 min, and 100° C. 60 min. Briefly, 9.3 M LiBr was prepared and allowed to sit at room temperature for at least 30 minutes. 5 mL of LiBr solution was added to 1.25 g of silk and placed in the 60° C. oven. Samples from each set were removed at 4, 6, 8, 12, 24, 168 and 192 hours.

In some embodiments, the silk solutions are prepared under various preparation condition parameters such as: 90° C. 30 min, 90° C. 60 min, 100° C. 30 min, and 100° C. 60 min. Briefly, 9.3 M LiBr solution was heated to one of four temperatures: 60° C., 80° C., 100° C. or boiling. 5 mL of hot LiBr solution was added to 1.25 g of silk and placed in the 60° C. oven. Samples from each set were removed at 1, 4 and 6 hours.

In some embodiments, the silk solutions are prepared under various preparation condition parameters such as: Four different silk extraction combinations were used: 90° C. 30 min, 90° C. 60 min, 100° C. 30 min, and 100° C. 60 min. Briefly, 9.3 M LiBr solution was heated to one of four temperatures: 60° C., 80° C., 100° C. or boiling. 5 mL of hot LiBr solution was added to 1.25 g of silk and placed in the oven at the same temperature of the LiBr. Samples from each set were removed at 1, 4 and 6 hours. 1 mL of each sample was added to 7.5 mL of 9.3 M LiBr and refrigerated for viscosity testing.

Molecular weight of the silk protein fragments may be controlled based upon the specific parameters utilized during the extraction step, including extraction time and temperature; specific parameters utilized during the dissolution step, including the LiBr temperature at the time of submersion of the silk in to the lithium bromide and time that the solution is maintained at specific temperatures; and specific parameters utilized during the filtration step. By controlling process parameters using the disclosed methods, it is possible to create silk fibroin protein fragment solutions with polydispersity equal to or lower than 2.5 at a variety of different molecular weight ranging from 5 kDa to 200 kDa, more preferably between 10 kDa and 80 kDa. By altering process parameters to achieve silk solutions with different molecular weights, a range of fragment mixture end products, with desired polydispersity of equal to or less than 2.5 may be targeted based upon the desired performance requirements. For example, a higher molecular weight silk film containing an ophthalmic drug may have a controlled slow release rate compared to a lower molecular weight film making it ideal for a delivery vehicle in eye care products. Additionally, the silk fibroin protein fragment solutions with a polydispersity of greater than 2.5 can be achieved. Further, two solutions with different average molecular weights and polydispersity can be mixed to create combination solutions. Alternatively, a liquid silk gland (100% sericin free silk protein) that has been removed directly from a worm could be used in combination with any of the silk fibroin protein fragment solutions of the present disclosure. Molecular weight of the pure silk fibroin protein fragment composition was determined using High Pressure Liquid Chromatography (HPLC) with a Refractive Index Detector (RID). Polydispersity was calculated using Cirrus GPC Online GPC/SEC Software Version 3.3 (Agilent).

Differences in the processing parameters can result in regenerated silk fibroins that vary in molecular weight, and peptide chain size distribution (polydispersity, PD). This, in turn, influences the regenerated silk fibroin performance, including mechanical strength, water solubility etc.

Parameters were varied during the processing of raw silk cocoons into the silk solution. Varying these parameters affected the MW of the resulting silk solution.

Parameters manipulated included (i) time and temperature of extraction, (ii) temperature of LiBr, (iii) temperature of dissolution oven, and (iv) dissolution time. Experiments were carried out to determine the effect of varying the extraction time. Tables 1-6 summarize the results. Below is a summary:
A sericin extraction time of 30 minutes resulted in larger molecular weight than a sericin extraction time of 60 minutes
Molecular weight decreases with time in the oven
140° C. LiBr and oven resulted in the low end of the confidence interval to be below a molecular weight of 9500 Da
30 min extraction at the 1 hour and 4 hour time points have undigested silk
30 min extraction at the 1 hour time point resulted in a significantly high molecular weight with the low end of the confidence interval being 35,000 Da
The range of molecular weight reached for the high end of the confidence interval was 18000 to 216000 Da (important for offering solutions with specified upper limit).

TABLE 1
The effect of extraction time (30 min vs 60 min) on molecular
weight of silk processed under the conditions of 100° C.
Extraction Temperature, 100° C. Lithium Bromide (LiBr)
and 100° C. Oven Dissolution (Oven/Dissolution Time was varied).

| Boil | Oven | Average | Std | Confidence | | |
|---|---|---|---|---|---|---|
| Time | Time | Mw | dev | Interval | | PD |
| 30 | 1 | 57247 | 12780 | 35093 | 93387 | 1.63 |
| 60 | 1 | 31520 | 1387 | 11633 | 85407 | 2.71 |
| 30 | 4 | 40973 | 2632 | 14268 | 117658 | 2.87 |
| 60 | 4 | 25082 | 1248 | 10520 | 59803 | 2.38 |
| 30 | 6 | 25604 | 1405 | 10252 | 63943 | 2.50 |
| 60 | 6 | 20980 | 1262 | 10073 | 43695 | 2.08 |

TABLE 2
The effect of extraction time (30 min vs 60 min) on molecular
weight of silk processed under the conditions of 100° C.
Extraction Temperature, boiling Lithium Bromide (LiBr)
and 60° C. Oven Dissolution for 4 hr.

| | Boil | Average | Std | Confidence | | |
|---|---|---|---|---|---|---|
| Sample | Time | Mw | dev | Interval | | PD |
| 30 min, 4 hr | 30 | 49656 | 4580 | 17306 | 142478 | 2.87 |
| 60 min, 4 hr | 60 | 30042 | 1536 | 11183 | 80705 | 2.69 |

TABLE 3
The effect of extraction time (30 min vs 60 min) on molecular weight
of silk processed under the conditions of 100° C. Extraction
Temperature, 60° C. Lithium Bromide (LiBr) and 60° C. Oven
Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 1 hr | 30 | 1 | 58436 | | 22201 | 153809 | 2.63 |
| 60 min, 1 hr | 60 | 1 | 31700 | | 11931 | 84224 | 2.66 |
| 30 min, 4 hr | 30 | 4 | 61956.5 | 13337 | 21463 | 178847 | 2.89 |
| 60 min, 4 hr | 60 | 4 | 25578.5 | 2446 | 9979 | 65564 | 2.56 |

TABLE 4
The effect of extraction time (30 min vs 60 min) on molecular weight
of silk processed under the conditions of 100° C. Extraction
Temperature, 80° C. Lithium Bromide (LiBr) and 80° C. Oven
Dissolution for 6 hr.

| | Boil | Average | Std | Confidence | | |
|---|---|---|---|---|---|---|
| Sample | Time | Mw | dev | Interval | | PD |
| 30 min, 6 hr | 30 | 63510 | | 18693 | 215775 | 3.40 |
| 60 min, 6 hr | 60 | 25164 | 238 | 9637 | 65706 | 2.61 |

TABLE 5
The effect of extraction time (30 min vs 60 min) on molecular weight
of silk processed under the conditions of 100° C. Extraction
Temperature, 80° C. Lithium Bromide (LiBr) and 60° C. Oven
Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 4 hr | 30 | 4 | 59202 | 14028 | 19073 | 183760 | 3.10 |
| 60 min, 4 hr | 60 | 4 | 26312.5 | 637 | 10266 | 67442 | 2.56 |
| 30 min, 6 hr | 30 | 6 | 46824 | | 18076 | 121293 | 2.59 |
| 60 min, 6 hr | 60 | 6 | 26353 | | 10168 | 68302 | 2.59 |

TABLE 6
The effect of extraction time (30 min vs 60 min) on molecular weight
of silk processed under the conditions of 100° C. Extraction
Temperature, 140° C. Lithium Bromide (LiBr) and 140° C.
Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 4 hr | 30 | 4 | 9024.5 | 1102 | 4493 | 18127 | 2.00865 |
| 60 min, 4 hr | 60 | 4 | 15548 | | 6954 | 34762 | 2.2358 |
| 30 min, 6 hr | 30 | 6 | 13021 | | 5987 | 28319 | 2.1749 |
| 60 min, 6 hr | 60 | 6 | 10888 | | 5364 | 22100 | 2.0298 |

Experiments were carried out to determine the effect of varying the extraction temperature. Table 7 summarizes the results. Below is a summary:
Sericin extraction at 90° C. resulted in higher MW than sericin extraction at 100° C. extraction
Both 90° C. and 100° C. show decreasing MW over time in the oven

TABLE 7
The effect of extraction temperature (90° C. vs. 100° C.) on molecular
weight of silk processed under the conditions of 60 min. Extraction
Temperature, 100° C. Lithium Bromide (LiBr) and 100° C.
Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 90° C., 4 hr | 60 | 4 | 37308 | 4204 | 13368 | 104119 | 2.79 |
| 100° C., 4 hr | 60 | 4 | 25082 | 1248 | 10520 | 59804 | 2.38 |
| 90° C., 6 hr | 60 | 6 | 34224 | 1135 | 12717 | 92100 | 2.69 |
| 100° C., 6 hr | 60 | 6 | 20980 | 1262 | 10073 | 43694 | 2.08 |

Experiments were carried out to determine the effect of varying the Lithium Bromide (LiBr) temperature when added to silk. Tables 8-9 summarize the results. Below is a summary:
No impact on molecular weight or confidence interval (all CI ˜10500-6500 Da)
Studies illustrated that the temperature of LiBr-silk dissolution, as LiBr is added and begins dissolving, rapidly drops below the original LiBr temperature due to the majority of the mass being silk at room temperature

TABLE 8
The effect of Lithium Bromide (LiBr) temperature on molecular
weight of silk processed under the conditions of 60 min. Extraction
Time., 100° C. Extraction Temperature and 60° C. Oven
Dissolution (Oven/Dissolution Time was varied).

| | LiBr | | | | | | |
|---|---|---|---|---|---|---|---|
| | Temp | Oven | Average | Std | Confidence | | |
| Sample | (° C.) | Time | Mw | dev | Interval | | PD |
| 60° C. LiBr, | 60 | 1 | 31700 | | 11931 | 84223 | 2.66 |
| 1 hr | | | | | | | |
| 100° C. LiBr, | 100 | 1 | 27907 | 200 | 10735 | 72552 | 2.60 |
| 1 hr | | | | | | | |
| RT LiBr, 4hr | RT | 4 | 29217 | 1082 | 10789 | 79119 | 2.71 |
| 60° C. LiBr, | 60 | 4 | 25578 | 2445 | 9978 | 65564 | 2.56 |
| 4 hr | | | | | | | |
| 80° C. LiBr, | 80 | 4 | 26312 | 637 | 10265 | 67441 | 2.56 |
| 4 hr | | | | | | | |
| 100° C. LiBr, | 100 | 4 | 27681 | 1729 | 11279 | 67931 | 2.45 |
| 4 hr | | | | | | | |
| Boil LiBr, 4 hr | Boil | 4 | 30042 | 1535 | 11183 | 80704 | 2.69 |
| RT LiBr, 6 hr | RT | 6 | 26543 | 1893 | 10783 | 65332 | 2.46 |
| 80° C. LiBr, | 80 | 6 | 26353 | | 10167 | 68301 | 2.59 |
| 6 hr | | | | | | | |
| 100° C. LiBr, | 100 | 6 | 27150 | 916 | 11020 | 66889 | 2.46 |
| 6 hr | | | | | | | |

TABLE 9
The effect of Lithium Bromide (LiBr) temperature on molecular
weight of silk processed under the conditions of 30 min. Extraction
Time, 100° C. Extraction Temperature and 60° C. Oven
Dissolution (Oven/Dissolution Time was varied).

| | LiBr | | | | | | |
|---|---|---|---|---|---|---|---|
| | Temp | Oven | Average | Std | Confidence | | |
| Sample | (° C.) | Time | Mw | dev | Interval | | PD |
| 60° C. LiBr, | 60 | 4 | 61956 | 13336 | 21463 | 178847 | 2.89 |
| 4 hr | | | | | | | |
| 80° C. LiBr, | 80 | 4 | 59202 | 14027 | 19073 | 183760 | 3.10 |
| 4 hr | | | | | | | |
| 100° C. LiBr, | 100 | 4 | 47853 | | 19757 | 115899 | 2.42 |
| 4 hr | | | | | | | |
| 80° C. LiBr, | 80 | 6 | 46824 | | 18075 | 121292 | 2.59 |
| 6 hr | | | | | | | |
| 100° C. LiBr, | 100 | 6 | 55421 | 8991 | 19152 | 160366 | 2.89 |
| 6 hr | | | | | | | |

Experiments were carried out to determine the effect of v oven/dissolution temperature. Tables 10-14 summarize the results. Below is a summary:
Oven temperature has less of an effect on 60 min extracted silk than 30 min extracted silk. Without wishing to be bound by theory, it is believed that the 30 min silk is less degraded during extraction and therefore the oven temperature has more of an effect on the larger MW, less degraded portion of the silk.
For 60° C. vs. 140° C. oven the 30 min extracted silk showed a very significant effect of lower MW at higher oven temp, while 60 min extracted silk had an effect but much less
The 140° C. oven resulted in a low end in the confidence interval at 6000 Da.

TABLE 10
The effect of oven/dissolution temperature on molecular weight
of silk processed under the conditions of 100° C. Extraction
Temperature, 30 min. Extraction Time, and 100° C. Lithium
Bromide (LiBr) (Oven/Dissolution Time was varied).

| | Oven | | | | | | |
|---|---|---|---|---|---|---|---|
| Boil | Temp | Oven | Average | Std | Confidence | | |
| Time | (° C.) | Time | Mw | dev | Interval | | PD |
| 30 | 60 | 4 | 47853 | | 19758 | 115900 | 2.42 |
| 30 | 100 | 4 | 40973 | 2632 | 14268 | 117658 | 2.87 |
| 30 | 60 | 6 | 55421 | 8992 | 19153 | 160366 | 2.89 |
| 30 | 100 | 6 | 25604 | 1405 | 10252 | 63943 | 2.50 |

TABLE 11
The effect of oven/dissolution temperature on molecular weight
of silk processed under the conditions of 100° C. Extraction
Temperature, 60 min. Extraction Time, and 100° C. Lithium
Bromide (LiBr) (Oven/Dissolution Time was varied).

| Boil Time | Oven | Oven | Average | Std | Confidence | | |
|---|---|---|---|---|---|---|---|
| (minutes) | Temp | Time | Mw | dev | Interval | | PD |
| 60 | 60 | 1 | 27908 | 200 | 10735 | 72552 | 2.60 |
| 60 | 100 | 1 | 31520 | 1387 | 11633 | 85407 | 2.71 |
| 60 | 60 | 4 | 27681 | 1730 | 11279 | 72552 | 2.62 |
| 60 | 100 | 4 | 25082 | 1248 | 10520 | 59803 | 2.38 |
| 60 | 60 | 6 | 27150 | 916 | 11020 | 66889 | 2.46 |
| 60 | 100 | 6 | 20980 | 1262 | 10073 | 43695 | 2.08 |

TABLE 12
The effect of oven/dissolution temperature on molecular weight
of silk processed under the conditions of 100° C. Extraction
Temperature, 60 min. Extraction Time, and 140° C. Lithium
Bromide (LiBr) (Oven/Dissolution Time was varied).

| Boil | Oven | | | | | | |
|---|---|---|---|---|---|---|---|
| Time | Temp | Oven | | Std | Confidence | | |
| (minutes) | (° C.) | Time | Average | dev | Interval | | PD |
| 60 | 60 | 4 | 30042 | 1536 | 11183 | 80705 | 2.69 |
| 60 | 140 | 4 | 15548 | | 7255 | 33322 | 2.14 |

TABLE 13
The effect of oven/dissolution temperature on molecular weight
of silk processed under the conditions of 100° C. Extraction
Temperature, 30 min. Extraction Time, and 140° C. Lithium
Bromide (LiBr) (Oven/Dissolution Time was varied).

| Boil | Oven | | | | | | |
|---|---|---|---|---|---|---|---|
| Time | Temp | Oven | Average | Std | Confidence | | |
| (minutes) | (° C.) | Time | Mw | dev | Interval | | PD |
| 30 | 60 | 4 | 49656 | 4580 | 17306 | 142478 | 2.87 |
| 30 | 140 | 4 | 9025 | 1102 | 4493 | 18127 | 2.01 |
| 30 | 60 | 6 | 59383 | 11640 | 17641 | 199889 | 3.37 |
| 30 | 140 | 6 | 13021 | | 5987 | 28319 | 2.17 |

TABLE 14
The effect of oven/dissolution temperature on molecular weight
of silk processed under the conditions of 100° C. Extraction
Temperature, 60 min. Extraction Time, and 80° C. Lithium
Bromide (LiBr) (Oven/Dissolution Time was varied).

| Boil | Oven | | | | | | |
|---|---|---|---|---|---|---|---|
| Time | Temp | Oven | Average | Std | Confidence | | |
| (minutes) | (° C.) | Time | Mw | dev | Interval | | PD |
| 60 | 60 | 4 | 26313 | 637 | 10266 | 67442 | 2.56 |
| 60 | 80 | 4 | 30308 | 4293 | 12279 | 74806 | 2.47 |
| 60 | 60 | 6 | 26353 | | 10168 | 68302 | 2.59 |
| 60 | 80 | 6 | 25164 | 238 | 9637 | 65706 | 2.61 |

The raw silk cocoons from the silkworm Bombyx mori was cut into pieces. The pieces of raw silk cocoons were boiled in an aqueous solution of Na₂CO₃ (about 100° C.) for a period of time between about 30 minutes to about 60 minutes to remove sericin (degumming). The volume of the water used equals about 0.4×raw silk weight and the amount of Na₂CO₃ is about 0.848×the weight of the raw silk cocoon pieces. The resulting degummed silk cocoon pieces were rinsed with deionized water three times at about 60° C. (20 minutes per rinse). The volume of rinse water for each cycle was 0.2 L×the weight of the raw silk cocoon pieces. The excess water from the degummed silk cocoon pieces was removed. After the DI water washing step, the wet degummed silk cocoon pieces were dried at room temperature. The degummed silk cocoon pieces were mixed with a LiBr solution, and the mixture was heated to about 100° C. The warmed mixture was placed in a dry oven and was heated at a temperature ranging from about 60° C. to about 140° C. for about 60 minutes to achieve complete dissolution of the native silk protein. The resulting solution was allowed to cool to room temperature and then was dialyzed to remove LiBr salts using a 3,500 Da MWCO membrane. Multiple exchanges were performed in Di water until Br⁻ ions were less than 1 ppm as determined in the hydrolyzed fibroin solution read on an Oakton Bromide (Br⁻) double-junction ion-selective electrode.

The resulting silk fibroin aqueous solution has a concentration of about 8.0% w/v containing pure silk fibroin protein fragments having an average weight average molecular weight ranging from about 6 kDa to about 16 kDa, about 17 kDa to about 39 kDa, and about 39 kDa to about 80 kDa and a polydispersity of between about 1.5 and about 3.0. The 8.0% w/v was diluted with DI water to provide a 1.0% w/v, 2.0% w/v, 3.0% w/v, 4.0% w/v, 5.0% w/v by the coating solution.

### Example 1b. Tangential Flow Filtration (TFF) to Remove Solvent from Dissolved Silk Solutions

A variety of % silk concentrations have been produced through the use of Tangential Flow Filtration (TFF). In all cases a 1% silk solution was used as the input feed. A range of 750-18,000 mL of 1% silk solution was used as the starting volume. Solution is diafiltered in the TFF to remove lithium bromide. Once below a specified level of residual LiBr, solution undergoes ultrafiltration to increase the concentration through removal of water. See examples below.

Six (6) silk solutions were utilized in standard silk structures with the following results:

Solution #1 is a silk concentration of 5.9 wt. %, average MW of 19.8 kDa and 2.2 PDI (made with a 60 min boil extraction, 100° C. LiBr dissolution for 1 hour).

Solution #2 is a silk concentration of 6.4 wt. % (made with a 30 min boil extraction, 60° C. LiBr dissolution for 4 hrs).

Solution #3 is a silk concentration of 6.17 wt. % (made with a 30 min boil extraction 100° C. LiBr dissolution for 1 hour).

Solution #4 is a silk concentration of 7.30 wt. %: A 7.30% silk solution was produced beginning with 30 minute extraction batches of 100 g silk cocoons per batch. Extracted silk fibers were then dissolved using 100° C. 9.3 M LiBr in a 100° C. oven for 1 hour. 100 g of silk fibers were dissolved per batch to create 20% silk in LiBr. Dissolved silk in LiBr was then diluted to 1% silk and filtered through a 5 μm filter to remove large debris. 15,500 mL of 1%, filtered silk solution was used as the starting volume/diafiltration volume for TFF. Once LiBr was removed, the solution was ultrafiltered to a volume around 1300 mL. 1262 mL of 7.30% silk was then collected. Water was added to the feed to help remove the remaining solution and 547 mL of 3.91% silk was then collected.

Solution #5 is a silk concentration of 6.44 wt. %: A 6.44 wt. % silk solution was produced beginning with 60 minute extraction batches of a mix of 25, 33, 50, 75 and 100 g silk cocoons per batch. Extracted silk fibers were then dissolved using 100° C. 9.3 M LiBr in a 100° C. oven for 1 hour. 35, 42, 50 and 71 g per batch of silk fibers were dissolved to create 20% silk in LiBr and combined. Dissolved silk in LiBr was then diluted to 1% silk and filtered through a 5 μm filter to remove large debris. 17,000 mL of 1%, filtered silk solution was used as the starting volume/diafiltration volume for TFF. Once LiBr was removed, the solution was ultrafiltered to a volume around 3000 mL. 1490 mL of 6.44% silk was then collected. Water was added to the feed to help remove the remaining solution and 1454 mL of 4.88% silk was then collected.

Solution #6 is a silk concentration of 2.70 wt. %: A 2.70% silk solution was produced beginning with 60-minute extraction batches of 25 g silk cocoons per batch. Extracted silk fibers were then dissolved using 100° C. 9.3 M LiBr in a 100° C. oven for 1 hour. 35.48 g of silk fibers were dissolved per batch to create 20% silk in LiBr. Dissolved silk in LiBr was then diluted to 1% silk and filtered through a 5 μm filter to remove large debris. 1000 mL of 1%, filtered silk solution was used as the starting volume/diafiltration volume for TFF. Once LiBr was removed, the solution was ultrafiltered to a volume around 300 mL. 312 mL of 2.7% silk was then collected.

The preparation of silk fibroin solutions with higher molecular weights is given in Table 15.

TABLE 15
Preparation and properties of silk fibroin solutions.

| | | | | | Average | |
|---|---|---|---|---|---|---|
| | | | | | weight | |
| | | | | | average | |
| | Extrac- | Extrac- | | | mole- | |
| | tion | tion | LiBr | Oven/ | cular | Average |
| Sample | Time | Temp | Temp | Sol'n | weight | poly- |
| Name | (mins) | (° C.) | (° C.) | Temp | (kDa) | dispersity |
| Group A | 60 | 100 | 100 | 100° C. | 34.7 | 2.94 |
| TFF | | | | oven | | |
| Group A | 60 | 100 | 100 | 100° C. | 44.7 | 3.17 |
| DIS | | | | oven | | |
| Group B | 60 | 100 | 100 | 100° C. | 41.6 | 3.07 |
| TFF | | | | sol'n | | |
| Group B | 60 | 100 | 100 | 100° C. | 44.0 | 3.12 |
| DIS | | | | sol'n | | |
| Group D | 30 | 90 | 60 | 60° C. | 129.7 | 2.56 |
| DIS | | | | sol'n | | |
| Group D | 30 | 90 | 60 | 60° C. | 144.2 | 2.73 |
| FIL | | | | sol'n | | |
| Group E | 15 | 100 | RT | 60° C. | 108.8 | 2.78 |
| DIS | | | | sol'n | | |
| Group E | 15 | 100 | RT | 60° C. | 94.8 | 2.62 |
| FIL | | | | sol'n | | |

Silk aqueous coating composition for application to fabrics are given in Tables 16 and 17 below.

TABLE 16
Silk Solution Characteristics

| | | | | | |
|---|---|---|---|---|---|
| | Molecular Weight: | 57 kDa | | | |
| | Polydispersity: | 1.6 | | | |
| | % Silk | 5.0% | 3.0% | 1.0% | 0.5% |
| Process | | | | | |
| Parameters | | | | | |
| | Extraction | | | | |
| | Boil Time: | 30 minutes | | | |
| | Boil Temperature: | 100° C. | | | |
| | Rinse Temperature: | 60° C. | | | |
| | Dissolution | | | | |
| | LiBr Temperature: | 100° C. | | | |
| | Oven Temperature: | 100° C. | | | |
| | Oven Time: | 60 minutes | | | |

TABLE 17
Silk Solution Characteristics

| | | | | | |
|---|---|---|---|---|---|
| | Molecular Weight: | 25 kDa | | | |
| | Polydispersity: | 2.4 | | | |
| | % Silk | 5.0% | 3.0% | 1.0% | 0.5% |
| Process | | | | | |
| Parameters | | | | | |
| | Extraction | | | | |
| | Boil Time: | 60 minutes | | | |
| | Boil Temperature: | 100° C. | | | |
| | Rinse Temperature: | 60° C. | | | |
| | Dissolution | | | | |
| | LiBr Temperature: | 100° C. | | | |
| | Oven Temperature: | 100° C. | | | |
| | Oven Time: | 60 minutes | | | |

Three (3) silk solutions were utilized in film making with the following results:

Solution #1 is a silk concentration of 5.9%, average MW of 19.8 kDa and 2.2 PD (made with a 60 min boil extraction, 100° C. LiBr dissolution for 1 hr).

Solution #2 is a silk concentration of 6.4% (made with a 30 min boil extraction, 60° C. LiBr dissolution for 4 hrs).

Solution #3 is a silk concentration of 6.17% (made with a 30 min boil extraction, 100° C. LiBr dissolution for 1 hour).

Films were made in accordance with Rockwood et al. (Nature Protocols; Vol. 6; No. 10; published on-line Sep. 22, 2011; doi:10.1038/nprot.2011.379). 4 mL of 1% or 2% (wt/vol) aqueous silk solution was added into 100 mm Petri dish (Volume of silk can be varied for thicker or thinner films and is not critical) and allowed to dry overnight uncovered. The bottom of a vacuum desiccator was filled with water. Dry films were placed in the desiccator and vacuum applied, allowing the films to water anneal for 4 hours prior to removal from the dish. Films cast from solution #1 did not result in a structurally continuous film; the film was cracked in several pieces. These pieces of film dissolved in water in spite of the water annealing treatment.

Silk solutions of various molecular weights and/or combinations of molecular weights can be optimized for gel applications. The following provides an example of this process but it not intended to be limiting in application or formulation. Three (3) silk solutions were utilized in gel making with the following results:

Solution #1 is a silk concentration of 5.9%, average MW of 19.8 kDa and 2.2 PD (made with a 60 min boil extraction, 100° C. LiBr dissolution for 1 hr).

Solution #2 is a silk concentration of 6.4% (made with a 30 min boil extraction, 60° C. LiBr dissolution for 4 hrs).

Solution #3 is a silk concentration of 6.17% (made with a 30 min boil extraction, 100° C. LiBr dissolution for 1 hour).

“Egel” is an electrogelation process as described in Rockwood of al. Briefly, 10 ml of aqueous silk solution is added to a 50 ml conical tube and a pair of platinum wire electrodes immersed into the silk solution. A 20 volt potential was applied to the platinum electrodes for 5 minutes, the power supply turned off and the gel collected. Solution #1 did not form an EGEL over the 5 minutes of applied electric current.

Solutions #2 and #3 were gelled in accordance with the published horseradish peroxidase (HRP) protocol. Behavior seemed typical of published solutions.

Gels were made following the sonication process in Rockwood et al. Briefly, 5 ml of silk solution was added to a 15 ml conical tube. The sonicating horn was immersed in the solution and the solution sonicated at 50% amplitude (21 W). Silk gels were made with 2%, 4% and 6% silk solutions. As compared to standard literature silk, Solutions #2 and #3 formed gels after a longer time, for example: (i) Standard literature silk: 5-8 min; (2) Solution #2: 20 min; (3) Solution #3: 120 min.

Water based, salt leached scaffolds were made in accordance with the published methods of Rockwood. Salt with particle sizes of interest was prepared by stacking the sieves with the largest mesh on top and the smallest mesh on the bottom. Salt was added and sieves shaken vigorously collecting the salt. With a 5 ml syringe, 6% (wt/vol) fibroin solution was aliquot into plastic containers, 2 ml per mold and 5-600 micron salt particles were slowly added on top of the fibroin solution in the mold while rotating the container so that the salt was uniform. The ratio of salt to silk in solution was maintained at 25:1.

The container was gently tapped on the bench to remove air bubbles, the cap closed and the solution allowed to settle overnight at room temperature. Once gelled, the lids were removed and the molds placed in a 2-liter beaker with ultrapure water (three containers per 2 liters of water). The beakers were transferred to a stir plate and stirred, changing the water 2-3 times per day for 2 d (4-5 washes in total). The scaffolds were removed from the molds and placed them in fresh water for an additional day. Both solution #2 & #3 formed scaffolds. The scaffolds made with Solution #3 appear softer than the ones made with Solution #2, but both scaffolds were homogeneous.

### Example 2: Determination of Silk Molecular Weight (MW) Procedure

Materials and Methods: the following equipment and material are used in determination of Silk Molecular weight: Agilent 1100 with chemstation software ver. 10.01; Refractive Index Detector (RID); analytical balance; volumetric flasks (1000 mL, 10 mL and 5 mL); HPLC grade water; ACS grade sodium chloride; ACS grade sodium phosphate dibasic heptahydrate; phosphoric acid; dextran MW Standards-Nominal Molecular Weights of 5 kDa, 11.6 kDa, 23.8 kDa, 48.6 kDa, and 148 kDa; 50 mL PET or polypropylene disposable centrifuge tubes; graduated pipettes; amber glass HPLC vials with Teflon caps; Phenomenex PolySep GFC P-4000 column (size: 7.8 mm×300 mm).

Procedural Steps:
A) Preparation of 1 L Mobile Phase (0.1 M Sodium Chloride solution in 0.0125 M Sodium phosphate buffer)

Take a 250 mL clean and dry beaker, place it on the balance and tare the weight. Add about 3.3509 g of sodium phosphate dibasic heptahydrate to the beaker. Note down the exact weight of sodium phosphate dibasic weighed. Dissolve the weighed sodium phosphate by adding 100 mL of HPLC water into the beaker. Take care not to spill any of the content of the beaker. Transfer the solution carefully into a clean and dry 1000 mL volumetric flask. Rinse the beaker and transfer the rinse into the volumetric flask. Repeat the rinse 4-5 times. In a separate clean and dry 250 mL beaker weigh exactly about 5.8440 g of sodium chloride. Dissolve the weighed sodium chloride in 50 mL of water and transfer the solution to the sodium phosphate solution in the volumetric flask. Rinse the beaker and transfer the rinse into the volumetric flask. Adjust the pH of the solution to 7.0±0.2 with phosphoric acid. Make up the volume in volumetric flask with HPLC water to 1000 mL and shake it vigorously to homogeneously mix the solution. Filter the solution through 0.45 μm polyamide membrane filter. Transfer the solution to a clean and dry solvent bottle and label the bottle. The volume of the solution can be varied to the requirement by correspondingly varying the amount of sodium phosphate dibasic heptahydrate and sodium chloride.
B) Preparation of Dextran Molecular Weight Standard solutions

At least five different molecular weight standards are used for each batch of samples that are run so that the expected value of the sample to be tested is bracketed by the value of the standard used. Label six 20 mL scintillation glass vials respective to the molecular weight standards. Weigh accurately about 5 mg of each of dextran molecular weight standards and record the weights. Dissolve the dextran molecular weight standards in 5 mL of mobile phase to make a 1 mg/mL standard solution.
C) Preparation of Sample solutions

When preparing sample solutions, if there are limitations on how much sample is available, the preparations may be scaled as long as the ratios are maintained. Depending on sample type and silk protein content in sample weigh enough sample in a 50 mL disposable centrifuge tube on an analytical balance to make a 1 mg/mL sample solution for analysis. Dissolve the sample in equivalent volume of mobile phase make a 1 mg/mL solution. Tightly cap the tubes and mix the samples (in solution). Leave the sample solution for 30 minutes at room temperature. Gently mix the sample solution again for 1 minute and centrifuge at 4000 RPM for 10 minutes.
D) HPLC analysis of the samples
   Transfer 1.0 mL of all the standards and sample solutions into individual HPLC vials. Inject the molecular weight standards (one injection each) and each sample in duplicate. Analyze all the standards and sample solutions using the following HPLC conditions:

| | |
|---|---|
| Column | PolySep GFC P-4000 (7.8 × 300 mm) |
| Column Temperature | 25° C. |
| Detector | Refractive Index Detector |
| | (Temperature @ 35° C.) |
| Injection Volume | 25.0 μL |
| Mobile Phase | 0.1M Sodium Chloride solution in |
| | 0.0125M sodium phosphate buffer |
| Flow Rate | 1.0 mL/min |
| RunTime | 20.0 min |

E) Data analysis and calculations—Calculation of Average Molecular Weight using Cirrus Software

Upload the chromatography data files of the standards and the analytical samples into Cirrus SEC data collection and molecular weight analysis software. Calculate the weight average molecular weight (M_{w}), number average molecular weight (Mₙ), peak average molecular weight (Mₚ), and polydispersity for each injection of the sample.

All patents, patent applications, and published references cited herein are hereby incorporated by reference in their entirety. While the methods of the present disclosure have been described in connection with the specific embodiments thereof, it will be understood that it is capable of further modification. Further, this application is intended to cover any variations, uses, or adaptations of the methods of the present disclosure, including such departures from the present disclosure as come within known or customary practice in the art to which the methods of the present disclosure pertain.

1. An article comprising recombinant silk proteins or fragments thereof having a weight average molecular weight, or average weight average molecular weight range of about 1 kDa to about 145 kDa.

2. The article of claim 1, wherein the recombinant silk is selected from a recombinant silk included in a composition selected from compositions #1001-2450, #3001-4450, and #5001-6595.

3. The article of claim 1, wherein the polydispersity of the recombinant silk-based proteins or fragments thereof is between 1 and about 5.

4. The article of claim 1, wherein the polydispersity of the recombinant silk-based proteins or fragments thereof is between about 1.5 and about 3.0.

5. The article of claim 1, wherein the recombinant silk proteins or fragments thereof comprise recombinant spider silk proteins or fragments thereof.

6. The article of claim 1, wherein the recombinant silk proteins or fragments thereof comprise recombinant silkworm silk proteins or fragments thereof.

7. The article of claim 1, wherein the recombinant silk proteins or fragments thereof further comprise a copolymer.

8. The article of claim 1, wherein the recombinant silk proteins or protein fragments thereof have an average weight average molecular weight range selected from the group consisting of about 5 kDa to about 10 kDa, about 6 kDa to about 16 kDa, about 17 kDa to about 38 kDa, about 39 kDa to about 80 kDa, about 60 to about 100 kDa, and about 80 kDa to about 144 kDa.

9. The article of claim 8, wherein the silk proteins or fragments thereof have a polydispersity of between 1 and about 5.

10. The article of claim 1, wherein the article is a coated fabric.

11. The article of claim 1, wherein the article is a coated leather.

12. The article of claim 1, wherein the article is a dermal filler.

13. The article of claim 1, wherein the article is a tissue filler.

14. The article of claim 1, wherein the article is a personal care article.

15. The article of claim 1, wherein the article is a cosmetic article.

16. The article of claim 1, wherein the article is a moisturizer.

17. (canceled)

18. (canceled)


SEQUENCE LISTING (53 sequences)

SEQ ID NO: 1 - protein, 6 aa, Artificial Sequence
  Synthetic protein-like multiblock polymer hexapeptide
  MISC_FEATURE (6)..(6): X can be is alanine, tyrosine, valine, or serine
GAGAGX

SEQ ID NO: 2 - protein, 6 aa, Artificial Sequence
  Synthetic protein-like multiblock polymer hexapeptide
GAGAGS

SEQ ID NO: 3 - protein, 5 aa, Artificial Sequence
  Synthetic mammalian elastin
VPGVG

SEQ ID NO: 4 - protein, 5 aa, Artificial Sequence
  Synthetic mammalian elastin
GVGVP

SEQ ID NO: 5 - protein, 6 aa, Artificial Sequence
  Synthetic linker repetitve fragment
GAGAGS

SEQ ID NO: 6 - protein, 44 aa, Artificial Sequence
  Synthetic protein
SGFGPVANGGSGEASSESDFGSSGFGPVANASSGEASSESDFAG

SEQ ID NO: 7 - protein, 5 aa, Artificial Sequence
  Synthetic consensus peptide
  misc_feature (4)..(4): Xaa can be any naturally occurring amino acid
  MISC_FEATURE (5)..(6): X can be alanine, serine, glycine, tyrocine, or proline
GPGXX

SEQ ID NO: 8 - protein, 9 aa, Artificial Sequence
  Synthetic pro-resilin elastomeric protein
GGRPSDTYG

SEQ ID NO: 9 - protein, 9 aa, Artificial Sequence
  Synthetic pro-resilin elastomeric protein
GGRPSSSYG

SEQ ID NO: 10 - protein, 35 aa, Araneus diadematus
GSSAAAAAAAASGPGGYGPENQGPSGPGGYGPGGP

SEQ ID NO: 11 - protein, 5 aa, Artificial Sequence
  Synthetic recombinant silk protein repetitve unit
GPGAS

SEQ ID NO: 12 - protein, 5 aa, Artificial Sequence
  Synthetic recombinant silk protein repetitve unit
GPGSG

SEQ ID NO: 13 - protein, 5 aa, Artificial Sequence
  Synthetic recombinant silk protein repetitve unit
GPGGY

SEQ ID NO: 14 - protein, 5 aa, Artificial Sequence
  Synthetic recombinant silk protein repetitve unit
GPGGP

SEQ ID NO: 15 - protein, 5 aa, Artificial Sequence
  Synthetic recombinant silk protein repetitve unit
GPGGA

SEQ ID NO: 16 - protein, 5 aa, Artificial Sequence
  Synthetic recombinant silk protein repetitve unit
GPGQQ

SEQ ID NO: 17 - protein, 5 aa, Artificial Sequence
  Synthetic recombinant silk protein repetitve unit
GPGGG

SEQ ID NO: 18 - protein, 5 aa, Artificial Sequence
  Synthetic recombinant silk protein repetitve unit
GPGQG

SEQ ID NO: 19 - protein, 5 aa, Artificial Sequence
  Synthetic recombinant silk protein repetitve unit
GPGGS

SEQ ID NO: 20 - protein, 5 aa, Artificial Sequence
  Synthetic recombinant silk protein repetitve unit
AAAAA

SEQ ID NO: 21 - protein, 6 aa, Artificial Sequence
  Synthetic recombinant silk protein repetitve unit
AAAAAA

SEQ ID NO: 22 - protein, 7 aa, Artificial Sequence
  Synthetic recombinant silk protein repetitve unit
AAAAAAA

SEQ ID NO: 23 - protein, 8 aa, Artificial Sequence
  Synthetic recombinant silk protein repetitve unit
AAAAAAAA

SEQ ID NO: 24 - protein, 9 aa, Artificial Sequence
  Synthetic recombinant silk protein repetitve unit
AAAAAAAAA

SEQ ID NO: 25 - protein, 10 aa, Artificial Sequence
  Synthetic recombinant silk protein repetitve unit
AAAAAAAAAA

SEQ ID NO: 26 - protein, 9 aa, Artificial Sequence
  Synthetic recombinant silk protein repetitve unit
GGRPSDTYG

SEQ ID NO: 27 - protein, 9 aa, Artificial Sequence
  Synthetic recombinant silk protein repetitve unit
GGRPSSSYG

SEQ ID NO: 28 - protein, 24 aa, Artificial Sequence
  Synthetic recombinant silk protein repetitve unit
GPYGPGASAAAAAAGGYGPGSGQQ

SEQ ID NO: 29 - protein, 35 aa, Artificial Sequence; identical to SEQ ID NO: 10
  Synthetic recombinant silk protein repetitve unit

SEQ ID NO: 30 - protein, 20 aa, Artificial Sequence
  Synthetic recombinant silk protein repetitve unit
GPGQQGPGQQGPGQQGPGQQ

SEQ ID NO: 31 - protein, 27 aa, Artificial Sequence
  Synthetic recombinant silk protein repetitve unit
GPGGAGGPYGPGGAGGPYGPGGAGGPY

SEQ ID NO: 32 - protein, 28 aa, Artificial Sequence
  Synthetic recombinant silk protein repetitve unit
GGTTIIEDLDITIDGADGPITISEELTI

SEQ ID NO: 33 - protein, 34 aa, Artificial Sequence
  Synthetic recombinant silk protein repetitve unit
PGSSAAAAAAAASGPGQGQGQGQGQGGRPSDTYG

SEQ ID NO: 34 - protein, 39 aa, Artificial Sequence
  Synthetic recombinant silk protein repetitve unit
SAAAAAAAAGPGGGNGGRPSDTYGAPGGGNGGRPSSSYG

SEQ ID NO: 35 - protein, 18 aa, Artificial Sequence
  Synthetic recombinant silk protein repetitve unit
GGAGGAGGAGGSGGAGGS

SEQ ID NO: 36 - protein, 30 aa, Artificial Sequence
  Synthetic recombinant silk protein repetitve unit
GPGGAGPGGYGPGGSGPGGYGPGGSGPGGY

SEQ ID NO: 37 - protein, 24 aa, Artificial Sequence
  Synthetic recombinant silk protein repetitve unit
GPYGPGASAAAAAAGGYGPGCGQQ

SEQ ID NO: 38 - protein, 24 aa, Artificial Sequence
  Synthetic recombinant silk protein repetitve unit
GPYGPGASAAAAAAGGYGPGKGQQ

SEQ ID NO: 39 - protein, 35 aa, Artificial Sequence
  Synthetic recombinant silk protein repetitve unit
GSSAAAAAAAASGPGGYGPENQGPCGPGGYGPGGP

SEQ ID NO: 40 - protein, 35 aa, Artificial Sequence
  Synthetic recombinant silk protein repetitve unit
GSSAAAAAAAASGPGGYGPKNQGPSGPGGYGPGGP

SEQ ID NO: 41 - protein, 35 aa, Artificial Sequence; identical to SEQ ID NO: 40
  Synthetic recombinant silk protein repetitve unit

SEQ ID NO: 42 - protein, 15 aa, Artificial Sequence
  Synthetic silk protein-like multiblock peptide
  MISC_FEATURE (1)..(1): X can be tyrosine or glutamine
  MISC_FEATURE (4)..(4): X can be tyrosine or glutamine
  MISC_FEATURE (8)..(8): X can be tyrosine or glutamine
XGGXGAGXGAGAGAG

SEQ ID NO: 43 - protein, 14 aa, Artificial Sequence
  Synthetic silk protein-like multiblock peptide
  MISC_FEATURE (11)..(12): X can be amino acid sequence GPS or GPG
GPGGYGPGQQXXSA

SEQ ID NO: 44 - protein, 11 aa, Artificial Sequence
  Synthetic silk protein-like multiblock peptide
  MISC_FEATURE (8)..(8): X can be tyrosine, glutamine, or alanine
GRGAAGGXGAA

SEQ ID NO: 45 - protein, 15 aa, Artificial Sequence
  Synthetic silk protein-like multiblock peptide
  MISC_FEATURE (1)..(1): X can be tyrosine or glutamine
  MISC_FEATURE (4)..(4): X can be tyrosine or glutamine
  MISC_FEATURE (8)..(8): X can be tyrosine or glutamine
XGGXGAGXGAGAGAG

SEQ ID NO: 46 - protein, 8 aa, Artificial Sequence
  Synthetic modified amino acid sequence
GCGGGGGG

SEQ ID NO: 47 - protein, 8 aa, Artificial Sequence
  Synthetic modified amino acid sequence
GKGGGGGG

SEQ ID NO: 48 - protein, 14 aa, Artificial Sequence
  Synthetic modified amino acid sequence
GCGGSGGGGSGGGG

SEQ ID NO: 49 - protein, 13 aa, Artificial Sequence
  Synthetic modified amino acid sequence
GKGGGGGGSGGGG

SEQ ID NO: 50 - protein, 13 aa, Artificial Sequence
  Synthetic modified amino acid sequence
GCGGGGGGSGGGG

SEQ ID NO: 51 - protein, 33 aa, Araneus diadematus
GSSAAAAAAAASGPGQGQGQGQGQGGRPSDTYG

SEQ ID NO: 52 - protein, 39 aa, Araneus diadematus; identical to SEQ ID NO: 34

SEQ ID NO: 53 - protein, 30 aa, Artificial Sequence; identical to SEQ ID NO: 36
  Synthetic recombinant silk protein repetitve fragment